(19) United States
(12) Patent Application Publication
(10) Pub. No.: US 2003/0073144 A1
(43) Pub. Date: Apr. 17, 2003

(54) Compositions and methods for the therapy and diagnosis of pancreatic cancer

(75) Inventors: Darin R. Benson, Seattle, WA (US); Michael D. Kalos, Seattle, WA (US); Michael J. Lodes, Seattle, WA (US); David H. Persing, Redmond, WA (US); William T. Hepler, Seattle, WA (US); Yuqiu Jiang, Kent, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Corixa Corporation, Seattle, WA (US)

(21) Appl. No.: 10/060,036

(22) Filed: Jan. 30, 2002

Related U.S. Application Data
(60) Provisional application No. 60/333,626, filed on Nov. 27, 2001.
(60) Provisional application No. 60/305,484, filed on Jul. 12, 2001.
(60) Provisional application No. 60/265,305, filed on Jan. 30, 2001.
(60) Provisional application No. 60/267,568, filed on Feb. 9, 2001.
(60) Provisional application No. 60/313,999, filed on Aug. 20, 2001.
(60) Provisional application No. 60/291,631, filed on May 16, 2001.
(60) Provisional application No. 60/287,112, filed on Apr. 28, 2001.
(60) Provisional application No. 60/278,651, filed on Mar. 21, 2001.
(60) Provisional application No. 60/265,682, filed on Jan. 31, 2001.

(51) Int. Cl. G01N 33/574; C07H 21/04; C12N 9/00; C12P 21/02; C12N 5/06

(52) U.S. Cl. 435/7.23; 435/69.1; 435/320.1; 435/325; 435/183; 536/23.2

(57) ABSTRACT

Compositions and methods for the therapy and diagnosis of cancer, particularly pancreatic cancer, are disclosed. Illustrative compositions comprise one or more pancreatic tumor polypeptides, immunogenic portions thereof, polynucleotides that encode such polypeptides, antigen presenting cell that expresses such polypeptides, and T cells that are specific for cells expressing such polypeptides. The disclosed compositions are useful, for example, in the diagnosis, prevention and/or treatment of diseases, particularly pancreatic cancer.

### STATEMENT REGARDING SEQUENCE LISTING

The Sequence Listing associated with this application is provided on CD-ROM in lieu of a paper copy, and is hereby incorporated by reference into the specification. Three CD-ROMs are provided, containing identical copies of the sequence listing: CD-ROM No. I is labeled COPY 1, contains the file 566.app which is 2.9 MB and created on Jan. 30, 2002; CD-ROM No.2 is labeled COPY 2, contains the file 566.app which is 2.9 MB and created on Jan. 30, 2002; CD-ROM No. 3 is labeled CRF, contains the file 566.app which is 2.9 MB and created on Jan. 30, 2002.

### BACKGROUND OF THE INVENTION

1. Field of the Invention

The present invention relates generally to therapy and diagnosis of cancer, such as pancreatic cancer. The invention is more specifically related to polypeptides, comprising at least a portion of a pancreatic tumor protein, and to polynucleotides encoding such polypeptides. Such polypeptides and polynucleotides are useful in pharmaceutical compositions, e.g., vaccines, and other compositions for the diagnosis and treatment of pancreatic cancer.

2. Description of the Related Art.

Cancer is a significant health problem throughout the world. Although advances have been made in detection and therapy of cancer, no vaccine or other universally successful method for prevention and/or treatment is currently available. Current therapies, which are generally based on a combination of chemotherapy or surgery and radiation, continue to prove inadequate in many patients.

Pancreatic cancer is the fifth leading cause of cancer death in the United States. Current therapies for this common and difficult-to-treat disease include surgery and/or chemotherapy. Although 5-year survival rates after surgical removal of the pancreas and a large portion of the duodenum have improved, the procedure is only used on 9% of patients. Of these, the highest reported 5-year survival rate is in the range of 20%. Patients with advanced pancreatic cancer are treated primarily by chemotherapy. The objective of such therapy is to prolong patient survival. Surgery and irradiation are used as well to relieve pain and reduce organ blockage.

In spite of considerable research, pancreatic cancer remains difficult to diagnose and treat effectively. Accordingly, there is a need in the art for improved methods for detecting and treating such cancers. The present invention fulfills these needs and further provides other related advantages.

### BRIEF SUMMARY OF THE INVENTION

In one aspect, the present invention provides polynucleotide compositions comprising a sequence selected from the group consisting of:

(a) sequences provided in SEQ ID NOs:1-66, 75-152, 174-177, 182, 184-452, and 454-4550;

(b) complements of the sequences provided in SEQ ID NOs:1-66, 75-152, 174-177, 182, 184-452, and 454-4550;

(c) sequences consisting of at least 20 contiguous residues of a sequence provided in SEQ ID NOs:1-66, 75-152, 174-177, 182, 184-452, and 454-4550;

(d) sequences that hybridize to a sequence provided in SEQ ID NOs:1-66, 75-152, 174-177, 182, 184-452, and 454-4550, under moderately stringent conditions;

(e) sequences having at least 75%, 80%, 85%, 90%, 95%, 96%, 97%, 98% or 99% identity to a sequence of SEQ ID NOs:1-66, 75-152, 174-177, 182, 184-452, and 454-4550;

(f) sequences having at least 90% identity to a sequence of SEQ ID NOs:1-66, 75-152, 174-177, 182, 184-452, and 454-4550; and

(g) degenerate variants of a sequence provided in SEQ ID NOs:1-66, 75-152, 174-177, 182, 184-452, and 454-4550.

In one preferred embodiment, the polynucleotide compositions of the invention are expressed in at least about 20%, more preferably in at least about 30%, and most preferably in at least about 50% of pancreatic tumor samples tested, at a level that is at least about 2-fold, preferably at least about 5-fold, and most preferably at least about 10-fold higher than that for normal tissues.

The present invention, in another aspect, provides polypeptide compositions comprising an amino acid sequence that is encoded by a polynucleotide sequence described above.

The present invention further provides polypeptide compositions comprising an amino acid sequence selected from the group consisting of sequences recited in SEQ ID NOs:67-74, 153-173, 178-181, 183,453,4551-4554, 4558-4560.

In certain preferred embodiments, the polypeptides and/or polynucleotides of the present invention are immunogenic, i.e., they are capable of eliciting an immune response, particularly a humoral and/or cellular immune response, as further described herein.

The present invention further provides fragments, variants and/or derivatives of the disclosed polypeptide and/or polynucleotide sequences, wherein the fragments, variants and/or derivatives preferably have a level of immunogenic activity of at least about 50%, preferably at least about 70% and more preferably at least about 90% of the level of immunogenic activity of a polypeptide sequence set forth in SEQ ID NOs:67-74, 153-173, 178-181, 183, 453, 4551-4554, 4558-4560 or a polypeptide sequence encoded by a polynucleotide sequence set forth in SEQ ID NOs:1-66, 75-152, 174-177, 182, 184-452, and 454-4550.

The present invention further provides polynucleotides that encode a polypeptide described above, expression vectors comprising such polynucleotides and host cells transformed or transfected with such expression vectors.

Within other aspects, the present invention provides pharmaceutical compositions comprising a polypeptide or polynucleotide as described above and a physiologically acceptable carrier.

Within a related aspect of the present invention, the pharmaceutical compositions, e.g., vaccine compositions, are provided for prophylactic or therapeutic applications. Such compositions generally comprise an immunogenic polypeptide or polynucleotide of the invention and an immunostimulant, such as an adjuvant.

The present invention further provides pharmaceutical compositions that comprise: (a) an antibody or antigen-binding fragment thereof that specifically binds to a polypeptide of the present invention, or a fragment thereof; and (b) a physiologically acceptable carrier.

Within further aspects, the present invention provides pharmaceutical compositions comprising: (a) an antigen presenting cell that expresses a polypeptide as described above and (b) a pharmaceutically acceptable carrier or excipient. Illustrative antigen presenting cells include dendritic cells, macrophages, monocytes, fibroblasts and B cells.

Within related aspects, pharmaceutical compositions are provided that comprise: (a) an antigen presenting cell that expresses a polypeptide as described above and (b) an immunostimulant.

The present invention further provides, in other aspects, fusion proteins that comprise at least one polypeptide as described above, as well as polynucleotides encoding such fusion proteins, typically in the form of pharmaceutical compositions, e.g., vaccine compositions, comprising a physiologically acceptable carrier and/or an immunostimulant. The fusions proteins may comprise multiple immunogenic polypeptides or portions/variants thereof, as described herein, and may further comprise one or more polypeptide segments for facilitating the expression, purification and/or immunogenicity of the polypeptide(s).

Within further aspects, the present invention provides methods for stimulating an immune response in a patient, preferably a T cell response in a human patient, comprising administering a pharmaceutical composition described herein. The patient may be afflicted with pancreatic cancer, in which case the methods provide treatment for the disease, or patient considered at risk for such a disease may be treated prophylactically.

Within further aspects, the present invention provides methods for inhibiting the development of a cancer in a patient, comprising administering to a patient a pharmaceutical composition as recited above. The patient may be afflicted with pancreatic cancer, in which case the methods provide treatment for the disease, or patient considered at risk for such a disease may be treated prophylactically.

The present invention further provides, within other aspects, methods for removing tumor cells from a biological sample, comprising contacting a biological sample with T cells that specifically react with a polypeptide of the present invention, wherein the step of contacting is performed under conditions and for a time sufficient to permit the removal of cells expressing the protein from the sample.

Within related aspects, methods are provided for inhibiting the development of a cancer in a patient, comprising administering to a patient a biological sample treated as described above.

Methods are further provided, within other aspects, for stimulating and/or expanding T cells specific for a polypeptide of the present invention, comprising contacting T cells with one or more of: (i) a polypeptide as described above; (ii) a polynucleotide encoding such a polypeptide; and/or (iii) an antigen presenting cell that expresses such a polypeptide; under conditions and for a time sufficient to permit the stimulation and/or expansion of T cells. Isolated T cell populations comprising T cells prepared as described above are also provided.

Within further aspects, the present invention provides methods for inhibiting the development of a cancer in a patient, comprising administering to a patient an effective amount of a T cell population as described above.

The present invention further provides methods for inhibiting the development of a cancer in a patient, comprising the steps of: (a) incubating CD4+ and/or CD8+ T cells isolated from a patient with one or more of: (i) a polypeptide comprising at least an immunogenic portion of polypeptide disclosed herein; (ii) a polynucleotide encoding such a polypeptide; and (iii) an antigen-presenting cell that expressed such a polypeptide; and (b) administering to the patient an effective amount of the proliferated T cells, and thereby inhibiting the development of a cancer in the patient. Proliferated cells may, but need not, be cloned prior to administration to the patient.

Within further aspects, the present invention provides methods for determining the presence or absence of a cancer, preferably a pancreatic cancer, in a patient comprising: (a) contacting a biological sample obtained from a patient with a binding agent that binds to a polypeptide as recited above; (b) detecting in the sample an amount of polypeptide that binds to the binding agent; and (c) comparing the amount of polypeptide with a predetermined cut-off value, and therefrom determining the presence or absence of a cancer in the patient. Within preferred embodiments, the binding agent is an antibody, more preferably a monoclonal antibody.

The present invention also provides, within other aspects, methods for monitoring the progression of a cancer in a patient. Such methods comprise the steps of: (a) contacting a biological sample obtained from a patient at a first point in time with a binding agent that binds to a polypeptide as recited above; (b) detecting in the sample an amount of polypeptide that binds to the binding agent; (c) repeating steps (a) and (b) using a biological sample obtained from the patient at a subsequent point in time; and (d) comparing the amount of polypeptide detected in step (c) with the amount detected in step (b) and therefrom monitoring the progression of the cancer in the patient.

The present invention further provides, within other aspects, methods for determining the presence or absence of a cancer in a patient, comprising the steps of: (a) contacting a biological sample, e.g., tumor sample, serum sample, etc., obtained from a patient with an oligonucleotide that hybridizes to a polynucleotide that encodes a polypeptide of the present invention; (b) detecting in the sample a level of a polynucleotide, preferably mRNA, that hybridizes to the oligonucleotide; and (c) comparing the level of polynucleotide that hybridizes to the oligonucleotide with a predetermined cut-off value, and therefrom determining the presence or absence of a cancer in the patient. Within certain embodiments, the amount of mRNA is detected via polymerase chain reaction using, for example, at least one oligonucleotide primer that hybridizes to a polynucleotide encoding a polypeptide as recited above, or a complement of such a polynucleotide. Within other embodiments, the amount of mRNA is detected using a hybridization technique, employing an oligonucleotide probe that hybridizes to a polynucleotide that encodes a polypeptide as recited above, or a complement of such a polynucleotide.

In related aspects, methods are provided for monitoring the progression of a cancer in a patient, comprising the steps of: (a) contacting a biological sample obtained from a patient with an oligonucleotide that hybridizes to a polynucleotide that encodes a polypeptide of the present invention; (b) detecting in the sample an amount of a polynucleotide that hybridizes to the oligonucleotide; (c) repeating steps (a) and (b) using a biological sample obtained from the patient at a subsequent point in time; and (d) comparing the amount of polynucleotide detected in step (c) with the amount detected in step (b) and therefrom monitoring the progression of the cancer in the patient.

Within further aspects, the present invention provides antibodies, such as monoclonal antibodies, that bind to a polypeptide as described above, as well as diagnostic kits comprising such antibodies. Diagnostic kits comprising one or more oligonucleotide probes or primers as described above are also provided.

These and other aspects of the present invention will become apparent upon reference to the following detailed description. All references disclosed herein are hereby incorporated by reference in their entirety as if each was incorporated individually.

### BRIEF DESCRIPTION OF THE SEQUENCE IDENTIFIERS

SEQ ID NO:1 is the determined cDNA sequence of clone PANC1-R1, ID NO. 68811.

SEQ ID NO:2 is the determined cDNA sequence of clone PANC1-R2, ID NO. 68812.

SEQ ID NO:3 is the determined cDNA sequence of clone PANC1-R3, ID NO. 68813.

SEQ ID NO:4 is the determined cDNA sequence of clone PANC1-R4, ID NO. 68814.

SEQ ID NO:5 is the determined cDNA sequence of clone PANC1-R5, ID NO. 68815.

SEQ ID NO:6 is the determined cDNA sequence of clone PANC1-R6, ID NO. 68816.

SEQ ID NO:7 is the determined cDNA sequence of clone PANC1-R8, ID NO. 68818.

SEQ ID NO:8 is the determined cDNA sequence of clone PANC1-R10, ID NO. 68820.

SEQ ID NO:9 is the determined cDNA sequence of clone PANC1-R11, ID NO. 68821.

SEQ ID NO:10 is the determined cDNA sequence of clone PANC1-R12, ID NO. 68822.

SEQ ID NO:11 is the determined cDNA sequence of clone PANC1-R13, ID NO. 68823.

SEQ ID NO:12 is the determined cDNA sequence of clone PANC1-R14, ID NO. 68824.

SEQ ID NO:13 is the determined cDNA sequence of clone PANC1-R15, ID NO. 68825.

SEQ ID NO:14 is the determined cDNA sequence of clone PANC1-R16, ID NO. 68826.

SEQ ID NO:15 is the determined cDNA sequence of clone PANC1-R17, ID NO. 68827.

SEQ ID NO:16 is the determined cDNA sequence of clone PANC1-R18, ID NO. 68828.

SEQ ID NO:17 is the determined cDNA sequence of clone PANC1-R19, ID NO. 68829.

SEQ ID NO:18 is the determined cDNA sequence of clone PANC1-R20, ID NO. 68830.

SEQ ID NO:19 is the determined cDNA sequence of clone PANC1-R21, ID NO. 68917.

SEQ ID NO:20 is the determined cDNA sequence of clone PANC1-R22, ID NO. 68918.

SEQ ID NO:21 is the determined cDNA sequence of clone PANC1-R23, ID NO. 68919.

SEQ ID NO:22 is the determined cDNA sequence of clone PANC1-R24, ID NO. 68920.

SEQ ID NO:23 is the determined cDNA sequence of clone PANC1-R25, ID NO. 68921.

SEQ ID NO:24 is the determined cDNA sequence of clone PANC1-R27, ID NO. 68923.

SEQ ID NO:25 is the determined cDNA sequence of clone PANC1-R28, ID NO. 68924.

SEQ ID NO:26 is the determined cDNA sequence of clone PANC1-R29, ID NO. 68925.

SEQ ID NO:27 is the determined cDNA sequence of clone PANC1-R30, ID NO. 68926.

SEQ ID NO:28 is the determined cDNA sequence of clone PANC1-R32, ID NO. 68928.

SEQ ID NO:29 is the determined cDNA sequence of clone PANC1-R33, ID NO. 68929.

SEQ ID NO:30 is the determined cDNA sequence of clone PANC1-R34, ID NO. 68930.

SEQ ID NO:31 is the determined cDNA sequence of clone PANC1-R36, ID NO. 68932.

SEQ ID NO:32 is the determined cDNA sequence of clone PANC1-R37, ID NO. 68933.

SEQ ID NO:33 is the determined cDNA sequence of clone PANC1-R39, ID NO. 68935.

SEQ ID NO:34 is the determined cDNA sequence of clone PANC1-R40, ID NO. 68936.

SEQ ID NO:35 is the determined cDNA sequence of clone PANC1-R43, ID NO. 69117.

SEQ ID NO:36 is the determined cDNA sequence of clone PANC1-R44, ID NO. 69118.

SEQ ID NO:37 is the determined cDNA sequence of clone PANC1-R45, ID NO. 69119.

SEQ ID NO:38 is the determined cDNA sequence of clone PANC1-R46, ID NO. 69120.

SEQ ID NO:39 is the determined cDNA sequence of clone PANC1-R47, ID NO. 69126.

SEQ ID NO:40 is the determined cDNA sequence of clone PANC1-R50, ID NO. 69133.

SEQ ID NO:41 is the determined cDNA sequence of clone PANC1-R51, ID NO. 69134.

SEQ ID NO:42 is the determined cDNA sequence of clone PANC1-R52, ID NO. 69135.

SEQ ID NO:43 is the determined cDNA sequence of clone PANC1-R53, ID NO. 69136.

SEQ ID NO:44 is the determined cDNA sequence of clone PANC1-R56, ID NO. 69139.

SEQ ID NO:45 is the determined cDNA sequence of clone PANC1-R59, ID NO. 69142.

SEQ ID NO:46 is the determined cDNA sequence of clone PANC1-R64, ID NO. 69292.

SEQ ID NO:47 is the determined cDNA sequence of clone PANC1-R66, ID NO. 69294.

SEQ ID NO:48 is the determined cDNA sequence of clone PANC1-R67, ID NO. 69295.

SEQ ID NO:49 is the determined cDNA sequence of clone PANC1-R69, ID NO. 69297.

SEQ ID NO:50 is the determined cDNA sequence of clone PANC1-R70, ID NO. 69298.

SEQ ID NO:51 is the determined cDNA sequence of clone PANC1-R71, ID NO. 69299.

SEQ ID NO:52 is the determined cDNA sequence of clone PANC1-R73, ID NO. 69301.

SEQ ID NO:53 is the determined cDNA sequence of clone PANC1-R76 A, ID NO. 69304.

SEQ ID NO:54 is the determined cDNA sequence of clone PANC1-R76 B, ID NO. 69304.

SEQ ID NO:55 is the determined cDNA sequence of clone PANC1-R78, ID NO. 69306.

SEQ ID NO:56 is the determined cDNA sequence of clone PANC1-R80, ID NO. 69308.

SEQ ID NO:57 is the determined cDNA sequence of clone PANC1-R82, ID NO. 69310.

SEQ ID NO:58 is the determined cDNA sequence of clone PANC1-R83, ID NO. 69311.

SEQ ID NO:59 is the determined cDNA sequence of clone PANC1-R84, ID NO. 69312.

SEQ ID NO:60 is the determined cDNA sequence of clone PANC1-R85, ID NO. 69313.

SEQ ID NO:61 is the determined cDNA sequence of clone PANC1-R86, ID NO. 69314.

SEQ ID NO:62 is the determined cDNA sequence of clone PANC1-R88, ID NO. 69316.

SEQ ID NO:63 is the determined cDNA sequence of clone PANC1-R89, ID NO. 69317.

SEQ ID NO:64 is the determined cDNA sequence of clone PANC1-R90, ID NO. 69318.

SEQ ID NO:65 is the determined cDNA sequence of clone PANC1-R91, ID NO. 69319.

SEQ ID NO:66 is the determined cDNA sequence of clone PANC1-R94, ID NO. 69322.

SEQ ID NO:67 is the predicted polypeptide sequence of clone PANC1-R44, ID NO. 69118.

SEQ ID NO:68 is the predicted polypeptide sequence of clone PANC1-R47, ID NO. 69126.

SEQ ID NO:69 is the predicted polypeptide sequence of clone PANC1-R64, ID NO. 69292.

SEQ ID NO:70 is the predicted polypeptide sequence of clone PANC1-R66, ID NO. 69294.

SEQ ID NO:71 is the predicted polypeptide sequence of clone PANC1-R76 A, ID NO. 69304.

SEQ ID NO:72 is the predicted polypeptide sequence of clone PANC1-R76 B, ID NO. 69304.

SEQ ID NO:73 is the predicted polypeptide sequence of clone PANC1-R85, ID NO. 69313.

SEQ ID NO:74 is the predicted polypeptide sequence of clone PANC1-R94, ID NO. 69322.

SEQ ID NO:75 is the determined cDNA sequence of clone 80150.1

SEQ ID NO:76 is the determined cDNA sequence of clone 80151.1

SEQ ID NO:77 is the determined cDNA sequence of clone 80152.1

SEQ ID NO:78 is the determined cDNA sequence of clone 80153.1

SEQ ID NO:79 is the determined cDNA sequence of clone 80154.1

SEQ ID NO:80 is the determined cDNA sequence of clone 80155.1

SEQ ID NO:81 is the determined cDNA sequence of clone 80156.1

SEQ ID NO:82 is the determined cDNA sequence of clone 80157.1

SEQ ID NO:83 is the determined cDNA sequence of clone 80158.1

SEQ ID NO:84 is the determined cDNA sequence of clone 80159.1

SEQ ID NO:85 is the determined cDNA sequence of clone 80161.1

SEQ ID NO:86 is the determined cDNA sequence of clone 80162.1

SEQ ID NO:87 is the determined cDNA sequence of clone 80163.1

SEQ ID NO:88 is the determined cDNA sequence of clone 80164.1

SEQ ID NO:89 is the determined cDNA sequence of clone 80165.1

SEQ ID NO:90 is the determined cDNA sequence of clone 80166.1

SEQ ID NO:91 is the determined cDNA sequence of clone 80167.1

SEQ ID NO:92 is the determined cDNA sequence of clone 80168.1

SEQ ID NO:93 is the determined cDNA sequence of clone 80169.1

SEQ ID NO:94 is the determined cDNA sequence of clone 80170.1

SEQ ID NO:95 is the determined cDNA sequence of clone 80171.1

SEQ ID NO:96 is the determined cDNA sequence of clone 80173.1

SEQ ID NO:97 is the determined cDNA sequence of clone 80175.1

SEQ ID NO:98 is the determined cDNA sequence of clone 80176.1

SEQ ID NO:99 is the determined cDNA sequence of clone 80178.1

SEQ ID NO:100 is the determined cDNA sequence of clone 80180.1

SEQ ID NO:101 is the determined cDNA sequence of clone 80181.1

SEQ ID NO:102 is the determined cDNA sequence of clone 80183.1

SEQ ID NO:103 is the determined cDNA sequence of clone 80184.1

SEQ ID NO:104 is the determined cDNA sequence of clone 80185.1

SEQ ID NO:105 is the determined cDNA sequence of clone 80186.1

SEQ ID NO:106 is the determined cDNA sequence of clone 80187.1

SEQ ID NO:107 is the determined cDNA sequence of clone 80188.1

SEQ ID NO:108 is the determined cDNA sequence of clone 80189.1

SEQ ID NO:109 is the determined cDNA sequence of clone 80191.1

SEQ ID NO:110 is the determined cDNA sequence of clone 80192.1

SEQ ID NO:111 is the determined cDNA sequence of clone 80193.1

SEQ ID NO:112 is the determined cDNA sequence of clone 80194.1

SEQ ID NO:113 is the determined cDNA sequence of clone 80195.1

SEQ ID NO:114 is the determined cDNA sequence of clone 80196.1

SEQ ID NO:115 is the determined cDNA sequence of clone 80197.1

SEQ ID NO:116 is the determined cDNA sequence of clone 80198.1

SEQ ID NO:117 is the determined cDNA sequence of clone 80199.1

SEQ ID NO:118 is the determined cDNA sequence of clone 80172.2

SEQ ID NO:119 is the determined cDNA sequence of clone 80174.2

SEQ ID NO:120 is the determined cDNA sequence of clone 80177.2

SEQ ID NO:121 is the determined cDNA sequence of clone 80179.2

SEQ ID NO:122 is the determined cDNA sequence of clone 80190.2

SEQ ID NO:123 is the determined cDNA sequence of clone 80200.2

SEQ ID NO:124 is the determined cDNA sequence of clone 80201.2

SEQ ID NO:125 is the determined cDNA sequence of clone 80203.2

SEQ ID NO:126 is the determined cDNA sequence of clone 80204.2

SEQ ID NO:127 is the determined cDNA sequence of clone 80205.2

SEQ ID NO:128 is the determined cDNA sequence of clone 80207.2

SEQ ID NO:129 is the determined cDNA sequence of clone 80208.2

SEQ ID NO:130 is the full-length determined cDNA sequence of clone PIGPC1

SEQ ID NO:131 is the full-length determined cDNA sequence of clone SERPINE1

SEQ ID NO:132 is the full-length determined cDNA sequence of clone KRT18

SEQ ID NO:133 is the full-length determined cDNA sequence of clone RABGGTB

SEQ ID NO:134 is the full-length determined cDNA sequence of clone hFAT

SEQ ID NO:135 is the full-length determined cDNA sequence of clone FBL

SEQ ID NO:136 is the full-length determined cDNA sequence of clone COL1a1

SEQ ID NO:137 is the full-length determined cDNA sequence of clone pM5

SEQ ID NO:138 is the full-length determined cDNA sequence of clone PSK-1

SEQ ID NO:139 is the full-length determined cDNA sequence of clone CD24

SEQ ID NO:140 is the determined cDNA sequence of clone sim.toHu.G6PD

SEQ ID NO:141 is the full-length determined cDNA sequence of clone GdX

SEQ ID NO:142 is the full-length determined cDNA sequence of clone PLS3

SEQ ID NO:143 is the full-length determined cDNA sequence of clone LISCH7

SEQ ID NO:144 is the full-length determined cDNA sequence of clone COL18A1

SEQ ID NO:145 is the full-length determined cDNA sequence of clone TFPI2

SEQ ID NO:146 is the full-length determined cDNA sequence of clone L6

SEQ ID NO:147 is the full-length determined cDNA sequence of clone SERF1A

SEQ ID NO:148 is the full-length determined cDNA sequence of clone SERF1B

SEQ ID NO:149 is the full-length determined cDNA sequence of clone THBS2

SEQ ID NO:150 is the full-length determined cDNA sequence of clone SEMA3C

SEQ ID NO:151 is the full-length determined cDNA sequence of clone CTGF

SEQ ID NO:152 is the full-length determined cDNA sequence of clone pHL-1

SEQ ID NO:153 is the full-length predicted amino acid sequence of clone PICPC1

SEQ ID NO:154 is the full-length predicted amino acid sequence of clone SERPINE1

SEQ ID NO:155 is the full-length predicted amino acid sequence of clone KRT18

SEQ ID NO:156 is the full-length predicted amino acid sequence of clone RABGGTB

SEQ ID NO:157 is the full-length predicted amino acid sequence of clone hFAT

SEQ ID NO:158 is the full-length predicted amino acid sequence of clone FBL

SEQ ID NO:159 is the full-length predicted amino acid sequence of clone COL1a1

SEQ ID NO:160 is the full-length predicted amino acid sequence of clone pM5

SEQ ID NO:161 is the full-length predicted amino acid sequence of clone PSK-1

SEQ ID NO:162 is the full-length predicted amino acid sequence of clone CD24

SEQ ID NO:163 is the full-length predicted amino acid sequence of clone GdX

SEQ ID NO:164 is the full-length predicted amino acid sequence of clone PLS3

SEQ ID NO:165 is the full-length predicted amino acid sequence of clone LISCH7

SEQ ID NO:166 is the full-length predicted amino acid sequence of clone COL18A1

SEQ ID NO:167 is the full-length predicted amino acid sequence of clone TFPI2

SEQ ID NO:168 is the full-length predicted amino acid sequence of clone L6

SEQ ID NO:169 is the full-length predicted amino acid sequence of clone SERF1A

SEQ ID NO:170 is the full-length predicted amino acid sequence of clone SERF1B

SEQ ID NO:171 is the full-length predicted amino acid sequence of clone THBS2

SEQ ID NO:172 is the full-length predicted amino acid sequence of clone SEMA3C

SEQ ID NO:173 is the full-length predicted amino acid sequence of clone CTGF

SEQ ID NO:174 is the full-length determined cDNA sequence of clone HECH

SEQ ID NO:175 is the full-length determined cDNA sequence of clone SCD

SEQ ID NO:176 is the full-length determined cDNA sequence of clone CHGB

SEQ ID NO:177 is the full-length determined cDNA sequence of clone FER1L3

SEQ ID NO:178 is the full-length predicted amino acid sequence of clone HECH

SEQ ID NO:179 is the full-length predicted amino acid sequence of clone SCD

SEQ ID NO:180 is the full-length predicted amino acid sequence of clone CHGB

SEQ ID NO:181 is the full-length predicted amino acid sequence of clone FER1L3

SEQ ID NO:182 is the full-length determined cDNA sequence of clone MGC15409

SEQ ID NO:183 is the full-length predicted amino acid sequence of clone MGC15409

SEQ ID NO:184 is the determined cDNA sequence of clone 71231.1

SEQ ID NO:185 is the determined cDNA sequence of clone 71232.1

SEQ ID NO:186 is the determined cDNA sequence of clone 71233.1

SEQ ID NO:187 is the determined cDNA sequence of clone 71234.1

SEQ ID NO:188 is the determined cDNA sequence of clone 71235.1

SEQ ID NO:189 is the determined cDNA sequence of clone 71236.1

SEQ ID NO:190 is the determined cDNA sequence of clone 71237.1

SEQ ID NO:191 is the determined cDNA sequence of clone 73408.1

SEQ ID NO:192 is the determined cDNA sequence of clone 73409.1

SEQ ID NO:193 is the determined cDNA sequence of clone 73410.1

SEQ ID NO:194 is the determined cDNA sequence of clone 71238.1

SEQ ID NO:195 is the determined cDNA sequence of clone 73411.1

SEQ ID NO:196 is the determined cDNA sequence of clone 71239.1

SEQ ID NO:197 is the determined cDNA sequence of clone 73412.3

SEQ ID NO:198 is the determined cDNA sequence of clone 73412.2

SEQ ID NO:199 is the determined cDNA sequence of clone 71240.1

SEQ ID NO:200 is the determined cDNA sequence of clone 71241.1

SEQ ID NO:201 is the determined cDNA sequence of clone 71242.1

SEQ ID NO:202 is the determined cDNA sequence of clone 73413.2

SEQ ID NO:203 is the determined cDNA sequence of clone 71243.1

SEQ ID NO:204 is the determined cDNA sequence of clone 71244.1

SEQ ID NO:205 is the determined cDNA sequence of clone 71245.1

SEQ ID NO:206 is the determined cDNA sequence of clone 71246.1

SEQ ID NO:207 is the determined cDNA sequence of clone 71247.1

SEQ ID NO:208 is the determined cDNA sequence of clone 71248.1

SEQ ID NO:209 is the determined cDNA sequence of clone 71249.1

SEQ ID NO:210 is the determined cDNA sequence of clone 73414.2

SEQ ID NO:211 is the determined cDNA sequence of clone 71250.1

SEQ ID NO:212 is the determined cDNA sequence of clone 71251.1

SEQ ID NO:213 is the determined cDNA sequence of clone 71252.1

SEQ ID NO:214 is the determined cDNA sequence of clone 71253.1

SEQ ID NO:215 is the determined cDNA sequence of clone 71254.3

SEQ ID NO:216 is the determined cDNA sequence of clone 71255.2

SEQ ID NO:217 is the determined cDNA sequence of clone 71255.3

SEQ ID NO:218 is the determined cDNA sequence of clone 71256.1

SEQ ID NO:219 is the determined cDNA sequence of clone 73415.1

SEQ ID NO:220 is the determined cDNA sequence of clone 71257.1

SEQ ID NO:221 is the determined cDNA sequence of clone 71258.1

SEQ ID NO:222 is the determined cDNA sequence of clone 71259.1

SEQ ID NO:223 is the determined cDNA sequence of clone 73416.1

SEQ ID NO:224 is the determined cDNA sequence of clone 71260.1

SEQ ID NO:225 is the determined cDNA sequence of clone 73376.1

SEQ ID NO:226 is the determined cDNA sequence of clone 73377.1

SEQ ID NO:227 is the determined cDNA sequence of clone 73378.1

SEQ ID NO:228 is the determined cDNA sequence of clone 73379.2

SEQ ID NO:229 is the determined cDNA sequence of clone 73381.3

SEQ ID NO:230 is the determined cDNA sequence of clone 73381.2

SEQ ID NO:231 is the determined cDNA sequence of clone 73382.1

SEQ ID NO:232 is the determined cDNA sequence of clone 73383.2

SEQ ID NO:233 is the determined cDNA sequence of clone 73383.3

SEQ ID NO:234 is the determined cDNA sequence of clone 73384.2

SEQ ID NO:235 is the determined cDNA sequence of clone 73384.3

SEQ ID NO:236 is the determined cDNA sequence of clone 73385.1

SEQ ID NO:237 is the determined cDNA sequence of clone 73386.1

SEQ ID NO:238 is the determined cDNA sequence of clone 73387.1

SEQ ID NO:239 is the determined cDNA sequence of clone 73388.2

SEQ ID NO:240 is the determined cDNA sequence of clone 73388.3

SEQ ID NO:241 is the determined cDNA sequence of clone 73389.2

SEQ ID NO:242 is the determined cDNA sequence of clone 73389.3

SEQ ID NO:243 is the determined cDNA sequence of clone 73390.1

SEQ ID NO:244 is the determined cDNA sequence of clone 73391.2

SEQ ID NO:245 is the determined cDNA sequence of clone 73417.2

SEQ ID NO:246 is the determined cDNA sequence of clone 73392.1

SEQ ID NO:247 is the determined cDNA sequence of clone 73393.3

SEQ ID NO:248 is the determined cDNA sequence of clone 73418.1

SEQ ID NO:249 is the determined cDNA sequence of clone 73395.1

SEQ ID NO:250 is the determined cDNA sequence of clone 73396.1

SEQ ID NO:251 is the determined cDNA sequence of clone 73397.1

SEQ ID NO:252 is the determined cDNA sequence of clone 73419.3

SEQ ID NO:253 is the determined cDNA sequence of clone 73419.2

SEQ ID NO:254 is the determined cDNA sequence of clone 73398.1

SEQ ID NO:255 is the determined cDNA sequence of clone 73399.3

SEQ ID NO:256 is the determined cDNA sequence of clone 73399.2

SEQ ID NO:257 is the determined cDNA sequence of clone 73400.1

SEQ ID NO:258 is the determined cDNA sequence of clone 73420.3

SEQ ID NO:259 is the determined cDNA sequence of clone 73420.2

SEQ ID NO:260 is the determined cDNA sequence of clone 73401.1

SEQ ID NO:261 is the determined cDNA sequence of clone 73404.3

SEQ ID NO:262 is the determined cDNA sequence of clone 73405.1

SEQ ID NO:263 is the determined cDNA sequence of clone 73407.3

SEQ ID NO:264 is the determined cDNA sequence of clone 73407.2

SEQ ID NO:265 is the determined cDNA sequence of clone 72174.1

SEQ ID NO:266 is the determined cDNA sequence of clone 72175.1

SEQ ID NO:267 is the determined cDNA sequence of clone 72176.1

SEQ ID NO:268 is the determined cDNA sequence of clone 72177.1

SEQ ID NO:269 is the determined cDNA sequence of clone 72178.1

SEQ ID NO:270 is the determined cDNA sequence of clone 72179.1

SEQ ID NO:271 is the determined cDNA sequence of clone 73421.3

SEQ ID NO:272 is the determined cDNA sequence of clone 73421.2

SEQ ID NO:273 is the determined cDNA sequence of clone 72180.1

SEQ ID NO:274 is the determined cDNA sequence of clone 72181.1

SEQ ID NO:275 is the determined cDNA sequence of clone 72182.1

SEQ ID NO:276 is the determined cDNA sequence of clone 72183.1

SEQ ID NO:277 is the determined cDNA sequence of clone 72184.1

SEQ ID NO:278 is the determined cDNA sequence of clone 72185.1

SEQ ID NO:279 is the determined cDNA sequence of clone 72186.1

SEQ ID NO:280 is the determined cDNA sequence of clone 72187.1

SEQ ID NO:281 is the determined cDNA sequence of clone 72188.1

SEQ ID NO:282 is the determined cDNA sequence of clone 72189.1

SEQ ID NO:283 is the determined cDNA sequence of clone 72190.1

SEQ ID NO:284 is the determined cDNA sequence of clone 72191.1

SEQ ID NO:285 is the determined cDNA sequence of clone 72192.1

SEQ ID NO:286 is the determined cDNA sequence of clone 72193.1

SEQ ID NO:287 is the determined cDNA sequence of clone 72194.1

SEQ ID NO:288 is the determined cDNA sequence of clone 72195.1

SEQ ID NO:289 is the determined cDNA sequence of clone 72196.1

SEQ ID NO:290 is the determined cDNA sequence of clone 72197.1

SEQ ID NO:291 is the determined cDNA sequence of clone 72198.1

SEQ ID NO:292 is the determined cDNA sequence of clone 72199.1

SEQ ID NO:293 is the determined cDNA sequence of clone 72200.1

SEQ ID NO:294 is the determined cDNA sequence of clone 72201.1

SEQ ID NO:295 is the determined cDNA sequence of clone 72202.1

SEQ ID NO:296 is the determined cDNA sequence of clone 72203.1

SEQ ID NO:297 is the determined cDNA sequence of clone 72204.1

SEQ ID NO:298 is the determined cDNA sequence of clone 72205.1

SEQ ID NO:299 is the determined cDNA sequence of clone 72206.3

SEQ ID NO:300 is the determined cDNA sequence of clone 72206.2

SEQ ID NO:301 is the determined cDNA sequence of clone 73422.1

SEQ ID NO:302 is the determined cDNA sequence of clone 73423.1

SEQ ID NO:303 is the determined cDNA sequence of clone 73424.1

SEQ ID NO:304 is the determined cDNA sequence of clone 73425.3

SEQ ID NO:305 is the determined cDNA sequence of clone 73425.2

SEQ ID NO:306 is the determined cDNA sequence of clone 74597.2

SEQ ID NO:307 is the determined cDNA sequence of clone 73426.3

SEQ ID NO:308 is the determined cDNA sequence of clone 73426.2

SEQ ID NO:309 is the determined cDNA sequence of clone 73427.3

SEQ ID NO:310 is the determined cDNA sequence of clone 73427.2

SEQ ID NO:311 is the determined cDNA sequence of clone 73428.3

SEQ ID NO:312 is the determined cDNA sequence of clone 73429.3

SEQ ID NO:313 is the determined cDNA sequence of clone 74598.1

SEQ ID NO:314 is the determined cDNA sequence of clone 74598.2

SEQ ID NO:315 is the determined cDNA sequence of clone 74599.2

SEQ ID NO:316 is the determined cDNA sequence of clone 73430.3

SEQ ID NO:317 is the determined cDNA sequence of clone 74600.1

SEQ ID NO:318 is the determined cDNA sequence of clone 74600.2

SEQ ID NO:319 is the determined cDNA sequence of clone 74601.1

SEQ ID NO:320 is the determined cDNA sequence of clone 74602.2

SEQ ID NO:321 is the determined cDNA sequence of clone 73437.2

SEQ ID NO:322 is the determined cDNA sequence of clone 73437.3

SEQ ID NO:323 is the determined cDNA sequence of clone 73438.2

SEQ ID NO:324 is the determined cDNA sequence of clone 73438.3

SEQ ID NO:325 is the determined cDNA sequence of clone 73439.2

SEQ ID NO:326 is the determined cDNA sequence of clone 73439.3

SEQ ID NO:327 is the determined cDNA sequence of clone 73440.2

SEQ ID NO:328 is the determined cDNA sequence of clone 73440.3

SEQ ID NO:329 is the determined cDNA sequence of clone 73441.3

SEQ ID NO:330 is the determined cDNA sequence of clone 73442.2

SEQ ID NO:331 is the determined cDNA sequence of clone 73442.3

SEQ ID NO:332 is the determined cDNA sequence of clone 73443.2

SEQ ID NO:333 is the determined cDNA sequence of clone 73443.3

SEQ ID NO:334 is the determined cDNA sequence of clone 73444.2

SEQ ID NO:335 is the determined cDNA sequence of clone 73444.3

SEQ ID NO:336 is the determined cDNA sequence of clone 74602.1

SEQ ID NO:337 is the determined cDNA sequence of clone 73445.1

SEQ ID NO:338 is the determined cDNA sequence of clone 73456.1

SEQ ID NO:339 is the determined cDNA sequence of clone 73585.3

SEQ ID NO:340 is the determined cDNA sequence of clone 73586.2

SEQ ID NO:341 is the determined cDNA sequence of clone 73586.3

SEQ ID NO:342 is the determined cDNA sequence of clone 73587.2

SEQ ID NO:343 is the determined cDNA sequence of clone 73587.3

SEQ ID NO:344 is the determined cDNA sequence of clone 73457.2

SEQ ID NO:345 is the determined cDNA sequence of clone 73457.3

SEQ ID NO:346 is the determined cDNA sequence of clone 74603.1

SEQ ID NO:347 is the determined cDNA sequence of clone 74603.2

SEQ ID NO:348 is the determined cDNA sequence of clone 73458.1

SEQ ID NO:349 is the determined cDNA sequence of clone 73459.2

SEQ ID NO:350 is the determined cDNA sequence of clone 73459.3

SEQ ID NO:351 is the determined cDNA sequence of clone 73460.2

SEQ ID NO:352 is the determined cDNA sequence of clone 73460.3

SEQ ID NO:353 is the determined cDNA sequence of clone 73461.1

SEQ ID NO:354 is the determined cDNA sequence of clone 74604.1

SEQ ID NO:355 is the determined cDNA sequence of clone 74604.2

SEQ ID NO:356 is the determined cDNA sequence of clone 74605.1

SEQ ID NO:357 is the determined cDNA sequence of clone 74605.2

SEQ ID NO:358 is the determined cDNA sequence of clone 74606.1

SEQ ID NO:359 is the determined cDNA sequence of clone 74606.2

SEQ ID NO:360 is the determined cDNA sequence of clone 74607.1

SEQ ID NO:361 is the determined cDNA sequence of clone 74607.2

SEQ ID NO:362 is the determined cDNA sequence of clone 74608.1

SEQ ID NO:363 is the determined cDNA sequence of clone 74608.2

SEQ ID NO:364 is the determined cDNA sequence of clone 74610.2

SEQ ID NO:365 is the determined cDNA sequence of clone 74611.1

SEQ ID NO:366 is the determined cDNA sequence of clone 74613.1

SEQ ID NO:367 is the determined cDNA sequence of clone 74613.2

SEQ ID NO:368 is the determined cDNA sequence of clone 74614.1

SEQ ID NO:369 is the determined cDNA sequence of clone 74614.2

SEQ ID NO:370 is the determined cDNA sequence of clone 74640.2

SEQ ID NO:371 is the determined cDNA sequence of clone 74640.1

SEQ ID NO:372 is the determined cDNA sequence of clone 74615.2

SEQ ID NO:373 is the determined cDNA sequence of clone 74615.1

SEQ ID NO:374 is the determined cDNA sequence of clone 74616.2

SEQ ID NO:375 is the determined cDNA sequence of clone 74616.1

SEQ ID NO:376 is the determined cDNA sequence of clone 74617.2

SEQ ID NO:377 is the determined cDNA sequence of clone 77101.1

SEQ ID NO:378 is the determined cDNA sequence of clone 77102.1

SEQ ID NO:379 is the determined cDNA sequence of clone 77104.1

SEQ ID NO:380 is the determined cDNA sequence of clone 74618.1

SEQ ID NO:381 is the determined cDNA sequence of clone 74618.2

SEQ ID NO:382 is the determined cDNA sequence of clone 74619.2

SEQ ID NO:383 is the determined cDNA sequence of clone 74620.1

SEQ ID NO:384 is the determined cDNA sequence of clone 74620.2

SEQ ID NO:385 is the determined cDNA sequence of clone 74621.1

SEQ ID NO:386 is the determined cDNA sequence of clone 74621.2

SEQ ID NO:387 is the determined cDNA sequence of clone 74623.1

SEQ ID NO:388 is the determined cDNA sequence of clone 74623.2

SEQ ID NO:389 is the determined cDNA sequence of clone 74624.1

SEQ ID NO:390 is the determined cDNA sequence of clone 74624.2

SEQ ID NO:391 is the determined cDNA sequence of clone 74625.1

SEQ ID NO:392 is the determined cDNA sequence of clone 74625.2

SEQ ID NO:393 is the determined cDNA sequence of clone 74631.2

SEQ ID NO:394 is the determined cDNA sequence of clone 74632.1

SEQ ID NO:395 is the determined cDNA sequence of clone 74632.2

SEQ ID NO:396 is the determined cDNA sequence of clone 77105.1

SEQ ID NO:397 is the determined cDNA sequence of clone 77108.1

SEQ ID NO:398 is the determined cDNA sequence of clone 77109.1

SEQ ID NO:399 is the determined cDNA sequence of clone 77114.1

SEQ ID NO:400 is the determined cDNA sequence of clone 77118.1

SEQ ID NO:401 is the determined cDNA sequence of clone 77120.1

SEQ ID NO:402 is the determined cDNA sequence of clone 77122.1

SEQ ID NO:403 is the determined cDNA sequence of clone 77123.1

SEQ ID NO:404 is the determined cDNA sequence of clone 77125.1

SEQ ID NO:405 is the determined cDNA sequence of clone 77127.1

SEQ ID NO:406 is the determined cDNA sequence of clone 77129.1

SEQ ID NO:407 is the determined cDNA sequence of clone 77130.1

SEQ ID NO:408 is the determined cDNA sequence of clone 77132.1

SEQ ID NO:409 is the determined cDNA sequence of clone 77134.1

SEQ ID NO:410 is the determined cDNA sequence of clone 77135.1

SEQ ID NO:411 is the determined cDNA sequence of clone 77136.1

SEQ ID NO:412 is the determined cDNA sequence of clone 77139.1

SEQ ID NO:413 is the determined cDNA sequence of clone 77140.1

SEQ ID NO:414 is the determined cDNA sequence of clone 77141.1

SEQ ID NO:415 is the determined cDNA sequence of clone 77144.1

SEQ ID NO:416 is the determined cDNA sequence of clone 77146.1

SEQ ID NO:417 is the determined cDNA sequence of clone 77149.1

SEQ ID NO:418 is the determined cDNA sequence of clone 77474.1

SEQ ID NO:419 is the determined cDNA sequence of clone 77153.1

SEQ ID NO:420 is the determined cDNA sequence of clone 77479.1

SEQ ID NO:421 is the determined cDNA sequence of clone 77154.1

SEQ ID NO:422 is the determined cDNA sequence of clone 77155.1

SEQ ID NO:423 is the determined cDNA sequence of clone 77157.1

SEQ ID NO:424 is the determined cDNA sequence of clone 77480.1

SEQ ID NO:425 is the determined cDNA sequence of clone 77485.1

SEQ ID NO:426 is the determined cDNA sequence of clone 77487.1

SEQ ID NO:427 is the determined cDNA sequence of clone 77488.1

SEQ ID NO:428 is the determined cDNA sequence of clone 77490.1

SEQ ID NO:429 is the determined cDNA sequence of clone 77494.1

SEQ ID NO:430 is the determined cDNA sequence of clone 77495.1

SEQ ID NO:431 is the determined cDNA sequence of clone 77499.1

SEQ ID NO:432 is the determined cDNA sequence of clone 77500.1

SEQ ID NO:433 is the determined cDNA sequence of clone 77160.1

SEQ ID NO:434 is the determined cDNA sequence of clone 77504.1

SEQ ID NO:435 is the determined cDNA sequence of clone 77506.1

SEQ ID NO:436 is the determined cDNA sequence of clone 77507.1

SEQ ID NO:437 is the determined cDNA sequence of clone 77508.1

SEQ ID NO:438 is the determined cDNA sequence of clone 77509.1

SEQ ID NO:439 is the determined cDNA sequence of clone 77162.1

SEQ ID NO:440 is the determined cDNA sequence of clone 77163.1

SEQ ID NO:441 is the determined cDNA sequence of clone 77165.1

SEQ ID NO:442 is the determined cDNA sequence of clone 77167.1

SEQ ID NO:443 is the determined cDNA sequence of clone 77169.1

SEQ ID NO:444 is the determined cDNA sequence of clone 77171.1

SEQ ID NO:445 is the determined cDNA sequence of clone 77172.1

SEQ ID NO:446 is the determined cDNA sequence of clone 77173.1

SEQ ID NO:447 is the determined cDNA sequence of clone 77175.1

SEQ ID NO:448 is the determined cDNA sequence of clone 77176.1

SEQ ID NO:449 is the determined cDNA sequence of clone 77178.1

SEQ ID NO:450 is the determined cDNA sequence of clone 77180.1

SEQ ID NO:451 is the determined cDNA sequence of clone 77510.1

SEQ ID NO:452 is the full-length determined cDNA sequence for coxIII

SEQ ID NO:453 is the full-length predicted amino acid sequence of coxIII

SEQ ID NO:454 is the determined full-length cDNA sequence of clone 80186 (also referred to as Pn80E), extending the sequence set forth in SEQ ID NO:105

SEQ ID NO:455 is the full-length cDNA sequence of Pn81E.

SEQ ID NO:456 is the determined cDNA sequence of clone PaSLBH2c1

SEQ ID NO:457 is the determined cDNA sequence of clone PaSLBH2c2

SEQ ID NO:458 is the determined cDNA sequence of clone PaSLBH2c3

SEQ ID NO:459 is the determined cDNA sequence of clone PaSLBH2c4

SEQ ID NO:460 is the determined cDNA sequence of clone PaSLBH2c5

SEQ ID NO:461 is the determined cDNA sequence of clone PaSLBH2c7

SEQ ID NO:462 is the determined cDNA sequence of clone PaSLBH2c8

SEQ ID NO:463 is the determined cDNA sequence of clone PaSLBH2c9

SEQ ID NO:464 is the determined cDNA sequence of clone PaSLBH2c10

SEQ ID NO:465 is the determined cDNA sequence of clone PaSLBH2c11

SEQ ID NO:466 is the determined cDNA sequence of clone PaSLBH2c12

SEQ ID NO:467 is the determined cDNA sequence of clone PaSLBH2c13

SEQ ID NO:468 is the determined cDNA sequence of clone PaSLBH2c14

SEQ ID NO:469 is the determined cDNA sequence of clone PaSLBH2c15

SEQ ID NO:470 is the determined cDNA sequence of clone PaSLBH2c16

SEQ ID NO:471 is the determined cDNA sequence of clone PaSLBH2c17

SEQ ID NO:472 is the determined cDNA sequence of clone PaSLBH2c18

SEQ ID NO:473 is the determined cDNA sequence of clone PaSLBH2c19

SEQ ID NO:474 is the determined cDNA sequence of clone PaSLBH2c20

SEQ ID NO:475 is the determined cDNA sequence of clone PaSLBH2c21

SEQ ID NO:476 is the determined cDNA sequence of clone PaSLBH2c22

SEQ ID NO:477 is the determined cDNA sequence of clone PaSLBH2c23

SEQ ID NO:478 is the determined cDNA sequence of clone PaSLBH2c24

SEQ ID NO:479 is the determined cDNA sequence of clone PaSLBH2c25

SEQ ID NO:480 is the determined cDNA sequence of clone PaSLBH2c26

SEQ ID NO:481 is the determined cDNA sequence of clone PaSLBH2c27

SEQ ID NO:482 is the determined cDNA sequence of clone PaSLBH2c28

SEQ ID NO:483 is the determined cDNA sequence of clone PaSLBH2c29

SEQ ID NO:484 is the determined cDNA sequence of clone PaSLBH2c30

SEQ ID NO:485 is the determined cDNA sequence of clone PaSLBH2c31

SEQ ID NO:486 is the determined cDNA sequence of clone PaSLBH2c32

SEQ ID NO:487 is the determined cDNA sequence of clone PaSLBH2c33

SEQ ID NO:488 is the determined cDNA sequence of clone PaSLBH2c34

SEQ ID NO:489 is the determined cDNA sequence of clone PaSLBH2c35

SEQ ID NO:490 is the determined cDNA sequence of clone PaSLBH2c36

SEQ ID NO:491 is the determined cDNA sequence of clone PaSLBH2c37

SEQ ID NO:492 is the determined cDNA sequence of clone PaSLBH2c38

SEQ ID NO:493 is the determined cDNA sequence of clone PaSLBH2c39

SEQ ID NO:494 is the determined cDNA sequence of clone PASLBH2bc1

SEQ ID NO:495 is the determined cDNA sequence of clone PASLBH2bc2

SEQ ID NO:496 is the determined cDNA sequence of clone PASLBH2bc3

SEQ ID NO:497 is the determined cDNA sequence of clone PASLBH2bc4

SEQ ID NO:498 is the determined cDNA sequence of clone PASLBH2bc5

SEQ ID NO:499 is the determined cDNA sequence of clone PASLBH2bc6

SEQ ID NO:500 is the determined cDNA sequence of clone PASLBH2bc7

SEQ ID NO:501 is the determined cDNA sequence of clone PASLBH2bc8

SEQ ID NO:502 is the determined cDNA sequence of clone PASLBH2bc10

SEQ ID NO:503 is the determined cDNA sequence of clone PASLBH2bc11

SEQ ID NO:504 is the determined cDNA sequence of clone PASLBH2bc12

SEQ ID NO:505 is the determined cDNA sequence of clone PASLBH2bc13

SEQ ID NO:506 is the determined cDNA sequence of clone PASLBH2bc14

SEQ ID NO:507 is the determined cDNA sequence of clone PASLBH2bc15

SEQ ID NO:508 is the determined cDNA sequence of clone PASLBH2bc16

SEQ ID NO:509 is the determined cDNA sequence of clone PASLBH2bc17

SEQ ID NO:510 is the determined cDNA sequence of clone PASLBH2bc18

SEQ ID NO:511 is the determined cDNA sequence of clone PASLBH2bc19

SEQ ID NO:512 is the determined cDNA sequence of clone PASLBH2bc20

SEQ ID NO:513 is the determined cDNA sequence of clone PASLBH2bc21

SEQ ID NO:514 is the determined cDNA sequence of clone PASLBH2bc22

SEQ ID NO:515 is the determined cDNA sequence of clone PASLBH2bc23

SEQ ID NO:516 is the determined cDNA sequence of clone PASLBH2bc24

SEQ ID NO:517 is the determined cDNA sequence of clone PASLBH2bc25

SEQ ID NO:518 is the determined cDNA sequence of clone PASLBH2bc26

SEQ ID NO:519 is the determined cDNA sequence of clone PASLBH2bc27

SEQ ID NO:520 is the determined cDNA sequence of clone PASLBH2bc28

SEQ ID NO:521 is the determined cDNA sequence of clone PASLBH2bc29

SEQ ID NO:522 is the determined cDNA sequence of clone PASLBH2bc30

SEQ ID NO:523 is the determined cDNA sequence of clone PASLBH2bc31

SEQ ID NO:524 is the determined cDNA sequence of clone PASLBH2bc32

SEQ ID NO:525 is the determined cDNA sequence of clone PASLBH2bc33

SEQ ID NO:526 is the determined cDNA sequence of clone PASLBH2bc35

SEQ ID NO:527 is the determined cDNA sequence of clone PASLBH2bc36

SEQ ID NO:528 is the determined cDNA sequence of clone PASLBH2bc37

SEQ ID NO:529 is the determined cDNA sequence of clone 61219685

SEQ ID NO:530 is the determined cDNA sequence of clone 61219686

SEQ ID NO:531 is the determined cDNA sequence of clone 61219688

SEQ ID NO:532 is the determined cDNA sequence of clone 61219689

SEQ ID NO:533 is the determined cDNA sequence of clone 61219690

SEQ ID NO:534 is the determined cDNA sequence of clone 61219691

SEQ ID NO:535 is the determined cDNA sequence of clone 61219692

SEQ ID NO:536 is the determined cDNA sequence of clone 61219693

SEQ ID NO:537 is the determined cDNA sequence of clone 61219694

SEQ ID NO:538 is the determined cDNA sequence of clone 61219695

SEQ ID NO:539 is the determined cDNA sequence of clone 61219696

SEQ ID NO:540 is the determined cDNA sequence of clone 61219697

SEQ ID NO:541 is the determined cDNA sequence of clone 61219698

SEQ ID NO:542 is the determined cDNA sequence of clone 61219699

SEQ ID NO:543 is the determined cDNA sequence of clone 61219700

SEQ ID NO:544 is the determined cDNA sequence of clone 61219701

SEQ ID NO:545 is the determined cDNA sequence of clone 61219704

SEQ ID NO:546 is the determined cDNA sequence of clone 61219705

SEQ ID NO:547 is the determined cDNA sequence of clone 61219706

SEQ ID NO:548 is the determined cDNA sequence of clone 61219708

SEQ ID NO:549 is the determined cDNA sequence of clone 61219709

SEQ ID NO:550 is the determined cDNA sequence of clone 61219710

SEQ ID NO:551 is the determined cDNA sequence of clone 61219711

SEQ ID NO:552 is the determined cDNA sequence of clone 61219712

SEQ ID NO:553 is the determined cDNA sequence of clone 61219713

SEQ ID NO:554 is the determined cDNA sequence of clone 61219714

SEQ ID NO:555 is the determined cDNA sequence of clone 61219715

SEQ ID NO:556 is the determined cDNA sequence of clone 61219716

SEQ ID NO:557 is the determined cDNA sequence of clone 61219717

SEQ ID NO:558 is the determined cDNA sequence of clone 61219718

SEQ ID NO:559 is the determined cDNA sequence of clone 61219720

SEQ ID NO:560 is the determined cDNA sequence of clone 61219721

SEQ ID NO:561 is the determined cDNA sequence of clone 61219723

SEQ ID NO:562 is the determined cDNA sequence of clone 61219725

SEQ ID NO:563 is the determined cDNA sequence of clone 61219726

SEQ ID NO:564 is the determined cDNA sequence of clone 61219727

SEQ ID NO:565 is the determined cDNA sequence of clone 61219728

SEQ ID NO:566 is the determined cDNA sequence of clone 61219729

SEQ ID NO:567 is the determined cDNA sequence of clone 61219730

SEQ ID NO:568 is the determined cDNA sequence of clone 61219731

SEQ ID NO:569 is the determined cDNA sequence of clone 61219732

SEQ ID NO:570 is the determined cDNA sequence of clone 61219733

SEQ ID NO:571 is the determined cDNA sequence of clone 61219735

SEQ ID NO:572 is the determined cDNA sequence of clone 61219736

SEQ ID NO:573 is the determined cDNA sequence of clone 61219738

SEQ ID NO:574 is the determined cDNA sequence of clone 61219739

SEQ ID NO:575 is the determined cDNA sequence of clone 61219740

SEQ ID NO:576 is the determined cDNA sequence of clone 61219741

SEQ ID NO:577 is the determined cDNA sequence of clone 61219742

SEQ ID NO:578 is the determined cDNA sequence of clone 61219744

SEQ ID NO:579 is the determined cDNA sequence of clone 61219745

SEQ ID NO:580 is the determined cDNA sequence of clone 61219747

SEQ ID NO:581 is the determined cDNA sequence of clone 61219748

SEQ ID NO:582 is the determined cDNA sequence of clone 61219749

SEQ ID NO:583 is the determined cDNA sequence of clone 61219750

SEQ ID NO:584 is the determined cDNA sequence of clone 61219752

SEQ ID NO:585 is the determined cDNA sequence of clone 61219753

SEQ ID NO:586 is the determined cDNA sequence of clone 61219755

SEQ ID NO:587 is the determined cDNA sequence of clone 61219756

SEQ ID NO:588 is the determined cDNA sequence of clone 61219757

SEQ ID NO:589 is the determined cDNA sequence of clone 61219759

SEQ ID NO:590 is the determined cDNA sequence of clone 61219761

SEQ ID NO:591 is the determined cDNA sequence of clone 61219762

SEQ ID NO:592 is the determined cDNA sequence of clone 61219763

SEQ ID NO:593 is the determined cDNA sequence of clone 61219764

SEQ ID NO:594 is the determined cDNA sequence of clone 61219765

SEQ ID NO:595 is the determined cDNA sequence of clone 61219766

SEQ ID NO:596 is the determined cDNA sequence of clone 61219767

SEQ ID NO:597 is the determined cDNA sequence of clone 61219768

SEQ ID NO:598 is the determined cDNA sequence of clone 61219771

SEQ ID NO:599 is the determined cDNA sequence of clone 61219772

SEQ ID NO:600 is the determined cDNA sequence of clone 61219773

SEQ ID NO:601 is the determined cDNA sequence of clone 61219774

SEQ ID NO:602 is the determined cDNA sequence of clone 61219775

SEQ ID NO:603 is the determined cDNA sequence of clone 61220056

SEQ ID NO:604 is the determined cDNA sequence of clone 61220058

SEQ ID NO:605 is the determined cDNA sequence of clone 61220059

SEQ ID NO:606 is the determined cDNA sequence of clone 61220060

SEQ ID NO:607 is the determined cDNA sequence of clone 61220062

SEQ ID NO:608 is the determined cDNA sequence of clone 61220064

SEQ ID NO:609 is the determined cDNA sequence of clone 61220065

SEQ ID NO:610 is the determined cDNA sequence of clone 61220066

SEQ ID NO:611 is the determined cDNA sequence of clone 61220067

SEQ ID NO:612 is the determined cDNA sequence of clone 61220068

SEQ ID NO:613 is the determined cDNA sequence of clone 61220069

SEQ ID NO:614 is the determined cDNA sequence of clone 61220070

SEQ ID NO:615 is the determined cDNA sequence of clone 61220071

SEQ ID NO:616 is the determined cDNA sequence of clone 61220072

SEQ ID NO:617 is the determined cDNA sequence of clone 61220073

SEQ ID NO:618 is the determined cDNA sequence of clone 61220074

SEQ ID NO:619 is the determined cDNA sequence of clone 61220075

SEQ ID NO:620 is the determined cDNA sequence of clone 61220076

SEQ ID NO:621 is the determined cDNA sequence of clone 61220077

SEQ ID NO:622 is the determined cDNA sequence of clone 61220078

SEQ ID NO:623 is the determined cDNA sequence of clone 61220080

SEQ ID NO:624 is the determined cDNA sequence of clone 61220081

SEQ ID NO:625 is the determined cDNA sequence of clone 61220082

SEQ ID NO:626 is the determined cDNA sequence of clone 61220083

SEQ ID NO:627 is the determined cDNA sequence of clone 61220084

SEQ ID NO:628 is the determined cDNA sequence of clone 61220085

SEQ ID NO:629 is the determined cDNA sequence of clone 61220086

SEQ ID NO:630 is the determined cDNA sequence of clone 61220087

SEQ ID NO:631 is the determined cDNA sequence of clone 61220088

SEQ ID NO:632 is the determined cDNA sequence of clone 61220089

SEQ ID NO:633 is the determined cDNA sequence of clone 61220090

SEQ ID NO:634 is the determined cDNA sequence of clone 61220091

SEQ ID NO:635 is the determined cDNA sequence of clone 61220093

SEQ ID NO:636 is the determined cDNA sequence of clone 61220094

SEQ ID NO:637 is the determined cDNA sequence of clone 61220095

SEQ ID NO:638 is the determined cDNA sequence of clone 61220096

SEQ ID NO:639 is the determined cDNA sequence of clone 61220097

SEQ ID NO:640 is the determined cDNA sequence of clone 61220099

SEQ ID NO:641 is the determined cDNA sequence of clone 61220100

SEQ ID NO:642 is the determined cDNA sequence of clone 61220101

SEQ ID NO:643 is the determined cDNA sequence of clone 61220104

SEQ ID NO:644 is the determined cDNA sequence of clone 61220105

SEQ ID NO:645 is the determined cDNA sequence of clone 61220106

SEQ ID NO:646 is the determined cDNA sequence of clone 61220107

SEQ ID NO:647 is the determined cDNA sequence of clone 61220108

SEQ ID NO:648 is the determined cDNA sequence of clone 61220109

SEQ ID NO:649 is the determined cDNA sequence of clone 61220110

SEQ ID NO:650 is the determined cDNA sequence of clone 61220112

SEQ ID NO:651 is the determined cDNA sequence of clone 61220113

SEQ ID NO:652 is the determined cDNA sequence of clone 61220114

SEQ ID NO:653 is the determined cDNA sequence of clone 61220115

SEQ ID NO:654 is the determined cDNA sequence of clone 61220116

SEQ ID NO:655 is the determined cDNA sequence of clone 61220117

SEQ ID NO:656 is the determined cDNA sequence of clone 61220118

SEQ ID NO:657 is the determined cDNA sequence of clone 61220119

SEQ ID NO:658 is the determined cDNA sequence of clone 61220120

SEQ ID NO:659 is the determined cDNA sequence of clone 61220121

SEQ ID NO:660 is the determined cDNA sequence of clone 61220122

SEQ ID NO:661 is the determined cDNA sequence of clone 61220124

SEQ ID NO:662 is the determined cDNA sequence of clone 61220125

SEQ ID NO:663 is the determined cDNA sequence of clone 61220126

SEQ ID NO:664 is the determined cDNA sequence of clone 61220127

SEQ ID NO:665 is the determined cDNA sequence of clone 61220128

SEQ ID NO:666 is the determined cDNA sequence of clone 61220129

SEQ ID NO:667 is the determined cDNA sequence of clone 61220130

SEQ ID NO:668 is the determined cDNA sequence of clone 61220133

SEQ ID NO:669 is the determined cDNA sequence of clone 61220134

SEQ ID NO:670 is the determined cDNA sequence of clone 61220135

SEQ ID NO:671 is the determined cDNA sequence of clone 61220137

SEQ ID NO:672 is the determined cDNA sequence of clone 61220139

SEQ ID NO:673 is the determined cDNA sequence of clone 61220140

SEQ ID NO:674 is the determined cDNA sequence of clone 61220141

SEQ ID NO:675 is the determined cDNA sequence of clone 61220142

SEQ ID NO:676 is the determined cDNA sequence of clone 61220143

SEQ ID NO:677 is the determined cDNA sequence of clone 61220144

SEQ ID NO:678 is the determined cDNA sequence of clone 61220146

SEQ ID NO:679 is the determined cDNA sequence of clone 61220147

SEQ ID NO:680 is the determined cDNA sequence of clone 61219777

SEQ ID NO:681 is the determined cDNA sequence of clone 61219778

SEQ ID NO:682 is the determined cDNA sequence of clone 61219779

SEQ ID NO:683 is the determined cDNA sequence of clone 61219780

SEQ ID NO:684 is the determined cDNA sequence of clone 61219781

SEQ ID NO:685 is the determined cDNA sequence of clone 61219782

SEQ ID NO:686 is the determined cDNA sequence of clone 61219783

SEQ ID NO:687 is the determined cDNA sequence of clone 61219784

SEQ ID NO:688 is the determined cDNA sequence of clone 61219785

SEQ ID NO:689 is the determined cDNA sequence of clone 61219786

SEQ ID NO:690 is the determined cDNA sequence of clone 61219787

SEQ ID NO:691 is the determined cDNA sequence of clone 61219788

SEQ ID NO:692 is the determined cDNA sequence of clone 61219789

SEQ ID NO:693 is the determined cDNA sequence of clone 61219790

SEQ ID NO:694 is the determined cDNA sequence of clone 61219791

SEQ ID NO:695 is the determined cDNA sequence of clone 61219792

SEQ ID NO:696 is the determined cDNA sequence of clone 61219793

SEQ ID NO:697 is the determined cDNA sequence of clone 61219794

SEQ ID NO:698 is the determined cDNA sequence of clone 61219795

SEQ ID NO:699 is the determined cDNA sequence of clone 61219797

SEQ ID NO:700 is the determined cDNA sequence of clone 61219798

SEQ ID NO:701 is the determined cDNA sequence of clone 61219799

SEQ ID NO:702 is the determined cDNA sequence of clone 61219800

SEQ ID NO:703 is the determined cDNA sequence of clone 61219801

SEQ ID NO:704 is the determined cDNA sequence of clone 61219802

SEQ ID NO:705 is the determined cDNA sequence of clone 61219803

SEQ ID NO:706 is the determined cDNA sequence of clone 61219804

SEQ ID NO:707 is the determined cDNA sequence of clone 61219805

SEQ ID NO:708 is the determined cDNA sequence of clone 61219806

SEQ ID NO:709 is the determined cDNA sequence of clone 61219807

SEQ ID NO:710 is the determined cDNA sequence of clone 61219808

SEQ ID NO:711 is the determined cDNA sequence of clone 61219809

SEQ ID NO:712 is the determined cDNA sequence of clone 61219810

SEQ ID NO:713 is the determined cDNA sequence of clone 61219812

SEQ ID NO:714 is the determined cDNA sequence of clone 61219814

SEQ ID NO:715 is the determined cDNA sequence of clone 61219815

SEQ ID NO:716 is the determined cDNA sequence of clone 61219816

SEQ ID NO:717 is the determined cDNA sequence of clone 61219817

SEQ ID NO:718 is the determined cDNA sequence of clone 61219818

SEQ ID NO:719 is the determined cDNA sequence of clone 61219819

SEQ ID NO:720 is the determined cDNA sequence of clone 61219820

SEQ ID NO:721 is the determined cDNA sequence of clone 61219821

SEQ ID NO:722 is the determined cDNA sequence of clone 61219822

SEQ ID NO:723 is the determined cDNA sequence of clone 61219823

SEQ ID NO:724 is the determined cDNA sequence of clone 61219824

SEQ ID NO:725 is the determined cDNA sequence of clone 61219825

SEQ ID NO:726 is the determined cDNA sequence of clone 61219826

SEQ ID NO:727 is the determined cDNA sequence of clone 61219827

SEQ ID NO:728 is the determined cDNA sequence of clone 61219828

SEQ ID NO:729 is the determined cDNA sequence of clone 61219829

SEQ ID NO:730 is the determined cDNA sequence of clone 61219830

SEQ ID NO:731 is the determined cDNA sequence of clone 61219831

SEQ ID NO:732 is the determined cDNA sequence of clone 61219832

SEQ ID NO:733 is the determined cDNA sequence of clone 61219833

SEQ ID NO:734 is the determined cDNA sequence of clone 61219834

SEQ ID NO:735 is the determined cDNA sequence of clone 61219835

SEQ ID NO:736 is the determined cDNA sequence of clone 61219836

SEQ ID NO:737 is the determined cDNA sequence of clone 61219838

SEQ ID NO:738 is the determined cDNA sequence of clone 61219839

SEQ ID NO:739 is the determined cDNA sequence of clone 61219840

SEQ ID NO:740 is the determined cDNA sequence of clone 61219841

SEQ ID NO:741 is the determined cDNA sequence of clone 61219842

SEQ ID NO:742 is the determined cDNA sequence of clone 61219843

SEQ ID NO:743 is the determined cDNA sequence of clone 61219844

SEQ ID NO:744 is the determined cDNA sequence of clone 61219845

SEQ ID NO:745 is the determined cDNA sequence of clone 61219846

SEQ ID NO:746 is the determined cDNA sequence of clone 61219848

SEQ ID NO:747 is the determined cDNA sequence of clone 61219850

SEQ ID NO:748 is the determined cDNA sequence of clone 61219851

SEQ ID NO:749 is the determined cDNA sequence of clone 61219853

SEQ ID NO:750 is the determined cDNA sequence of clone 61219854

SEQ ID NO:751 is the determined cDNA sequence of clone 61219857

SEQ ID NO:752 is the determined cDNA sequence of clone 61219858

SEQ ID NO:753 is the determined cDNA sequence of clone 61219859

SEQ ID NO:754 is the determined cDNA sequence of clone 61219860

SEQ ID NO:755 is the determined cDNA sequence of clone 61219861

SEQ ID NO:756 is the determined cDNA sequence of clone 61219862

SEQ ID NO:757 is the determined cDNA sequence of clone 61219863

SEQ ID NO:758 is the determined cDNA sequence of clone 61219864

SEQ ID NO:759 is the determined cDNA sequence of clone 61219865

SEQ ID NO:760 is the determined cDNA sequence of clone 61219866

SEQ ID NO:761 is the determined cDNA sequence of clone 61219867

SEQ ID NO:762 is the determined cDNA sequence of clone 61219869

SEQ ID NO:763 is the determined cDNA sequence of clone 61220149

SEQ ID NO:764 is the determined cDNA sequence of clone 61220150

SEQ ID NO:765 is the determined cDNA sequence of clone 61220151

SEQ ID NO:766 is the determined cDNA sequence of clone 61220152

SEQ ID NO:767 is the determined cDNA sequence of clone 61220153

SEQ ID NO:768 is the determined cDNA sequence of clone 61220154

SEQ ID NO:769 is the determined cDNA sequence of clone 61220156

SEQ ID NO:770 is the determined cDNA sequence of clone 61220158

SEQ ID NO:771 is the determined cDNA sequence of clone 61220159

SEQ ID NO:772 is the determined cDNA sequence of clone 61220160

SEQ ID NO:773 is the determined cDNA sequence of clone 61220161

SEQ ID NO:774 is the determined cDNA sequence of clone 61220162

SEQ ID NO:775 is the determined cDNA sequence of clone 61220163

SEQ ID NO:776 is the determined cDNA sequence of clone 61220164

SEQ ID NO:777 is the determined cDNA sequence of clone 61220167

SEQ ID NO:778 is the determined cDNA sequence of clone 61220168

SEQ ID NO:779 is the determined cDNA sequence of clone 61220169

SEQ ID NO:780 is the determined cDNA sequence of clone 61220170

SEQ ID NO:781 is the determined cDNA sequence of clone 61220171

SEQ ID NO:782 is the determined cDNA sequence of clone 61220172

SEQ ID NO:783 is the determined cDNA sequence of clone 61220173

SEQ ID NO:784 is the determined cDNA sequence of clone 61220175

SEQ ID NO:785 is the determined cDNA sequence of clone 61220176

SEQ ID NO:786 is the determined cDNA sequence of clone 61220177

SEQ ID NO:787 is the determined cDNA sequence of clone 61220178

SEQ ID NO:788 is the determined cDNA sequence of clone 61220179

SEQ ID NO:789 is the determined cDNA sequence of clone 61220180

SEQ ID NO:790 is the determined cDNA sequence of clone 61220181

SEQ ID NO:791 is the determined cDNA sequence of clone 61220182

SEQ ID NO:792 is the determined cDNA sequence of clone 61220183

SEQ ID NO:793 is the determined cDNA sequence of clone 61220184

SEQ ID NO:794 is the determined cDNA sequence of clone 61220185

SEQ ID NO:795 is the determined cDNA sequence of clone 61220186

SEQ ID NO:796 is the determined cDNA sequence of clone 61220187

SEQ ID NO:797 is the determined cDNA sequence of clone 61220188

SEQ ID NO:798 is the determined cDNA sequence of clone 61220189

SEQ ID NO:799 is the determined cDNA sequence of clone 61220190

SEQ ID NO:800 is the determined cDNA sequence of clone 61220192

SEQ ID NO:801 is the determined cDNA sequence of clone 61220193

SEQ ID NO:802 is the determined cDNA sequence of clone 61220194

SEQ ID NO:803 is the determined cDNA sequence of clone 61220195

SEQ ID NO:804 is the determined cDNA sequence of clone 61220196

SEQ ID NO:805 is the determined cDNA sequence of clone 61220198

SEQ ID NO:806 is the determined cDNA sequence of clone 61220199

SEQ ID NO:807 is the determined cDNA sequence of clone 61220200

SEQ ID NO:808 is the determined cDNA sequence of clone 61220203

SEQ ID NO:809 is the determined cDNA sequence of clone 61220204

SEQ ID NO:810 is the determined cDNA sequence of clone 61220205

SEQ ID NO:811 is the determined cDNA sequence of clone 61220207

SEQ ID NO:812 is the determined cDNA sequence of clone 61220208

SEQ ID NO:813 is the determined cDNA sequence of clone 61220209

SEQ ID NO:814 is the determined cDNA sequence of clone 61220210

SEQ ID NO:815 is the determined cDNA sequence of clone 61220211

SEQ ID NO:816 is the determined cDNA sequence of clone 61220213

SEQ ID NO:817 is the determined cDNA sequence of clone 61220214

SEQ ID NO:818 is the determined cDNA sequence of clone 61220216

SEQ ID NO:819 is the determined cDNA sequence of clone 61220217

SEQ ID NO:820 is the determined cDNA sequence of clone 61220219

SEQ ID NO:821 is the determined cDNA sequence of clone 61220220

SEQ ID NO:822 is the determined cDNA sequence of clone 61220221

SEQ ID NO:823 is the determined cDNA sequence of clone 61220222

SEQ ID NO:824 is the determined cDNA sequence of clone 61220223

SEQ ID NO:825 is the determined cDNA sequence of clone 61220224

SEQ ID NO:826 is the determined cDNA sequence of clone 61220225

SEQ ID NO:827 is the determined cDNA sequence of clone 61220226

SEQ ID NO:828 is the determined cDNA sequence of clone 61220227

SEQ ID NO:829 is the determined cDNA sequence of clone 61220228

SEQ ID NO:830 is the determined cDNA sequence of clone 61220230

SEQ ID NO:831 is the determined cDNA sequence of clone 61220231

SEQ ID NO:832 is the determined cDNA sequence of clone 61220232

SEQ ID NO:833 is the determined cDNA sequence of clone 61220233

SEQ ID NO:834 is the determined cDNA sequence of clone 61220234

SEQ ID NO:835 is the determined cDNA sequence of clone 61220235

SEQ ID NO:836 is the determined cDNA sequence of clone 61220236

SEQ ID NO:837 is the determined cDNA sequence of clone 61220237

SEQ ID NO:838 is the determined cDNA sequence of clone 61220239

SEQ ID NO:839 is the determined cDNA sequence of clone 61220240

SEQ ID NO:840 is the determined cDNA sequence of clone 61220241

SEQ ID NO:841 is the determined cDNA sequence of clone 61220242

SEQ ID NO:842 is the determined cDNA sequence of clone 61220244

SEQ ID NO:843 is the determined cDNA sequence of clone 61220245

SEQ ID NO:844 is the determined cDNA sequence of clone 61220246

SEQ ID NO:845 is the determined cDNA sequence of clone 61220247

SEQ ID NO:846 is the determined cDNA sequence of clone 61220248

SEQ ID NO:847 is the determined cDNA sequence of clone 61220249

SEQ ID NO:848 is the determined cDNA sequence of clone 61220250

SEQ ID NO:849 is the determined cDNA sequence of clone 61220251

SEQ ID NO:850 is the determined cDNA sequence of clone 61220252

SEQ ID NO:851 is the determined cDNA sequence of clone 61220253

SEQ ID NO:852 is the determined cDNA sequence of clone 61220254

SEQ ID NO:853 is the determined cDNA sequence of clone 61220255

SEQ ID NO:854 is the determined cDNA sequence of clone 61220256

SEQ ID NO:855 is the determined cDNA sequence of clone 61220258

SEQ ID NO:856 is the determined cDNA sequence of clone 61220259

SEQ ID NO:857 is the determined cDNA sequence of clone 61220260

SEQ ID NO:858 is the determined cDNA sequence of clone 61220261

SEQ ID NO:859 is the determined cDNA sequence of clone 61220262

SEQ ID NO:860 is the determined cDNA sequence of clone 61220263

SEQ ID NO:861 is the determined cDNA sequence of clone 61220264

SEQ ID NO:862 is the determined cDNA sequence of clone 61220265

SEQ ID NO:863 is the determined cDNA sequence of clone 61220267

SEQ ID NO:864 is the determined cDNA sequence of clone 61220268

SEQ ID NO:865 is the determined cDNA sequence of clone 61220269

SEQ ID NO:866 is the determined cDNA sequence of clone 61220270

SEQ ID NO:867 is the determined cDNA sequence of clone 61220271

SEQ ID NO:868 is the determined cDNA sequence of clone 61220272

SEQ ID NO:869 is the determined cDNA sequence of clone 61220273

SEQ ID NO:870 is the determined cDNA sequence of clone 61220274

SEQ ID NO:871 is the determined cDNA sequence of clone 61220275

SEQ ID NO:872 is the determined cDNA sequence of clone 61220278

SEQ ID NO:873 is the determined cDNA sequence of clone 61220279

SEQ ID NO:874 is the determined cDNA sequence of clone 61220280

SEQ ID NO:875 is the determined cDNA sequence of clone 61220281

SEQ ID NO:876 is the determined cDNA sequence of clone 61220282

SEQ ID NO:877 is the determined cDNA sequence of clone 61220283

SEQ ID NO:878 is the determined cDNA sequence of clone 61220284

SEQ ID NO:879 is the determined cDNA sequence of clone 61220285

SEQ ID NO:880 is the determined cDNA sequence of clone 61220287

SEQ ID NO:881 is the determined cDNA sequence of clone 61220288

SEQ ID NO:882 is the determined cDNA sequence of clone 61220290

SEQ ID NO:883 is the determined cDNA sequence of clone 61220291

SEQ ID NO:884 is the determined cDNA sequence of clone 61220292

SEQ ID NO:885 is the determined cDNA sequence of clone 61220293

SEQ ID NO:886 is the determined cDNA sequence of clone 61220294

SEQ ID NO:887 is the determined cDNA sequence of clone 61220295

SEQ ID NO:888 is the determined cDNA sequence of clone 61220296

SEQ ID NO:889 is the determined cDNA sequence of clone 61220298

SEQ ID NO:890 is the determined cDNA sequence of clone 61220299

SEQ ID NO:891 is the determined cDNA sequence of clone 61220300

SEQ ID NO:892 is the determined cDNA sequence of clone 61220301

SEQ ID NO:893 is the determined cDNA sequence of clone 61220302

SEQ ID NO:894 is the determined cDNA sequence of clone 61220303

SEQ ID NO:895 is the determined cDNA sequence of clone 61220304

SEQ ID NO:896 is the determined cDNA sequence of clone 61220305

SEQ ID NO:897 is the determined cDNA sequence of clone 61220307

SEQ ID NO:898 is the determined cDNA sequence of clone 61220308

SEQ ID NO:899 is the determined cDNA sequence of clone 61220309

SEQ ID NO:900 is the determined cDNA sequence of clone 61220310

SEQ ID NO:901 is the determined cDNA sequence of clone 61220314

SEQ ID NO:902 is the determined cDNA sequence of clone 61220316

SEQ ID NO:903 is the determined cDNA sequence of clone 61220317

SEQ ID NO:904 is the determined cDNA sequence of clone 61220318

SEQ ID NO:905 is the determined cDNA sequence of clone 61220319

SEQ ID NO:906 is the determined cDNA sequence of clone 61220320

SEQ ID NO:907 is the determined cDNA sequence of clone 61220321

SEQ ID NO:908 is the determined cDNA sequence of clone 61220322

SEQ ID NO:909 is the determined cDNA sequence of clone 61220323

SEQ ID NO:910 is the determined cDNA sequence of clone 61220324

SEQ ID NO:911 is the determined cDNA sequence of clone 61220325

SEQ ID NO:912 is the determined cDNA sequence of clone 61220327

SEQ ID NO:913 is the determined cDNA sequence of clone 61220328

SEQ ID NO:914 is the determined cDNA sequence of clone 61220330

SEQ ID NO:915 is the determined cDNA sequence of clone 61220331

SEQ ID NO:916 is the determined cDNA sequence of clone 61220332

SEQ ID NO:917 is the determined cDNA sequence of clone 61220333

SEQ ID NO:918 is the determined cDNA sequence of clone 61220334

SEQ ID NO:919 is the determined cDNA sequence of clone 61219870

SEQ ID NO:920 is the determined cDNA sequence of clone 61219871

SEQ ID NO:921 is the determined cDNA sequence of clone 61219872

SEQ ID NO:922 is the determined cDNA sequence of clone 61219874

SEQ ID NO:923 is the determined cDNA sequence of clone 61219875

SEQ ID NO:924 is the determined cDNA sequence of clone 61219877

SEQ ID NO:925 is the determined cDNA sequence of clone 61219878

SEQ ID NO:926 is the determined cDNA sequence of clone 61219879

SEQ ID NO:927 is the determined cDNA sequence of clone 61219880

SEQ ID NO:928 is the determined cDNA sequence of clone 61219881

SEQ ID NO:929 is the determined cDNA sequence of clone 61219882

SEQ ID NO:930 is the determined cDNA sequence of clone 61219883

SEQ ID NO:931 is the determined cDNA sequence of clone 61219884

SEQ ID NO:932 is the determined cDNA sequence of clone 61219885

SEQ ID NO:933 is the determined cDNA sequence of clone 61219886

SEQ ID NO:934 is the determined cDNA sequence of clone 61219887

SEQ ID NO:935 is the determined cDNA sequence of clone 61219888

SEQ ID NO:936 is the determined cDNA sequence of clone 61219889

SEQ ID NO:937 is the determined cDNA sequence of clone 61219892

SEQ ID NO:938 is the determined cDNA sequence of clone 61219893

SEQ ID NO:939 is the determined cDNA sequence of clone 61219894

SEQ ID NO:940 is the determined cDNA sequence of clone 61219895

SEQ ID NO:941 is the determined cDNA sequence of clone 61219897

SEQ ID NO:942 is the determined cDNA sequence of clone 61219898

SEQ ID NO:943 is the determined cDNA sequence of clone 61219899

SEQ ID NO:944 is the determined cDNA sequence of clone 61219900

SEQ ID NO:945 is the determined cDNA sequence of clone 61219901

SEQ ID NO:946 is the determined cDNA sequence of clone 61219902

SEQ ID NO:947 is the determined cDNA sequence of clone 61219903

SEQ ID NO:948 is the determined cDNA sequence of clone 61219905

SEQ ID NO:949 is the determined cDNA sequence of clone 61219906

SEQ ID NO:950 is the determined cDNA sequence of clone 61219907

SEQ ID NO:951 is the determined cDNA sequence of clone 61219908

SEQ ID NO:952 is the determined cDNA sequence of clone 61219909

SEQ ID NO:953 is the determined cDNA sequence of clone 61219910

SEQ ID NO:954 is the determined cDNA sequence of clone 61219911

SEQ ID NO:955 is the determined cDNA sequence of clone 61219912

SEQ ID NO:956 is the determined cDNA sequence of clone 61219914

SEQ ID NO:957 is the determined cDNA sequence of clone 61219915

SEQ ID NO:958 is the determined cDNA sequence of clone 61219916

SEQ ID NO:959 is the determined cDNA sequence of clone 61219917

SEQ ID NO:960 is the determined cDNA sequence of clone 61219918

SEQ ID NO:961 is the determined cDNA sequence of clone 61219919

SEQ ID NO:962 is the determined cDNA sequence of clone 61219920

SEQ ID NO:963 is the determined cDNA sequence of clone 61219921

SEQ ID NO:964 is the determined cDNA sequence of clone 61219922

SEQ ID NO:965 is the determined cDNA sequence of clone 61219923

SEQ ID NO:966 is the determined cDNA sequence of clone 61219927

SEQ ID NO:967 is the determined cDNA sequence of clone 61219928

SEQ ID NO:968 is the determined cDNA sequence of clone 61219929

SEQ ID NO:969 is the determined cDNA sequence of clone 61219932

SEQ ID NO:970 is the determined cDNA sequence of clone 61219933

SEQ ID NO:971 is the determined cDNA sequence of clone 61219934

SEQ ID NO:972 is the determined cDNA sequence of clone 61219935

SEQ ID NO:973 is the determined cDNA sequence of clone 61219936

SEQ ID NO:974 is the determined cDNA sequence of clone 61219937

SEQ ID NO:975 is the determined cDNA sequence of clone 61219938

SEQ ID NO:976 is the determined cDNA sequence of clone 61219940

SEQ ID NO:977 is the determined cDNA sequence of clone 61219941

SEQ ID NO:978 is the determined cDNA sequence of clone 61219942

SEQ ID NO:979 is the determined cDNA sequence of clone 61219943

SEQ ID NO:980 is the determined cDNA sequence of clone 61219944

SEQ ID NO:981 is the determined cDNA sequence of clone 61219946

SEQ ID NO:982 is the determined cDNA sequence of clone 61219947

SEQ ID NO:983 is the determined cDNA sequence of clone 61219948

SEQ ID NO:984 is the determined cDNA sequence of clone 61219949

SEQ ID NO:985 is the determined cDNA sequence of clone 61219951

SEQ ID NO:986 is the determined cDNA sequence of clone 61219952

SEQ ID NO:987 is the determined cDNA sequence of clone 61219953

SEQ ID NO:988 is the determined cDNA sequence of clone 61219955

SEQ ID NO:989 is the determined cDNA sequence of clone 61219956

SEQ ID NO:990 is the determined cDNA sequence of clone 61219957

SEQ ID NO:991 is the determined cDNA sequence of clone 61219959

SEQ ID NO:992 is the determined cDNA sequence of clone 61219961

SEQ ID NO:993 is the determined cDNA sequence of clone 61219962

SEQ ID NO:994 is the determined cDNA sequence of clone 61220338

SEQ ID NO:995 is the determined cDNA sequence of clone 61220339

SEQ ID NO:996 is the determined cDNA sequence of clone 61220340

SEQ ID NO:997 is the determined cDNA sequence of clone 61220341

SEQ ID NO:998 is the determined cDNA sequence of clone 61220342

SEQ ID NO:999 is the determined cDNA sequence of clone 61220343

SEQ ID NO:1000 is the determined cDNA sequence of clone 61220344

SEQ ID NO:1001 is the determined cDNA sequence of clone 61220345

SEQ ID NO:1002 is the determined cDNA sequence of clone 61220346

SEQ ID NO:1003 is the determined cDNA sequence of clone 61220347

SEQ ID NO:1004 is the determined cDNA sequence of clone 61220348

SEQ ID NO:1005 is the determined cDNA sequence of clone 61220349

SEQ ID NO:1006 is the determined cDNA sequence of clone 61220350

SEQ ID NO:1007 is the determined cDNA sequence of clone 61220351

SEQ ID NO:1008 is the determined cDNA sequence of clone 61220352

SEQ ID NO:1009 is the determined cDNA sequence of clone 61220353

SEQ ID NO:1010 is the determined cDNA sequence of clone 61220354

SEQ ID NO:1011 is the determined cDNA sequence of clone 61220356

SEQ ID NO:1012 is the determined cDNA sequence of clone 61220357

SEQ ID NO:1013 is the determined cDNA sequence of clone 61220358

SEQ ID NO:1014 is the determined cDNA sequence of clone 61220359

SEQ ID NO:1015 is the determined cDNA sequence of clone 61220360

SEQ ID NO:1016 is the determined cDNA sequence of clone 61220361

SEQ ID NO:1017 is the determined cDNA sequence of clone 61220362

SEQ ID NO:1018 is the determined cDNA sequence of clone 61220363

SEQ ID NO:1019 is the determined cDNA sequence of clone 61220364

SEQ ID NO:1020 is the determined cDNA sequence of clone 61220365

SEQ ID NO:1021 is the determined cDNA sequence of clone 61220366

SEQ ID NO:1022 is the determined cDNA sequence of clone 61220367

SEQ ID NO:1023 is the determined cDNA sequence of clone 61220368

SEQ ID NO:1024 is the determined cDNA sequence of clone 61220369

SEQ ID NO:1025 is the determined cDNA sequence of clone 61220370

SEQ ID NO:1026 is the determined cDNA sequence of clone 61220372

SEQ ID NO:1027 is the determined cDNA sequence of clone 61220373

SEQ ID NO:1028 is the determined cDNA sequence of clone 61220374

SEQ ID NO:1029 is the determined cDNA sequence of clone 61220375

SEQ ID NO:1030 is the determined cDNA sequence of clone 61220376

SEQ ID NO:1031 is the determined cDNA sequence of clone 61220377

SEQ ID NO:1032 is the determined cDNA sequence of clone 61220378

SEQ ID NO:1033 is the determined cDNA sequence of clone 61220380

SEQ ID NO:1034 is the determined cDNA sequence of clone 61220381

SEQ ID NO:1035 is the determined cDNA sequence of clone 61220382

SEQ ID NO:1036 is the determined cDNA sequence of clone 61220383

SEQ ID NO:1037 is the determined cDNA sequence of clone 61220385

SEQ ID NO:1038 is the determined cDNA sequence of clone 61220386

SEQ ID NO:1039 is the determined cDNA sequence of clone 61220387

SEQ ID NO:1040 is the determined cDNA sequence of clone 61220388

SEQ ID NO:1041 is the determined cDNA sequence of clone 61220389

SEQ ID NO:1042 is the determined cDNA sequence of clone 61220390

SEQ ID NO:1043 is the determined cDNA sequence of clone 61220391

SEQ ID NO:1044 is the determined cDNA sequence of clone 61220392

SEQ ID NO:1045 is the determined cDNA sequence of clone 61220393

SEQ ID NO:1046 is the determined cDNA sequence of clone 61220394

SEQ ID NO:1047 is the determined cDNA sequence of clone 61220395

SEQ ID NO:1048 is the determined cDNA sequence of clone 61220396

SEQ ID NO:1049 is the determined cDNA sequence of clone 61220398

SEQ ID NO:1050 is the determined cDNA sequence of clone 61220399

SEQ ID NO:1051 is the determined cDNA sequence of clone 61220402

SEQ ID NO:1052 is the determined cDNA sequence of clone 61220403

SEQ ID NO:1053 is the determined cDNA sequence of clone 61220404

SEQ ID NO:1054 is the determined cDNA sequence of clone 61220405

SEQ ID NO:1055 is the determined cDNA sequence of clone 61220406

SEQ ID NO:1056 is the determined cDNA sequence of clone 61220407

SEQ ID NO:1057 is the determined cDNA sequence of clone 61220408

SEQ ID NO:1058 is the determined cDNA sequence of clone 61220409

SEQ ID NO:1059 is the determined cDNA sequence of clone 61220410

SEQ ID NO:1060 is the determined cDNA sequence of clone 61220411

SEQ ID NO:1061 is the determined cDNA sequence of clone 61220413

SEQ ID NO:1062 is the determined cDNA sequence of clone 61220414

SEQ ID NO:1063 is the determined cDNA sequence of clone 61220415

SEQ ID NO:1064 is the determined cDNA sequence of clone 61220418

SEQ ID NO:1065 is the determined cDNA sequence of clone 61220419

SEQ ID NO:1066 is the determined cDNA sequence of clone 61220421

SEQ ID NO:1067 is the determined cDNA sequence of clone 61220422

SEQ ID NO:1068 is the determined cDNA sequence of clone 61220423

SEQ ID NO:1069 is the determined cDNA sequence of clone 61220424

SEQ ID NO:1070 is the determined cDNA sequence of clone 61220426

SEQ ID NO:1071 is the determined cDNA sequence of clone 61220427

SEQ ID NO:1072 is the determined cDNA sequence of clone 61219963

SEQ ID NO:1073 is the determined cDNA sequence of clone 61219964

SEQ ID NO:1074 is the determined cDNA sequence of clone 61219966

SEQ ID NO:1075 is the determined cDNA sequence of clone 61219967

SEQ ID NO:1076 is the determined cDNA sequence of clone 61219968

SEQ ID NO:1077 is the determined cDNA sequence of clone 61219969

SEQ ID NO:1078 is the determined cDNA sequence of clone 61219970

SEQ ID NO:1079 is the determined cDNA sequence of clone 61219971

SEQ ID NO:1080 is the determined cDNA sequence of clone 61219973

SEQ ID NO:1081 is the determined cDNA sequence of clone 61219974

SEQ ID NO:1082 is the determined cDNA sequence of clone 61219975

SEQ ID NO:1083 is the determined cDNA sequence of clone 61219978

SEQ ID NO:1084 is the determined cDNA sequence of clone 61219979

SEQ ID NO:1085 is the determined cDNA sequence of clone 61219980

SEQ ID NO:1086 is the determined cDNA sequence of clone 61219981

SEQ ID NO:1087 is the determined cDNA sequence of clone 61219982

SEQ ID NO:1088 is the determined cDNA sequence of clone 61219983

SEQ ID NO:1089 is the determined cDNA sequence of clone 61219984

SEQ ID NO:1090 is the determined cDNA sequence of clone 61219985

SEQ ID NO:1091 is the determined cDNA sequence of clone 61219986

SEQ ID NO:1092 is the determined cDNA sequence of clone 61219987

SEQ ID NO:1093 is the determined cDNA sequence of clone 61219988

SEQ ID NO:1094 is the determined cDNA sequence of clone 61219990

SEQ ID NO:1095 is the determined cDNA sequence of clone 61219991

SEQ ID NO:1096 is the determined cDNA sequence of clone 61219992

SEQ ID NO:1097 is the determined cDNA sequence of clone 61219993

SEQ ID NO:1098 is the determined cDNA sequence of clone 61219994

SEQ ID NO:1099 is the determined cDNA sequence of clone 61219995

SEQ ID NO:1100 is the determined cDNA sequence of clone 61219996

SEQ ID NO:1101 is the determined cDNA sequence of clone 61219997

SEQ ID NO:1102 is the determined cDNA sequence of clone 61219999

SEQ ID NO:1103 is the determined cDNA sequence of clone 61220000

SEQ ID NO:1104 is the determined cDNA sequence of clone 61220001

SEQ ID NO:1105 is the determined cDNA sequence of clone 61220002

SEQ ID NO:1106 is the determined cDNA sequence of clone 61220003

SEQ ID NO:1107 is the determined cDNA sequence of clone 61220004

SEQ ID NO:1108 is the determined cDNA sequence of clone 61220005

SEQ ID NO:1109 is the determined cDNA sequence of clone 61220006

SEQ ID NO:1110 is the determined cDNA sequence of clone 61220007

SEQ ID NO:1111 is the determined cDNA sequence of clone 61220008

SEQ ID NO:1112 is the determined cDNA sequence of clone 61220009

SEQ ID NO:1113 is the determined cDNA sequence of clone 61220011

SEQ ID NO:1114 is the determined cDNA sequence of clone 61220012

SEQ ID NO:1115 is the determined cDNA sequence of clone 61220014

SEQ ID NO:1116 is the determined cDNA sequence of clone 61220015

SEQ ID NO:1117 is the determined cDNA sequence of clone 61220016

SEQ ID NO:1118 is the determined cDNA sequence of clone 61220017

SEQ ID NO:1119 is the determined cDNA sequence of clone 61220018

SEQ ID NO:1120 is the determined cDNA sequence of clone 61220020

SEQ ID NO:1121 is the determined cDNA sequence of clone 61220022

SEQ ID NO:1122 is the determined cDNA sequence of clone 61220023

SEQ ID NO:1123 is the determined cDNA sequence of clone 61220025

SEQ ID NO:1124 is the determined cDNA sequence of clone 61220026

SEQ ID NO:1125 is the determined cDNA sequence of clone 61220027

SEQ ID NO:1126 is the determined cDNA sequence of clone 61220028

SEQ ID NO:1127 is the determined cDNA sequence of clone 61220029

SEQ ID NO:1128 is the determined cDNA sequence of clone 61220032

SEQ ID NO:1129 is the determined cDNA sequence of clone 61220033

SEQ ID NO:1130 is the determined cDNA sequence of clone 61220034

SEQ ID NO:1131 is the determined cDNA sequence of clone 61220035

SEQ ID NO:1132 is the determined cDNA sequence of clone 61220036

SEQ ID NO:1133 is the determined cDNA sequence of clone 61220037

SEQ ID NO:1134 is the determined cDNA sequence of clone 61220038

SEQ ID NO:1135 is the determined cDNA sequence of clone 61220040

SEQ ID NO:1136 is the determined cDNA sequence of clone 61220043

SEQ ID NO:1137 is the determined cDNA sequence of clone 61220044

SEQ ID NO:1138 is the determined cDNA sequence of clone 61220045

SEQ ID NO:1139 is the determined cDNA sequence of clone 61220046

SEQ ID NO:1140 is the determined cDNA sequence of clone 61220051

SEQ ID NO:1141 is the determined cDNA sequence of clone 61220052

SEQ ID NO:1142 is the determined cDNA sequence of clone 61220053

SEQ ID NO:1143 is the determined cDNA sequence of clone 61220054

SEQ ID NO:1144 is the determined cDNA sequence of clone 61220055

SEQ ID NO:1145 is the determined cDNA sequence of clone 61546633

SEQ ID NO:1146 is the determined cDNA sequence of clone 61546634

SEQ ID NO:1147 is the determined cDNA sequence of clone 61546635

SEQ ID NO:1148 is the determined cDNA sequence of clone 61546636

SEQ ID NO:1149 is the determined cDNA sequence of clone 61546637

SEQ ID NO:1150 is the determined cDNA sequence of clone 61546639

SEQ ID NO:1151 is the determined cDNA sequence of clone 61546642

SEQ ID NO:1152 is the determined cDNA sequence of clone 61546643

SEQ ID NO:1153 is the determined cDNA sequence of clone 61546644

SEQ ID NO:1154 is the determined cDNA sequence of clone 61546645

SEQ ID NO:1155 is the determined cDNA sequence of clone 61546646

SEQ ID NO:1156 is the determined cDNA sequence of clone 61546647

SEQ ID NO:1157 is the determined cDNA sequence of clone 61546648

SEQ ID NO:1158 is the determined cDNA sequence of clone 61546649

SEQ ID NO:1159 is the determined cDNA sequence of clone 61546650

SEQ ID NO:1160 is the determined cDNA sequence of clone 61546651

SEQ ID NO:1161 is the determined cDNA sequence of clone 61546652

SEQ ID NO:1162 is the determined cDNA sequence of clone 61546653

SEQ ID NO:1163 is the determined cDNA sequence of clone 61546654

SEQ ID NO:1164 is the determined cDNA sequence of clone 61546656

SEQ ID NO:1165 is the determined cDNA sequence of clone 61546657

SEQ ID NO:1166 is the determined cDNA sequence of clone 61546658

SEQ ID NO:1167 is the determined cDNA sequence of clone 61546659

SEQ ID NO:1168 is the determined cDNA sequence of clone 61546660

SEQ ID NO:1169 is the determined cDNA sequence of clone 61546661

SEQ ID NO:1170 is the determined cDNA sequence of clone 61546662

SEQ ID NO:1171 is the determined cDNA sequence of clone 61546663

SEQ ID NO:1172 is the determined cDNA sequence of clone 61546664

SEQ ID NO:1173 is the determined cDNA sequence of clone 61546665

SEQ ID NO:1174 is the determined cDNA sequence of clone 61546667

SEQ ID NO:1175 is the determined cDNA sequence of clone 61546668

SEQ ID NO:1176 is the determined cDNA sequence of clone 61546669

SEQ ID NO:1177 is the determined cDNA sequence of clone 61546670

SEQ ID NO:1178 is the determined cDNA sequence of clone 61546671

SEQ ID NO:1179 is the determined cDNA sequence of clone 61546672

SEQ ID NO:1180 is the determined cDNA sequence of clone 61546674

SEQ ID NO:1181 is the determined cDNA sequence of clone 61546675

SEQ ID NO:1182 is the determined cDNA sequence of clone 61546676

SEQ ID NO:1183 is the determined cDNA sequence of clone 61546677

SEQ ID NO:1184 is the determined cDNA sequence of clone 61546679

SEQ ID NO:1185 is the determined cDNA sequence of clone 61546680

SEQ ID NO:1186 is the determined cDNA sequence of clone 61546682

SEQ ID NO:1187 is the determined cDNA sequence of clone 61546683

SEQ ID NO:1188 is the determined cDNA sequence of clone 61546684

SEQ ID NO:1189 is the determined cDNA sequence of clone 61546685

SEQ ID NO:1190 is the determined cDNA sequence of clone 61546686

SEQ ID NO:1191 is the determined cDNA sequence of clone 61546687

SEQ ID NO:1192 is the determined cDNA sequence of clone 61546688

SEQ ID NO:1193 is the determined cDNA sequence of clone 61546689

SEQ ID NO:1194 is the determined cDNA sequence of clone 61546690

SEQ ID NO:1195 is the determined cDNA sequence of clone 61546691

SEQ ID NO:1196 is the determined cDNA sequence of clone 61546692

SEQ ID NO:1197 is the determined cDNA sequence of clone 61546693

SEQ ID NO:1198 is the determined cDNA sequence of clone 61546694

SEQ ID NO:1199 is the determined cDNA sequence of clone 61546695

SEQ ID NO:1200 is the determined cDNA sequence of clone 61546697

SEQ ID NO:1201 is the determined cDNA sequence of clone 61546698

SEQ ID NO:1202 is the determined cDNA sequence of clone 61546699

SEQ ID NO:1203 is the determined cDNA sequence of clone 61546700

SEQ ID NO:1204 is the determined cDNA sequence of clone 61546701

SEQ ID NO:1205 is the determined cDNA sequence of clone 61546702

SEQ ID NO:1206 is the determined cDNA sequence of clone 61546703

SEQ ID NO:1207 is the determined cDNA sequence of clone 61546704

SEQ ID NO:1208 is the determined cDNA sequence of clone 61546705

SEQ ID NO:1209 is the determined cDNA sequence of clone 61546706

SEQ ID NO:1210 is the determined cDNA sequence of clone 61546707

SEQ ID NO:1211 is the determined cDNA sequence of clone 61546708

SEQ ID NO:1212 is the determined cDNA sequence of clone 61546709

SEQ ID NO:1213 is the determined cDNA sequence of clone 61546710

SEQ ID NO:1214 is the determined cDNA sequence of clone 61546711

SEQ ID NO:1215 is the determined cDNA sequence of clone 61546712

SEQ ID NO:1216 is the determined cDNA sequence of clone 61546714

SEQ ID NO:1217 is the determined cDNA sequence of clone 61546716

SEQ ID NO:1218 is the determined cDNA sequence of clone 61546718

SEQ ID NO:1219 is the determined cDNA sequence of clone 61546719

SEQ ID NO:1220 is the determined cDNA sequence of clone 61546720

SEQ ID NO:1221 is the determined cDNA sequence of clone 61546722

SEQ ID NO:1222 is the determined cDNA sequence of clone 61546724

SEQ ID NO:1223 is the determined cDNA sequence of clone 61220428

SEQ ID NO:1224 is the determined cDNA sequence of clone 61220430

SEQ ID NO:1225 is the determined cDNA sequence of clone 61220431

SEQ ID NO:1226 is the determined cDNA sequence of clone 61220432

SEQ ID NO:1227 is the determined cDNA sequence of clone 61220433

SEQ ID NO:1228 is the determined cDNA sequence of clone 61220434

SEQ ID NO:1229 is the determined cDNA sequence of clone 61220435

SEQ ID NO:1230 is the determined cDNA sequence of clone 61220436

SEQ ID NO:1231 is the determined cDNA sequence of clone 61220437

SEQ ID NO:1232 is the determined cDNA sequence of clone 61220439

SEQ ID NO:1233 is the determined cDNA sequence of clone 61220440

SEQ ID NO:1234 is the determined cDNA sequence of clone 61220441

SEQ ID NO:1235 is the determined cDNA sequence of clone 61220442

SEQ ID NO:1236 is the determined cDNA sequence of clone 61220443

SEQ ID NO:1237 is the determined cDNA sequence of clone 61220444

SEQ ID NO:1238 is the determined cDNA sequence of clone 61220445

SEQ ID NO:1239 is the determined cDNA sequence of clone 61220446

SEQ ID NO:1240 is the determined cDNA sequence of clone 61220447

SEQ ID NO:1241 is the determined cDNA sequence of clone 61220448

SEQ ID NO:1242 is the determined cDNA sequence of clone 61220452

SEQ ID NO:1243 is the determined cDNA sequence of clone 61220453

SEQ ID NO:1244 is the determined cDNA sequence of clone 61220454

SEQ ID NO:1245 is the determined cDNA sequence of clone 61220455

SEQ ID NO:1246 is the determined cDNA sequence of clone 61220456

SEQ ID NO:1247 is the determined cDNA sequence of clone 61220457

SEQ ID NO:1248 is the determined cDNA sequence of clone 61220458

SEQ ID NO:1249 is the determined cDNA sequence of clone 61220459

SEQ ID NO:1250 is the determined cDNA sequence of clone 61220460

SEQ ID NO:1251 is the determined cDNA sequence of clone 61220461

SEQ ID NO:1252 is the determined cDNA sequence of clone 61220462

SEQ ID NO:1253 is the determined cDNA sequence of clone 61220464

SEQ ID NO:1254 is the determined cDNA sequence of clone 61220465

SEQ ID NO:1255 is the determined cDNA sequence of clone 61220466

SEQ ID NO:1256 is the determined cDNA sequence of clone 61220467

SEQ ID NO:1257 is the determined cDNA sequence of clone 61220468

SEQ ID NO:1258 is the determined cDNA sequence of clone 61220469

SEQ ID NO:1259 is the determined cDNA sequence of clone 61220470

SEQ ID NO:1260 is the determined cDNA sequence of clone 61220471

SEQ ID NO:1261 is the determined cDNA sequence of clone 61220473

SEQ ID NO:1262 is the determined cDNA sequence of clone 61220474

SEQ ID NO:1263 is the determined cDNA sequence of clone 61220475

SEQ ID NO:1264 is the determined cDNA sequence of clone 61220476

SEQ ID NO:1265 is the determined cDNA sequence of clone 61220477

SEQ ID NO:1266 is the determined cDNA sequence of clone 61220478

SEQ ID NO:1267 is the determined cDNA sequence of clone 61220480

SEQ ID NO:1268 is the determined cDNA sequence of clone 61220481

SEQ ID NO:1269 is the determined cDNA sequence of clone 61220482

SEQ ID NO:1270 is the determined cDNA sequence of clone 61220483

SEQ ID NO:1271 is the determined cDNA sequence of clone 61220484

SEQ ID NO:1272 is the determined cDNA sequence of clone 61220485

SEQ ID NO:1273 is the determined cDNA sequence of clone 61220486

SEQ ID NO:1274 is the determined cDNA sequence of clone 61220487

SEQ ID NO:1275 is the determined cDNA sequence of clone 61220488

SEQ ID NO:1276 is the determined cDNA sequence of clone 61220489

SEQ ID NO:1277 is the determined cDNA sequence of clone 61220490

SEQ ID NO:1278 is the determined cDNA sequence of clone 61220491

SEQ ID NO:1279 is the determined cDNA sequence of clone 61220492

SEQ ID NO:1280 is the determined cDNA sequence of clone 61220493

SEQ ID NO:1281 is the determined cDNA sequence of clone 61220494

SEQ ID NO:1282 is the determined cDNA sequence of clone 61220495

SEQ ID NO:1283 is the determined cDNA sequence of clone 61220496

SEQ ID NO:1284 is the determined cDNA sequence of clone 61220497

SEQ ID NO:1285 is the determined cDNA sequence of clone 61220498

SEQ ID NO:1286 is the determined cDNA sequence of clone 61220499

SEQ ID NO:1287 is the determined cDNA sequence of clone 61220500

SEQ ID NO:1288 is the determined cDNA sequence of clone 61220501

SEQ ID NO:1289 is the determined cDNA sequence of clone 61220502

SEQ ID NO:1290 is the determined cDNA sequence of clone 61220503

SEQ ID NO:1291 is the determined cDNA sequence of clone 61220504

SEQ ID NO:1292 is the determined cDNA sequence of clone 61220505

SEQ ID NO:1293 is the determined cDNA sequence of clone 61220506

SEQ ID NO:1294 is the determined cDNA sequence of clone 61220507

SEQ ID NO:1295 is the determined cDNA sequence of clone 61220508

SEQ ID NO:1296 is the determined cDNA sequence of clone 61220509

SEQ ID NO:1297 is the determined cDNA sequence of clone 61220511

SEQ ID NO:1298 is the determined cDNA sequence of clone 61220512

SEQ ID NO:1299 is the determined cDNA sequence of clone 61220513

SEQ ID NO:1300 is the determined cDNA sequence of clone 61220514

SEQ ID NO:1301 is the determined cDNA sequence of clone 61220515

SEQ ID NO:1302 is the determined cDNA sequence of clone 61220516

SEQ ID NO:1303 is the determined cDNA sequence of clone 61220517

SEQ ID NO:1304 is the determined cDNA sequence of clone 61220518

SEQ ID NO:1305 is the determined cDNA sequence of clone 61220519

SEQ ID NO:1306 is the determined cDNA sequence of clone 61220520

SEQ ID NO:1307 is the determined cDNA sequence of clone 61547443

SEQ ID NO:1308 is the determined cDNA sequence of clone 61547444

SEQ ID NO:1309 is the determined cDNA sequence of clone 61547445

SEQ ID NO:1310 is the determined cDNA sequence of clone 61547446

SEQ ID NO:1311 is the determined cDNA sequence of clone 61547447

SEQ ID NO:1312 is the determined cDNA sequence of clone 61547448

SEQ ID NO:1313 is the determined cDNA sequence of clone 61547449

SEQ ID NO:1314 is the determined cDNA sequence of clone 61547451

SEQ ID NO:1315 is the determined cDNA sequence of clone 61547452

SEQ ID NO:1316 is the determined cDNA sequence of clone 61547453

SEQ ID NO:1317 is the determined cDNA sequence of clone 61547454

SEQ ID NO:1318 is the determined cDNA sequence of clone 61547455

SEQ ID NO:1319 is the determined cDNA sequence of clone 61547456

SEQ ID NO:1320 is the determined cDNA sequence of clone 61547457

SEQ ID NO:1321 is the determined cDNA sequence of clone 61547458

SEQ ID NO:1322 is the determined cDNA sequence of clone 61547459

SEQ ID NO:1323 is the determined cDNA sequence of clone 61547460

SEQ ID NO:1324 is the determined cDNA sequence of clone 61547461

SEQ ID NO:1325 is the determined cDNA sequence of clone 61547462

SEQ ID NO:1326 is the determined cDNA sequence of clone 61547463

SEQ ID NO:1327 is the determined cDNA sequence of clone 61547464

SEQ ID NO:1328 is the determined cDNA sequence of clone 61547465

SEQ ID NO:1329 is the determined cDNA sequence of clone 61547466

SEQ ID NO:1330 is the determined cDNA sequence of clone 61547467

SEQ ID NO:1331 is the determined cDNA sequence of clone 61547468

SEQ ID NO:1332 is the determined cDNA sequence of clone 61547469

SEQ ID NO:1333 is the determined cDNA sequence of clone 61547470

SEQ ID NO:1334 is the determined cDNA sequence of clone 61547471

SEQ ID NO:1335 is the determined cDNA sequence of clone 61547472

SEQ ID NO:1336 is the determined cDNA sequence of clone 61547473

SEQ ID NO:1337 is the determined cDNA sequence of clone 61547474

SEQ ID NO:1338 is the determined cDNA sequence of clone 61547475

SEQ ID NO:1339 is the determined cDNA sequence of clone 61547476

SEQ ID NO:1340 is the determined cDNA sequence of clone 61547477

SEQ ID NO:1341 is the determined cDNA sequence of clone 61547478

SEQ ID NO:1342 is the determined cDNA sequence of clone 61547479

SEQ ID NO:1343 is the determined cDNA sequence of clone 61547480

SEQ ID NO:1344 is the determined cDNA sequence of clone 61547481

SEQ ID NO:1345 is the determined cDNA sequence of clone 61547483

SEQ ID NO:1346 is the determined cDNA sequence of clone 61547484

SEQ ID NO:1347 is the determined cDNA sequence of clone 61547485

SEQ ID NO:1348 is the determined cDNA sequence of clone 61547486

SEQ ID NO:1349 is the determined cDNA sequence of clone 61547487

SEQ ID NO:1350 is the determined cDNA sequence of clone 61547488

SEQ ID NO:1351 is the determined cDNA sequence of clone 61547489

SEQ ID NO:1352 is the determined cDNA sequence of clone 61547490

SEQ ID NO:1353 is the determined cDNA sequence of clone 61547491

SEQ ID NO:1354 is the determined cDNA sequence of clone 61547492

SEQ ID NO:1355 is the determined cDNA sequence of clone 61547494

SEQ ID NO:1356 is the determined cDNA sequence of clone 61547495

SEQ ID NO:1357 is the determined cDNA sequence of clone 61547498

SEQ ID NO:1358 is the determined cDNA sequence of clone 61547499

SEQ ID NO:1359 is the determined cDNA sequence of clone 61547500

SEQ ID NO:1360 is the determined cDNA sequence of clone 61547501

SEQ ID NO:1361 is the determined cDNA sequence of clone 61547502

SEQ ID NO:1362 is the determined cDNA sequence of clone 61547503

SEQ ID NO:1363 is the determined cDNA sequence of clone 61547504

SEQ ID NO:1364 is the determined cDNA sequence of clone 61547505

SEQ ID NO:1365 is the determined cDNA sequence of clone 61547507

SEQ ID NO:1366 is the determined cDNA sequence of clone 61547508

SEQ ID NO:1367 is the determined cDNA sequence of clone 61547509

SEQ ID NO:1368 is the determined cDNA sequence of clone 61547510

SEQ ID NO:1369 is the determined cDNA sequence of clone 61547511

SEQ ID NO:1370 is the determined cDNA sequence of clone 61547512

SEQ ID NO:1371 is the determined cDNA sequence of clone 61547513

SEQ ID NO:1372 is the determined cDNA sequence of clone 61547514

SEQ ID NO:1373 is the determined cDNA sequence of clone 61547515

SEQ ID NO:1374 is the determined cDNA sequence of clone 61547516

SEQ ID NO:1375 is the determined cDNA sequence of clone 61547517

SEQ ID NO:1376 is the determined cDNA sequence of clone 61547518

SEQ ID NO:1377 is the determined cDNA sequence of clone 61547519

SEQ ID NO:1378 is the determined cDNA sequence of clone 61547520

SEQ ID NO:1379 is the determined cDNA sequence of clone 61547522

SEQ ID NO:1380 is the determined cDNA sequence of clone 61547523

SEQ ID NO:1381 is the determined cDNA sequence of clone 61547524

SEQ ID NO:1382 is the determined cDNA sequence of clone 61547525

SEQ ID NO:1383 is the determined cDNA sequence of clone 61547526

SEQ ID NO:1384 is the determined cDNA sequence of clone 61547527

SEQ ID NO:1385 is the determined cDNA sequence of clone 61547528

SEQ ID NO:1386 is the determined cDNA sequence of clone 61547529

SEQ ID NO:1387 is the determined cDNA sequence of clone 61547530

SEQ ID NO:1388 is the determined cDNA sequence of clone 61547532

SEQ ID NO:1389 is the determined cDNA sequence of clone 61547533

SEQ ID NO:1390 is the determined cDNA sequence of clone 61547534

SEQ ID NO:1391 is the determined cDNA sequence of clone 61547535

SEQ ID NO:1392 is the determined cDNA sequence of clone 61438573

SEQ ID NO:1393 is the determined cDNA sequence of clone 61438574

SEQ ID NO:1394 is the determined cDNA sequence of clone 61438575

SEQ ID NO:1395 is the determined cDNA sequence of clone 61438576

SEQ ID NO:1396 is the determined cDNA sequence of clone 61438577

SEQ ID NO:1397 is the determined cDNA sequence of clone 61438579

SEQ ID NO:1398 is the determined cDNA sequence of clone 61438581

SEQ ID NO:1399 is the determined cDNA sequence of clone 61438582

SEQ ID NO:1400 is the determined cDNA sequence of clone 61438583

SEQ ID NO:1401 is the determined cDNA sequence of clone 61438584

SEQ ID NO:1402 is the determined cDNA sequence of clone 61438585

SEQ ID NO:1403 is the determined cDNA sequence of clone 61438586

SEQ ID NO:1404 is the determined cDNA sequence of clone 61438588

SEQ ID NO:1405 is the determined cDNA sequence of clone 61438589

SEQ ID NO:1406 is the determined cDNA sequence of clone 61438590

SEQ ID NO:1407 is the determined cDNA sequence of clone 61438591

SEQ ID NO:1408 is the determined cDNA sequence of clone 61438592

SEQ ID NO:1409 is the determined cDNA sequence of clone 61438593

SEQ ID NO:1410 is the determined cDNA sequence of clone 61438594

SEQ ID NO:1411 is the determined cDNA sequence of clone 61438595

SEQ ID NO:1412 is the determined cDNA sequence of clone 61438596

SEQ ID NO:1413 is the determined cDNA sequence of clone 61438597

SEQ ID NO:1414 is the determined cDNA sequence of clone 61438598

SEQ ID NO:1415 is the determined cDNA sequence of clone 61438599

SEQ ID NO:1416 is the determined cDNA sequence of clone 61438600

SEQ ID NO:1417 is the determined cDNA sequence of clone 61438601

SEQ ID NO:1418 is the determined cDNA sequence of clone 61438602

SEQ ID NO:1419 is the determined cDNA sequence of clone 61438603

SEQ ID NO:1420 is the determined cDNA sequence of clone 61438604

SEQ ID NO:1421 is the determined cDNA sequence of clone 61438607

SEQ ID NO:1422 is the determined cDNA sequence of clone 61438608

SEQ ID NO:1423 is the determined cDNA sequence of clone 61438609

SEQ ID NO:1424 is the determined cDNA sequence of clone 61438610

SEQ ID NO:1425 is the determined cDNA sequence of clone 61438611

SEQ ID NO:1426 is the determined cDNA sequence of clone 61438612

SEQ ID NO:1427 is the determined cDNA sequence of clone 61438613

SEQ ID NO:1428 is the determined cDNA sequence of clone 61438615

SEQ ID NO:1429 is the determined cDNA sequence of clone 61438616

SEQ ID NO:1430 is the determined cDNA sequence of clone 61438617

SEQ ID NO:1431 is the determined cDNA sequence of clone 61438618

SEQ ID NO:1432 is the determined cDNA sequence of clone 61438620

SEQ ID NO:1433 is the determined cDNA sequence of clone 61438621

SEQ ID NO:1434 is the determined cDNA sequence of clone 61438622

SEQ ID NO:1435 is the determined cDNA sequence of clone 61438623

SEQ ID NO:1436 is the determined cDNA sequence of clone 61438624

SEQ ID NO:1437 is the determined cDNA sequence of clone 61438625

SEQ ID NO:1438 is the determined cDNA sequence of clone 61438626

SEQ ID NO:1439 is the determined cDNA sequence of clone 61438627

SEQ ID NO:1440 is the determined cDNA sequence of clone 61438629

SEQ ID NO:1441 is the determined cDNA sequence of clone 61438630

SEQ ID NO:1442 is the determined cDNA sequence of clone 61438631

SEQ ID NO:1443 is the determined cDNA sequence of clone 61438632

SEQ ID NO:1444 is the determined cDNA sequence of clone 61438633

SEQ ID NO:1445 is the determined cDNA sequence of clone 61438634

SEQ ID NO:1446 is the determined cDNA sequence of clone 61438635

SEQ ID NO:1447 is the determined cDNA sequence of clone 61438637

SEQ ID NO:1448 is the determined cDNA sequence of clone 61438638

SEQ ID NO:1449 is the determined cDNA sequence of clone 61438639

SEQ ID NO:1450 is the determined cDNA sequence of clone 61438640

SEQ ID NO:1451 is the determined cDNA sequence of clone 61438641

SEQ ID NO:1452 is the determined cDNA sequence of clone 61438642

SEQ ID NO:1453 is the determined cDNA sequence of clone 61438643

SEQ ID NO:1454 is the determined cDNA sequence of clone 61438644

SEQ ID NO:1455 is the determined cDNA sequence of clone 61438645

SEQ ID NO:1456 is the determined cDNA sequence of clone 61438646

SEQ ID NO:1457 is the determined cDNA sequence of clone 61438647

SEQ ID NO:1458 is the determined cDNA sequence of clone 61438648

SEQ ID NO:1459 is the determined cDNA sequence of clone 61438649

SEQ ID NO:1460 is the determined cDNA sequence of clone 61438650

SEQ ID NO:1461 is the determined cDNA sequence of clone 61438651

SEQ ID NO:1462 is the determined cDNA sequence of clone 61438652

SEQ ID NO:1463 is the determined cDNA sequence of clone 61438653

SEQ ID NO:1464 is the determined cDNA sequence of clone 61438654

SEQ ID NO:1465 is the determined cDNA sequence of clone 61438655

SEQ ID NO:1466 is the determined cDNA sequence of clone 61438656

SEQ ID NO:1467 is the determined cDNA sequence of clone 61438657

SEQ ID NO:1468 is the determined cDNA sequence of clone 61438658

SEQ ID NO:1469 is the determined cDNA sequence of clone 61438659

SEQ ID NO:1470 is the determined cDNA sequence of clone 61438660

SEQ ID NO:1471 is the determined cDNA sequence of clone 61438661

SEQ ID NO:1472 is the determined cDNA sequence of clone 61438662

SEQ ID NO:1473 is the determined cDNA sequence of clone 61438663

SEQ ID NO:1474 is the determined cDNA sequence of clone 61438664

SEQ ID NO:1475 is the determined cDNA sequence of clone 61438665

SEQ ID NO:1476 is the determined cDNA sequence of clone 61165980

SEQ ID NO:1477 is the determined cDNA sequence of clone 61165981

SEQ ID NO:1478 is the determined cDNA sequence of clone 61165982

SEQ ID NO:1479 is the determined cDNA sequence of clone 61165983

SEQ ID NO:1480 is the determined cDNA sequence of clone 61165984

SEQ ID NO:1481 is the determined cDNA sequence of clone 61165985

SEQ ID NO:1482 is the determined cDNA sequence of clone 61165986

SEQ ID NO:1483 is the determined cDNA sequence of clone 61165987

SEQ ID NO:1484 is the determined cDNA sequence of clone 61165988

SEQ ID NO:1485 is the determined cDNA sequence of clone 61165989

SEQ ID NO:1486 is the determined cDNA sequence of clone 61165990

SEQ ID NO:1487 is the determined cDNA sequence of clone 61165991

SEQ ID NO:1488 is the determined cDNA sequence of clone 61165992

SEQ ID NO:1489 is the determined cDNA sequence of clone 61165994

SEQ ID NO:1490 is the determined cDNA sequence of clone 61165995

SEQ ID NO:1491 is the determined cDNA sequence of clone 61165996

SEQ ID NO:1492 is the determined cDNA sequence of clone 61165997

SEQ ID NO:1493 is the determined cDNA sequence of clone 61165998

SEQ ID NO:1494 is the determined cDNA sequence of clone 61165999

SEQ ID NO:1495 is the determined cDNA sequence of clone 61166001

SEQ ID NO:1496 is the determined cDNA sequence of clone 61166002

SEQ ID NO:1497 is the determined cDNA sequence of clone 61166003

SEQ ID NO:1498 is the determined cDNA sequence of clone 61166004

SEQ ID NO:1499 is the determined cDNA sequence of clone 61166005

SEQ ID NO:1500 is the determined cDNA sequence of clone 61166006

SEQ ID NO:1501 is the determined cDNA sequence of clone 61166007

SEQ ID NO:1502 is the determined cDNA sequence of clone 61166008

SEQ ID NO:1503 is the determined cDNA sequence of clone 61166009

SEQ ID NO:1504 is the determined cDNA sequence of clone 61166010

SEQ ID NO:1505 is the determined cDNA sequence of clone 61166012

SEQ ID NO:1506 is the determined cDNA sequence of clone 61166014

SEQ ID NO:1507 is the determined cDNA sequence of clone 61166015

SEQ ID NO:1508 is the determined cDNA sequence of clone 61166016

SEQ ID NO:1509 is the determined cDNA sequence of clone 61166017

SEQ ID NO:1510 is the determined cDNA sequence of clone 61166018

SEQ ID NO:1511 is the determined cDNA sequence of clone 61166019

SEQ ID NO:1512 is the determined cDNA sequence of clone 61166020

SEQ ID NO:1513 is the determined cDNA sequence of clone 61166021

SEQ ID NO:1514 is the determined cDNA sequence of clone 61166022

SEQ ID NO:1515 is the determined cDNA sequence of clone 61166023

SEQ ID NO:1516 is the determined cDNA sequence of clone 61166024

SEQ ID NO:1517 is the determined cDNA sequence of clone 61166025

SEQ ID NO:1518 is the determined cDNA sequence of clone 61166027

SEQ ID NO:1519 is the determined cDNA sequence of clone 61166028

SEQ ID NO:1520 is the determined cDNA sequence of clone 61166029

SEQ ID NO:1521 is the determined cDNA sequence of clone 61166030

SEQ ID NO:1522 is the determined cDNA sequence of clone 61166031

SEQ ID NO:1523 is the determined cDNA sequence of clone 61166032

SEQ ID NO:1524 is the determined cDNA sequence of clone 61166033

SEQ ID NO:1525 is the determined cDNA sequence of clone 61166035

SEQ ID NO:1526 is the determined cDNA sequence of clone 61166036

SEQ ID NO:1527 is the determined cDNA sequence of clone 61166039

SEQ ID NO:1528 is the determined cDNA sequence of clone 61166040

SEQ ID NO:1529 is the determined cDNA sequence of clone 61166041

SEQ ID NO:1530 is the determined cDNA sequence of clone 61166042

SEQ ID NO:1531 is the determined cDNA sequence of clone 61166043

SEQ ID NO:1532 is the determined cDNA sequence of clone 61166044

SEQ ID NO:1533 is the determined cDNA sequence of clone 61166045

SEQ ID NO:1534 is the determined cDNA sequence of clone 61166047

SEQ ID NO:1535 is the determined cDNA sequence of clone 61166048

SEQ ID NO:1536 is the determined cDNA sequence of clone 61166049

SEQ ID NO:1537 is the determined cDNA sequence of clone 61166050

SEQ ID NO:1538 is the determined cDNA sequence of clone 61166053

SEQ ID NO:1539 is the determined cDNA sequence of clone 61166054

SEQ ID NO:1540 is the determined cDNA sequence of clone 61166055

SEQ ID NO:1541 is the determined cDNA sequence of clone 61166056

SEQ ID NO:1542 is the determined cDNA sequence of clone 61166057

SEQ ID NO:1543 is the determined cDNA sequence of clone 61166058

SEQ ID NO:1544 is the determined cDNA sequence of clone 61166059

SEQ ID NO:1545 is the determined cDNA sequence of clone 61166060

SEQ ID NO:1546 is the determined cDNA sequence of clone 61166061

SEQ ID NO:1547 is the determined cDNA sequence of clone 61166062

SEQ ID NO:1548 is the determined cDNA sequence of clone 61166064

SEQ ID NO:1549 is the determined cDNA sequence of clone 61166065

SEQ ID NO:1550 is the determined cDNA sequence of clone 61166066

SEQ ID NO:1551 is the determined cDNA sequence of clone 61166067

SEQ ID NO:1552 is the determined cDNA sequence of clone 61166068

SEQ ID NO:1553 is the determined cDNA sequence of clone 61166069

SEQ ID NO:1554 is the determined cDNA sequence of clone 61166071

SEQ ID NO:1555 is the determined cDNA sequence of clone 61559232

SEQ ID NO:1556 is the determined cDNA sequence of clone 61559233

SEQ ID NO:1557 is the determined cDNA sequence of clone 61559234

SEQ ID NO:1558 is the determined cDNA sequence of clone 61559235

SEQ ID NO:1559 is the determined cDNA sequence of clone 61559236

SEQ ID NO:1560 is the determined cDNA sequence of clone 61559237

SEQ ID NO:1561 is the determined cDNA sequence of clone 61559238

SEQ ID NO:1562 is the determined cDNA sequence of clone 61559239

SEQ ID NO:1563 is the determined cDNA sequence of clone 61559240

SEQ ID NO:1564 is the determined cDNA sequence of clone 61559241

SEQ ID NO:1565 is the determined cDNA sequence of clone 61559242

SEQ ID NO:1566 is the determined cDNA sequence of clone 61559243

SEQ ID NO:1567 is the determined cDNA sequence of clone 61559244

SEQ ID NO:1568 is the determined cDNA sequence of clone 61559246

SEQ ID NO:1569 is the determined cDNA sequence of clone 61559247

SEQ ID NO:1570 is the determined cDNA sequence of clone 61559248

SEQ ID NO:1571 is the determined cDNA sequence of clone 61559249

SEQ ID NO:1572 is the determined cDNA sequence of clone 61559250

SEQ ID NO:1573 is the determined cDNA sequence of clone 61559251

SEQ ID NO:1574 is the determined cDNA sequence of clone 61559254

SEQ ID NO:1575 is the determined cDNA sequence of clone 61559255

SEQ ID NO:1576 is the determined cDNA sequence of clone 61559256

SEQ ID NO:1577 is the determined cDNA sequence of clone 61559257

SEQ ID NO:1578 is the determined cDNA sequence of clone 61559258

SEQ ID NO:1579 is the determined cDNA sequence of clone 61559259

SEQ ID NO:1580 is the determined cDNA sequence of clone 61559260

SEQ ID NO:1581 is the determined cDNA sequence of clone 61559261

SEQ ID NO:1582 is the determined cDNA sequence of clone 61559262

SEQ ID NO:1583 is the determined cDNA sequence of clone 61559263

SEQ ID NO:1584 is the determined cDNA sequence of clone 61559264

SEQ ID NO:1585 is the determined cDNA sequence of clone 61559265

SEQ ID NO:1586 is the determined cDNA sequence of clone 61559266

SEQ ID NO:1587 is the determined cDNA sequence of clone 61559267

SEQ ID NO:1588 is the determined cDNA sequence of clone 61559268

SEQ ID NO:1589 is the determined cDNA sequence of clone 61559270

SEQ ID NO:1590 is the determined cDNA sequence of clone 61559271

SEQ ID NO:1591 is the determined cDNA sequence of clone 61559272

SEQ ID NO:1592 is the determined cDNA sequence of clone 61559273

SEQ ID NO:1593 is the determined cDNA sequence of clone 61559274

SEQ ID NO:1594 is the determined cDNA sequence of clone 61559275

SEQ ID NO:1595 is the determined cDNA sequence of clone 61559276

SEQ ID NO:1596 is the determined cDNA sequence of clone 61559277

SEQ ID NO:1597 is the determined cDNA sequence of clone 61559278

SEQ ID NO:1598 is the determined cDNA sequence of clone 61559279

SEQ ID NO:1599 is the determined cDNA sequence of clone 61559280

SEQ ID NO:1600 is the determined cDNA sequence of clone 61559281

SEQ ID NO:1601 is the determined cDNA sequence of clone 61559282

SEQ ID NO:1602 is the determined cDNA sequence of clone 61559283

SEQ ID NO:1603 is the determined cDNA sequence of clone 61559284

SEQ ID NO:1604 is the determined cDNA sequence of clone 61559285

SEQ ID NO:1605 is the determined cDNA sequence of clone 61559286

SEQ ID NO:1606 is the determined cDNA sequence of clone 61559287

SEQ ID NO:1607 is the determined cDNA sequence of clone 61559288

SEQ ID NO:1608 is the determined cDNA sequence of clone 61559289

SEQ ID NO:1609 is the determined cDNA sequence of clone 61559290

SEQ ID NO:1610 is the determined cDNA sequence of clone 61559291

SEQ ID NO:1611 is the determined cDNA sequence of clone 61559292

SEQ ID NO:1612 is the determined cDNA sequence of clone 61559293

SEQ ID NO:1613 is the determined cDNA sequence of clone 61559294

SEQ ID NO:1614 is the determined cDNA sequence of clone 61559295

SEQ ID NO:1615 is the determined cDNA sequence of clone 61559296

SEQ ID NO:1616 is the determined cDNA sequence of clone 61559297

SEQ ID NO:1617 is the determined cDNA sequence of clone 61559298

SEQ ID NO:1618 is the determined cDNA sequence of clone 61559299

SEQ ID NO:1619 is the determined cDNA sequence of clone 61559300

SEQ ID NO:1620 is the determined cDNA sequence of clone 61559302

SEQ ID NO:1621 is the determined cDNA sequence of clone 61559303

SEQ ID NO:1622 is the determined cDNA sequence of clone 61559304

SEQ ID NO:1623 is the determined cDNA sequence of clone 61559305

SEQ ID NO:1624 is the determined cDNA sequence of clone 61559306

SEQ ID NO:1625 is the determined cDNA sequence of clone 61559308

SEQ ID NO:1626 is the determined cDNA sequence of clone 61559310

SEQ ID NO:1627 is the determined cDNA sequence of clone 61559311

SEQ ID NO:1628 is the determined cDNA sequence of clone 61559312

SEQ ID NO:1629 is the determined cDNA sequence of clone 61559313

SEQ ID NO:1630 is the determined cDNA sequence of clone 61559314

SEQ ID NO:1631 is the determined cDNA sequence of clone 61559315

SEQ ID NO:1632 is the determined cDNA sequence of clone 61559316

SEQ ID NO:1633 is the determined cDNA sequence of clone 61559318

SEQ ID NO:1634 is the determined cDNA sequence of clone 61559319

SEQ ID NO:1635 is the determined cDNA sequence of clone 61559320

SEQ ID NO:1636 is the determined cDNA sequence of clone 61559321

SEQ ID NO:1637 is the determined cDNA sequence of clone 61559322

SEQ ID NO:1638 is the determined cDNA sequence of clone 61559324

SEQ ID NO:1639 is the determined cDNA sequence of clone 61438387

SEQ ID NO:1640 is the determined cDNA sequence of clone 61438388

SEQ ID NO:1641 is the determined cDNA sequence of clone 61438390

SEQ ID NO:1642 is the determined cDNA sequence of clone 61438391

SEQ ID NO:1643 is the determined cDNA sequence of clone 61438394

SEQ ID NO:1644 is the determined cDNA sequence of clone 61438397

SEQ ID NO:1645 is the determined cDNA sequence of clone 61438398

SEQ ID NO:1646 is the determined cDNA sequence of clone 61438399

SEQ ID NO:1647 is the determined cDNA sequence of clone 61438400

SEQ ID NO:1648 is the determined cDNA sequence of clone 61438401

SEQ ID NO:1649 is the determined cDNA sequence of clone 61438403

SEQ ID NO:1650 is the determined cDNA sequence of clone 61438404

SEQ ID NO:1651 is the determined cDNA sequence of clone 61438405

SEQ ID NO:1652 is the determined cDNA sequence of clone 61438406

SEQ ID NO:1653 is the determined cDNA sequence of clone 61438407

SEQ ID NO:1654 is the determined cDNA sequence of clone 61438408

SEQ ID NO:1655 is the determined cDNA sequence of clone 61438409

SEQ ID NO:1656 is the determined cDNA sequence of clone 61438410

SEQ ID NO:1657 is the determined cDNA sequence of clone 61438411

SEQ ID NO:1658 is the determined cDNA sequence of clone 61438412

SEQ ID NO:1659 is the determined cDNA sequence of clone 61438414

SEQ ID NO:1660 is the determined cDNA sequence of clone 61438415

SEQ ID NO:1661 is the determined cDNA sequence of clone 61438416

SEQ ID NO:1662 is the determined cDNA sequence of clone 61438417

SEQ ID NO:1663 is the determined cDNA sequence of clone 61438418

SEQ ID NO:1664 is the determined cDNA sequence of clone 61438419

SEQ ID NO:1665 is the determined cDNA sequence of clone 61438420

SEQ ID NO:1666 is the determined cDNA sequence of clone 61438421

SEQ ID NO:1667 is the determined cDNA sequence of clone 61438422

SEQ ID NO:1668 is the determined cDNA sequence of clone 61438423

SEQ ID NO:1669 is the determined cDNA sequence of clone 61438424

SEQ ID NO:1670 is the determined cDNA sequence of clone 61438425

SEQ ID NO:1671 is the determined cDNA sequence of clone 61438426

SEQ ID NO:1672 is the determined cDNA sequence of clone 61438427

SEQ ID NO:1673 is the determined cDNA sequence of clone 61438428

SEQ ID NO:1674 is the determined cDNA sequence of clone 61438429

SEQ ID NO:1675 is the determined cDNA sequence of clone 61438430

SEQ ID NO:1676 is the determined cDNA sequence of clone 61438431

SEQ ID NO:1677 is the determined cDNA sequence of clone 61438432

SEQ ID NO:1678 is the determined cDNA sequence of clone 61438435

SEQ ID NO:1679 is the determined cDNA sequence of clone 61438436

SEQ ID NO:1680 is the determined cDNA sequence of clone 61438437

SEQ ID NO:1681 is the determined cDNA sequence of clone 61438438

SEQ ID NO:1682 is the determined cDNA sequence of clone 61438439

SEQ ID NO:1683 is the determined cDNA sequence of clone 61438440

SEQ ID NO:1684 is the determined cDNA sequence of clone 61438441

SEQ ID NO:1685 is the determined cDNA sequence of clone 61438442

SEQ ID NO:1686 is the determined cDNA sequence of clone 61438443

SEQ ID NO:1687 is the determined cDNA sequence of clone 61438444

SEQ ID NO:1688 is the determined cDNA sequence of clone 61438446

SEQ ID NO:1689 is the determined cDNA sequence of clone 61438448

SEQ ID NO:1690 is the determined cDNA sequence of clone 61438449

SEQ ID NO:1691 is the determined cDNA sequence of clone 61438450

SEQ ID NO:1692 is the determined cDNA sequence of clone 61438451

SEQ ID NO:1693 is the determined cDNA sequence of clone 61438452

SEQ ID NO:1694 is the determined cDNA sequence of clone 61438453

SEQ ID NO:1695 is the determined cDNA sequence of clone 61438454

SEQ ID NO:1696 is the determined cDNA sequence of clone 61438455

SEQ ID NO:1697 is the determined cDNA sequence of clone 61438456

SEQ ID NO:1698 is the determined cDNA sequence of clone 61438458

SEQ ID NO:1699 is the determined cDNA sequence of clone 61438459

SEQ ID NO:1700 is the determined cDNA sequence of clone 61438463

SEQ ID NO:1701 is the determined cDNA sequence of clone 61438465

SEQ ID NO:1702 is the determined cDNA sequence of clone 61438466

SEQ ID NO:1703 is the determined cDNA sequence of clone 61438467

SEQ ID NO:1704 is the determined cDNA sequence of clone 61438468

SEQ ID NO:1705 is the determined cDNA sequence of clone 61438469

SEQ ID NO:1706 is the determined cDNA sequence of clone 61438470

SEQ ID NO:1707 is the determined cDNA sequence of clone 61438471

SEQ ID NO:1708 is the determined cDNA sequence of clone 61438472

SEQ ID NO:1709 is the determined cDNA sequence of clone 61438474

SEQ ID NO:1710 is the determined cDNA sequence of clone 61438475

SEQ ID NO:1711 is the determined cDNA sequence of clone 61438476

SEQ ID NO:1712 is the determined cDNA sequence of clone 61438478

SEQ ID NO:1713 is the determined cDNA sequence of clone 61438479

SEQ ID NO:1714 is the determined cDNA sequence of clone 61524872

SEQ ID NO:1715 is the determined cDNA sequence of clone 61524873

SEQ ID NO:1716 is the determined cDNA sequence of clone 61524874

SEQ ID NO:1717 is the determined cDNA sequence of clone 61524876

SEQ ID NO:1718 is the determined cDNA sequence of clone 61524877

SEQ ID NO:1719 is the determined cDNA sequence of clone 61524878

SEQ ID NO:1720 is the determined cDNA sequence of clone 61524879

SEQ ID NO:1721 is the determined cDNA sequence of clone 61524880

SEQ ID NO:1722 is the determined cDNA sequence of clone 61524881

SEQ ID NO:1723 is the determined cDNA sequence of clone 61524882

SEQ ID NO:1724 is the determined cDNA sequence of clone 61524883

SEQ ID NO:1725 is the determined cDNA sequence of clone 61524884

SEQ ID NO:1726 is the determined cDNA sequence of clone 61524886

SEQ ID NO:1727 is the determined cDNA sequence of clone 61524887

SEQ ID NO:1728 is the determined cDNA sequence of clone 61524888

SEQ ID NO:1729 is the determined cDNA sequence of clone 61524889

SEQ ID NO:1730 is the determined cDNA sequence of clone 61524890

SEQ ID NO:1731 is the determined cDNA sequence of clone 61524891

SEQ ID NO:1732 is the determined cDNA sequence of clone 61524892

SEQ ID NO:1733 is the determined cDNA sequence of clone 61524893

SEQ ID NO:1734 is the determined cDNA sequence of clone 61524894

SEQ ID NO:1735 is the determined cDNA sequence of clone 61524895

SEQ ID NO:1736 is the determined cDNA sequence of clone 61524897

SEQ ID NO:1737 is the determined cDNA sequence of clone 61524898

SEQ ID NO:1738 is the determined cDNA sequence of clone 61524899

SEQ ID NO:1739 is the determined cDNA sequence of clone 61524901

SEQ ID NO:1740 is the determined cDNA sequence of clone 61524902

SEQ ID NO:1741 is the determined cDNA sequence of clone 61524903

SEQ ID NO:1742 is the determined cDNA sequence of clone 61524904

SEQ ID NO:1743 is the determined cDNA sequence of clone 61524905

SEQ ID NO:1744 is the determined cDNA sequence of clone 61524906

SEQ ID NO:1745 is the determined cDNA sequence of clone 61524908

SEQ ID NO:1746 is the determined cDNA sequence of clone 61524909

SEQ ID NO:1747 is the determined cDNA sequence of clone 61524910

SEQ ID NO:1748 is the determined cDNA sequence of clone 61524912

SEQ ID NO:1749 is the determined cDNA sequence of clone 61524913

SEQ ID NO:1750 is the determined cDNA sequence of clone 61524914

SEQ ID NO:1751 is the determined cDNA sequence of clone 61524915

SEQ ID NO:1752 is the determined cDNA sequence of clone 61524916

SEQ ID NO:1753 is the determined cDNA sequence of clone 61524918

SEQ ID NO:1754 is the determined cDNA sequence of clone 61524919

SEQ ID NO:1755 is the determined cDNA sequence of clone 61524920

SEQ ID NO:1756 is the determined cDNA sequence of clone 61524923

SEQ ID NO:1757 is the determined cDNA sequence of clone 61524924

SEQ ID NO:1758 is the determined cDNA sequence of clone 61524925

SEQ ID NO:1759 is the determined cDNA sequence of clone 61524926

SEQ ID NO:1760 is the determined cDNA sequence of clone 61524927

SEQ ID NO:1761 is the determined cDNA sequence of clone 61524933

SEQ ID NO:1762 is the determined cDNA sequence of clone 61524935

SEQ ID NO:1763 is the determined cDNA sequence of clone 61524937

SEQ ID NO: 1764 is the determined cDNA sequence of clone 61524939

SEQ ID NO:1765 is the determined cDNA sequence of clone 61524941

SEQ ID NO:1766 is the determined cDNA sequence of clone 61524942

SEQ ID NO:1767 is the determined cDNA sequence of clone 61524943

SEQ ID NO:1768 is the determined cDNA sequence of clone 61524944

SEQ ID NO:1769 is the determined cDNA sequence of clone 61524945

SEQ ID NO:1770 is the determined cDNA sequence of clone 61524946

SEQ ID NO:1771 is the determined cDNA sequence of clone 61524947

SEQ ID NO:1772 is the determined cDNA sequence of clone 61524948

SEQ ID NO:1773 is the determined cDNA sequence of clone 61524949

SEQ ID NO:1774 is the determined cDNA sequence of clone 61524950

SEQ ID NO:1775 is the determined cDNA sequence of clone 61524952

SEQ ID NO:1776 is the determined cDNA sequence of clone 61524953

SEQ ID NO:1777 is the determined cDNA sequence of clone 61524954

SEQ ID NO:1778 is the determined cDNA sequence of clone 61524956

SEQ ID NO:1779 is the determined cDNA sequence of clone 61524957

SEQ ID NO:1780 is the determined cDNA sequence of clone 61524958

SEQ ID NO:1781 is the determined cDNA sequence of clone 61524961

SEQ ID NO:1782 is the determined cDNA sequence of clone 61524962

SEQ ID NO:1783 is the determined cDNA sequence of clone 61524963

SEQ ID NO:1784 is the determined cDNA sequence of clone 61524964

SEQ ID NO:1785 is the determined cDNA sequence of clone 61547629

SEQ ID NO:1786 is the determined cDNA sequence of clone 61547630

SEQ ID NO:1787 is the determined cDNA sequence of clone 61547631

SEQ ID NO:1788 is the determined cDNA sequence of clone 61547632

SEQ ID NO:1789 is the determined cDNA sequence of clone 61547634

SEQ ID NO:1790 is the determined cDNA sequence of clone 61547635

SEQ ID NO:1791 is the determined cDNA sequence of clone 61547636

SEQ ID NO:1792 is the determined cDNA sequence of clone 61547637

SEQ ID NO:1793 is the determined cDNA sequence of clone 61547638

SEQ ID NO:1794 is the determined cDNA sequence of clone 61547639

SEQ ID NO:1795 is the determined cDNA sequence of clone 61547640

SEQ ID NO:1796 is the determined cDNA sequence of clone 61547644

SEQ ID NO:1797 is the determined cDNA sequence of clone 61547646

SEQ ID NO:1798 is the determined cDNA sequence of clone 61547647

SEQ ID NO:1799 is the determined cDNA sequence of clone 61547648

SEQ ID NO:1800 is the determined cDNA sequence of clone 61547652

SEQ ID NO:1801 is the determined cDNA sequence of clone 61547653

SEQ ID NO:1802 is the determined cDNA sequence of clone 61547654

SEQ ID NO:1803 is the determined cDNA sequence of clone 61547656

SEQ ID NO:1804 is the determined cDNA sequence of clone 61547657

SEQ ID NO:1805 is the determined cDNA sequence of clone 61547658

SEQ ID NO:1806 is the determined cDNA sequence of clone 61547659

SEQ ID NO:1807 is the determined cDNA sequence of clone 61547660

SEQ ID NO:1808 is the determined cDNA sequence of clone 61547661

SEQ ID NO:1809 is the determined cDNA sequence of clone 61547662

SEQ ID NO:1810 is the determined cDNA sequence of clone 61547664

SEQ ID NO:1811 is the determined cDNA sequence of clone 61547665

SEQ ID NO:1812 is the determined cDNA sequence of clone 61547666

SEQ ID NO:1813 is the determined cDNA sequence of clone 61547667

SEQ ID NO:1814 is the determined cDNA sequence of clone 61547669

SEQ ID NO:1815 is the determined cDNA sequence of clone 61547670

SEQ ID NO:1816 is the determined cDNA sequence of clone 61547671

SEQ ID NO:1817 is the determined cDNA sequence of clone 61547672

SEQ ID NO:1818 is the determined cDNA sequence of clone 61547673

SEQ ID NO:1819 is the determined cDNA sequence of clone 61547674

SEQ ID NO:1820 is the determined cDNA sequence of clone 61547675

SEQ ID NO:1821 is the determined cDNA sequence of clone 61547678

SEQ ID NO:1822 is the determined cDNA sequence of clone 61547679

SEQ ID NO:1823 is the determined cDNA sequence of clone 61547680

SEQ ID NO:1824 is the determined cDNA sequence of clone 61547681

SEQ ID NO:1825 is the determined cDNA sequence of clone 61547682

SEQ ID NO:1826 is the determined cDNA sequence of clone 61547683

SEQ ID NO:1827 is the determined cDNA sequence of clone 61547685

SEQ ID NO:1828 is the determined cDNA sequence of clone 61547686

SEQ ID NO:1829 is the determined cDNA sequence of clone 61547687

SEQ ID NO:1830 is the determined cDNA sequence of clone 61547689

SEQ ID NO:1831 is the determined cDNA sequence of clone 61547690

SEQ ID NO:1832 is the determined cDNA sequence of clone 61547691

SEQ ID NO:1833 is the determined cDNA sequence of clone 61547692

SEQ ID NO:1834 is the determined cDNA sequence of clone 61547695

SEQ ID NO:1835 is the determined cDNA sequence of clone 61547696

SEQ ID NO:1836 is the determined cDNA sequence of clone 61547697

SEQ ID NO:1837 is the determined cDNA sequence of clone 61547698

SEQ ID NO:1838 is the determined cDNA sequence of clone 61547699

SEQ ID NO:1839 is the determined cDNA sequence of clone 61547700

SEQ ID NO:1840 is the determined cDNA sequence of clone 61547701

SEQ ID NO:1841 is the determined cDNA sequence of clone 61547702

SEQ ID NO:1842 is the determined cDNA sequence of clone 61547703

SEQ ID NO:1843 is the determined cDNA sequence of clone 61547704

SEQ ID NO:1844 is the determined cDNA sequence of clone 61547705

SEQ ID NO:1845 is the determined cDNA sequence of clone 61547706

SEQ ID NO:1846 is the determined cDNA sequence of clone 61547707

SEQ ID NO:1847 is the determined cDNA sequence of clone 61547708

SEQ ID NO:1848 is the determined cDNA sequence of clone 61547710

SEQ ID NO:1849 is the determined cDNA sequence of clone 61547711

SEQ ID NO:1850 is the determined cDNA sequence of clone 61547712

SEQ ID NO:1851 is the determined cDNA sequence of clone 61547713

SEQ ID NO:1852 is the determined cDNA sequence of clone 61547714

SEQ ID NO:1853 is the determined cDNA sequence of clone 61547716

SEQ ID NO:1854 is the determined cDNA sequence of clone 61547718

SEQ ID NO:1855 is the determined cDNA sequence of clone 61547720

SEQ ID NO:1856 is the determined cDNA sequence of clone 61547722

SEQ ID NO:1857 is the determined cDNA sequence of clone 61547724

SEQ ID NO:1858 is the determined cDNA sequence of clone 61547725

SEQ ID NO:1859 is the determined cDNA sequence of clone 61547727

SEQ ID NO:1860 is the determined cDNA sequence of clone 61547728

SEQ ID NO:1861 is the determined cDNA sequence of clone 61547729

SEQ ID NO:1862 is the determined cDNA sequence of clone 61547730

SEQ ID NO:1863 is the determined cDNA sequence of clone 61547731

SEQ ID NO:1864 is the determined cDNA sequence of clone 61547733

SEQ ID NO:1865 is the determined cDNA sequence of clone 61547734

SEQ ID NO:1866 is the determined cDNA sequence of clone 61547735

SEQ ID NO:1867 is the determined cDNA sequence of clone 61547736

SEQ ID NO:1868 is the determined cDNA sequence of clone 61547737

SEQ ID NO:1869 is the determined cDNA sequence of clone 61547738

SEQ ID NO:1870 is the determined cDNA sequence of clone 61547739

SEQ ID NO:1871 is the determined cDNA sequence of clone 61547740

SEQ ID NO:1872 is the determined cDNA sequence of clone 61547741

SEQ ID NO:1873 is the determined cDNA sequence of clone 61547742

SEQ ID NO:1874 is the determined cDNA sequence of clone 61547743

SEQ ID NO:1875 is the determined cDNA sequence of clone 61547745

SEQ ID NO:1876 is the determined cDNA sequence of clone 61547746

SEQ ID NO:1877 is the determined cDNA sequence of clone 61547747

SEQ ID NO:1878 is the determined cDNA sequence of clone 61547748

SEQ ID NO:1879 is the determined cDNA sequence of clone 61547749

SEQ ID NO:1880 is the determined cDNA sequence of clone 61547750

SEQ ID NO:1881 is the determined cDNA sequence of clone 61547753

SEQ ID NO:1882 is the determined cDNA sequence of clone 61547754

SEQ ID NO:1883 is the determined cDNA sequence of clone 61547755

SEQ ID NO:1884 is the determined cDNA sequence of clone 61547756

SEQ ID NO:1885 is the determined cDNA sequence of clone 61547757

SEQ ID NO:1886 is the determined cDNA sequence of clone 61547759

SEQ ID NO:1887 is the determined cDNA sequence of clone 61547760

SEQ ID NO:1888 is the determined cDNA sequence of clone 61547761

SEQ ID NO:1889 is the determined cDNA sequence of clone 61547763

SEQ ID NO:1890 is the determined cDNA sequence of clone 61547764

SEQ ID NO:1891 is the determined cDNA sequence of clone 61547765

SEQ ID NO:1892 is the determined cDNA sequence of clone 61547766

SEQ ID NO:1893 is the determined cDNA sequence of clone 61547767

SEQ ID NO:1894 is the determined cDNA sequence of clone 61547769

SEQ ID NO:1895 is the determined cDNA sequence of clone 61547774

SEQ ID NO:1896 is the determined cDNA sequence of clone 61547775

SEQ ID NO:1897 is the determined cDNA sequence of clone 61547776

SEQ ID NO:1898 is the determined cDNA sequence of clone 61547777

SEQ ID NO:1899 is the determined cDNA sequence of clone 61547778

SEQ ID NO:1900 is the determined cDNA sequence of clone 61547779

SEQ ID NO:1901 is the determined cDNA sequence of clone 61547782

SEQ ID NO:1902 is the determined cDNA sequence of clone 61547783

SEQ ID NO:1903 is the determined cDNA sequence of clone 61547785

SEQ ID NO:1904 is the determined cDNA sequence of clone 61547786

SEQ ID NO:1905 is the determined cDNA sequence of clone 61547787

SEQ ID NO:1906 is the determined cDNA sequence of clone 61547788

SEQ ID NO:1907 is the determined cDNA sequence of clone 61547789

SEQ ID NO:1908 is the determined cDNA sequence of clone 61547790

SEQ ID NO:1909 is the determined cDNA sequence of clone 61547791

SEQ ID NO:1910 is the determined cDNA sequence of clone 61547792

SEQ ID NO:1911 is the determined cDNA sequence of clone 61547793

SEQ ID NO:1912 is the determined cDNA sequence of clone 61547795

SEQ ID NO:1913 is the determined cDNA sequence of clone 61547796

SEQ ID NO:1914 is the determined cDNA sequence of clone 61547797

SEQ ID NO:1915 is the determined cDNA sequence of clone 61547800

SEQ ID NO:1916 is the determined cDNA sequence of clone 61547801

SEQ ID NO:1917 is the determined cDNA sequence of clone 61547804

SEQ ID NO:1918 is the determined cDNA sequence of clone 61547805

SEQ ID NO:1919 is the determined cDNA sequence of clone 61547806

SEQ ID NO:1920 is the determined cDNA sequence of clone 61547808

SEQ ID NO:1921 is the determined cDNA sequence of clone 61547809

SEQ ID NO:1922 is the determined cDNA sequence of clone 61547810

SEQ ID NO:1923 is the determined cDNA sequence of clone 61547811

SEQ ID NO:1924 is the determined cDNA sequence of clone 61547812

SEQ ID NO:1925 is the determined cDNA sequence of clone 61547813

SEQ ID NO:1926 is the determined cDNA sequence of clone 61547814

SEQ ID NO:1927 is the determined cDNA sequence of clone 61496272

SEQ ID NO:1928 is the determined cDNA sequence of clone 61496273

SEQ ID NO:1929 is the determined cDNA sequence of clone 61496274

SEQ ID NO:1930 is the determined cDNA sequence of clone 61496275

SEQ ID NO:1931 is the determined cDNA sequence of clone 61496276

SEQ ID NO:1932 is the determined cDNA sequence of clone 61496277

SEQ ID NO:1933 is the determined cDNA sequence of clone 61496278

SEQ ID NO:1934 is the determined cDNA sequence of clone 61496279

SEQ ID NO:1935 is the determined cDNA sequence of clone 61496280

SEQ ID NO:1936 is the determined cDNA sequence of clone 61496281

SEQ ID NO:1937 is the determined cDNA sequence of clone 61496282

SEQ ID NO:1938 is the determined cDNA sequence of clone 61496283

SEQ ID NO:1939 is the determined cDNA sequence of clone 61496285

SEQ ID NO:1940 is the determined cDNA sequence of clone 61496286

SEQ ID NO:1941 is the determined cDNA sequence of clone 61496287

SEQ ID NO:1942 is the determined cDNA sequence of clone 61496288

SEQ ID NO:1943 is the determined cDNA sequence of clone 61496289

SEQ ID NO:1944 is the determined cDNA sequence of clone 61496290

SEQ ID NO:1945 is the determined cDNA sequence of clone 61496291

SEQ ID NO:1946 is the determined cDNA sequence of clone 61496292

SEQ ID NO:1947 is the determined cDNA sequence of clone 61496293

SEQ ID NO:1948 is the determined cDNA sequence of clone 61496294

SEQ ID NO:1949 is the determined cDNA sequence of clone 61496295

SEQ ID NO:1950 is the determined cDNA sequence of clone 61496298

SEQ ID NO:1951 is the determined cDNA sequence of clone 61496299

SEQ ID NO:1952 is the determined cDNA sequence of clone 61496300

SEQ ID NO:1953 is the determined cDNA sequence of clone 61496301

SEQ ID NO:1954 is the determined cDNA sequence of clone 61496302

SEQ ID NO:1955 is the determined cDNA sequence of clone 61496303

SEQ ID NO:1956 is the determined cDNA sequence of clone 61496304

SEQ ID NO:1957 is the determined cDNA sequence of clone 61496305

SEQ ID NO:1958 is the determined cDNA sequence of clone 61496308

SEQ ID NO:1959 is the determined cDNA sequence of clone 61496309

SEQ ID NO:1960 is the determined cDNA sequence of clone 61496310

SEQ ID NO:1961 is the determined cDNA sequence of clone 61496312

SEQ ID NO:1962 is the determined cDNA sequence of clone 61496313

SEQ ID NO:1963 is the determined cDNA sequence of clone 61496314

SEQ ID NO:1964 is the determined cDNA sequence of clone 61496315

SEQ ID NO:1965 is the determined cDNA sequence of clone 61496316

SEQ ID NO:1966 is the determined cDNA sequence of clone 61496317

SEQ ID NO:1967 is the determined cDNA sequence of clone 61496318

SEQ ID NO:1968 is the determined cDNA sequence of clone 61496319

SEQ ID NO:1969 is the determined cDNA sequence of clone 61496320

SEQ ID NO:1970 is the determined cDNA sequence of clone 61496322

SEQ ID NO:1971 is the determined cDNA sequence of clone 61496323

SEQ ID NO:1972 is the determined cDNA sequence of clone 61496324

SEQ ID NO:1973 is the determined cDNA sequence of clone 61496325

SEQ ID NO:1974 is the determined cDNA sequence of clone 61496326

SEQ ID NO:1975 is the determined cDNA sequence of clone 61496327

SEQ ID NO:1976 is the determined cDNA sequence of clone 61496328

SEQ ID NO:1977 is the determined cDNA sequence of clone 61496329

SEQ ID NO:1978 is the determined cDNA sequence of clone 61496330

SEQ ID NO:1979 is the determined cDNA sequence of clone 61496331

SEQ ID NO:1980 is the determined cDNA sequence of clone 61496332

SEQ ID NO:1981 is the determined cDNA sequence of clone 61496333

SEQ ID NO:1982 is the determined cDNA sequence of clone 61496334

SEQ ID NO:1983 is the determined cDNA sequence of clone 61496335

SEQ ID NO:1984 is the determined cDNA sequence of clone 61496336

SEQ ID NO:1985 is the determined cDNA sequence of clone 61496337

SEQ ID NO:1986 is the determined cDNA sequence of clone 61496338

SEQ ID NO:1987 is the determined cDNA sequence of clone 61496340

SEQ ID NO:1988 is the determined cDNA sequence of clone 61496341

SEQ ID NO:1989 is the determined cDNA sequence of clone 61496342

SEQ ID NO:1990 is the determined cDNA sequence of clone 61496343

SEQ ID NO:1991 is the determined cDNA sequence of clone 61496345

SEQ ID NO:1992 is the determined cDNA sequence of clone 61496346

SEQ ID NO:1993 is the determined cDNA sequence of clone 61496347

SEQ ID NO:1994 is the determined cDNA sequence of clone 61496348

SEQ ID NO:1995 is the determined cDNA sequence of clone 61496349

SEQ ID NO:1996 is the determined cDNA sequence of clone 61496351

SEQ ID NO:1997 is the determined cDNA sequence of clone 61496352

SEQ ID NO:1998 is the determined cDNA sequence of clone 61496353

SEQ ID NO:1999 is the determined cDNA sequence of clone 61496355

SEQ ID NO:2000 is the determined cDNA sequence of clone 61496356

SEQ ID NO:2001 is the determined cDNA sequence of clone 61496357

SEQ ID NO:2002 is the determined cDNA sequence of clone 61496358

SEQ ID NO:2003 is the determined cDNA sequence of clone 61496359

SEQ ID NO:2004 is the determined cDNA sequence of clone 61496360

SEQ ID NO:2005 is the determined cDNA sequence of clone 61496362

SEQ ID NO:2006 is the determined cDNA sequence of clone 61496363

SEQ ID NO:2007 is the determined cDNA sequence of clone 61496364

SEQ ID NO:2008 is the determined cDNA sequence of clone 61496373

SEQ ID NO:2009 is the determined cDNA sequence of clone 61496374

SEQ ID NO:2010 is the determined cDNA sequence of clone 61496375

SEQ ID NO:2011 is the determined cDNA sequence of clone 61496376

SEQ ID NO:2012 is the determined cDNA sequence of clone 61496378

SEQ ID NO:2013 is the determined cDNA sequence of clone 61496379

SEQ ID NO:2014 is the determined cDNA sequence of clone 61496380

SEQ ID NO:2015 is the determined cDNA sequence of clone 61496381

SEQ ID NO:2016 is the determined cDNA sequence of clone 61496383

SEQ ID NO:2017 is the determined cDNA sequence of clone 61496384

SEQ ID NO:2018 is the determined cDNA sequence of clone 61496386

SEQ ID NO:2019 is the determined cDNA sequence of clone 61496388

SEQ ID NO:2020 is the determined cDNA sequence of clone 61496390

SEQ ID NO:2021 is the determined cDNA sequence of clone 61496391

SEQ ID NO:2022 is the determined cDNA sequence of clone 61496394

SEQ ID NO:2023 is the determined cDNA sequence of clone 61496395

SEQ ID NO:2024 is the determined cDNA sequence of clone 61496398

SEQ ID NO:2025 is the determined cDNA sequence of clone 61496399

SEQ ID NO:2026 is the determined cDNA sequence of clone 61496400

SEQ ID NO:2027 is the determined cDNA sequence of clone 61496401

SEQ ID NO:2028 is the determined cDNA sequence of clone 61496402

SEQ ID NO:2029 is the determined cDNA sequence of clone 61496403

SEQ ID NO:2030 is the determined cDNA sequence of clone 61496404

SEQ ID NO:2031 is the determined cDNA sequence of clone 61496405

SEQ ID NO:2032 is the determined cDNA sequence of clone 61496406

SEQ ID NO:2033 is the determined cDNA sequence of clone 61496407

SEQ ID NO:2034 is the determined cDNA sequence of clone 61496408

SEQ ID NO:2035 is the determined cDNA sequence of clone 61496410

SEQ ID NO:2036 is the determined cDNA sequence of clone 61496411

SEQ ID NO:2037 is the determined cDNA sequence of clone 61496412

SEQ ID NO:2038 is the determined cDNA sequence of clone 61496413

SEQ ID NO:2039 is the determined cDNA sequence of clone 61496414

SEQ ID NO:2040 is the determined cDNA sequence of clone 61496416

SEQ ID NO:2041 is the determined cDNA sequence of clone 61496417

SEQ ID NO:2042 is the determined cDNA sequence of clone 61496418

SEQ ID NO:2043 is the determined cDNA sequence of clone 61496419

SEQ ID NO:2044 is the determined cDNA sequence of clone 61496420

SEQ ID NO:2045 is the determined cDNA sequence of clone 61496422

SEQ ID NO:2046 is the determined cDNA sequence of clone 61496423

SEQ ID NO:2047 is the determined cDNA sequence of clone 61496426

SEQ ID NO:2048 is the determined cDNA sequence of clone 61496427

SEQ ID NO:2049 is the determined cDNA sequence of clone 61496429

SEQ ID NO:2050 is the determined cDNA sequence of clone 61496430

SEQ ID NO:2051 is the determined cDNA sequence of clone 61496431

SEQ ID NO:2052 is the determined cDNA sequence of clone 61496432

SEQ ID NO:2053 is the determined cDNA sequence of clone 61496435

SEQ ID NO:2054 is the determined cDNA sequence of clone 61496436

SEQ ID NO:2055 is the determined cDNA sequence of clone 61496437

SEQ ID NO:2056 is the determined cDNA sequence of clone 61496438

SEQ ID NO:2057 is the determined cDNA sequence of clone 61496439

SEQ ID NO:2058 is the determined cDNA sequence of clone 61496440

SEQ ID NO:2059 is the determined cDNA sequence of clone 61496441

SEQ ID NO:2060 is the determined cDNA sequence of clone 61496442

SEQ ID NO:2061 is the determined cDNA sequence of clone 61496443

SEQ ID NO:2062 is the determined cDNA sequence of clone 61496444

SEQ ID NO:2063 is the determined cDNA sequence of clone 61496445

SEQ ID NO:2064 is the determined cDNA sequence of clone 61496446

SEQ ID NO:2065 is the determined cDNA sequence of clone 61496447

SEQ ID NO:2066 is the determined cDNA sequence of clone 61496449

SEQ ID NO:2067 is the determined cDNA sequence of clone 61496451

SEQ ID NO:2068 is the determined cDNA sequence of clone 61496452

SEQ ID NO:2069 is the determined cDNA sequence of clone 61496454

SEQ ID NO:2070 is the determined cDNA sequence of clone 61496455

SEQ ID NO:2071 is the determined cDNA sequence of clone 61496456

SEQ ID NO:2072 is the determined cDNA sequence of clone 61496458

SEQ ID NO:2073 is the determined cDNA sequence of clone 61496459

SEQ ID NO:2074 is the determined cDNA sequence of clone 61496461

SEQ ID NO:2075 is the determined cDNA sequence of clone 61496463

SEQ ID NO:2076 is the determined cDNA sequence of clone 61496464

SEQ ID NO:2077 is the determined cDNA sequence of clone 61496465

SEQ ID NO:2078 is the determined cDNA sequence of clone 61438480

SEQ ID NO:2079 is the determined cDNA sequence of clone 61438482

SEQ ID NO:2080 is the determined cDNA sequence of clone 61438484

SEQ ID NO:2081 is the determined cDNA sequence of clone 61438485

SEQ ID NO:2082 is the determined cDNA sequence of clone 61438486

SEQ ID NO:2083 is the determined cDNA sequence of clone 61438488

SEQ ID NO:2084 is the determined cDNA sequence of clone 61438489

SEQ ID NO:2085 is the determined cDNA sequence of clone 61438492

SEQ ID NO:2086 is the determined cDNA sequence of clone 61438493

SEQ ID NO:2087 is the determined cDNA sequence of clone 61438495

SEQ ID NO:2088 is the determined cDNA sequence of clone 61438496

SEQ ID NO:2089 is the determined cDNA sequence of clone 61438497

SEQ ID NO:2090 is the determined cDNA sequence of clone 61438500

SEQ ID NO:2091 is the determined cDNA sequence of clone 61438501

SEQ ID NO:2092 is the determined cDNA sequence of clone 61438503

SEQ ID NO:2093 is the determined cDNA sequence of clone 61438505

SEQ ID NO:2094 is the determined cDNA sequence of clone 61438506

SEQ ID NO:2095 is the determined cDNA sequence of clone 61438507

SEQ ID NO:2096 is the determined cDNA sequence of clone 61438508

SEQ ID NO:2097 is the determined cDNA sequence of clone 61438509

SEQ ID NO:2098 is the determined cDNA sequence of clone 61438510

SEQ ID NO:2099 is the determined cDNA sequence of clone 61438512

SEQ ID NO:2100 is the determined cDNA sequence of clone 61438513

SEQ ID NO:2101 is the determined cDNA sequence of clone 61438514

SEQ ID NO:2102 is the determined cDNA sequence of clone 61438516

SEQ ID NO:2103 is the determined cDNA sequence of clone 61438518

SEQ ID NO:2104 is the determined cDNA sequence of clone 61438519

SEQ ID NO:2105 is the determined cDNA sequence of clone 61438520

SEQ ID NO:2106 is the determined cDNA sequence of clone 61438521

SEQ ID NO:2107 is the determined cDNA sequence of clone 61438522

SEQ ID NO:2108 is the determined cDNA sequence of clone 61438523

SEQ ID NO:2109 is the determined cDNA sequence of clone 61438524

SEQ ID NO:2110 is the determined cDNA sequence of clone 61438525

SEQ ID NO:2111 is the determined cDNA sequence of clone 61438527

SEQ ID NO:2112 is the determined cDNA sequence of clone 61438528

SEQ ID NO:2113 is the determined cDNA sequence of clone 61438530

SEQ ID NO:2114 is the determined cDNA sequence of clone 61438531

SEQ ID NO:2115 is the determined cDNA sequence of clone 61438533

SEQ ID NO:2116 is the determined cDNA sequence of clone 61438535

SEQ ID NO:2117 is the determined cDNA sequence of clone 61438536

SEQ ID NO:2118 is the determined cDNA sequence of clone 61438537

SEQ ID NO:2119 is the determined cDNA sequence of clone 61438538

SEQ ID NO:2120 is the determined cDNA sequence of clone 61438539

SEQ ID NO:2121 is the determined cDNA sequence of clone 61438540

SEQ ID NO:2122 is the determined cDNA sequence of clone 61438543

SEQ ID NO:2123 is the determined cDNA sequence of clone 61438544

SEQ ID NO:2124 is the determined cDNA sequence of clone 61438545

SEQ ID NO:2125 is the determined cDNA sequence of clone 61438547

SEQ ID NO:2126 is the determined cDNA sequence of clone 61438550

SEQ ID NO:2127 is the determined cDNA sequence of clone 61438551

SEQ ID NO:2128 is the determined cDNA sequence of clone 61438552

SEQ ID NO:2129 is the determined cDNA sequence of clone 61438553

SEQ ID NO:2130 is the determined cDNA sequence of clone 61438554

SEQ ID NO:2131 is the determined cDNA sequence of clone 61438555

SEQ ID NO:2132 is the determined cDNA sequence of clone 61438557

SEQ ID NO:2133 is the determined cDNA sequence of clone 61438558

SEQ ID NO:2134 is the determined cDNA sequence of clone 61438559

SEQ ID NO:2135 is the determined cDNA sequence of clone 61438560

SEQ ID NO:2136 is the determined cDNA sequence of clone 61438562

SEQ ID NO:2137 is the determined cDNA sequence of clone 61438563

SEQ ID NO:2138 is the determined cDNA sequence of clone 61438564

SEQ ID NO:2139 is the determined cDNA sequence of clone 61438565

SEQ ID NO:2140 is the determined cDNA sequence of clone 61438566

SEQ ID NO:2141 is the determined cDNA sequence of clone 61438567

SEQ ID NO:2142 is the determined cDNA sequence of clone 61438568

SEQ ID NO:2143 is the determined cDNA sequence of clone 61438569

SEQ ID NO:2144 is the determined cDNA sequence of clone 61438570

SEQ ID NO:2145 is the determined cDNA sequence of clone 61438571

SEQ ID NO:2146 is the determined cDNA sequence of clone 61438572

SEQ ID NO:2147 is the determined cDNA sequence of clone 61496495

SEQ ID NO:2148 is the determined cDNA sequence of clone 61496496

SEQ ID NO:2149 is the determined cDNA sequence of clone 61496497

SEQ ID NO:2150 is the determined cDNA sequence of clone 61496498

SEQ ID NO:2151 is the determined cDNA sequence of clone 61496500

SEQ ID NO:2152 is the determined cDNA sequence of clone 61496501

SEQ ID NO:2153 is the determined cDNA sequence of clone 61496502

SEQ ID NO:2154 is the determined cDNA sequence of clone 61496505

SEQ ID NO:2155 is the determined cDNA sequence of clone 61496508

SEQ ID NO:2156 is the determined cDNA sequence of clone 61496510

SEQ ID NO:2157 is the determined cDNA sequence of clone 61496511

SEQ ID NO:2158 is the determined cDNA sequence of clone 61496512

SEQ ID NO:2159 is the determined cDNA sequence of clone 61496513

SEQ ID NO:2160 is the determined cDNA sequence of clone 61496515

SEQ ID NO:2161 is the determined cDNA sequence of clone 61496516

SEQ ID NO:2162 is the determined cDNA sequence of clone 61496517

SEQ ID NO:2163 is the determined cDNA sequence of clone 61496518

SEQ ID NO:2164 is the determined cDNA sequence of clone 61496519

SEQ ID NO:2165 is the determined cDNA sequence of clone 61496520

SEQ ID NO:2166 is the determined cDNA sequence of clone 61496521

SEQ ID NO:2167 is the determined cDNA sequence of clone 61496522

SEQ ID NO:2168 is the determined cDNA sequence of clone 61496523

SEQ ID NO:2169 is the determined cDNA sequence of clone 61496524

SEQ ID NO:2170 is the determined cDNA sequence of clone 61496526

SEQ ID NO:2171 is the determined cDNA sequence of clone 61496528

SEQ ID NO:2172 is the determined cDNA sequence of clone 61496529

SEQ ID NO:2173 is the determined cDNA sequence of clone 61496530

SEQ ID NO:2174 is the determined cDNA sequence of clone 61496532

SEQ ID NO:2175 is the determined cDNA sequence of clone 61496534

SEQ ID NO:2176 is the determined cDNA sequence of clone 61496536

SEQ ID NO:2177 is the determined cDNA sequence of clone 61496537

SEQ ID NO:2178 is the determined cDNA sequence of clone 61496539

SEQ ID NO:2179 is the determined cDNA sequence of clone 61496543

SEQ ID NO:2180 is the determined cDNA sequence of clone 61496544

SEQ ID NO:2181 is the determined cDNA sequence of clone 61496545

SEQ ID NO:2182 is the determined cDNA sequence of clone 61496546

SEQ ID NO:2183 is the determined cDNA sequence of clone 61496547

SEQ ID NO:2184 is the determined cDNA sequence of clone 61496549

SEQ ID NO:2185 is the determined cDNA sequence of clone 61496551

SEQ ID NO:2186 is the determined cDNA sequence of clone 61496552

SEQ ID NO:2187 is the determined cDNA sequence of clone 61496553

SEQ ID NO:2188 is the determined cDNA sequence of clone 61496554

SEQ ID NO:2189 is the determined cDNA sequence of clone 61496555

SEQ ID NO:2190 is the determined cDNA sequence of clone 61496556

SEQ ID NO:2191 is the determined cDNA sequence of clone 61496557

SEQ ID NO:2192 is the determined cDNA sequence of clone 61496558

SEQ ID NO:2193 is the determined cDNA sequence of clone 61496559

SEQ ID NO:2194 is the determined cDNA sequence of clone 61496560

SEQ ID NO:2195 is the determined cDNA sequence of clone 61496561

SEQ ID NO:2196 is the determined cDNA sequence of clone 61496562

SEQ ID NO:2197 is the determined cDNA sequence of clone 61496564

SEQ ID NO:2198 is the determined cDNA sequence of clone 61496565

SEQ ID NO:2199 is the determined cDNA sequence of clone 61496566

SEQ ID NO:2200 is the determined cDNA sequence of clone 61496567

SEQ ID NO:2201 is the determined cDNA sequence of clone 61496568

SEQ ID NO:2202 is the determined cDNA sequence of clone 61496569

SEQ ID NO:2203 is the determined cDNA sequence of clone 61496570

SEQ ID NO:2204 is the determined cDNA sequence of clone 61496571

SEQ ID NO:2205 is the determined cDNA sequence of clone 61496572

SEQ ID NO:2206 is the determined cDNA sequence of clone 61496573

SEQ ID NO:2207 is the determined cDNA sequence of clone 61496574

SEQ ID NO:2208 is the determined cDNA sequence of clone 61496575

SEQ ID NO:2209 is the determined cDNA sequence of clone 61496576

SEQ ID NO:2210 is the determined cDNA sequence of clone 61496577

SEQ ID NO:2211 is the determined cDNA sequence of clone 61496578

SEQ ID NO:2212 is the determined cDNA sequence of clone 61496581

SEQ ID NO:2213 is the determined cDNA sequence of clone 61496582

SEQ ID NO:2214 is the determined cDNA sequence of clone 61496583

SEQ ID NO:2215 is the determined cDNA sequence of clone 61496584

SEQ ID NO:2216 is the determined cDNA sequence of clone 61496585

SEQ ID NO:2217 is the determined cDNA sequence of clone 61496586

SEQ ID NO:2218 is the determined cDNA sequence of clone 61496587

SEQ ID NO:2219 is the determined cDNA sequence of clone 61496700

SEQ ID NO:2220 is the determined cDNA sequence of clone 61496701

SEQ ID NO:2221 is the determined cDNA sequence of clone 61496702

SEQ ID NO:2222 is the determined cDNA sequence of clone 61496704

SEQ ID NO:2223 is the determined cDNA sequence of clone 61496705

SEQ ID NO:2224 is the determined cDNA sequence of clone 61496709

SEQ ID NO:2225 is the determined cDNA sequence of clone 61496710

SEQ ID NO:2226 is the determined cDNA sequence of clone 61496713

SEQ ID NO:2227 is the determined cDNA sequence of clone 61496715

SEQ ID NO:2228 is the determined cDNA sequence of clone 61496716

SEQ ID NO:2229 is the determined cDNA sequence of clone 61496717

SEQ ID NO:2230 is the determined cDNA sequence of clone 61496718

SEQ ID NO:2231 is the determined cDNA sequence of clone 61496719

SEQ ID NO:2232 is the determined cDNA sequence of clone 61496720

SEQ ID NO:2233 is the determined cDNA sequence of clone 61496721

SEQ ID NO:2234 is the determined cDNA sequence of clone 61496722

SEQ ID NO:2235 is the determined cDNA sequence of clone 61496723

SEQ ID NO:2236 is the determined cDNA sequence of clone 61496725

SEQ ID NO:2237 is the determined cDNA sequence of clone 61496726

SEQ ID NO:2238 is the determined cDNA sequence of clone 61496727

SEQ ID NO:2239 is the determined cDNA sequence of clone 61496729

SEQ ID NO:2240 is the determined cDNA sequence of clone 61496731

SEQ ID NO:2241 is the determined cDNA sequence of clone 61496733

SEQ ID NO:2242 is the determined cDNA sequence of clone 61496735

SEQ ID NO:2243 is the determined cDNA sequence of clone 61496737

SEQ ID NO:2244 is the determined cDNA sequence of clone 61496738

SEQ ID NO:2245 is the determined cDNA sequence of clone 61496739

SEQ ID NO:2246 is the determined cDNA sequence of clone 61496740

SEQ ID NO:2247 is the determined cDNA sequence of clone 61496741

SEQ ID NO:2248 is the determined cDNA sequence of clone 61496743

SEQ ID NO:2249 is the determined cDNA sequence of clone 61496745

SEQ ID NO:2250 is the determined cDNA sequence of clone 61496746

SEQ ID NO:2251 is the determined cDNA sequence of clone 61496747

SEQ ID NO:2252 is the determined cDNA sequence of clone 61496748

SEQ ID NO:2253 is the determined cDNA sequence of clone 61496749

SEQ ID NO:2254 is the determined cDNA sequence of clone 61496750

SEQ ID NO:2255 is the determined cDNA sequence of clone 61496751

SEQ ID NO:2256 is the determined cDNA sequence of clone 61496753

SEQ ID NO:2257 is the determined cDNA sequence of clone 61496754

SEQ ID NO:2258 is the determined cDNA sequence of clone 61496756

SEQ ID NO:2259 is the determined cDNA sequence of clone 61496757

SEQ ID NO:2260 is the determined cDNA sequence of clone 61496758

SEQ ID NO:2261 is the determined cDNA sequence of clone 61496759

SEQ ID NO:2262 is the determined cDNA sequence of clone 61496761

SEQ ID NO:2263 is the determined cDNA sequence of clone 61496762

SEQ ID NO:2264 is the determined cDNA sequence of clone 61496763

SEQ ID NO:2265 is the determined cDNA sequence of clone 61496764

SEQ ID NO:2266 is the determined cDNA sequence of clone 61496765

SEQ ID NO:2267 is the determined cDNA sequence of clone 61496766

SEQ ID NO:2268 is the determined cDNA sequence of clone 61496768

SEQ ID NO:2269 is the determined cDNA sequence of clone 61496769

SEQ ID NO:2270 is the determined cDNA sequence of clone 61496770

SEQ ID NO:2271 is the determined cDNA sequence of clone 61496771

SEQ ID NO:2272 is the determined cDNA sequence of clone 61496772

SEQ ID NO:2273 is the determined cDNA sequence of clone 61496774

SEQ ID NO:2274 is the determined cDNA sequence of clone 61496776

SEQ ID NO:2275 is the determined cDNA sequence of clone 61496777

SEQ ID NO:2276 is the determined cDNA sequence of clone 61496778

SEQ ID NO:2277 is the determined cDNA sequence of clone 61496780

SEQ ID NO:2278 is the determined cDNA sequence of clone 61496781

SEQ ID NO:2279 is the determined cDNA sequence of clone 61496782

SEQ ID NO:2280 is the determined cDNA sequence of clone 61496783

SEQ ID NO:2281 is the determined cDNA sequence of clone 61496784

SEQ ID NO:2282 is the determined cDNA sequence of clone 61496785

SEQ ID NO:2283 is the determined cDNA sequence of clone 61496786

SEQ ID NO:2284 is the determined cDNA sequence of clone 61496787

SEQ ID NO:2285 is the determined cDNA sequence of clone 61496788

SEQ ID NO:2286 is the determined cDNA sequence of clone 61496789

SEQ ID NO:2287 is the determined cDNA sequence of clone 61496790

SEQ ID NO:2288 is the determined cDNA sequence of clone 61497422

SEQ ID NO:2289 is the determined cDNA sequence of clone 61497424

SEQ ID NO:2290 is the determined cDNA sequence of clone 61497425

SEQ ID NO:2291 is the determined cDNA sequence of clone 61497426

SEQ ID NO:2292 is the determined cDNA sequence of clone 61497429

SEQ ID NO:2293 is the determined cDNA sequence of clone 61497430

SEQ ID NO:2294 is the determined cDNA sequence of clone 61497432

SEQ ID NO:2295 is the determined cDNA sequence of clone 61497433

SEQ ID NO:2296 is the determined cDNA sequence of clone 61497435

SEQ ID NO:2297 is the determined cDNA sequence of clone 61497436

SEQ ID NO:2298 is the determined cDNA sequence of clone 61497437

SEQ ID NO:2299 is the determined cDNA sequence of clone 61497438

SEQ ID NO:2300 is the determined cDNA sequence of clone 61497439

SEQ ID NO:2301 is the determined cDNA sequence of clone 61497440

SEQ ID NO:2302 is the determined cDNA sequence of clone 61497441

SEQ ID NO:2303 is the determined cDNA sequence of clone 61497443

SEQ ID NO:2304 is the determined cDNA sequence of clone 61497444

SEQ ID NO:2305 is the determined cDNA sequence of clone 61497446

SEQ ID NO:2306 is the determined cDNA sequence of clone 61497447

SEQ ID NO:2307 is the determined cDNA sequence of clone 61497448

SEQ ID NO:2308 is the determined cDNA sequence of clone 61497450

SEQ ID NO:2309 is the determined cDNA sequence of clone 61497451

SEQ ID NO:2310 is the determined cDNA sequence of clone 61497452

SEQ ID NO:2311 is the determined cDNA sequence of clone 61497454

SEQ ID NO:2312 is the determined cDNA sequence of clone 61497455

SEQ ID NO:2313 is the determined cDNA sequence of clone 61497456

SEQ ID NO:2314 is the determined cDNA sequence of clone 61497457

SEQ ID NO:2315 is the determined cDNA sequence of clone 61497458

SEQ ID NO:2316 is the determined cDNA sequence of clone 61497459

SEQ ID NO:2317 is the determined cDNA sequence of clone 61497460

SEQ ID NO:2318 is the determined cDNA sequence of clone 61497462

SEQ ID NO:2319 is the determined cDNA sequence of clone 61497463

SEQ ID NO:2320 is the determined cDNA sequence of clone 61497464

SEQ ID NO:2321 is the determined cDNA sequence of clone 61497466

SEQ ID NO:2322 is the determined cDNA sequence of clone 61497468

SEQ ID NO:2323 is the determined cDNA sequence of clone 61497469

SEQ ID NO:2324 is the determined cDNA sequence of clone 61497470

SEQ ID NO:2325 is the determined cDNA sequence of clone 61497471

SEQ ID NO:2326 is the determined cDNA sequence of clone 61497473

SEQ ID NO:2327 is the determined cDNA sequence of clone 61497474

SEQ ID NO:2328 is the determined cDNA sequence of clone 61497477

SEQ ID NO:2329 is the determined cDNA sequence of clone 61497478

SEQ ID NO:2330 is the determined cDNA sequence of clone 61497479

SEQ ID NO:2331 is the determined cDNA sequence of clone 61497480

SEQ ID NO:2332 is the determined cDNA sequence of clone 61497481

SEQ ID NO:2333 is the determined cDNA sequence of clone 61497482

SEQ ID NO:2334 is the determined cDNA sequence of clone 61497483

SEQ ID NO:2335 is the determined cDNA sequence of clone 61497484

SEQ ID NO:2336 is the determined cDNA sequence of clone 61497486

SEQ ID NO:2337 is the determined cDNA sequence of clone 61497487

SEQ ID NO:2338 is the determined cDNA sequence of clone 61497488

SEQ ID NO:2339 is the determined cDNA sequence of clone 61497489

SEQ ID NO:2340 is the determined cDNA sequence of clone 61497490

SEQ ID NO:2341 is the determined cDNA sequence of clone 61497491

SEQ ID NO:2342 is the determined cDNA sequence of clone 61497493

SEQ ID NO:2343 is the determined cDNA sequence of clone 61497494

SEQ ID NO:2344 is the determined cDNA sequence of clone 61497495

SEQ ID NO:2345 is the determined cDNA sequence of clone 61497496

SEQ ID NO:2346 is the determined cDNA sequence of clone 61497497

SEQ ID NO:2347 is the determined cDNA sequence of clone 61497498

SEQ ID NO:2348 is the determined cDNA sequence of clone 61497499

SEQ ID NO:2349 is the determined cDNA sequence of clone 61497500

SEQ ID NO:2350 is the determined cDNA sequence of clone 61497501

SEQ ID NO:2351 is the determined cDNA sequence of clone 61497502

SEQ ID NO:2352 is the determined cDNA sequence of clone 61497503

SEQ ID NO:2353 is the determined cDNA sequence of clone 61497504

SEQ ID NO:2354 is the determined cDNA sequence of clone 61497505

SEQ ID NO:2355 is the determined cDNA sequence of clone 61497507

SEQ ID NO:2356 is the determined cDNA sequence of clone 61497509

SEQ ID NO:2357 is the determined cDNA sequence of clone 61497511

SEQ ID NO:2358 is the determined cDNA sequence of clone 61497512

SEQ ID NO:2359 is the determined cDNA sequence of clone 61497513

SEQ ID NO:2360 is the determined cDNA sequence of clone 61497514

SEQ ID NO:2361 is the determined cDNA sequence of clone 61497515

SEQ ID NO:2362 is the determined cDNA sequence of clone 61497516

SEQ ID NO:2363 is the determined cDNA sequence of clone 61497517

SEQ ID NO:2364 is the determined cDNA sequence of clone 61497518

SEQ ID NO:2365 is the determined cDNA sequence of clone 61497519

SEQ ID NO:2366 is the determined cDNA sequence of clone 61497520

SEQ ID NO:2367 is the determined cDNA sequence of clone 61497521

SEQ ID NO:2368 is the determined cDNA sequence of clone 61497522

SEQ ID NO:2369 is the determined cDNA sequence of clone 61497523

SEQ ID NO:2370 is the determined cDNA sequence of clone 61497524

SEQ ID NO:2371 is the determined cDNA sequence of clone 61497526

SEQ ID NO:2372 is the determined cDNA sequence of clone 61497527

SEQ ID NO:2373 is the determined cDNA sequence of clone 61497528

SEQ ID NO:2374 is the determined cDNA sequence of clone 61497529

SEQ ID NO:2375 is the determined cDNA sequence of clone 61497530

SEQ ID NO:2376 is the determined cDNA sequence of clone 61497531

SEQ ID NO:2377 is the determined cDNA sequence of clone 61497532

SEQ ID NO:2378 is the determined cDNA sequence of clone 61497533

SEQ ID NO:2379 is the determined cDNA sequence of clone 61497534

SEQ ID NO:2380 is the determined cDNA sequence of clone 61497535

SEQ ID NO:2381 is the determined cDNA sequence of clone 61497536

SEQ ID NO:2382 is the determined cDNA sequence of clone 61497537

SEQ ID NO:2383 is the determined cDNA sequence of clone 61497538

SEQ ID NO:2384 is the determined cDNA sequence of clone 61497539

SEQ ID NO:2385 is the determined cDNA sequence of clone 61497540

SEQ ID NO:2386 is the determined cDNA sequence of clone 61497541

SEQ ID NO:2387 is the determined cDNA sequence of clone 61497542

SEQ ID NO:2388 is the determined cDNA sequence of clone 61497543

SEQ ID NO:2389 is the determined cDNA sequence of clone 61497544

SEQ ID NO:2390 is the determined cDNA sequence of clone 61497545

SEQ ID NO:2391 is the determined cDNA sequence of clone 61497546

SEQ ID NO:2392 is the determined cDNA sequence of clone 61497547

SEQ ID NO:2393 is the determined cDNA sequence of clone 61497549

SEQ ID NO:2394 is the determined cDNA sequence of clone 61497551

SEQ ID NO:2395 is the determined cDNA sequence of clone 61497552

SEQ ID NO:2396 is the determined cDNA sequence of clone 61497553

SEQ ID NO:2397 is the determined cDNA sequence of clone 61497554

SEQ ID NO:2398 is the determined cDNA sequence of clone 61497556

SEQ ID NO:2399 is the determined cDNA sequence of clone 61497557

SEQ ID NO:2400 is the determined cDNA sequence of clone 61497560

SEQ ID NO:2401 is the determined cDNA sequence of clone 61497561

SEQ ID NO:2402 is the determined cDNA sequence of clone 61497562

SEQ ID NO:2403 is the determined cDNA sequence of clone 61497563

SEQ ID NO:2404 is the determined cDNA sequence of clone 61497564

SEQ ID NO:2405 is the determined cDNA sequence of clone 61497565

SEQ ID NO:2406 is the determined cDNA sequence of clone 61497566

SEQ ID NO:2407 is the determined cDNA sequence of clone 61497567

SEQ ID NO:2408 is the determined cDNA sequence of clone 61497568

SEQ ID NO:2409 is the determined cDNA sequence of clone 61497569

SEQ ID NO:2410 is the determined cDNA sequence of clone 61497570

SEQ ID NO:2411 is the determined cDNA sequence of clone 61497571

SEQ ID NO:2412 is the determined cDNA sequence of clone 61497572

SEQ ID NO:2413 is the determined cDNA sequence of clone 61497574

SEQ ID NO:2414 is the determined cDNA sequence of clone 61497575

SEQ ID NO:2415 is the determined cDNA sequence of clone 61497576

SEQ ID NO:2416 is the determined cDNA sequence of clone 61497577

SEQ ID NO:2417 is the determined cDNA sequence of clone 61497578

SEQ ID NO:2418 is the determined cDNA sequence of clone 61497579

SEQ ID NO:2419 is the determined cDNA sequence of clone 61497580

SEQ ID NO:2420 is the determined cDNA sequence of clone 61497581

SEQ ID NO:2421 is the determined cDNA sequence of clone 61497582

SEQ ID NO:2422 is the determined cDNA sequence of clone 61497583

SEQ ID NO:2423 is the determined cDNA sequence of clone 61497584

SEQ ID NO:2424 is the determined cDNA sequence of clone 61497585

SEQ ID NO:2425 is the determined cDNA sequence of clone 61497587

SEQ ID NO:2426 is the determined cDNA sequence of clone 61497588

SEQ ID NO:2427 is the determined cDNA sequence of clone 61497589

SEQ ID NO:2428 is the determined cDNA sequence of clone 61497590

SEQ ID NO:2429 is the determined cDNA sequence of clone 61497591

SEQ ID NO:2430 is the determined cDNA sequence of clone 61497592

SEQ ID NO:2431 is the determined cDNA sequence of clone 61497593

SEQ ID NO:2432 is the determined cDNA sequence of clone 61497594

SEQ ID NO:2433 is the determined cDNA sequence of clone 61497595

SEQ ID NO:2434 is the determined cDNA sequence of clone 61497596

SEQ ID NO:2435 is the determined cDNA sequence of clone 61497597

SEQ ID NO:2436 is the determined cDNA sequence of clone 61497598

SEQ ID NO:2437 is the determined cDNA sequence of clone 61497599

SEQ ID NO:2438 is the determined cDNA sequence of clone 61497600

SEQ ID NO:2439 is the determined cDNA sequence of clone 61497601

SEQ ID NO:2440 is the determined cDNA sequence of clone 61497602

SEQ ID NO:2441 is the determined cDNA sequence of clone 61497603

SEQ ID NO:2442 is the determined cDNA sequence of clone 61497604

SEQ ID NO:2443 is the determined cDNA sequence of clone 61497605

SEQ ID NO:2444 is the determined cDNA sequence of clone 61497606

SEQ ID NO:2445 is the determined cDNA sequence of clone 61497607

SEQ ID NO:2446 is the determined cDNA sequence of clone 61497608

SEQ ID NO:2447 is the determined cDNA sequence of clone 61497609

SEQ ID NO:2448 is the determined cDNA sequence of clone 61497610

SEQ ID NO:2449 is the determined cDNA sequence of clone 61497615

SEQ ID NO:2450 is the determined cDNA sequence of clone 61497616

SEQ ID NO:2451 is the determined cDNA sequence of clone 61497618

SEQ ID NO:2452 is the determined cDNA sequence of clone 61497619

SEQ ID NO:2453 is the determined cDNA sequence of clone 61497623

SEQ ID NO:2454 is the determined cDNA sequence of clone 61497624

SEQ ID NO:2455 is the determined cDNA sequence of clone 61497625

SEQ ID NO:2456 is the determined cDNA sequence of clone 61497626

SEQ ID NO:2457 is the determined cDNA sequence of clone 61497627

SEQ ID NO:2458 is the determined cDNA sequence of clone 61497628

SEQ ID NO:2459 is the determined cDNA sequence of clone 61497630

SEQ ID NO:2460 is the determined cDNA sequence of clone 61497631

SEQ ID NO:2461 is the determined cDNA sequence of clone 61497632

SEQ ID NO:2462 is the determined cDNA sequence of clone 61497633

SEQ ID NO:2463 is the determined cDNA sequence of clone 61497635

SEQ ID NO:2464 is the determined cDNA sequence of clone 61497636

SEQ ID NO:2465 is the determined cDNA sequence of clone 61497637

SEQ ID NO:2466 is the determined cDNA sequence of clone 61497638

SEQ ID NO:2467 is the determined cDNA sequence of clone 61497639

SEQ ID NO:2468 is the determined cDNA sequence of clone 61497640

SEQ ID NO:2469 is the determined cDNA sequence of clone 61497641

SEQ ID NO:2470 is the determined cDNA sequence of clone 61497643

SEQ ID NO:2471 is the determined cDNA sequence of clone 61497645

SEQ ID NO:2472 is the determined cDNA sequence of clone 61497646

SEQ ID NO:2473 is the determined cDNA sequence of clone 61497648

SEQ ID NO:2474 is the determined cDNA sequence of clone 61497649

SEQ ID NO:2475 is the determined cDNA sequence of clone 61497650

SEQ ID NO:2476 is the determined cDNA sequence of clone 61497652

SEQ ID NO:2477 is the determined cDNA sequence of clone 61497653

SEQ ID NO:2478 is the determined cDNA sequence of clone 61497655

SEQ ID NO:2479 is the determined cDNA sequence of clone 61497656

SEQ ID NO:2480 is the determined cDNA sequence of clone 61497657

SEQ ID NO:2481 is the determined cDNA sequence of clone 61497659

SEQ ID NO:2482 is the determined cDNA sequence of clone 61497660

SEQ ID NO:2483 is the determined cDNA sequence of clone 61497661

SEQ ID NO:2484 is the determined cDNA sequence of clone 61497662

SEQ ID NO:2485 is the determined cDNA sequence of clone 61497663

SEQ ID NO:2486 is the determined cDNA sequence of clone 61497664

SEQ ID NO:2487 is the determined cDNA sequence of clone 61497665

SEQ ID NO:2488 is the determined cDNA sequence of clone 61497666

SEQ ID NO:2489 is the determined cDNA sequence of clone 61497669

SEQ ID NO:2490 is the determined cDNA sequence of clone 61497670

SEQ ID NO:2491 is the determined cDNA sequence of clone 61497671

SEQ ID NO:2492 is the determined cDNA sequence of clone 61497673

SEQ ID NO:2493 is the determined cDNA sequence of clone 61497675

SEQ ID NO:2494 is the determined cDNA sequence of clone 61497676

SEQ ID NO:2495 is the determined cDNA sequence of clone 61497677

SEQ ID NO:2496 is the determined cDNA sequence of clone 61497678

SEQ ID NO:2497 is the determined cDNA sequence of clone 61497681

SEQ ID NO:2498 is the determined cDNA sequence of clone 61497682

SEQ ID NO:2499 is the determined cDNA sequence of clone 61497683

SEQ ID NO:2500 is the determined cDNA sequence of clone 61497684

SEQ ID NO:2501 is the determined cDNA sequence of clone 61497686

SEQ ID NO:2502 is the determined cDNA sequence of clone 61497687

SEQ ID NO:2503 is the determined cDNA sequence of clone 61497688

SEQ ID NO:2504 is the determined cDNA sequence of clone 61497691

SEQ ID NO:2505 is the determined cDNA sequence of clone 61497694

SEQ ID NO:2506 is the determined cDNA sequence of clone 61497695

SEQ ID NO:2507 is the determined cDNA sequence of clone 61524967

SEQ ID NO:2508 is the determined cDNA sequence of clone 61524969

SEQ ID NO:2509 is the determined cDNA sequence of clone 61524970

SEQ ID NO:2510 is the determined cDNA sequence of clone 61524971

SEQ ID NO:2511 is the determined cDNA sequence of clone 61524975

SEQ ID NO:2512 is the determined cDNA sequence of clone 61524976

SEQ ID NO:2513 is the determined cDNA sequence of clone 61524977

SEQ ID NO:2514 is the determined cDNA sequence of clone 61524979

SEQ ID NO:2515 is the determined cDNA sequence of clone 61524982

SEQ ID NO:2516 is the determined cDNA sequence of clone 61524983

SEQ ID NO:2517 is the determined cDNA sequence of clone 61524985

SEQ ID NO:2518 is the determined cDNA sequence of clone 61524986

SEQ ID NO:2519 is the determined cDNA sequence of clone 61524987

SEQ ID NO:2520 is the determined cDNA sequence of clone 61524988

SEQ ID NO:2521 is the determined cDNA sequence of clone 61524989

SEQ ID NO:2522 is the determined cDNA sequence of clone 61524990

SEQ ID NO:2523 is the determined cDNA sequence of clone 61524991

SEQ ID NO:2524 is the determined cDNA sequence of clone 61524993

SEQ ID NO:2525 is the determined cDNA sequence of clone 61524994

SEQ ID NO:2526 is the determined cDNA sequence of clone 61524995

SEQ ID NO:2527 is the determined cDNA sequence of clone 61524996

SEQ ID NO:2528 is the determined cDNA sequence of clone 61524997

SEQ ID NO:2529 is the determined cDNA sequence of clone 61524999

SEQ ID NO:2530 is the determined cDNA sequence of clone 61525000

SEQ ID NO:2531 is the determined cDNA sequence of clone 61525001

SEQ ID NO:2532 is the determined cDNA sequence of clone 61525003

SEQ ID NO:2533 is the determined cDNA sequence of clone 61525004

SEQ ID NO:2534 is the determined cDNA sequence of clone 61525005

SEQ ID NO:2535 is the determined cDNA sequence of clone 61525006

SEQ ID NO:2536 is the determined cDNA sequence of clone 61525007

SEQ ID NO:2537 is the determined cDNA sequence of clone 61525008

SEQ ID NO:2538 is the determined cDNA sequence of clone 61525009

SEQ ID NO:2539 is the determined cDNA sequence of clone 61525011

SEQ ID NO:2540 is the determined cDNA sequence of clone 61525013

SEQ ID NO:2541 is the determined cDNA sequence of clone 61525014

SEQ ID NO:2542 is the determined cDNA sequence of clone 61525015

SEQ ID NO:2543 is the determined cDNA sequence of clone 61525017

SEQ ID NO:2544 is the determined cDNA sequence of clone 61525020

SEQ ID NO:2545 is the determined cDNA sequence of clone 61525021

SEQ ID NO:2546 is the determined cDNA sequence of clone 61525022

SEQ ID NO:2547 is the determined cDNA sequence of clone 61525023

SEQ ID NO:2548 is the determined cDNA sequence of clone 61525026

SEQ ID NO:2549 is the determined cDNA sequence of clone 61525027

SEQ ID NO:2550 is the determined cDNA sequence of clone 61525029

SEQ ID NO:2551 is the determined cDNA sequence of clone 61525031

SEQ ID NO:2552 is the determined cDNA sequence of clone 61525034

SEQ ID NO:2553 is the determined cDNA sequence of clone 61525035

SEQ ID NO:2554 is the determined cDNA sequence of clone 61525038

SEQ ID NO:2555 is the determined cDNA sequence of clone 61525039

SEQ ID NO:2556 is the determined cDNA sequence of clone 61525040

SEQ ID NO:2557 is the determined cDNA sequence of clone 61525041

SEQ ID NO:2558 is the determined cDNA sequence of clone 61525042

SEQ ID NO:2559 is the determined cDNA sequence of clone 61525043

SEQ ID NO:2560 is the determined cDNA sequence of clone 61525044

SEQ ID NO:2561 is the determined cDNA sequence of clone 61525045

SEQ ID NO:2562 is the determined cDNA sequence of clone 61525047

SEQ ID NO:2563 is the determined cDNA sequence of clone 61525048

SEQ ID NO:2564 is the determined cDNA sequence of clone 61525049

SEQ ID NO:2565 is the determined cDNA sequence of clone 61525050

SEQ ID NO:2566 is the determined cDNA sequence of clone 61525052

SEQ ID NO:2567 is the determined cDNA sequence of clone 61525054

SEQ ID NO:2568 is the determined cDNA sequence of clone 61525055

SEQ ID NO:2569 is the determined cDNA sequence of clone 61525057

SEQ ID NO:2570 is the determined cDNA sequence of clone 61546433

SEQ ID NO:2571 is the determined cDNA sequence of clone 61546435

SEQ ID NO:2572 is the determined cDNA sequence of clone 61546436

SEQ ID NO:2573 is the determined cDNA sequence of clone 61546437

SEQ ID NO:2574 is the determined cDNA sequence of clone 61546439

SEQ ID NO:2575 is the determined cDNA sequence of clone 61546440

SEQ ID NO:2576 is the determined cDNA sequence of clone 61546442

SEQ ID NO:2577 is the determined cDNA sequence of clone 61546443

SEQ ID NO:2578 is the determined cDNA sequence of clone 61546444

SEQ ID NO:2579 is the determined cDNA sequence of clone 61546446

SEQ ID NO:2580 is the determined cDNA sequence of clone 61546447

SEQ ID NO:2581 is the determined cDNA sequence of clone 61546450

SEQ ID NO:2582 is the determined cDNA sequence of clone 61546451

SEQ ID NO:2583 is the determined cDNA sequence of clone 61546454

SEQ ID NO:2584 is the determined cDNA sequence of clone 61546455

SEQ ID NO:2585 is the determined cDNA sequence of clone 61546457

SEQ ID NO:2586 is the determined cDNA sequence of clone 61546458

SEQ ID NO:2587 is the determined cDNA sequence of clone 61546460

SEQ ID NO:2588 is the determined cDNA sequence of clone 61546461

SEQ ID NO:2589 is the determined cDNA sequence of clone 61546462

SEQ ID NO:2590 is the determined cDNA sequence of clone 61546463

SEQ ID NO:2591 is the determined cDNA sequence of clone 61546465

SEQ ID NO:2592 is the determined cDNA sequence of clone 61546466

SEQ ID NO:2593 is the determined cDNA sequence of clone 61546469

SEQ ID NO:2594 is the determined cDNA sequence of clone 61546471

SEQ ID NO:2595 is the determined cDNA sequence of clone 61546472

SEQ ID NO:2596 is the determined cDNA sequence of clone 61546473

SEQ ID NO:2597 is the determined cDNA sequence of clone 61546474

SEQ ID NO:2598 is the determined cDNA sequence of clone 61546475

SEQ ID NO:2599 is the determined cDNA sequence of clone 61546476

SEQ ID NO:2600 is the determined cDNA sequence of clone 61546477

SEQ ID NO:2601 is the determined cDNA sequence of clone 61546478

SEQ ID NO:2602 is the determined cDNA sequence of clone 61546480

SEQ ID NO:2603 is the determined cDNA sequence of clone 61546481

SEQ ID NO:2604 is the determined cDNA sequence of clone 61546482

SEQ ID NO:2605 is the determined cDNA sequence of clone 61546484

SEQ ID NO:2606 is the determined cDNA sequence of clone 61546487

SEQ ID NO:2607 is the determined cDNA sequence of clone 61546488

SEQ ID NO:2608 is the determined cDNA sequence of clone 61546490

SEQ ID NO:2609 is the determined cDNA sequence of clone 61546491

SEQ ID NO:2610 is the determined cDNA sequence of clone 61546492

SEQ ID NO:2611 is the determined cDNA sequence of clone 61546493

SEQ ID NO:2612 is the determined cDNA sequence of clone 61546496

SEQ ID NO:2613 is the determined cDNA sequence of clone 61546497

SEQ ID NO:2614 is the determined cDNA sequence of clone 61546498

SEQ ID NO:2615 is the determined cDNA sequence of clone 61546499

SEQ ID NO:2616 is the determined cDNA sequence of clone 61546500

SEQ ID NO:2617 is the determined cDNA sequence of clone 61546502

SEQ ID NO:2618 is the determined cDNA sequence of clone 61546503

SEQ ID NO:2619 is the determined cDNA sequence of clone 61546505

SEQ ID NO:2620 is the determined cDNA sequence of clone 61546507

SEQ ID NO:2621 is the determined cDNA sequence of clone 61546508

SEQ ID NO:2622 is the determined cDNA sequence of clone 61546509

SEQ ID NO:2623 is the determined cDNA sequence of clone 61546510

SEQ ID NO:2624 is the determined cDNA sequence of clone 61546512

SEQ ID NO:2625 is the determined cDNA sequence of clone 61546513

SEQ ID NO:2626 is the determined cDNA sequence of clone 61546516

SEQ ID NO:2627 is the determined cDNA sequence of clone 61546517

SEQ ID NO:2628 is the determined cDNA sequence of clone 61546518

SEQ ID NO:2629 is the determined cDNA sequence of clone 61546519

SEQ ID NO:2630 is the determined cDNA sequence of clone 61546520

SEQ ID NO:2631 is the determined cDNA sequence of clone 61546521

SEQ ID NO:2632 is the determined cDNA sequence of clone 61546522

SEQ ID NO:2633 is the determined cDNA sequence of clone 61546523

SEQ ID NO:2634 is the determined cDNA sequence of clone 61546524

SEQ ID NO:2635 is the determined cDNA sequence of clone 61814355

SEQ ID NO:2636 is the determined cDNA sequence of clone 61814356

SEQ ID NO:2637 is the determined cDNA sequence of clone 61814357

SEQ ID NO:2638 is the determined cDNA sequence of clone 61814360

SEQ ID NO:2639 is the determined cDNA sequence of clone 61814361

SEQ ID NO:2640 is the determined cDNA sequence of clone 61814362

SEQ ID NO:2641 is the determined cDNA sequence of clone 61814363

SEQ ID NO:2642 is the determined cDNA sequence of clone 61814364

SEQ ID NO:2643 is the determined cDNA sequence of clone 61814365

SEQ ID NO:2644 is the determined cDNA sequence of clone 61814366

SEQ ID NO:2645 is the determined cDNA sequence of clone 61814368

SEQ ID NO:2646 is the determined cDNA sequence of clone 61814369

SEQ ID NO:2647 is the determined cDNA sequence of clone 61814370

SEQ ID NO:2648 is the determined cDNA sequence of clone 61814371

SEQ ID NO:2649 is the determined cDNA sequence of clone 61814372

SEQ ID NO:2650 is the determined cDNA sequence of clone 61814373

SEQ ID NO:2651 is the determined cDNA sequence of clone 61814374

SEQ ID NO:2652 is the determined cDNA sequence of clone 61814376

SEQ ID NO:2653 is the determined cDNA sequence of clone 61814377

SEQ ID NO:2654 is the determined cDNA sequence of clone 61814378

SEQ ID NO:2655 is the determined cDNA sequence of clone 61814379

SEQ ID NO:2656 is the determined cDNA sequence of clone 61814381

SEQ ID NO:2657 is the determined cDNA sequence of clone 61814382

SEQ ID NO:2658 is the determined cDNA sequence of clone 61814383

SEQ ID NO:2659 is the determined cDNA sequence of clone 61814384

SEQ ID NO:2660 is the determined cDNA sequence of clone 61814385

SEQ ID NO:2661 is the determined cDNA sequence of clone 61814386

SEQ ID NO:2662 is the determined cDNA sequence of clone 61814387

SEQ ID NO:2663 is the determined cDNA sequence of clone 61814388

SEQ ID NO:2664 is the determined cDNA sequence of clone 61814389

SEQ ID NO:2665 is the determined cDNA sequence of clone 61814390

SEQ ID NO:2666 is the determined cDNA sequence of clone 61814391

SEQ ID NO:2667 is the determined cDNA sequence of clone 61814393

SEQ ID NO:2668 is the determined cDNA sequence of clone 61814394

SEQ ID NO:2669 is the determined cDNA sequence of clone 61814395

SEQ ID NO:2670 is the determined cDNA sequence of clone 61814396

SEQ ID NO:2671 is the determined cDNA sequence of clone 61814397

SEQ ID NO:2672 is the determined cDNA sequence of clone 61814398

SEQ ID NO:2673 is the determined cDNA sequence of clone 61814399

SEQ ID NO:2674 is the determined cDNA sequence of clone 61814400

SEQ ID NO:2675 is the determined cDNA sequence of clone 61814401

SEQ ID NO:2676 is the determined cDNA sequence of clone 61814402

SEQ ID NO:2677 is the determined cDNA sequence of clone 61814403

SEQ ID NO:2678 is the determined cDNA sequence of clone 61814405

SEQ ID NO:2679 is the determined cDNA sequence of clone 61814406

SEQ ID NO:2680 is the determined cDNA sequence of clone 61814407

SEQ ID NO:2681 is the determined cDNA sequence of clone 61814408

SEQ ID NO:2682 is the determined cDNA sequence of clone 61814409

SEQ ID NO:2683 is the determined cDNA sequence of clone 61814410

SEQ ID NO:2684 is the determined cDNA sequence of clone 61814411

SEQ ID NO:2685 is the determined cDNA sequence of clone 61814413

SEQ ID NO:2686 is the determined cDNA sequence of clone 61814414

SEQ ID NO:2687 is the determined cDNA sequence of clone 61814415

SEQ ID NO:2688 is the determined cDNA sequence of clone 61814416

SEQ ID NO:2689 is the determined cDNA sequence of clone 61814418

SEQ ID NO:2690 is the determined cDNA sequence of clone 61814419

SEQ ID NO:2691 is the determined cDNA sequence of clone 61814420

SEQ ID NO:2692 is the determined cDNA sequence of clone 61814421

SEQ ID NO:2693 is the determined cDNA sequence of clone 61814422

SEQ ID NO:2694 is the determined cDNA sequence of clone 61814423

SEQ ID NO:2695 is the determined cDNA sequence of clone 61814424

SEQ ID NO:2696 is the determined cDNA sequence of clone 61814426

SEQ ID NO:2697 is the determined cDNA sequence of clone 61814427

SEQ ID NO:2698 is the determined cDNA sequence of clone 61814428

SEQ ID NO:2699 is the determined cDNA sequence of clone 61814429

SEQ ID NO:2700 is the determined cDNA sequence of clone 61814431

SEQ ID NO:2701 is the determined cDNA sequence of clone 61814433

SEQ ID NO:2702 is the determined cDNA sequence of clone 61814434

SEQ ID NO:2703 is the determined cDNA sequence of clone 61814435

SEQ ID NO:2704 is the determined cDNA sequence of clone 61814436

SEQ ID NO:2705 is the determined cDNA sequence of clone 61814437

SEQ ID NO:2706 is the determined cDNA sequence of clone 61814438

SEQ ID NO:2707 is the determined cDNA sequence of clone 61814439

SEQ ID NO:2708 is the determined cDNA sequence of clone 61814440

SEQ ID NO:2709 is the determined cDNA sequence of clone 61814441

SEQ ID NO:2710 is the determined cDNA sequence of clone 61814442

SEQ ID NO:2711 is the determined cDNA sequence of clone 61814443

SEQ ID NO:2712 is the determined cDNA sequence of clone 61814444

SEQ ID NO:2713 is the determined cDNA sequence of clone 61814445

SEQ ID NO:2714 is the determined cDNA sequence of clone 61814446

SEQ ID NO:2715 is the determined cDNA sequence of clone 61814447

SEQ ID NO:2716 is the determined cDNA sequence of clone 61823768

SEQ ID NO:2717 is the determined cDNA sequence of clone 61823769

SEQ ID NO:2718 is the determined cDNA sequence of clone 61823770

SEQ ID NO:2719 is the determined cDNA sequence of clone 61823772

SEQ ID NO:2720 is the determined cDNA sequence of clone 61823773

SEQ ID NO:2721 is the determined cDNA sequence of clone 61823774

SEQ ID NO:2722 is the determined cDNA sequence of clone 61823775

SEQ ID NO:2723 is the determined cDNA sequence of clone 61823777

SEQ ID NO:2724 is the determined cDNA sequence of clone 61823778

SEQ ID NO:2725 is the determined cDNA sequence of clone 61823779

SEQ ID NO:2726 is the determined cDNA sequence of clone 61823780

SEQ ID NO:2727 is the determined cDNA sequence of clone 61823781

SEQ ID NO:2728 is the determined cDNA sequence of clone 61823782

SEQ ID NO:2729 is the determined cDNA sequence of clone 61823784

SEQ ID NO:2730 is the determined cDNA sequence of clone 61823785

SEQ ID NO:2731 is the determined cDNA sequence of clone 61823786

SEQ ID NO:2732 is the determined cDNA sequence of clone 61823787

SEQ ID NO:2733 is the determined cDNA sequence of clone 61823788

SEQ ID NO:2734 is the determined cDNA sequence of clone 61823789

SEQ ID NO:2735 is the determined cDNA sequence of clone 61823790

SEQ ID NO:2736 is the determined cDNA sequence of clone 61823791

SEQ ID NO:2737 is the determined cDNA sequence of clone 61823792

SEQ ID NO:2738 is the determined cDNA sequence of clone 61823793

SEQ ID NO:2739 is the determined cDNA sequence of clone 61823794

SEQ ID NO:2740 is the determined cDNA sequence of clone 61823797

SEQ ID NO:2741 is the determined cDNA sequence of clone 61823798

SEQ ID NO:2742 is the determined cDNA sequence of clone 61823799

SEQ ID NO:2743 is the determined cDNA sequence of clone 61823800

SEQ ID NO:2744 is the determined cDNA sequence of clone 61823801

SEQ ID NO:2745 is the determined cDNA sequence of clone 61823802

SEQ ID NO:2746 is the determined cDNA sequence of clone 61823803

SEQ ID NO:2747 is the determined cDNA sequence of clone 61823804

SEQ ID NO:2748 is the determined cDNA sequence of clone 61823805

SEQ ID NO:2749 is the determined cDNA sequence of clone 61823807

SEQ ID NO:2750 is the determined cDNA sequence of clone 61823808

SEQ ID NO:2751 is the determined cDNA sequence of clone 61823809

SEQ ID NO:2752 is the determined cDNA sequence of clone 61823810

SEQ ID NO:2753 is the determined cDNA sequence of clone 61823811

SEQ ID NO:2754 is the determined cDNA sequence of clone 61823812

SEQ ID NO:2755 is the determined cDNA sequence of clone 61823813

SEQ ID NO:2756 is the determined cDNA sequence of clone 61823814

SEQ ID NO:2757 is the determined cDNA sequence of clone 61823815

SEQ ID NO:2758 is the determined cDNA sequence of clone 61823816

SEQ ID NO:2759 is the determined cDNA sequence of clone 61823817

SEQ ID NO:2760 is the determined cDNA sequence of clone 61823818

SEQ ID NO:2761 is the determined cDNA sequence of clone 61823820

SEQ ID NO:2762 is the determined cDNA sequence of clone 61823821

SEQ ID NO:2763 is the determined cDNA sequence of clone 61823822

SEQ ID NO:2764 is the determined cDNA sequence of clone 61823823

SEQ ID NO:2765 is the determined cDNA sequence of clone 61823824

SEQ ID NO:2766 is the determined cDNA sequence of clone 61823825

SEQ ID NO:2767 is the determined cDNA sequence of clone 61823826

SEQ ID NO:2768 is the determined cDNA sequence of clone 61823827

SEQ ID NO:2769 is the determined cDNA sequence of clone 61823829

SEQ ID NO:2770 is the determined cDNA sequence of clone 61823830

SEQ ID NO:2771 is the determined cDNA sequence of clone 61823831

SEQ ID NO:2772 is the determined cDNA sequence of clone 61823833

SEQ ID NO:2773 is the determined cDNA sequence of clone 61823835

SEQ ID NO:2774 is the determined cDNA sequence of clone 61823837

SEQ ID NO:2775 is the determined cDNA sequence of clone 61823839

SEQ ID NO:2776 is the determined cDNA sequence of clone 61823840

SEQ ID NO:2777 is the determined cDNA sequence of clone 61823841

SEQ ID NO:2778 is the determined cDNA sequence of clone 61823843

SEQ ID NO:2779 is the determined cDNA sequence of clone 61823844

SEQ ID NO:2780 is the determined cDNA sequence of clone 61823845

SEQ ID NO:2781 is the determined cDNA sequence of clone 61823847

SEQ ID NO:2782 is the determined cDNA sequence of clone 61823849

SEQ ID NO:2783 is the determined cDNA sequence of clone 61823850

SEQ ID NO:2784 is the determined cDNA sequence of clone 61823851

SEQ ID NO:2785 is the determined cDNA sequence of clone 61823852

SEQ ID NO:2786 is the determined cDNA sequence of clone 61823854

SEQ ID NO:2787 is the determined cDNA sequence of clone 61823855

SEQ ID NO:2788 is the determined cDNA sequence of clone 61823856

SEQ ID NO:2789 is the determined cDNA sequence of clone 61823858

SEQ ID NO:2790 is the determined cDNA sequence of clone 61823859

SEQ ID NO:2791 is the determined cDNA sequence of clone 62342414

SEQ ID NO:2792 is the determined cDNA sequence of clone 62342416

SEQ ID NO:2793 is the determined cDNA sequence of clone 62342417

SEQ ID NO:2794 is the determined cDNA sequence of clone 62342422

SEQ ID NO:2795 is the determined cDNA sequence of clone 62342423

SEQ ID NO:2796 is the determined cDNA sequence of clone 62342425

SEQ ID NO:2797 is the determined cDNA sequence of clone 62342426

SEQ ID NO:2798 is the determined cDNA sequence of clone 62342428

SEQ ID NO:2799 is the determined cDNA sequence of clone 62342429

SEQ ID NO:2800 is the determined cDNA sequence of clone 62342430

SEQ ID NO:2801 is the determined cDNA sequence of clone 62342433

SEQ ID NO:2802 is the determined cDNA sequence of clone 62342434

SEQ ID NO:2803 is the determined cDNA sequence of clone 62342436

SEQ ID NO:2804 is the determined cDNA sequence of clone 62342437

SEQ ID NO:2805 is the determined cDNA sequence of clone 62342438

SEQ ID NO:2806 is the determined cDNA sequence of clone 62342439

SEQ ID NO:2807 is the determined cDNA sequence of clone 62342440

SEQ ID NO:2808 is the determined cDNA sequence of clone 62342442

SEQ ID NO:2809 is the determined cDNA sequence of clone 62342443

SEQ ID NO:2810 is the determined cDNA sequence of clone 62342447

SEQ ID NO:2811 is the determined cDNA sequence of clone 62342449

SEQ ID NO:2812 is the determined cDNA sequence of clone 62342452

SEQ ID NO:2813 is the determined cDNA sequence of clone 62342453

SEQ ID NO:2814 is the determined cDNA sequence of clone 62342454

SEQ ID NO:2815 is the determined cDNA sequence of clone 62342455

SEQ ID NO:2816 is the determined cDNA sequence of clone 62342456

SEQ ID NO:2817 is the determined cDNA sequence of clone 62342457

SEQ ID NO:2818 is the determined cDNA sequence of clone 62342458

SEQ ID NO:2819 is the determined cDNA sequence of clone 62342460

SEQ ID NO:2820 is the determined cDNA sequence of clone 62342462

SEQ ID NO:2821 is the determined cDNA sequence of clone 62342464

SEQ ID NO:2822 is the determined cDNA sequence of clone 62342467

SEQ ID NO:2823 is the determined cDNA sequence of clone 62342468

SEQ ID NO:2824 is the determined cDNA sequence of clone 62342469

SEQ ID NO:2825 is the determined cDNA sequence of clone 62342471

SEQ ID NO:2826 is the determined cDNA sequence of clone 62342476

SEQ ID NO:2827 is the determined cDNA sequence of clone 62342477

SEQ ID NO:2828 is the determined cDNA sequence of clone 62342480

SEQ ID NO:2829 is the determined cDNA sequence of clone 62342481

SEQ ID NO:2830 is the determined cDNA sequence of clone 62342484

SEQ ID NO:2831 is the determined cDNA sequence of clone 62342485

SEQ ID NO:2832 is the determined cDNA sequence of clone 62342488

SEQ ID NO:2833 is the determined cDNA sequence of clone 62342489

SEQ ID NO:2834 is the determined cDNA sequence of clone 62342490

SEQ ID NO:2835 is the determined cDNA sequence of clone 62342492

SEQ ID NO:2836 is the determined cDNA sequence of clone 62342493

SEQ ID NO:2837 is the determined cDNA sequence of clone 62342495

SEQ ID NO:2838 is the determined cDNA sequence of clone 62342497

SEQ ID NO:2839 is the determined cDNA sequence of clone 62342498

SEQ ID NO:2840 is the determined cDNA sequence of clone 62342504

SEQ ID NO:2841 is the determined cDNA sequence of clone 61814542

SEQ ID NO:2842 is the determined cDNA sequence of clone 61814543

SEQ ID NO:2843 is the determined cDNA sequence of clone 61814546

SEQ ID NO:2844 is the determined cDNA sequence of clone 61814548

SEQ ID NO:2845 is the determined cDNA sequence of clone 61814549

SEQ ID NO:2846 is the determined cDNA sequence of clone 61814550

SEQ ID NO:2847 is the determined cDNA sequence of clone 61814551

SEQ ID NO:2848 is the determined cDNA sequence of clone 61814552

SEQ ID NO:2849 is the determined cDNA sequence of clone 61814553

SEQ ID NO:2850 is the determined cDNA sequence of clone 61814554

SEQ ID NO:2851 is the determined cDNA sequence of clone 61814555

SEQ ID NO:2852 is the determined cDNA sequence of clone 61814556

SEQ ID NO:2853 is the determined cDNA sequence of clone 61814561

SEQ ID NO:2854 is the determined cDNA sequence of clone 61814562

SEQ ID NO:2855 is the determined cDNA sequence of clone 61814566

SEQ ID NO:2856 is the determined cDNA sequence of clone 61814568

SEQ ID NO:2857 is the determined cDNA sequence of clone 61814569

SEQ ID NO:2858 is the determined cDNA sequence of clone 61814570

SEQ ID NO:2859 is the determined cDNA sequence of clone 61814571

SEQ ID NO:2860 is the determined cDNA sequence of clone 61814572

SEQ ID NO:2861 is the determined cDNA sequence of clone 61814573

SEQ ID NO:2862 is the determined cDNA sequence of clone 61814575

SEQ ID NO:2863 is the determined cDNA sequence of clone 61814576

SEQ ID NO:2864 is the determined cDNA sequence of clone 61814577

SEQ ID NO:2865 is the determined cDNA sequence of clone 61814579

SEQ ID NO:2866 is the determined cDNA sequence of clone 61814580

SEQ ID NO:2867 is the determined cDNA sequence of clone 61814581

SEQ ID NO:2868 is the determined cDNA sequence of clone 61814583

SEQ ID NO:2869 is the determined cDNA sequence of clone 61814584

SEQ ID NO:2870 is the determined cDNA sequence of clone 61814585

SEQ ID NO:2871 is the determined cDNA sequence of clone 61814586

SEQ ID NO:2872 is the determined cDNA sequence of clone 61814587

SEQ ID NO:2873 is the determined cDNA sequence of clone 61814588

SEQ ID NO:2874 is the determined cDNA sequence of clone 61814589

SEQ ID NO:2875 is the determined cDNA sequence of clone 61814590

SEQ ID NO:2876 is the determined cDNA sequence of clone 61814591

SEQ ID NO:2877 is the determined cDNA sequence of clone 61814592

SEQ ID NO:2878 is the determined cDNA sequence of clone 61814595

SEQ ID NO:2879 is the determined cDNA sequence of clone 61814596

SEQ ID NO:2880 is the determined cDNA sequence of clone 61814598

SEQ ID NO:2881 is the determined cDNA sequence of clone 61814600

SEQ ID NO:2882 is the determined cDNA sequence of clone 61814601

SEQ ID NO:2883 is the determined cDNA sequence of clone 61814602

SEQ ID NO:2884 is the determined cDNA sequence of clone 61814604

SEQ ID NO:2885 is the determined cDNA sequence of clone 61814605

SEQ ID NO:2886 is the determined cDNA sequence of clone 61814606

SEQ ID NO:2887 is the determined cDNA sequence of clone 61814607

SEQ ID NO:2888 is the determined cDNA sequence of clone 61814608

SEQ ID NO:2889 is the determined cDNA sequence of clone 61814609

SEQ ID NO:2890 is the determined cDNA sequence of clone 61814610

SEQ ID NO:2891 is the determined cDNA sequence of clone 61814611

SEQ ID NO:2892 is the determined cDNA sequence of clone 61814612

SEQ ID NO:2893 is the determined cDNA sequence of clone 61814613

SEQ ID NO:2894 is the determined cDNA sequence of clone 61814614

SEQ ID NO:2895 is the determined cDNA sequence of clone 61814615

SEQ ID NO:2896 is the determined cDNA sequence of clone 61814616

SEQ ID NO:2897 is the determined cDNA sequence of clone 61814617

SEQ ID NO:2898 is the determined cDNA sequence of clone 61814618

SEQ ID NO:2899 is the determined cDNA sequence of clone 61814619

SEQ ID NO:2900 is the determined cDNA sequence of clone 61814620

SEQ ID NO:2901 is the determined cDNA sequence of clone 61814622

SEQ ID NO:2902 is the determined cDNA sequence of clone 61814623

SEQ ID NO:2903 is the determined cDNA sequence of clone 61814624

SEQ ID NO:2904 is the determined cDNA sequence of clone 61814625

SEQ ID NO:2905 is the determined cDNA sequence of clone 61814626

SEQ ID NO:2906 is the determined cDNA sequence of clone 61814627

SEQ ID NO:2907 is the determined cDNA sequence of clone 61814628

SEQ ID NO:2908 is the determined cDNA sequence of clone 61814629

SEQ ID NO:2909 is the determined cDNA sequence of clone 61814630

SEQ ID NO:2910 is the determined cDNA sequence of clone 61814631

SEQ ID NO:2911 is the determined cDNA sequence of clone 61814633

SEQ ID NO:2912 is the determined cDNA sequence of clone 61814449

SEQ ID NO:2913 is the determined cDNA sequence of clone 61814450

SEQ ID NO:2914 is the determined cDNA sequence of clone 61814452

SEQ ID NO:2915 is the determined cDNA sequence of clone 61814453

SEQ ID NO:2916 is the determined cDNA sequence of clone 61814454

SEQ ID NO:2917 is the determined cDNA sequence of clone 61814456

SEQ ID NO:2918 is the determined cDNA sequence of clone 61814457

SEQ ID NO:2919 is the determined cDNA sequence of clone 61814458

SEQ ID NO:2920 is the determined cDNA sequence of clone 61814459

SEQ ID NO:2921 is the determined cDNA sequence of clone 61814460

SEQ ID NO:2922 is the determined cDNA sequence of clone 61814461

SEQ ID NO:2923 is the determined cDNA sequence of clone 61814462

SEQ ID NO:2924 is the determined cDNA sequence of clone 61814466

SEQ ID NO:2925 is the determined cDNA sequence of clone 61814467

SEQ ID NO:2926 is the determined cDNA sequence of clone 61814468

SEQ ID NO:2927 is the determined cDNA sequence of clone 61814470

SEQ ID NO:2928 is the determined cDNA sequence of clone 61814471

SEQ ID NO:2929 is the determined cDNA sequence of clone 61814472

SEQ ID NO:2930 is the determined cDNA sequence of clone 61814473

SEQ ID NO:2931 is the determined cDNA sequence of clone 61814474

SEQ ID NO:2932 is the determined cDNA sequence of clone 61814476

SEQ ID NO:2933 is the determined cDNA sequence of clone 61814478

SEQ ID NO:2934 is the determined cDNA sequence of clone 61814483

SEQ ID NO:2935 is the determined cDNA sequence of clone 61814484

SEQ ID NO:2936 is the determined cDNA sequence of clone 61814485

SEQ ID NO:2937 is the determined cDNA sequence of clone 61814486

SEQ ID NO:2938 is the determined cDNA sequence of clone 61814487

SEQ ID NO:2939 is the determined cDNA sequence of clone 61814489

SEQ ID NO:2940 is the determined cDNA sequence of clone 61814490

SEQ ID NO:2941 is the determined cDNA sequence of clone 61814492

SEQ ID NO:2942 is the determined cDNA sequence of clone 61814494

SEQ ID NO:2943 is the determined cDNA sequence of clone 61814495

SEQ ID NO:2944 is the determined cDNA sequence of clone 61814496

SEQ ID NO:2945 is the determined cDNA sequence of clone 61814497

SEQ ID NO:2946 is the determined cDNA sequence of clone 61814498

SEQ ID NO:2947 is the determined cDNA sequence of clone 61814499

SEQ ID NO:2948 is the determined cDNA sequence of clone 61814500

SEQ ID NO:2949 is the determined cDNA sequence of clone 61814502

SEQ ID NO:2950 is the determined cDNA sequence of clone 61814503

SEQ ID NO:2951 is the determined cDNA sequence of clone 61814505

SEQ ID NO:2952 is the determined cDNA sequence of clone 61814506

SEQ ID NO:2953 is the determined cDNA sequence of clone 61814508

SEQ ID NO:2954 is the determined cDNA sequence of clone 61814509

SEQ ID NO:2955 is the determined cDNA sequence of clone 61814510

SEQ ID NO:2956 is the determined cDNA sequence of clone 61814512

SEQ ID NO:2957 is the determined cDNA sequence of clone 61814513

SEQ ID NO:2958 is the determined cDNA sequence of clone 61814514

SEQ ID NO:2959 is the determined cDNA sequence of clone 61814517

SEQ ID NO:2960 is the determined cDNA sequence of clone 61814518

SEQ ID NO:2961 is the determined cDNA sequence of clone 61814520

SEQ ID NO:2962 is the determined cDNA sequence of clone 61814521

SEQ ID NO:2963 is the determined cDNA sequence of clone 61814522

SEQ ID NO:2964 is the determined cDNA sequence of clone 61814523

SEQ ID NO:2965 is the determined cDNA sequence of clone 61814524

SEQ ID NO:2966 is the determined cDNA sequence of clone 61814525

SEQ ID NO:2967 is the determined cDNA sequence of clone 61814526

SEQ ID NO:2968 is the determined cDNA sequence of clone 61814528

SEQ ID NO:2969 is the determined cDNA sequence of clone 61814530

SEQ ID NO:2970 is the determined cDNA sequence of clone 61814531

SEQ ID NO:2971 is the determined cDNA sequence of clone 61814532

SEQ ID NO:2972 is the determined cDNA sequence of clone 61814534

SEQ ID NO:2973 is the determined cDNA sequence of clone 61814535

SEQ ID NO:2974 is the determined cDNA sequence of clone 61814536

SEQ ID NO:2975 is the determined cDNA sequence of clone 61814537

SEQ ID NO:2976 is the determined cDNA sequence of clone 61814538

SEQ ID NO:2977 is the determined cDNA sequence of clone 61814539

SEQ ID NO:2978 is the determined cDNA sequence of clone 61814540

SEQ ID NO:2979 is the determined cDNA sequence of clone 62210387

SEQ ID NO:2980 is the determined cDNA sequence of clone 62210388

SEQ ID NO:2981 is the determined cDNA sequence of clone 62210389

SEQ ID NO:2982 is the determined cDNA sequence of clone 62210390

SEQ ID NO:2983 is the determined cDNA sequence of clone 62210391

SEQ ID NO:2984 is the determined cDNA sequence of clone 62210394

SEQ ID NO:2985 is the determined cDNA sequence of clone 62210395

SEQ ID NO:2986 is the determined cDNA sequence of clone 62210397

SEQ ID NO:2987 is the determined cDNA sequence of clone 62210398

SEQ ID NO:2988 is the determined cDNA sequence of clone 62210399

SEQ ID NO:2989 is the determined cDNA sequence of clone 62210401

SEQ ID NO:2990 is the determined cDNA sequence of clone 62210403

SEQ ID NO:2991 is the determined cDNA sequence of clone 62210404

SEQ ID NO:2992 is the determined cDNA sequence of clone 63231658

SEQ ID NO:2993 is the determined cDNA sequence of clone 63231659

SEQ ID NO:2994 is the determined cDNA sequence of clone 63231660

SEQ ID NO:2995 is the determined cDNA sequence of clone 63231661

SEQ ID NO:2996 is the determined cDNA sequence of clone 63231662

SEQ ID NO:2997 is the determined cDNA sequence of clone 63231664

SEQ ID NO:2998 is the determined cDNA sequence of clone 63231665

SEQ ID NO:2999 is the determined cDNA sequence of clone 63231666

SEQ ID NO:3000 is the determined cDNA sequence of clone 63231667

SEQ ID NO:3001 is the determined cDNA sequence of clone 63231668

SEQ ID NO:3002 is the determined cDNA sequence of clone 63231669

SEQ ID NO:3003 is the determined cDNA sequence of clone 63231670

SEQ ID NO:3004 is the determined cDNA sequence of clone 63231671

SEQ ID NO:3005 is the determined cDNA sequence of clone 63231672

SEQ ID NO:3006 is the determined cDNA sequence of clone 63231673

SEQ ID NO:3007 is the determined cDNA sequence of clone 63231674

SEQ ID NO:3008 is the determined cDNA sequence of clone 63231675

SEQ ID NO:3009 is the determined cDNA sequence of clone 63231676

SEQ ID NO:3010 is the determined cDNA sequence of clone 63231677

SEQ ID NO:3011 is the determined cDNA sequence of clone 63231678

SEQ ID NO:3012 is the determined cDNA sequence of clone 63231679

SEQ ID NO:3013 is the determined cDNA sequence of clone 63231680

SEQ ID NO:3014 is the determined cDNA sequence of clone 63231681

SEQ ID NO:3015 is the determined cDNA sequence of clone 63231682

SEQ ID NO:3016 is the determined cDNA sequence of clone 63231683

SEQ ID NO:3017 is the determined cDNA sequence of clone 63231684

SEQ ID NO:3018 is the determined cDNA sequence of clone 63231685

SEQ ID NO:3019 is the determined cDNA sequence of clone 63231686

SEQ ID NO:3020 is the determined cDNA sequence of clone 63231687

SEQ ID NO:3021 is the determined cDNA sequence of clone 63231688

SEQ ID NO:3022 is the determined cDNA sequence of clone 63231689

SEQ ID NO:3023 is the determined cDNA sequence of clone 63231690

SEQ ID NO:3024 is the determined cDNA sequence of clone 63231691

SEQ ID NO:3025 is the determined cDNA sequence of clone 63231692

SEQ ID NO:3026 is the determined cDNA sequence of clone 63231693

SEQ ID NO:3027 is the determined cDNA sequence of clone 63231694

SEQ ID NO:3028 is the determined cDNA sequence of clone 63231695

SEQ ID NO:3029 is the determined cDNA sequence of clone 63231696

SEQ ID NO:3030 is the determined cDNA sequence of clone 63231697

SEQ ID NO:3031 is the determined cDNA sequence of clone 63231698

SEQ ID NO:3032 is the determined cDNA sequence of clone 63231699

SEQ ID NO:3033 is the determined cDNA sequence of clone 63231700

SEQ ID NO:3034 is the determined cDNA sequence of clone 63231701

SEQ ID NO:3035 is the determined cDNA sequence of clone 63231702

SEQ ID NO:3036 is the determined cDNA sequence of clone 63231703

SEQ ID NO:3037 is the determined cDNA sequence of clone 63231704

SEQ ID NO:3038 is the determined cDNA sequence of clone 63231705

SEQ ID NO:3039 is the determined cDNA sequence of clone 63231706

SEQ ID NO:3040 is the determined cDNA sequence of clone 63231707

SEQ ID NO:3041 is the determined cDNA sequence of clone 63231708

SEQ ID NO:3042 is the determined cDNA sequence of clone 63231709

SEQ ID NO:3043 is the determined cDNA sequence of clone 63231711

SEQ ID NO:3044 is the determined cDNA sequence of clone 63231713

SEQ ID NO:3045 is the determined cDNA sequence of clone 63231715

SEQ ID NO:3046 is the determined cDNA sequence of clone 63231716

SEQ ID NO:3047 is the determined cDNA sequence of clone 63231717

SEQ ID NO:3048 is the determined cDNA sequence of clone 63231718

SEQ ID NO:3049 is the determined cDNA sequence of clone 63231719

SEQ ID NO:3050 is the determined cDNA sequence of clone 63231720

SEQ ID NO:3051 is the determined cDNA sequence of clone 63231721

SEQ ID NO:3052 is the determined cDNA sequence of clone 63231723

SEQ ID NO:3053 is the determined cDNA sequence of clone 63231724

SEQ ID NO:3054 is the determined cDNA sequence of clone 63231725

SEQ ID NO:3055 is the determined cDNA sequence of clone 63231726

SEQ ID NO:3056 is the determined cDNA sequence of clone 63231727

SEQ ID NO:3057 is the determined cDNA sequence of clone 63231729

SEQ ID NO:3058 is the determined cDNA sequence of clone 63231730

SEQ ID NO:3059 is the determined cDNA sequence of clone 63231731

SEQ ID NO:3060 is the determined cDNA sequence of clone 63231732

SEQ ID NO:3061 is the determined cDNA sequence of clone 63138223

SEQ ID NO:3062 is the determined cDNA sequence of clone 63138224

SEQ ID NO:3063 is the determined cDNA sequence of clone 63138225

SEQ ID NO:3064 is the determined cDNA sequence of clone 63138226

SEQ ID NO:3065 is the determined cDNA sequence of clone 63138227

SEQ ID NO:3066 is the determined cDNA sequence of clone 63138228

SEQ ID NO:3067 is the determined cDNA sequence of clone 63138229

SEQ ID NO:3068 is the determined cDNA sequence of clone 63138230

SEQ ID NO:3069 is the determined cDNA sequence of clone 63138231

SEQ ID NO:3070 is the determined cDNA sequence of clone 63138232

SEQ ID NO:3071 is the determined cDNA sequence of clone 63138233

SEQ ID NO:3072 is the determined cDNA sequence of clone 63138234

SEQ ID NO:3073 is the determined cDNA sequence of clone 63138235

SEQ ID NO:3074 is the determined cDNA sequence of clone 63138237

SEQ ID NO:3075 is the determined cDNA sequence of clone 63138239

SEQ ID NO:3076 is the determined cDNA sequence of clone 63138240

SEQ ID NO:3077 is the determined cDNA sequence of clone 63138241

SEQ ID NO:3078 is the determined cDNA sequence of clone 63138242

SEQ ID NO:3079 is the determined cDNA sequence of clone 63138243

SEQ ID NO:3080 is the determined cDNA sequence of clone 63138244

SEQ ID NO:3081 is the determined cDNA sequence of clone 63138246

SEQ ID NO:3082 is the determined cDNA sequence of clone 63138249

SEQ ID NO:3083 is the determined cDNA sequence of clone 63138250

SEQ ID NO:3084 is the determined cDNA sequence of clone 63138251

SEQ ID NO:3085 is the determined cDNA sequence of clone 63138252

SEQ ID NO:3086 is the determined cDNA sequence of clone 63138253

SEQ ID NO:3087 is the determined cDNA sequence of clone 63138254

SEQ ID NO:3088 is the determined cDNA sequence of clone 63138255

SEQ ID NO:3089 is the determined cDNA sequence of clone 63138256

SEQ ID NO:3090 is the determined cDNA sequence of clone 63138257

SEQ ID NO:3091 is the determined cDNA sequence of clone 63138258

SEQ ID NO:3092 is the determined cDNA sequence of clone 63138259

SEQ ID NO:3093 is the determined cDNA sequence of clone 63138260

SEQ ID NO:3094 is the determined cDNA sequence of clone 63138262

SEQ ID NO:3095 is the determined cDNA sequence of clone 63138264

SEQ ID NO:3096 is the determined cDNA sequence of clone 63138265

SEQ ID NO:3097 is the determined cDNA sequence of clone 63138266

SEQ ID NO:3098 is the determined cDNA sequence of clone 63138267

SEQ ID NO:3099 is the determined cDNA sequence of clone 63138268

SEQ ID NO:3100 is the determined cDNA sequence of clone 63138269

SEQ ID NO:3101 is the determined cDNA sequence of clone 63138270

SEQ ID NO:3102 is the determined cDNA sequence of clone 63138271

SEQ ID NO:3103 is the determined cDNA sequence of clone 63138272

SEQ ID NO:3104 is the determined cDNA sequence of clone 63138273

SEQ ID NO:3105 is the determined cDNA sequence of clone 63138274

SEQ ID NO:3106 is the determined cDNA sequence of clone 63138275

SEQ ID NO:3107 is the determined cDNA sequence of clone 63138276

SEQ ID NO:3108 is the determined cDNA sequence of clone 63138277

SEQ ID NO:3109 is the determined cDNA sequence of clone 63138278

SEQ ID NO:3110 is the determined cDNA sequence of clone 63138279

SEQ ID NO:3111 is the determined cDNA sequence of clone 63138280

SEQ ID NO:3112 is the determined cDNA sequence of clone 63138281

SEQ ID NO:3113 is the determined cDNA sequence of clone 63138282

SEQ ID NO:3114 is the determined cDNA sequence of clone 63138283

SEQ ID NO:3115 is the determined cDNA sequence of clone 63138284

SEQ ID NO:3116 is the determined cDNA sequence of clone 63138285

SEQ ID NO:3117 is the determined cDNA sequence of clone 63138286

SEQ ID NO:3118 is the determined cDNA sequence of clone 63138287

SEQ ID NO:3119 is the determined cDNA sequence of clone 63138288

SEQ ID NO:3120 is the determined cDNA sequence of clone 63138289

SEQ ID NO:3121 is the determined cDNA sequence of clone 63138290

SEQ ID NO:3122 is the determined cDNA sequence of clone 63138291

SEQ ID NO:3123 is the determined cDNA sequence of clone 63138292

SEQ ID NO:3124 is the determined cDNA sequence of clone 63138294

SEQ ID NO:3125 is the determined cDNA sequence of clone 63138295

SEQ ID NO:3126 is the determined cDNA sequence of clone 63138296

SEQ ID NO:3127 is the determined cDNA sequence of clone 63138297

SEQ ID NO:3128 is the determined cDNA sequence of clone 63138298

SEQ ID NO:3129 is the determined cDNA sequence of clone 63138299

SEQ ID NO:3130 is the determined cDNA sequence of clone 63138300

SEQ ID NO:3131 is the determined cDNA sequence of clone 63138302

SEQ ID NO:3132 is the determined cDNA sequence of clone 63138303

SEQ ID NO:3133 is the determined cDNA sequence of clone 63138304

SEQ ID NO:3134 is the determined cDNA sequence of clone 63138305

SEQ ID NO:3135 is the determined cDNA sequence of clone 63138306

SEQ ID NO:3136 is the determined cDNA sequence of clone 63138308

SEQ ID NO:3137 is the determined cDNA sequence of clone 63138309

SEQ ID NO:3138 is the determined cDNA sequence of clone 63138311

SEQ ID NO:3139 is the determined cDNA sequence of clone 63138312

SEQ ID NO:3140 is the determined cDNA sequence of clone 63138313

SEQ ID NO:3141 is the determined cDNA sequence of clone 63138314

SEQ ID NO:3142 is the determined cDNA sequence of clone 63298516

SEQ ID NO:3143 is the determined cDNA sequence of clone 63298517

SEQ ID NO:3144 is the determined cDNA sequence of clone 63298518

SEQ ID NO:3145 is the determined cDNA sequence of clone 63298519

SEQ ID NO:3146 is the determined cDNA sequence of clone 63298520

SEQ ID NO:3147 is the determined cDNA sequence of clone 63298521

SEQ ID NO:3148 is the determined cDNA sequence of clone 63298522

SEQ ID NO:3149 is the determined cDNA sequence of clone 63298523

SEQ ID NO:3150 is the determined cDNA sequence of clone 63298524

SEQ ID NO:3151 is the determined cDNA sequence of clone 63298526

SEQ ID NO:3152 is the determined cDNA sequence of clone 63298527

SEQ ID NO:3153 is the determined cDNA sequence of clone 63298528

SEQ ID NO:3154 is the determined cDNA sequence of clone 63298529

SEQ ID NO:3155 is the determined cDNA sequence of clone 63298530

SEQ ID NO:3156 is the determined cDNA sequence of clone 63298531

SEQ ID NO:3157 is the determined cDNA sequence of clone 63298532

SEQ ID NO:3158 is the determined cDNA sequence of clone 63298533

SEQ ID NO:3159 is the determined cDNA sequence of clone 63298534

SEQ ID NO:3160 is the determined cDNA sequence of clone 63298535

SEQ ID NO:3161 is the determined cDNA sequence of clone 63298537

SEQ ID NO:3162 is the determined cDNA sequence of clone 63298538

SEQ ID NO:3163 is the determined cDNA sequence of clone 63298539

SEQ ID NO:3164 is the determined cDNA sequence of clone 63298540

SEQ ID NO:3165 is the determined cDNA sequence of clone 63298541

SEQ ID NO:3166 is the determined cDNA sequence of clone 63298542

SEQ ID NO:3167 is the determined cDNA sequence of clone 63298543

SEQ ID NO:3168 is the determined cDNA sequence of clone 63298544

SEQ ID NO:3169 is the determined cDNA sequence of clone 63298545

SEQ ID NO:3170 is the determined cDNA sequence of clone 63298546

SEQ ID NO:3171 is the determined cDNA sequence of clone 63298547

SEQ ID NO:3172 is the determined cDNA sequence of clone 63298548

SEQ ID NO:3173 is the determined cDNA sequence of clone 63298549

SEQ ID NO:3174 is the determined cDNA sequence of clone 63298550

SEQ ID NO:3175 is the determined cDNA sequence of clone 63298551

SEQ ID NO:3176 is the determined cDNA sequence of clone 63298552

SEQ ID NO:3177 is the determined cDNA sequence of clone 63298554

SEQ ID NO:3178 is the determined cDNA sequence of clone 63298555

SEQ ID NO:3179 is the determined cDNA sequence of clone 63298556

SEQ ID NO:3180 is the determined cDNA sequence of clone 63298557

SEQ ID NO:3181 is the determined cDNA sequence of clone 63298558

SEQ ID NO:3182 is the determined cDNA sequence of clone 63298559

SEQ ID NO:3183 is the determined cDNA sequence of clone 63298560

SEQ ID NO:3184 is the determined cDNA sequence of clone 63298561

SEQ ID NO:3185 is the determined cDNA sequence of clone 63298562

SEQ ID NO:3186 is the determined cDNA sequence of clone 63298563

SEQ ID NO:3187 is the determined cDNA sequence of clone 63298564

SEQ ID NO:3188 is the determined cDNA sequence of clone 63298565

SEQ ID NO:3189 is the determined cDNA sequence of clone 63298566

SEQ ID NO:3190 is the determined cDNA sequence of clone 63298567

SEQ ID NO:3191 is the determined cDNA sequence of clone 63298568

SEQ ID NO:3192 is the determined cDNA sequence of clone 63298569

SEQ ID NO:3193 is the determined cDNA sequence of clone 63298570

SEQ ID NO:3194 is the determined cDNA sequence of clone 63298571

SEQ ID NO:3195 is the determined cDNA sequence of clone 63298572

SEQ ID NO:3196 is the determined cDNA sequence of clone 63298573

SEQ ID NO:3197 is the determined cDNA sequence of clone 63298574

SEQ ID NO:3198 is the determined cDNA sequence of clone 63298575

SEQ ID NO:3199 is the determined cDNA sequence of clone 63298576

SEQ ID NO:3200 is the determined cDNA sequence of clone 63298577

SEQ ID NO:3201 is the determined cDNA sequence of clone 63298579

SEQ ID NO:3202 is the determined cDNA sequence of clone 63298580

SEQ ID NO:3203 is the determined cDNA sequence of clone 63298581

SEQ ID NO:3204 is the determined cDNA sequence of clone 63298582

SEQ ID NO:3205 is the determined cDNA sequence of clone 63298583

SEQ ID NO:3206 is the determined cDNA sequence of clone 63298584

SEQ ID NO:3207 is the determined cDNA sequence of clone 63298585

SEQ ID NO:3208 is the determined cDNA sequence of clone 63298586

SEQ ID NO:3209 is the determined cDNA sequence of clone 63298588

SEQ ID NO:3210 is the determined cDNA sequence of clone 63298589

SEQ ID NO:3211 is the determined cDNA sequence of clone 63298590

SEQ ID NO:3212 is the determined cDNA sequence of clone 63298591

SEQ ID NO:3213 is the determined cDNA sequence of clone 63298592

SEQ ID NO:3214 is the determined cDNA sequence of clone 63298593

SEQ ID NO:3215 is the determined cDNA sequence of clone 63298594

SEQ ID NO:3216 is the determined cDNA sequence of clone 63298595

SEQ ID NO:3217 is the determined cDNA sequence of clone 63298596

SEQ ID NO:3218 is the determined cDNA sequence of clone 63298597

SEQ ID NO:3219 is the determined cDNA sequence of clone 63298599

SEQ ID NO:3220 is the determined cDNA sequence of clone 63298600

SEQ ID NO:3221 is the determined cDNA sequence of clone 63298601

SEQ ID NO:3222 is the determined cDNA sequence of clone 63298602

SEQ ID NO:3223 is the determined cDNA sequence of clone 63298603

SEQ ID NO:3224 is the determined cDNA sequence of clone 63298604

SEQ ID NO:3225 is the determined cDNA sequence of clone 63298605

SEQ ID NO:3226 is the determined cDNA sequence of clone 63298606

SEQ ID NO:3227 is the determined cDNA sequence of clone 63298607

SEQ ID NO:3228 is the determined cDNA sequence of clone 63298608

SEQ ID NO:3229 is the determined cDNA sequence of clone 63231735

SEQ ID NO:3230 is the determined cDNA sequence of clone 63231736

SEQ ID NO:3231 is the determined cDNA sequence of clone 63231737

SEQ ID NO:3232 is the determined cDNA sequence of clone 63231738

SEQ ID NO:3233 is the determined cDNA sequence of clone 63231739

SEQ ID NO:3234 is the determined cDNA sequence of clone 63231740

SEQ ID NO:3235 is the determined cDNA sequence of clone 63231741

SEQ ID NO:3236 is the determined cDNA sequence of clone 63231742

SEQ ID NO:3237 is the determined cDNA sequence of clone 63231743

SEQ ID NO:3238 is the determined cDNA sequence of clone 63231746

SEQ ID NO:3239 is the determined cDNA sequence of clone 63231755

SEQ ID NO:3240 is the determined cDNA sequence of clone 63231756

SEQ ID NO:3241 is the determined cDNA sequence of clone 63231757

SEQ ID NO:3242 is the determined cDNA sequence of clone 63231758

SEQ ID NO:3243 is the determined cDNA sequence of clone 63231759

SEQ ID NO:3244 is the determined cDNA sequence of clone 63231760

SEQ ID NO:3245 is the determined cDNA sequence of clone 63231761

SEQ ID NO:3246 is the determined cDNA sequence of clone 63231762

SEQ ID NO:3247 is the determined cDNA sequence of clone 63231763

SEQ ID NO:3248 is the determined cDNA sequence of clone 63231764

SEQ ID NO:3249 is the determined cDNA sequence of clone 63231765

SEQ ID NO:3250 is the determined cDNA sequence of clone 63231766

SEQ ID NO:3251 is the determined cDNA sequence of clone 63231767

SEQ ID NO:3252 is the determined cDNA sequence of clone 63231768

SEQ ID NO:3253 is the determined cDNA sequence of clone 63231769

SEQ ID NO:3254 is the determined cDNA sequence of clone 63231770

SEQ ID NO:3255 is the determined cDNA sequence of clone 63231771

SEQ ID NO:3256 is the determined cDNA sequence of clone 63231772

SEQ ID NO:3257 is the determined cDNA sequence of clone 63231773

SEQ ID NO:3258 is the determined cDNA sequence of clone 63231774

SEQ ID NO:3259 is the determined cDNA sequence of clone 63231775

SEQ ID NO:3260 is the determined cDNA sequence of clone 63231776

SEQ ID NO:3261 is the determined cDNA sequence of clone 63231778

SEQ ID NO:3262 is the determined cDNA sequence of clone 63231779

SEQ ID NO:3263 is the determined cDNA sequence of clone 63231780

SEQ ID NO:3264 is the determined cDNA sequence of clone 63231781

SEQ ID NO:3265 is the determined cDNA sequence of clone 63231783

SEQ ID NO:3266 is the determined cDNA sequence of clone 63231784

SEQ ID NO:3267 is the determined cDNA sequence of clone 63231785

SEQ ID NO:3268 is the determined cDNA sequence of clone 63231786

SEQ ID NO:3269 is the determined cDNA sequence of clone 63231787

SEQ ID NO:3270 is the determined cDNA sequence of clone 63231788

SEQ ID NO:3271 is the determined cDNA sequence of clone 63231789

SEQ ID NO:3272 is the determined cDNA sequence of clone 63231790

SEQ ID NO:3273 is the determined cDNA sequence of clone 63231791

SEQ ID NO:3274 is the determined cDNA sequence of clone 63231792

SEQ ID NO:3275 is the determined cDNA sequence of clone 63231793

SEQ ID NO:3276 is the determined cDNA sequence of clone 63231794

SEQ ID NO:3277 is the determined cDNA sequence of clone 63231795

SEQ ID NO:3278 is the determined cDNA sequence of clone 63231796

SEQ ID NO:3279 is the determined cDNA sequence of clone 63231797

SEQ ID NO:3280 is the determined cDNA sequence of clone 63231798

SEQ ID NO:3281 is the determined cDNA sequence of clone 63231799

SEQ ID NO:3282 is the determined cDNA sequence of clone 63231800

SEQ ID NO:3283 is the determined cDNA sequence of clone 63231801

SEQ ID NO:3284 is the determined cDNA sequence of clone 63231802

SEQ ID NO:3285 is the determined cDNA sequence of clone 63231803

SEQ ID NO:3286 is the determined cDNA sequence of clone 63231804

SEQ ID NO:3287 is the determined cDNA sequence of clone 63231805

SEQ ID NO:3288 is the determined cDNA sequence of clone 63231806

SEQ ID NO:3289 is the determined cDNA sequence of clone 63231809

SEQ ID NO:3290 is the determined cDNA sequence of clone 63231810

SEQ ID NO:3291 is the determined cDNA sequence of clone 63231811

SEQ ID NO:3292 is the determined cDNA sequence of clone 63231812

SEQ ID NO:3293 is the determined cDNA sequence of clone 63231813

SEQ ID NO:3294 is the determined cDNA sequence of clone 63231814

SEQ ID NO:3295 is the determined cDNA sequence of clone 63231815

SEQ ID NO:3296 is the determined cDNA sequence of clone 63231816

SEQ ID NO:3297 is the determined cDNA sequence of clone 63231817

SEQ ID NO:3298 is the determined cDNA sequence of clone 63231818

SEQ ID NO:3299 is the determined cDNA sequence of clone 63231819

SEQ ID NO:3300 is the determined cDNA sequence of clone 63231821

SEQ ID NO:3301 is the determined cDNA sequence of clone 63231822

SEQ ID NO:3302 is the determined cDNA sequence of clone 63231823

SEQ ID NO:3303 is the determined cDNA sequence of clone 63231824

SEQ ID NO:3304 is the determined cDNA sequence of clone 63231825

SEQ ID NO:3305 is the determined cDNA sequence of clone 63250511

SEQ ID NO:3306 is the determined cDNA sequence of clone 63250512

SEQ ID NO:3307 is the determined cDNA sequence of clone 63250513

SEQ ID NO:3308 is the determined cDNA sequence of clone 63250514

SEQ ID NO:3309 is the determined cDNA sequence of clone 63250516

SEQ ID NO:3310 is the determined cDNA sequence of clone 63250517

SEQ ID NO:3311 is the determined cDNA sequence of clone 63250518

SEQ ID NO:3312 is the determined cDNA sequence of clone 63250519

SEQ ID NO:3313 is the determined cDNA sequence of clone 63250520

SEQ ID NO:3314 is the determined cDNA sequence of clone 63250521

SEQ ID NO:3315 is the determined cDNA sequence of clone 63250525

SEQ ID NO:3316 is the determined cDNA sequence of clone 63250526

SEQ ID NO:3317 is the determined cDNA sequence of clone 63250527

SEQ ID NO:3318 is the determined cDNA sequence of clone 63250528

SEQ ID NO:3319 is the determined cDNA sequence of clone 63250529

SEQ ID NO:3320 is the determined cDNA sequence of clone 63250530

SEQ ID NO:3321 is the determined cDNA sequence of clone 63250531

SEQ ID NO:3322 is the determined cDNA sequence of clone 63250532

SEQ ID NO:3323 is the determined cDNA sequence of clone 63250533

SEQ ID NO:3324 is the determined cDNA sequence of clone 63250535

SEQ ID NO:3325 is the determined cDNA sequence of clone 63250536

SEQ ID NO:3326 is the determined cDNA sequence of clone 63250537

SEQ ID NO:3327 is the determined cDNA sequence of clone 63250538

SEQ ID NO:3328 is the determined cDNA sequence of clone 63250540

SEQ ID NO:3329 is the determined cDNA sequence of clone 63250541

SEQ ID NO:3330 is the determined cDNA sequence of clone 63250542

SEQ ID NO:3331 is the determined cDNA sequence of clone 63250543

SEQ ID NO:3332 is the determined cDNA sequence of clone 63250544

SEQ ID NO:3333 is the determined cDNA sequence of clone 63250545

SEQ ID NO:3334 is the determined cDNA sequence of clone 63250546

SEQ ID NO:3335 is the determined cDNA sequence of clone 63250547

SEQ ID NO:3336 is the determined cDNA sequence of clone 63250548

SEQ ID NO:3337 is the determined cDNA sequence of clone 63250549

SEQ ID NO:3338 is the determined cDNA sequence of clone 63250550

SEQ ID NO:3339 is the determined cDNA sequence of clone 63250551

SEQ ID NO:3340 is the determined cDNA sequence of clone 63250552

SEQ ID NO:3341 is the determined cDNA sequence of clone 63250553

SEQ ID NO:3342 is the determined cDNA sequence of clone 63250554

SEQ ID NO:3343 is the determined cDNA sequence of clone 63250555

SEQ ID NO:3344 is the determined cDNA sequence of clone 63250556

SEQ ID NO:3345 is the determined cDNA sequence of clone 63250558

SEQ ID NO:3346 is the determined cDNA sequence of clone 63250559

SEQ ID NO:3347 is the determined cDNA sequence of clone 63250560

SEQ ID NO:3348 is the determined cDNA sequence of clone 63250561

SEQ ID NO:3349 is the determined cDNA sequence of clone 63250562

SEQ ID NO:3350 is the determined cDNA sequence of clone 63250563

SEQ ID NO:3351 is the determined cDNA sequence of clone 63250564

SEQ ID NO:3352 is the determined cDNA sequence of clone 63250566

SEQ ID NO:3353 is the determined cDNA sequence of clone 63250567

SEQ ID NO:3354 is the determined cDNA sequence of clone 63250568

SEQ ID NO:3355 is the determined cDNA sequence of clone 63250569

SEQ ID NO:3356 is the determined cDNA sequence of clone 63250570

SEQ ID NO:3357 is the determined cDNA sequence of clone 63250572

SEQ ID NO:3358 is the determined cDNA sequence of clone 63250573

SEQ ID NO:3359 is the determined cDNA sequence of clone 63250574

SEQ ID NO:3360 is the determined cDNA sequence of clone 63250575

SEQ ID NO:3361 is the determined cDNA sequence of clone 63250576

SEQ ID NO:3362 is the determined cDNA sequence of clone 63250577

SEQ ID NO:3363 is the determined cDNA sequence of clone 63250578

SEQ ID NO:3364 is the determined cDNA sequence of clone 63250579

SEQ ID NO:3365 is the determined cDNA sequence of clone 63250580

SEQ ID NO:3366 is the determined cDNA sequence of clone 63250581

SEQ ID NO:3367 is the determined cDNA sequence of clone 63250582

SEQ ID NO:3368 is the determined cDNA sequence of clone 63250583

SEQ ID NO:3369 is the determined cDNA sequence of clone 63250584

SEQ ID NO:3370 is the determined cDNA sequence of clone 63250585

SEQ ID NO:3371 is the determined cDNA sequence of clone 63250586

SEQ ID NO:3372 is the determined cDNA sequence of clone 63250587

SEQ ID NO:3373 is the determined cDNA sequence of clone 63250588

SEQ ID NO:3374 is the determined cDNA sequence of clone 63250589

SEQ ID NO:3375 is the determined cDNA sequence of clone 63250590

SEQ ID NO:3376 is the determined cDNA sequence of clone 63250591

SEQ ID NO:3377 is the determined cDNA sequence of clone 63250592

SEQ ID NO:3378 is the determined cDNA sequence of clone 63250593

SEQ ID NO:3379 is the determined cDNA sequence of clone 63250594

SEQ ID NO:3380 is the determined cDNA sequence of clone 63250595

SEQ ID NO:3381 is the determined cDNA sequence of clone 63250596

SEQ ID NO:3382 is the determined cDNA sequence of clone 63250597

SEQ ID NO:3383 is the determined cDNA sequence of clone 63250598

SEQ ID NO:3384 is the determined cDNA sequence of clone 63250599

SEQ ID NO:3385 is the determined cDNA sequence of clone 63250600

SEQ ID NO:3386 is the determined cDNA sequence of clone 63250601

SEQ ID NO:3387 is the determined cDNA sequence of clone 63250602

SEQ ID NO:3388 is the determined cDNA sequence of clone 63250603

SEQ ID NO:3389 is the determined cDNA sequence of clone 63231826

SEQ ID NO:3390 is the determined cDNA sequence of clone 63231827

SEQ ID NO:3391 is the determined cDNA sequence of clone 63231828

SEQ ID NO:3392 is the determined cDNA sequence of clone 63231829

SEQ ID NO:3393 is the determined cDNA sequence of clone 63231830

SEQ ID NO:3394 is the determined cDNA sequence of clone 63231831

SEQ ID NO:3395 is the determined cDNA sequence of clone 63231832

SEQ ID NO:3396 is the determined cDNA sequence of clone 63231833

SEQ ID NO:3397 is the determined cDNA sequence of clone 63231834

SEQ ID NO:3398 is the determined cDNA sequence of clone 63231835

SEQ ID NO:3399 is the determined cDNA sequence of clone 63231836

SEQ ID NO:3400 is the determined cDNA sequence of clone 63231837

SEQ ID NO:3401 is the determined cDNA sequence of clone 63231838

SEQ ID NO:3402 is the determined cDNA sequence of clone 63231839

SEQ ID NO:3403 is the determined cDNA sequence of clone 63231840

SEQ ID NO:3404 is the determined cDNA sequence of clone 63231841

SEQ ID NO:3405 is the determined cDNA sequence of clone 63231842

SEQ ID NO:3406 is the determined cDNA sequence of clone 63231843

SEQ ID NO:3407 is the determined cDNA sequence of clone 63231844

SEQ ID NO:3408 is the determined cDNA sequence of clone 63231845

SEQ ID NO:3409 is the determined cDNA sequence of clone 63231846

SEQ ID NO:3410 is the determined cDNA sequence of clone 63231847

SEQ ID NO:3411 is the determined cDNA sequence of clone 63231848

SEQ ID NO:3412 is the determined cDNA sequence of clone 63231849

SEQ ID NO:3413 is the determined cDNA sequence of clone 63231850

SEQ ID NO:3414 is the determined cDNA sequence of clone 63231851

SEQ ID NO:3415 is the determined cDNA sequence of clone 63231852

SEQ ID NO:3416 is the determined cDNA sequence of clone 63231853

SEQ ID NO:3417 is the determined cDNA sequence of clone 63231854

SEQ ID NO:3418 is the determined cDNA sequence of clone 63231855

SEQ ID NO:3419 is the determined cDNA sequence of clone 63231856

SEQ ID NO:3420 is the determined cDNA sequence of clone 63231857

SEQ ID NO:3421 is the determined cDNA sequence of clone 63231858

SEQ ID NO:3422 is the determined cDNA sequence of clone 63231859

SEQ ID NO:3423 is the determined cDNA sequence of clone 63231860

SEQ ID NO:3424 is the determined cDNA sequence of clone 63231861

SEQ ID NO:3425 is the determined cDNA sequence of clone 63231862

SEQ ID NO:3426 is the determined cDNA sequence of clone 63231863

SEQ ID NO:3427 is the determined cDNA sequence of clone 63231864

SEQ ID NO:3428 is the determined cDNA sequence of clone 63231865

SEQ ID NO:3429 is the determined cDNA sequence of clone 63231866

SEQ ID NO:3430 is the determined cDNA sequence of clone 63231867

SEQ ID NO:3431 is the determined cDNA sequence of clone 63231868

SEQ ID NO:3432 is the determined cDNA sequence of clone 63231869

SEQ ID NO:3433 is the determined cDNA sequence of clone 63231871

SEQ ID NO:3434 is the determined cDNA sequence of clone 63231872

SEQ ID NO:3435 is the determined cDNA sequence of clone 63231873

SEQ ID NO:3436 is the determined cDNA sequence of clone 63231875

SEQ ID NO:3437 is the determined cDNA sequence of clone 63231876

SEQ ID NO:3438 is the determined cDNA sequence of clone 63231877

SEQ ID NO:3439 is the determined cDNA sequence of clone 63231878

SEQ ID NO:3440 is the determined cDNA sequence of clone 63231879

SEQ ID NO:3441 is the determined cDNA sequence of clone 63231880

SEQ ID NO:3442 is the determined cDNA sequence of clone 63231881

SEQ ID NO:3443 is the determined cDNA sequence of clone 63231882

SEQ ID NO:3444 is the determined cDNA sequence of clone 63231883

SEQ ID NO:3445 is the determined cDNA sequence of clone 63231884

SEQ ID NO:3446 is the determined cDNA sequence of clone 63231885

SEQ ID NO:3447 is the determined cDNA sequence of clone 63231886

SEQ ID NO:3448 is the determined cDNA sequence of clone 63231887

SEQ ID NO:3449 is the determined cDNA sequence of clone 63231888

SEQ ID NO:3450 is the determined cDNA sequence of clone 63231889

SEQ ID NO:3451 is the determined cDNA sequence of clone 63231890

SEQ ID NO:3452 is the determined cDNA sequence of clone 63231891

SEQ ID NO:3453 is the determined cDNA sequence of clone 63231892

SEQ ID NO:3454 is the determined cDNA sequence of clone 63231893

SEQ ID NO:3455 is the determined cDNA sequence of clone 63231894

SEQ ID NO:3456 is the determined cDNA sequence of clone 63231895

SEQ ID NO:3457 is the determined cDNA sequence of clone 63231898

SEQ ID NO:3458 is the determined cDNA sequence of clone 63231899

SEQ ID NO:3459 is the determined cDNA sequence of clone 63231900

SEQ ID NO:3460 is the determined cDNA sequence of clone 63231901

SEQ ID NO:3461 is the determined cDNA sequence of clone 63231902

SEQ ID NO:3462 is the determined cDNA sequence of clone 63231903

SEQ ID NO:3463 is the determined cDNA sequence of clone 63231904

SEQ ID NO:3464 is the determined cDNA sequence of clone 63231905

SEQ ID NO:3465 is the determined cDNA sequence of clone 63231906

SEQ ID NO:3466 is the determined cDNA sequence of clone 63231907

SEQ ID NO:3467 is the determined cDNA sequence of clone 63231908

SEQ ID NO:3468 is the determined cDNA sequence of clone 63231910

SEQ ID NO:3469 is the determined cDNA sequence of clone 63231911

SEQ ID NO:3470 is the determined cDNA sequence of clone 63231913

SEQ ID NO:3471 is the determined cDNA sequence of clone 63231914

SEQ ID NO:3472 is the determined cDNA sequence of clone 63231915

SEQ ID NO:3473 is the determined cDNA sequence of clone 63231917

SEQ ID NO:3474 is the determined cDNA sequence of clone 63231918

SEQ ID NO:3475 is the determined cDNA sequence of clone 63231548

SEQ ID NO:3476 is the determined cDNA sequence of clone 63231549

SEQ ID NO:3477 is the determined cDNA sequence of clone 63231550

SEQ ID NO:3478 is the determined cDNA sequence of clone 63231551

SEQ ID NO:3479 is the determined cDNA sequence of clone 63231552

SEQ ID NO:3480 is the determined cDNA sequence of clone 63231553

SEQ ID NO:3481 is the determined cDNA sequence of clone 63231554

SEQ ID NO:3482 is the determined cDNA sequence of clone 63231555

SEQ ID NO:3483 is the determined cDNA sequence of clone 63231557

SEQ ID NO:3484 is the determined cDNA sequence of clone 63231558

SEQ ID NO:3485 is the determined cDNA sequence of clone 63231559

SEQ ID NO:3486 is the determined cDNA sequence of clone 63231560

SEQ ID NO:3487 is the determined cDNA sequence of clone 63231561

SEQ ID NO:3488 is the determined cDNA sequence of clone 63231562

SEQ ID NO:3489 is the determined cDNA sequence of clone 63231563

SEQ ID NO:3490 is the determined cDNA sequence of clone 63231564

SEQ ID NO:3491 is the determined cDNA sequence of clone 63231565

SEQ ID NO:3492 is the determined cDNA sequence of clone 63231566

SEQ ID NO:3493 is the determined cDNA sequence of clone 63231567

SEQ ID NO:3494 is the determined cDNA sequence of clone 63231568

SEQ ID NO:3495 is the determined cDNA sequence of clone 63231569

SEQ ID NO:3496 is the determined cDNA sequence of clone 63231571

SEQ ID NO:3497 is the determined cDNA sequence of clone 63231572

SEQ ID NO:3498 is the determined cDNA sequence of clone 63231573

SEQ ID NO:3499 is the determined cDNA sequence of clone 63231574

SEQ ID NO:3500 is the determined cDNA sequence of clone 63231575

SEQ ID NO:3501 is the determined cDNA sequence of clone 63231576

SEQ ID NO:3502 is the determined cDNA sequence of clone 63231577

SEQ ID NO:3503 is the determined cDNA sequence of clone 63231578

SEQ ID NO:3504 is the determined cDNA sequence of clone 63231579

SEQ ID NO:3505 is the determined cDNA sequence of clone 63231580

SEQ ID NO:3506 is the determined cDNA sequence of clone 63231581

SEQ ID NO:3507 is the determined cDNA sequence of clone 63231582

SEQ ID NO:3508 is the determined cDNA sequence of clone 63231583

SEQ ID NO:3509 is the determined cDNA sequence of clone 63231584

SEQ ID NO:3510 is the determined cDNA sequence of clone 63231585

SEQ ID NO:3511 is the determined cDNA sequence of clone 63231586

SEQ ID NO:3512 is the determined cDNA sequence of clone 63231587

SEQ ID NO:3513 is the determined cDNA sequence of clone 63231588

SEQ ID NO:3514 is the determined cDNA sequence of clone 63231589

SEQ ID NO:3515 is the determined cDNA sequence of clone 63231590

SEQ ID NO:3516 is the determined cDNA sequence of clone 63231591

SEQ ID NO:3517 is the determined cDNA sequence of clone 63231592

SEQ ID NO:3518 is the determined cDNA sequence of clone 63231593

SEQ ID NO:3519 is the determined cDNA sequence of clone 63231594

SEQ ID NO:3520 is the determined cDNA sequence of clone 63231595

SEQ ID NO:3521 is the determined cDNA sequence of clone 63231596

SEQ ID NO:3522 is the determined cDNA sequence of clone 63231597

SEQ ID NO:3523 is the determined cDNA sequence of clone 63231598

SEQ ID NO:3524 is the determined cDNA sequence of clone 63231599

SEQ ID NO:3525 is the determined cDNA sequence of clone 63231600

SEQ ID NO:3526 is the determined cDNA sequence of clone 63231601

SEQ ID NO:3527 is the determined cDNA sequence of clone 63231602

SEQ ID NO:3528 is the determined cDNA sequence of clone 63231603

SEQ ID NO:3529 is the determined cDNA sequence of clone 63231604

SEQ ID NO:3530 is the determined cDNA sequence of clone 63231605

SEQ ID NO:3531 is the determined cDNA sequence of clone 63231606

SEQ ID NO:3532 is the determined cDNA sequence of clone 63231607

SEQ ID NO:3533 is the determined cDNA sequence of clone 63231608

SEQ ID NO:3534 is the determined cDNA sequence of clone 63231609

SEQ ID NO:3535 is the determined cDNA sequence of clone 63231610

SEQ ID NO:3536 is the determined cDNA sequence of clone 63231611

SEQ ID NO:3537 is the determined cDNA sequence of clone 63231612

SEQ ID NO:3538 is the determined cDNA sequence of clone 63231613

SEQ ID NO:3539 is the determined cDNA sequence of clone 63231614

SEQ ID NO:3540 is the determined cDNA sequence of clone 63231615

SEQ ID NO:3541 is the determined cDNA sequence of clone 63231616

SEQ ID NO:3542 is the determined cDNA sequence of clone 63231617

SEQ ID NO:3543 is the determined cDNA sequence of clone 63231618

SEQ ID NO:3544 is the determined cDNA sequence of clone 63231619

SEQ ID NO:3545 is the determined cDNA sequence of clone 63231620

SEQ ID NO:3546 is the determined cDNA sequence of clone 63231621

SEQ ID NO:3547 is the determined cDNA sequence of clone 63231623

SEQ ID NO:3548 is the determined cDNA sequence of clone 63231625

SEQ ID NO:3549 is the determined cDNA sequence of clone 63231626

SEQ ID NO:3550 is the determined cDNA sequence of clone 63231627

SEQ ID NO:3551 is the determined cDNA sequence of clone 63231628

SEQ ID NO:3552 is the determined cDNA sequence of clone 63231629

SEQ ID NO:3553 is the determined cDNA sequence of clone 63231630

SEQ ID NO:3554 is the determined cDNA sequence of clone 63231631

SEQ ID NO:3555 is the determined cDNA sequence of clone 63231632

SEQ ID NO:3556 is the determined cDNA sequence of clone 63231633

SEQ ID NO:3557 is the determined cDNA sequence of clone 63231634

SEQ ID NO:3558 is the determined cDNA sequence of clone 63231635

SEQ ID NO:3559 is the determined cDNA sequence of clone 63231636

SEQ ID NO:3560 is the determined cDNA sequence of clone 63231637

SEQ ID NO:3561 is the determined cDNA sequence of clone 63231638

SEQ ID NO:3562 is the determined cDNA sequence of clone 63231639

SEQ ID NO:3563 is the determined cDNA sequence of clone 63298237

SEQ ID NO:3564 is the determined cDNA sequence of clone 63298238

SEQ ID NO:3565 is the determined cDNA sequence of clone 63298239

SEQ ID NO:3566 is the determined cDNA sequence of clone 63298240

SEQ ID NO:3567 is the determined cDNA sequence of clone 63298241

SEQ ID NO:3568 is the determined cDNA sequence of clone 63298242

SEQ ID NO:3569 is the determined cDNA sequence of clone 63298243

SEQ ID NO:3570 is the determined cDNA sequence of clone 63298244

SEQ ID NO:3571 is the determined cDNA sequence of clone 63298245

SEQ ID NO:3572 is the determined cDNA sequence of clone 63298246

SEQ ID NO:3573 is the determined cDNA sequence of clone 63298247

SEQ ID NO:3574 is the determined cDNA sequence of clone 63298248

SEQ ID NO:3575 is the determined cDNA sequence of clone 63298249

SEQ ID NO:3576 is the determined cDNA sequence of clone 63298250

SEQ ID NO:3577 is the determined cDNA sequence of clone 63298251

SEQ ID NO:3578 is the determined cDNA sequence of clone 63298252

SEQ ID NO:3579 is the determined cDNA sequence of clone 63298253

SEQ ID NO:3580 is the determined cDNA sequence of clone 63298254

SEQ ID NO:3581 is the determined cDNA sequence of clone 63298255

SEQ ID NO:3582 is the determined cDNA sequence of clone 63298256

SEQ ID NO:3583 is the determined cDNA sequence of clone 63298257

SEQ ID NO:3584 is the determined cDNA sequence of clone 63298258

SEQ ID NO:3585 is the determined cDNA sequence of clone 63298259

SEQ ID NO:3586 is the determined cDNA sequence of clone 63298261

SEQ ID NO:3587 is the determined cDNA sequence of clone 63298262

SEQ ID NO:3588 is the determined cDNA sequence of clone 63298263

SEQ ID NO:3589 is the determined cDNA sequence of clone 63298264

SEQ ID NO:3590 is the determined cDNA sequence of clone 63298265

SEQ ID NO:3591 is the determined cDNA sequence of clone 63298266

SEQ ID NO:3592 is the determined cDNA sequence of clone 63298267

SEQ ID NO:3593 is the determined cDNA sequence of clone 63298268

SEQ ID NO:3594 is the determined cDNA sequence of clone 63298269

SEQ ID NO:3595 is the determined cDNA sequence of clone 63298270

SEQ ID NO:3596 is the determined cDNA sequence of clone 63298271

SEQ ID NO:3597 is the determined cDNA sequence of clone 63298273

SEQ ID NO:3598 is the determined cDNA sequence of clone 63298274

SEQ ID NO:3599 is the determined cDNA sequence of clone 63298275

SEQ ID NO:3600 is the determined cDNA sequence of clone 63298276

SEQ ID NO:3601 is the determined cDNA sequence of clone 63298278

SEQ ID NO:3602 is the determined cDNA sequence of clone 63298279

SEQ ID NO:3603 is the determined cDNA sequence of clone 63298280

SEQ ID NO:3604 is the determined cDNA sequence of clone 63298281

SEQ ID NO:3605 is the determined cDNA sequence of clone 63298282

SEQ ID NO:3606 is the determined cDNA sequence of clone 63298284

SEQ ID NO:3607 is the determined cDNA sequence of clone 63298287

SEQ ID NO:3608 is the determined cDNA sequence of clone 63298288

SEQ ID NO:3609 is the determined cDNA sequence of clone 63298289

SEQ ID NO:3610 is the determined cDNA sequence of clone 63298290

SEQ ID NO:3611 is the determined cDNA sequence of clone 63298291

SEQ ID NO:3612 is the determined cDNA sequence of clone 63298292

SEQ ID NO:3613 is the determined cDNA sequence of clone 63298293

SEQ ID NO:3614 is the determined cDNA sequence of clone 63298294

SEQ ID NO:3615 is the determined cDNA sequence of clone 63298295

SEQ ID NO:3616 is the determined cDNA sequence of clone 63298296

SEQ ID NO:3617 is the determined cDNA sequence of clone 63298297

SEQ ID NO:3618 is the determined cDNA sequence of clone 63298299

SEQ ID NO:3619 is the determined cDNA sequence of clone 63298300

SEQ ID NO:3620 is the determined cDNA sequence of clone 63298301

SEQ ID NO:3621 is the determined cDNA sequence of clone 63298303

SEQ ID NO:3622 is the determined cDNA sequence of clone 63298304

SEQ ID NO:3623 is the determined cDNA sequence of clone 63298307

SEQ ID NO:3624 is the determined cDNA sequence of clone 63298308

SEQ ID NO:3625 is the determined cDNA sequence of clone 63298309

SEQ ID NO:3626 is the determined cDNA sequence of clone 63298310

SEQ ID NO:3627 is the determined cDNA sequence of clone 63298311

SEQ ID NO:3628 is the determined cDNA sequence of clone 63298313

SEQ ID NO:3629 is the determined cDNA sequence of clone 63298314

SEQ ID NO:3630 is the determined cDNA sequence of clone 63298315

SEQ ID NO:3631 is the determined cDNA sequence of clone 63298316

SEQ ID NO:3632 is the determined cDNA sequence of clone 63298317

SEQ ID NO:3633 is the determined cDNA sequence of clone 63298318

SEQ ID NO:3634 is the determined cDNA sequence of clone 63298319

SEQ ID NO:3635 is the determined cDNA sequence of clone 63298321

SEQ ID NO:3636 is the determined cDNA sequence of clone 63298323

SEQ ID NO:3637 is the determined cDNA sequence of clone 63298324

SEQ ID NO:3638 is the determined cDNA sequence of clone 63298325

SEQ ID NO:3639 is the determined cDNA sequence of clone 63298326

SEQ ID NO:3640 is the determined cDNA sequence of clone 63298327

SEQ ID NO:3641 is the determined cDNA sequence of clone 63298328

SEQ ID NO:3642 is the determined cDNA sequence of clone 63298329

SEQ ID NO:3643 is the determined cDNA sequence of clone 63298424

SEQ ID NO:3644 is the determined cDNA sequence of clone 63298425

SEQ ID NO:3645 is the determined cDNA sequence of clone 63298426

SEQ ID NO:3646 is the determined cDNA sequence of clone 63298427

SEQ ID NO:3647 is the determined cDNA sequence of clone 63298428

SEQ ID NO:3648 is the determined cDNA sequence of clone 63298429

SEQ ID NO:3649 is the determined cDNA sequence of clone 63298430

SEQ ID NO:3650 is the determined cDNA sequence of clone 63298431

SEQ ID NO:3651 is the determined cDNA sequence of clone 63298432

SEQ ID NO:3652 is the determined cDNA sequence of clone 63298433

SEQ ID NO:3653 is the determined cDNA sequence of clone 63298434

SEQ ID NO:3654 is the determined cDNA sequence of clone 63298435

SEQ ID NO:3655 is the determined cDNA sequence of clone 63298436

SEQ ID NO:3656 is the determined cDNA sequence of clone 63298437

SEQ ID NO:3657 is the determined cDNA sequence of clone 63298438

SEQ ID NO:3658 is the determined cDNA sequence of clone 63298439

SEQ ID NO:3659 is the determined cDNA sequence of clone 63298440

SEQ ID NO:3660 is the determined cDNA sequence of clone 63298441

SEQ ID NO:3661 is the determined cDNA sequence of clone 63298443

SEQ ID NO:3662 is the determined cDNA sequence of clone 63298444

SEQ ID NO:3663 is the determined cDNA sequence of clone 63298445

SEQ ID NO:3664 is the determined cDNA sequence of clone 63298446

SEQ ID NO:3665 is the determined cDNA sequence of clone 63298447

SEQ ID NO:3666 is the determined cDNA sequence of clone 63298448

SEQ ID NO:3667 is the determined cDNA sequence of clone 63298449

SEQ ID NO:3668 is the determined cDNA sequence of clone 63298451

SEQ ID NO:3669 is the determined cDNA sequence of clone 63298452

SEQ ID NO:3670 is the determined cDNA sequence of clone 63298453

SEQ ID NO:3671 is the determined cDNA sequence of clone 63298454

SEQ ID NO:3672 is the determined cDNA sequence of clone 63298455

SEQ ID NO:3673 is the determined cDNA sequence of clone 63298456

SEQ ID NO:3674 is the determined cDNA sequence of clone 63298457

SEQ ID NO:3675 is the determined cDNA sequence of clone 63298458

SEQ ID NO:3676 is the determined cDNA sequence of clone 63298459

SEQ ID NO:3677 is the determined cDNA sequence of clone 63298460

SEQ ID NO:3678 is the determined cDNA sequence of clone 63298461

SEQ ID NO:3679 is the determined cDNA sequence of clone 63298462

SEQ ID NO:3680 is the determined cDNA sequence of clone 63298463

SEQ ID NO:3681 is the determined cDNA sequence of clone 63298464

SEQ ID NO:3682 is the determined cDNA sequence of clone 63298465

SEQ ID NO:3683 is the determined cDNA sequence of clone 63298466

SEQ ID NO:3684 is the determined cDNA sequence of clone 63298468

SEQ ID NO:3685 is the determined cDNA sequence of clone 63298469

SEQ ID NO:3686 is the determined cDNA sequence of clone 63298470

SEQ ID NO:3687 is the determined cDNA sequence of clone 63298471

SEQ ID NO:3688 is the determined cDNA sequence of clone 63298472

SEQ ID NO:3689 is the determined cDNA sequence of clone 63298473

SEQ ID NO:3690 is the determined cDNA sequence of clone 63298474

SEQ ID NO:3691 is the determined cDNA sequence of clone 63298475

SEQ ID NO:3692 is the determined cDNA sequence of clone 63298476

SEQ ID NO:3693 is the determined cDNA sequence of clone 63298477

SEQ ID NO:3694 is the determined cDNA sequence of clone 63298478

SEQ ID NO:3695 is the determined cDNA sequence of clone 63298481

SEQ ID NO:3696 is the determined cDNA sequence of clone 63298482

SEQ ID NO:3697 is the determined cDNA sequence of clone 63298483

SEQ ID NO:3698 is the determined cDNA sequence of clone 63298484

SEQ ID NO:3699 is the determined cDNA sequence of clone 63298485

SEQ ID NO:3700 is the determined cDNA sequence of clone 63298486

SEQ ID NO:3701 is the determined cDNA sequence of clone 63298487

SEQ ID NO:3702 is the determined cDNA sequence of clone 63298488

SEQ ID NO:3703 is the determined cDNA sequence of clone 63298489

SEQ ID NO:3704 is the determined cDNA sequence of clone 63298490

SEQ ID NO:3705 is the determined cDNA sequence of clone 63298491

SEQ ID NO:3706 is the determined cDNA sequence of clone 63298492

SEQ ID NO:3707 is the determined cDNA sequence of clone 63298493

SEQ ID NO:3708 is the determined cDNA sequence of clone 63298494

SEQ ID NO:3709 is the determined cDNA sequence of clone 63298495

SEQ ID NO:3710 is the determined cDNA sequence of clone 63298496

SEQ ID NO:3711 is the determined cDNA sequence of clone 63298497

SEQ ID NO:3712 is the determined cDNA sequence of clone 63298498

SEQ ID NO:3713 is the determined cDNA sequence of clone 63298499

SEQ ID NO:3714 is the determined cDNA sequence of clone 63298500

SEQ ID NO:3715 is the determined cDNA sequence of clone 63298501

SEQ ID NO:3716 is the determined cDNA sequence of clone 63298502

SEQ ID NO:3717 is the determined cDNA sequence of clone 63298504

SEQ ID NO:3718 is the determined cDNA sequence of clone 63298505

SEQ ID NO:3719 is the determined cDNA sequence of clone 63298506

SEQ ID NO:3720 is the determined cDNA sequence of clone 63298507

SEQ ID NO:3721 is the determined cDNA sequence of clone 63298508

SEQ ID NO:3722 is the determined cDNA sequence of clone 63298509

SEQ ID NO:3723 is the determined cDNA sequence of clone 63298510

SEQ ID NO:3724 is the determined cDNA sequence of clone 63298511

SEQ ID NO:3725 is the determined cDNA sequence of clone 63298512

SEQ ID NO:3726 is the determined cDNA sequence of clone 63298513

SEQ ID NO:3727 is the determined cDNA sequence of clone 63298514

SEQ ID NO:3728 is the determined cDNA sequence of clone 63298515

SEQ ID NO:3729 is the determined cDNA sequence of clone 63299075

SEQ ID NO:3730 is the determined cDNA sequence of clone 63299076

SEQ ID NO:3731 is the determined cDNA sequence of clone 63299077

SEQ ID NO:3732 is the determined cDNA sequence of clone 63299078

SEQ ID NO:3733 is the determined cDNA sequence of clone 63299079

SEQ ID NO:3734 is the determined cDNA sequence of clone 63299080

SEQ ID NO:3735 is the determined cDNA sequence of clone 63299081

SEQ ID NO:3736 is the determined cDNA sequence of clone 63299082

SEQ ID NO:3737 is the determined cDNA sequence of clone 63299083

SEQ ID NO:3738 is the determined cDNA sequence of clone 63299084

SEQ ID NO:3739 is the determined cDNA sequence of clone 63299085

SEQ ID NO:3740 is the determined cDNA sequence of clone 63299086

SEQ ID NO:3741 is the determined cDNA sequence of clone 63299087

SEQ ID NO:3742 is the determined cDNA sequence of clone 63299088

SEQ ID NO:3743 is the determined cDNA sequence of clone 63299089

SEQ ID NO:3744 is the determined cDNA sequence of clone 63299090

SEQ ID NO:3745 is the determined cDNA sequence of clone 63299092

SEQ ID NO:3746 is the determined cDNA sequence of clone 63299093

SEQ ID NO:3747 is the determined cDNA sequence of clone 63299094

SEQ ID NO:3748 is the determined cDNA sequence of clone 63299095

SEQ ID NO:3749 is the determined cDNA sequence of clone 63299096

SEQ ID NO:3750 is the determined cDNA sequence of clone 63299097

SEQ ID NO:3751 is the determined cDNA sequence of clone 63299099

SEQ ID NO:3752 is the determined cDNA sequence of clone 63299100

SEQ ID NO:3753 is the determined cDNA sequence of clone 63299101

SEQ ID NO:3754 is the determined cDNA sequence of clone 63299102

SEQ ID NO:3755 is the determined cDNA sequence of clone 63299104

SEQ ID NO:3756 is the determined cDNA sequence of clone 63299105

SEQ ID NO:3757 is the determined cDNA sequence of clone 63299106

SEQ ID NO:3758 is the determined cDNA sequence of clone 63299107

SEQ ID NO:3759 is the determined cDNA sequence of clone 63299108

SEQ ID NO:3760 is the determined cDNA sequence of clone 63299109

SEQ ID NO:3761 is the determined cDNA sequence of clone 63299110

SEQ ID NO:3762 is the determined cDNA sequence of clone 63299111

SEQ ID NO:3763 is the determined cDNA sequence of clone 63299112

SEQ ID NO:3764 is the determined cDNA sequence of clone 63299113

SEQ ID NO:3765 is the determined cDNA sequence of clone 63299114

SEQ ID NO:3766 is the determined cDNA sequence of clone 63299116

SEQ ID NO:3767 is the determined cDNA sequence of clone 63299117

SEQ ID NO:3768 is the determined cDNA sequence of clone 63299118

SEQ ID NO:3769 is the determined cDNA sequence of clone 63299120

SEQ ID NO:3770 is the determined cDNA sequence of clone 63299122

SEQ ID NO:3771 is the determined cDNA sequence of clone 63299123

SEQ ID NO:3772 is the determined cDNA sequence of clone 63299125

SEQ ID NO:3773 is the determined cDNA sequence of clone 63299126

SEQ ID NO:3774 is the determined cDNA sequence of clone 63299128

SEQ ID NO:3775 is the determined cDNA sequence of clone 63299129

SEQ ID NO:3776 is the determined cDNA sequence of clone 63299130

SEQ ID NO:3777 is the determined cDNA sequence of clone 63299131

SEQ ID NO:3778 is the determined cDNA sequence of clone 63299132

SEQ ID NO:3779 is the determined cDNA sequence of clone 63299134

SEQ ID NO:3780 is the determined cDNA sequence of clone 63299135

SEQ ID NO:3781 is the determined cDNA sequence of clone 63299136

SEQ ID NO:3782 is the determined cDNA sequence of clone 63299137

SEQ ID NO:3783 is the determined cDNA sequence of clone 63299138

SEQ ID NO:3784 is the determined cDNA sequence of clone 63299139

SEQ ID NO:3785 is the determined cDNA sequence of clone 63299140

SEQ ID NO:3786 is the determined cDNA sequence of clone 63299141

SEQ ID NO:3787 is the determined cDNA sequence of clone 63299142

SEQ ID NO:3788 is the determined cDNA sequence of clone 63299143

SEQ ID NO:3789 is the determined cDNA sequence of clone 63299144

SEQ ID NO:3790 is the determined cDNA sequence of clone 63299146

SEQ ID NO:3791 is the determined cDNA sequence of clone 63299147

SEQ ID NO:3792 is the determined cDNA sequence of clone 63299148

SEQ ID NO:3793 is the determined cDNA sequence of clone 63299152

SEQ ID NO:3794 is the determined cDNA sequence of clone 63299153

SEQ ID NO:3795 is the determined cDNA sequence of clone 63299155

SEQ ID NO:3796 is the determined cDNA sequence of clone 63299156

SEQ ID NO:3797 is the determined cDNA sequence of clone 63299157

SEQ ID NO:3798 is the determined cDNA sequence of clone 63299158

SEQ ID NO:3799 is the determined cDNA sequence of clone 63299159

SEQ ID NO:3800 is the determined cDNA sequence of clone 63299160

SEQ ID NO:3801 is the determined cDNA sequence of clone 63299164

SEQ ID NO:3802 is the determined cDNA sequence of clone 63299165

SEQ ID NO:3803 is the determined cDNA sequence of clone 63299166

SEQ ID NO:3804 is the determined cDNA sequence of clone 63298889

SEQ ID NO:3805 is the determined cDNA sequence of clone 63298890

SEQ ID NO:3806 is the determined cDNA sequence of clone 63298891

SEQ ID NO:3807 is the determined cDNA sequence of clone 63298892

SEQ ID NO:3808 is the determined cDNA sequence of clone 63298893

SEQ ID NO:3809 is the determined cDNA sequence of clone 63298894

SEQ ID NO:3810 is the determined cDNA sequence of clone 63298895

SEQ ID NO:3811 is the determined cDNA sequence of clone 63298896

SEQ ID NO:3812 is the determined cDNA sequence of clone 63298897

SEQ ID NO:3813 is the determined cDNA sequence of clone 63298898

SEQ ID NO:3814 is the determined cDNA sequence of clone 63298899

SEQ ID NO:3815 is the determined cDNA sequence of clone 63298900

SEQ ID NO:3816 is the determined cDNA sequence of clone 63298901

SEQ ID NO:3817 is the determined cDNA sequence of clone 63298902

SEQ ID NO:3818 is the determined cDNA sequence of clone 63298905

SEQ ID NO:3819 is the determined cDNA sequence of clone 63298906

SEQ ID NO:3820 is the determined cDNA sequence of clone 63298907

SEQ ID NO:3821 is the determined cDNA sequence of clone 63298908

SEQ ID NO:3822 is the determined cDNA sequence of clone 63298909

SEQ ID NO:3823 is the determined cDNA sequence of clone 63298911

SEQ ID NO:3824 is the determined cDNA sequence of clone 63298912

SEQ ID NO:3825 is the determined cDNA sequence of clone 63298913

SEQ ID NO:3826 is the determined cDNA sequence of clone 63298914

SEQ ID NO:3827 is the determined cDNA sequence of clone 63298915

SEQ ID NO:3828 is the determined cDNA sequence of clone 63298917

SEQ ID NO:3829 is the determined cDNA sequence of clone 63298918

SEQ ID NO:3830 is the determined cDNA sequence of clone 63298920

SEQ ID NO:3831 is the determined cDNA sequence of clone 63298921

SEQ ID NO:3832 is the determined cDNA sequence of clone 63298922

SEQ ID NO:3833 is the determined cDNA sequence of clone 63298923

SEQ ID NO:3834 is the determined cDNA sequence of clone 63298924

SEQ ID NO:3835 is the determined cDNA sequence of clone 63298926

SEQ ID NO:3836 is the determined cDNA sequence of clone 63298928

SEQ ID NO:3837 is the determined cDNA sequence of clone 63298930

SEQ ID NO:3838 is the determined cDNA sequence of clone 63298931

SEQ ID NO:3839 is the determined cDNA sequence of clone 63298932

SEQ ID NO:3840 is the determined cDNA sequence of clone 63298933

SEQ ID NO:3841 is the determined cDNA sequence of clone 63298934

SEQ ID NO:3842 is the determined cDNA sequence of clone 63298935

SEQ ID NO:3843 is the determined cDNA sequence of clone 63298936

SEQ ID NO:3844 is the determined cDNA sequence of clone 63298937

SEQ ID NO:3845 is the determined cDNA sequence of clone 63298938

SEQ ID NO:3846 is the determined cDNA sequence of clone 63298939

SEQ ID NO:3847 is the determined cDNA sequence of clone 63298940

SEQ ID NO:3848 is the determined cDNA sequence of clone 63298941

SEQ ID NO:3849 is the determined cDNA sequence of clone 63298942

SEQ ID NO:3850 is the determined cDNA sequence of clone 63298943

SEQ ID NO:3851 is the determined cDNA sequence of clone 63298944

SEQ ID NO:3852 is the determined cDNA sequence of clone 63298945

SEQ ID NO:3853 is the determined cDNA sequence of clone 63298946

SEQ ID NO:3854 is the determined cDNA sequence of clone 63298947

SEQ ID NO:3855 is the determined cDNA sequence of clone 63298948

SEQ ID NO:3856 is the determined cDNA sequence of clone 63298951

SEQ ID NO:3857 is the determined cDNA sequence of clone 63298952

SEQ ID NO:3858 is the determined cDNA sequence of clone 63298954

SEQ ID NO:3859 is the determined cDNA sequence of clone 63298957

SEQ ID NO:3860 is the determined cDNA sequence of clone 63298959

SEQ ID NO:3861 is the determined cDNA sequence of clone 63298960

SEQ ID NO:3862 is the determined cDNA sequence of clone 63298961

SEQ ID NO:3863 is the determined cDNA sequence of clone 63298962

SEQ ID NO:3864 is the determined cDNA sequence of clone 63298963

SEQ ID NO:3865 is the determined cDNA sequence of clone 63298964

SEQ ID NO:3866 is the determined cDNA sequence of clone 63298965

SEQ ID NO:3867 is the determined cDNA sequence of clone 63298966

SEQ ID NO:3868 is the determined cDNA sequence of clone 63298968

SEQ ID NO:3869 is the determined cDNA sequence of clone 63298969

SEQ ID NO:3870 is the determined cDNA sequence of clone 63298970

SEQ ID NO:3871 is the determined cDNA sequence of clone 63298971

SEQ ID NO:3872 is the determined cDNA sequence of clone 63298972

SEQ ID NO:3873 is the determined cDNA sequence of clone 63298973

SEQ ID NO:3874 is the determined cDNA sequence of clone 63298974

SEQ ID NO:3875 is the determined cDNA sequence of clone 63298975

SEQ ID NO:3876 is the determined cDNA sequence of clone 63298977

SEQ ID NO:3877 is the determined cDNA sequence of clone 63298978

SEQ ID NO:3878 is the determined cDNA sequence of clone 63298980

SEQ ID NO:3879 is the determined cDNA sequence of clone 63298330

SEQ ID NO:3880 is the determined cDNA sequence of clone 63298331

SEQ ID NO:3881 is the determined cDNA sequence of clone 63298332

SEQ ID NO:3882 is the determined cDNA sequence of clone 63298333

SEQ ID NO:3883 is the determined cDNA sequence of clone 63298334

SEQ ID NO:3884 is the determined cDNA sequence of clone 63298335

SEQ ID NO:3885 is the determined cDNA sequence of clone 63298336

SEQ ID NO:3886 is the determined cDNA sequence of clone 63298337

SEQ ID NO:3887 is the determined cDNA sequence of clone 63298338

SEQ ID NO:3888 is the determined cDNA sequence of clone 63298339

SEQ ID NO:3889 is the determined cDNA sequence of clone 63298340

SEQ ID NO:3890 is the determined cDNA sequence of clone 63298341

SEQ ID NO:3891 is the determined cDNA sequence of clone 63298342

SEQ ID NO:3892 is the determined cDNA sequence of clone 63298343

SEQ ID NO:3893 is the determined cDNA sequence of clone 63298344

SEQ ID NO:3894 is the determined cDNA sequence of clone 63298345

SEQ ID NO:3895 is the determined cDNA sequence of clone 63298346

SEQ ID NO:3896 is the determined cDNA sequence of clone 63298347

SEQ ID NO:3897 is the determined cDNA sequence of clone 63298348

SEQ ID NO:3898 is the determined cDNA sequence of clone 63298349

SEQ ID NO:3899 is the determined cDNA sequence of clone 63298350

SEQ ID NO:3900 is the determined cDNA sequence of clone 63298352

SEQ ID NO:3901 is the determined cDNA sequence of clone 63298353

SEQ ID NO:3902 is the determined cDNA sequence of clone 63298354

SEQ ID NO:3903 is the determined cDNA sequence of clone 63298355

SEQ ID NO:3904 is the determined cDNA sequence of clone 63298356

SEQ ID NO:3905 is the determined cDNA sequence of clone 63298358

SEQ ID NO:3906 is the determined cDNA sequence of clone 63298359

SEQ ID NO:3907 is the determined cDNA sequence of clone 63298360

SEQ ID NO:3908 is the determined cDNA sequence of clone 63298361

SEQ ID NO:3909 is the determined cDNA sequence of clone 63298363

SEQ ID NO:3910 is the determined cDNA sequence of clone 63298364

SEQ ID NO:3911 is the determined cDNA sequence of clone 63298365

SEQ ID NO:3912 is the determined cDNA sequence of clone 63298366

SEQ ID NO:3913 is the determined cDNA sequence of clone 63298367

SEQ ID NO:3914 is the determined cDNA sequence of clone 63298368

SEQ ID NO:3915 is the determined cDNA sequence of clone 63298370

SEQ ID NO:3916 is the determined cDNA sequence of clone 63298371

SEQ ID NO:3917 is the determined cDNA sequence of clone 63298372

SEQ ID NO:3918 is the determined cDNA sequence of clone 63298373

SEQ ID NO:3919 is the determined cDNA sequence of clone 63298374

SEQ ID NO:3920 is the determined cDNA sequence of clone 63298375

SEQ ID NO:3921 is the determined cDNA sequence of clone 63298376

SEQ ID NO:3922 is the determined cDNA sequence of clone 63298377

SEQ ID NO:3923 is the determined cDNA sequence of clone 63298378

SEQ ID NO:3924 is the determined cDNA sequence of clone 63298379

SEQ ID NO:3925 is the determined cDNA sequence of clone 63298380

SEQ ID NO:3926 is the determined cDNA sequence of clone 63298382

SEQ ID NO:3927 is the determined cDNA sequence of clone 63298383

SEQ ID NO:3928 is the determined cDNA sequence of clone 63298384

SEQ ID NO:3929 is the determined cDNA sequence of clone 63298385

SEQ ID NO:3930 is the determined cDNA sequence of clone 63298386

SEQ ID NO:3931 is the determined cDNA sequence of clone 63298387

SEQ ID NO:3932 is the determined cDNA sequence of clone 63298388

SEQ ID NO:3933 is the determined cDNA sequence of clone 63298389

SEQ ID NO:3934 is the determined cDNA sequence of clone 63298390

SEQ ID NO:3935 is the determined cDNA sequence of clone 63298391

SEQ ID NO:3936 is the determined cDNA sequence of clone 63298392

SEQ ID NO:3937 is the determined cDNA sequence of clone 63298394

SEQ ID NO:3938 is the determined cDNA sequence of clone 63298395

SEQ ID NO:3939 is the determined cDNA sequence of clone 63298396

SEQ ID NO:3940 is the determined cDNA sequence of clone 63298397

SEQ ID NO:3941 is the determined cDNA sequence of clone 63298398

SEQ ID NO:3942 is the determined cDNA sequence of clone 63298399

SEQ ID NO:3943 is the determined cDNA sequence of clone 63298401

SEQ ID NO:3944 is the determined cDNA sequence of clone 63298402

SEQ ID NO:3945 is the determined cDNA sequence of clone 63298403

SEQ ID NO:3946 is the determined cDNA sequence of clone 63298404

SEQ ID NO:3947 is the determined cDNA sequence of clone 63298405

SEQ ID NO:3948 is the determined cDNA sequence of clone 63298406

SEQ ID NO:3949 is the determined cDNA sequence of clone 63298407

SEQ ID NO:3950 is the determined cDNA sequence of clone 63298408

SEQ ID NO:3951 is the determined cDNA sequence of clone 63298409

SEQ ID NO:3952 is the determined cDNA sequence of clone 63298410

SEQ ID NO:3953 is the determined cDNA sequence of clone 63298411

SEQ ID NO:3954 is the determined cDNA sequence of clone 63298412

SEQ ID NO:3955 is the determined cDNA sequence of clone 63298414

SEQ ID NO:3956 is the determined cDNA sequence of clone 63298415

SEQ ID NO:3957 is the determined cDNA sequence of clone 63298416

SEQ ID NO:3958 is the determined cDNA sequence of clone 63298417

SEQ ID NO:3959 is the determined cDNA sequence of clone 63298418

SEQ ID NO:3960 is the determined cDNA sequence of clone 63298419

SEQ ID NO:3961 is the determined cDNA sequence of clone 63298420

SEQ ID NO:3962 is the determined cDNA sequence of clone 63298421

SEQ ID NO:3963 is the determined cDNA sequence of clone 63250604

SEQ ID NO:3964 is the determined cDNA sequence of clone 63250605

SEQ ID NO:3965 is the determined cDNA sequence of clone 63250606

SEQ ID NO:3966 is the determined cDNA sequence of clone 63250607

SEQ ID NO:3967 is the determined cDNA sequence of clone 63250608

SEQ ID NO:3968 is the determined cDNA sequence of clone 63250609

SEQ ID NO:3969 is the determined cDNA sequence of clone 63250610

SEQ ID NO:3970 is the determined cDNA sequence of clone 63250611

SEQ ID NO:3971 is the determined cDNA sequence of clone 63250612

SEQ ID NO:3972 is the determined cDNA sequence of clone 63250613

SEQ ID NO:3973 is the determined cDNA sequence of clone 63250614

SEQ ID NO:3974 is the determined cDNA sequence of clone 63250615

SEQ ID NO:3975 is the determined cDNA sequence of clone 63250616

SEQ ID NO:3976 is the determined cDNA sequence of clone 63250617

SEQ ID NO:3977 is the determined cDNA sequence of clone 63250618

SEQ ID NO:3978 is the determined cDNA sequence of clone 63250619

SEQ ID NO:3979 is the determined cDNA sequence of clone 63250620

SEQ ID NO:3980 is the determined cDNA sequence of clone 63250621

SEQ ID NO:3981 is the determined cDNA sequence of clone 63250622

SEQ ID NO:3982 is the determined cDNA sequence of clone 63250623

SEQ ID NO:3983 is the determined cDNA sequence of clone 63250624

SEQ ID NO:3984 is the determined cDNA sequence of clone 63250625

SEQ ID NO:3985 is the determined cDNA sequence of clone 63250626

SEQ ID NO:3986 is the determined cDNA sequence of clone 63250627

SEQ ID NO:3987 is the determined cDNA sequence of clone 63250628

SEQ ID NO:3988 is the determined cDNA sequence of clone 63250629

SEQ ID NO:3989 is the determined cDNA sequence of clone 63250630

SEQ ID NO:3990 is the determined cDNA sequence of clone 63250631

SEQ ID NO:3991 is the determined cDNA sequence of clone 63250632

SEQ ID NO:3992 is the determined cDNA sequence of clone 63250633

SEQ ID NO:3993 is the determined cDNA sequence of clone 63250634

SEQ ID NO:3994 is the determined cDNA sequence of clone 63250635

SEQ ID NO:3995 is the determined cDNA sequence of clone 63250636

SEQ ID NO:3996 is the determined cDNA sequence of clone 63250637

SEQ ID NO:3997 is the determined cDNA sequence of clone 63250638

SEQ ID NO:3998 is the determined cDNA sequence of clone 63250639

SEQ ID NO:3999 is the determined cDNA sequence of clone 63250640

SEQ ID NO:4000 is the determined cDNA sequence of clone 63250641

SEQ ID NO:4001 is the determined cDNA sequence of clone 63250642

SEQ ID NO:4002 is the determined cDNA sequence of clone 63250643

SEQ ID NO:4003 is the determined cDNA sequence of clone 63250644

SEQ ID NO:4004 is the determined cDNA sequence of clone 63250645

SEQ ID NO:4005 is the determined cDNA sequence of clone 63250646

SEQ ID NO:4006 is the determined cDNA sequence of clone 63250647

SEQ ID NO:4007 is the determined cDNA sequence of clone 63250648

SEQ ID NO:4008 is the determined cDNA sequence of clone 63250649

SEQ ID NO:4009 is the determined cDNA sequence of clone 63250650

SEQ ID NO:4010 is the determined cDNA sequence of clone 63250651

SEQ ID NO:4011 is the determined cDNA sequence of clone 63250652

SEQ ID NO:4012 is the determined cDNA sequence of clone 63250653

SEQ ID NO:4013 is the determined cDNA sequence of clone 63250654

SEQ ID NO:4014 is the determined cDNA sequence of clone 63250656

SEQ ID NO:4015 is the determined cDNA sequence of clone 63250657

SEQ ID NO:4016 is the determined cDNA sequence of clone 63250658

SEQ ID NO:4017 is the determined cDNA sequence of clone 63250660

SEQ ID NO:4018 is the determined cDNA sequence of clone 63250661

SEQ ID NO:4019 is the determined cDNA sequence of clone 63250662

SEQ ID NO:4020 is the determined cDNA sequence of clone 63250663

SEQ ID NO:4021 is the determined cDNA sequence of clone 63250664

SEQ ID NO:4022 is the determined cDNA sequence of clone 63250665

SEQ ID NO:4023 is the determined cDNA sequence of clone 63250667

SEQ ID NO:4024 is the determined cDNA sequence of clone 63250668

SEQ ID NO:4025 is the determined cDNA sequence of clone 63250669

SEQ ID NO:4026 is the determined cDNA sequence of clone 63250670

SEQ ID NO:4027 is the determined cDNA sequence of clone 63250671

SEQ ID NO:4028 is the determined cDNA sequence of clone 63250672

SEQ ID NO:4029 is the determined cDNA sequence of clone 63250673

SEQ ID NO:4030 is the determined cDNA sequence of clone 63250674

SEQ ID NO:4031 is the determined cDNA sequence of clone 63250676

SEQ ID NO:4032 is the determined cDNA sequence of clone 63250677

SEQ ID NO:4033 is the determined cDNA sequence of clone 63250678

SEQ ID NO:4034 is the determined cDNA sequence of clone 63250679

SEQ ID NO:4035 is the determined cDNA sequence of clone 63250681

SEQ ID NO:4036 is the determined cDNA sequence of clone 63250682

SEQ ID NO:4037 is the determined cDNA sequence of clone 63250683

SEQ ID NO:4038 is the determined cDNA sequence of clone 63250684

SEQ ID NO:4039 is the determined cDNA sequence of clone 63250685

SEQ ID NO:4040 is the determined cDNA sequence of clone 63250686

SEQ ID NO:4041 is the determined cDNA sequence of clone 63250687

SEQ ID NO:4042 is the determined cDNA sequence of clone 63250688

SEQ ID NO:4043 is the determined cDNA sequence of clone 63250689

SEQ ID NO:4044 is the determined cDNA sequence of clone 63250690

SEQ ID NO:4045 is the determined cDNA sequence of clone 63250691

SEQ ID NO:4046 is the determined cDNA sequence of clone 63250692

SEQ ID NO:4047 is the determined cDNA sequence of clone 63250693

SEQ ID NO:4048 is the determined cDNA sequence of clone 63250694

SEQ ID NO:4049 is the determined cDNA sequence of clone 63250696

SEQ ID NO:4050 is the determined cDNA sequence of clone 63138129

SEQ ID NO:4051 is the determined cDNA sequence of clone 63138130

SEQ ID NO:4052 is the determined cDNA sequence of clone 63138131

SEQ ID NO:4053 is the determined cDNA sequence of clone 63138132

SEQ ID NO:4054 is the determined cDNA sequence of clone 63138133

SEQ ID NO:4055 is the determined cDNA sequence of clone 63138134

SEQ ID NO:4056 is the determined cDNA sequence of clone 63138135

SEQ ID NO:4057 is the determined cDNA sequence of clone 63138136

SEQ ID NO:4058 is the determined cDNA sequence of clone 63138137

SEQ ID NO:4059 is the determined cDNA sequence of clone 63138138

SEQ ID NO:4060 is the determined cDNA sequence of clone 63138139

SEQ ID NO:4061 is the determined cDNA sequence of clone 63138140

SEQ ID NO:4062 is the determined cDNA sequence of clone 63138141

SEQ ID NO:4063 is the determined cDNA sequence of clone 63138142

SEQ ID NO:4064 is the determined cDNA sequence of clone 63138144

SEQ ID NO:4065 is the determined cDNA sequence of clone 63138145

SEQ ID NO:4066 is the determined cDNA sequence of clone 63138146

SEQ ID NO:4067 is the determined cDNA sequence of clone 63138147

SEQ ID NO:4068 is the determined cDNA sequence of clone 63138148

SEQ ID NO:4069 is the determined cDNA sequence of clone 63138149

SEQ ID NO:4070 is the determined cDNA sequence of clone 63138150

SEQ ID NO:4071 is the determined cDNA sequence of clone 63138151

SEQ ID NO:4072 is the determined cDNA sequence of clone 63138153

SEQ ID NO:4073 is the determined cDNA sequence of clone 63138154

SEQ ID NO:4074 is the determined cDNA sequence of clone 63138155

SEQ ID NO:4075 is the determined cDNA sequence of clone 63138156

SEQ ID NO:4076 is the determined cDNA sequence of clone 63138157

SEQ ID NO:4077 is the determined cDNA sequence of clone 63138158

SEQ ID NO:4078 is the determined cDNA sequence of clone 63138159

SEQ ID NO:4079 is the determined cDNA sequence of clone 63138161

SEQ ID NO:4080 is the determined cDNA sequence of clone 63138162

SEQ ID NO:4081 is the determined cDNA sequence of clone 63138163

SEQ ID NO:4082 is the determined cDNA sequence of clone 63138165

SEQ ID NO:4083 is the determined cDNA sequence of clone 63138166

SEQ ID NO:4084 is the determined cDNA sequence of clone 63138167

SEQ ID NO:4085 is the determined cDNA sequence of clone 63138169

SEQ ID NO:4086 is the determined cDNA sequence of clone 63138170

SEQ ID NO:4087 is the determined cDNA sequence of clone 63138171

SEQ ID NO:4088 is the determined cDNA sequence of clone 63138172

SEQ ID NO:4089 is the determined cDNA sequence of clone 63138173

SEQ ID NO:4090 is the determined cDNA sequence of clone 63138174

SEQ ID NO:4091 is the determined cDNA sequence of clone 63138175

SEQ ID NO:4092 is the determined cDNA sequence of clone 63138176

SEQ ID NO:4093 is the determined cDNA sequence of clone 63138177

SEQ ID NO:4094 is the determined cDNA sequence of clone 63138178

SEQ ID NO:4095 is the determined cDNA sequence of clone 63138179

SEQ ID NO:4096 is the determined cDNA sequence of clone 63138181

SEQ ID NO:4097 is the determined cDNA sequence of clone 63138182

SEQ ID NO:4098 is the determined cDNA sequence of clone 63138183

SEQ ID NO:4099 is the determined cDNA sequence of clone 63138184

SEQ ID NO:4100 is the determined cDNA sequence of clone 63138185

SEQ ID NO:4101 is the determined cDNA sequence of clone 63138186

SEQ ID NO:4102 is the determined cDNA sequence of clone 63138187

SEQ ID NO:4103 is the determined cDNA sequence of clone 63138189

SEQ ID NO:4104 is the determined cDNA sequence of clone 63138191

SEQ ID NO:4105 is the determined cDNA sequence of clone 63138192

SEQ ID NO:4106 is the determined cDNA sequence of clone 63138193

SEQ ID NO:4107 is the determined cDNA sequence of clone 63138194

SEQ ID NO:4108 is the determined cDNA sequence of clone 63138195

SEQ ID NO:4109 is the determined cDNA sequence of clone 63138196

SEQ ID NO:4110 is the determined cDNA sequence of clone 63138197

SEQ ID NO:4111 is the determined cDNA sequence of clone 63138198

SEQ ID NO:4112 is the determined cDNA sequence of clone 63138199

SEQ ID NO:4113 is the determined cDNA sequence of clone 63138201

SEQ ID NO:4114 is the determined cDNA sequence of clone 63138202

SEQ ID NO:4115 is the determined cDNA sequence of clone 63138203

SEQ ID NO:4116 is the determined cDNA sequence of clone 63138204

SEQ ID NO:4117 is the determined cDNA sequence of clone 63138205

SEQ ID NO:4118 is the determined cDNA sequence of clone 63138206

SEQ ID NO:4119 is the determined cDNA sequence of clone 63138208

SEQ ID NO:4120 is the determined cDNA sequence of clone 63138209

SEQ ID NO:4121 is the determined cDNA sequence of clone 63138210

SEQ ID NO:4122 is the determined cDNA sequence of clone 63138211

SEQ ID NO:4123 is the determined cDNA sequence of clone 63138212

SEQ ID NO:4124 is the determined cDNA sequence of clone 63138213

SEQ ID NO:4125 is the determined cDNA sequence of clone 63138214

SEQ ID NO:4126 is the determined cDNA sequence of clone 63138215

SEQ ID NO:4127 is the determined cDNA sequence of clone 63138217

SEQ ID NO:4128 is the determined cDNA sequence of clone 63138218

SEQ ID NO:4129 is the determined cDNA sequence of clone 63138220

SEQ ID NO:4130 is the determined cDNA sequence of clone 63138221

SEQ ID NO:4131 is the determined cDNA sequence of clone 63299167

SEQ ID NO:4132 is the determined cDNA sequence of clone 63299168

SEQ ID NO:4133 is the determined cDNA sequence of clone 63299169

SEQ ID NO:4134 is the determined cDNA sequence of clone 63299171

SEQ ID NO:4135 is the determined cDNA sequence of clone 63299172

SEQ ID NO:4136 is the determined cDNA sequence of clone 63299173

SEQ ID NO:4137 is the determined cDNA sequence of clone 63299174

SEQ ID NO:4138 is the determined cDNA sequence of clone 63299175

SEQ ID NO:4139 is the determined cDNA sequence of clone 63299176

SEQ ID NO:4140 is the determined cDNA sequence of clone 63299177

SEQ ID NO:4141 is the determined cDNA sequence of clone 63299178

SEQ ID NO:4142 is the determined cDNA sequence of clone 63299179

SEQ ID NO:4143 is the determined cDNA sequence of clone 63299180

SEQ ID NO:4144 is the determined cDNA sequence of clone 63299181

SEQ ID NO:4145 is the determined cDNA sequence of clone 63299182

SEQ ID NO:4146 is the determined cDNA sequence of clone 63299183

SEQ ID NO:4147 is the determined cDNA sequence of clone 63299185

SEQ ID NO:4148 is the determined cDNA sequence of clone 63299187

SEQ ID NO:4149 is the determined cDNA sequence of clone 63299188

SEQ ID NO:4150 is the determined cDNA sequence of clone 63299189

SEQ ID NO:4151 is the determined cDNA sequence of clone 63299191

SEQ ID NO:4152 is the determined cDNA sequence of clone 63299193

SEQ ID NO:4153 is the determined cDNA sequence of clone 63299194

SEQ ID NO:4154 is the determined cDNA sequence of clone 63299195

SEQ ID NO:4155 is the determined cDNA sequence of clone 63299197

SEQ ID NO:4156 is the determined cDNA sequence of clone 63299198

SEQ ID NO:4157 is the determined cDNA sequence of clone 63299199

SEQ ID NO:4158 is the determined cDNA sequence of clone 63299200

SEQ ID NO:4159 is the determined cDNA sequence of clone 63299201

SEQ ID NO:4160 is the determined cDNA sequence of clone 63299203

SEQ ID NO:4161 is the determined cDNA sequence of clone 63299204

SEQ ID NO:4162 is the determined cDNA sequence of clone 63299206

SEQ ID NO:4163 is the determined cDNA sequence of clone 63299209

SEQ ID NO:4164 is the determined cDNA sequence of clone 63299210

SEQ ID NO:4165 is the determined cDNA sequence of clone 63299211

SEQ ID NO:4166 is the determined cDNA sequence of clone 63299212

SEQ ID NO:4167 is the determined cDNA sequence of clone 63299213

SEQ ID NO:4168 is the determined cDNA sequence of clone 63299214

SEQ ID NO:4169 is the determined cDNA sequence of clone 63299215

SEQ ID NO:4170 is the determined cDNA sequence of clone 63299216

SEQ ID NO:4171 is the determined cDNA sequence of clone 63299217

SEQ ID NO:4172 is the determined cDNA sequence of clone 63299218

SEQ ID NO:4173 is the determined cDNA sequence of clone 63299221

SEQ ID NO:4174 is the determined cDNA sequence of clone 63299222

SEQ ID NO:4175 is the determined cDNA sequence of clone 63299223

SEQ ID NO:4176 is the determined cDNA sequence of clone 63299224

SEQ ID NO:4177 is the determined cDNA sequence of clone 63299226

SEQ ID NO:4178 is the determined cDNA sequence of clone 63299227

SEQ ID NO:4179 is the determined cDNA sequence of clone 63299228

SEQ ID NO:4180 is the determined cDNA sequence of clone 63299229

SEQ ID NO:4181 is the determined cDNA sequence of clone 63299230

SEQ ID NO:4182 is the determined cDNA sequence of clone 63299231

SEQ ID NO:4183 is the determined cDNA sequence of clone 63299233

SEQ ID NO:4184 is the determined cDNA sequence of clone 63299235

SEQ ID NO:4185 is the determined cDNA sequence of clone 63299236

SEQ ID NO:4186 is the determined cDNA sequence of clone 63299237

SEQ ID NO:4187 is the determined cDNA sequence of clone 63299239

SEQ ID NO:4188 is the determined cDNA sequence of clone 63299240

SEQ ID NO:4189 is the determined cDNA sequence of clone 63299242

SEQ ID NO:4190 is the determined cDNA sequence of clone 63299243

SEQ ID NO:4191 is the determined cDNA sequence of clone 63299245

SEQ ID NO:4192 is the determined cDNA sequence of clone 63299247

SEQ ID NO:4193 is the determined cDNA sequence of clone 63299248

SEQ ID NO:4194 is the determined cDNA sequence of clone 63299249

SEQ ID NO:4195 is the determined cDNA sequence of clone 63299250

SEQ ID NO:4196 is the determined cDNA sequence of clone 63299251

SEQ ID NO:4197 is the determined cDNA sequence of clone 63299252

SEQ ID NO:4198 is the determined cDNA sequence of clone 63299253

SEQ ID NO:4199 is the determined cDNA sequence of clone 63299254

SEQ ID NO:4200 is the determined cDNA sequence of clone 63299256

SEQ ID NO:4201 is the determined cDNA sequence of clone 63299257

SEQ ID NO:4202 is the determined cDNA sequence of clone 63299259

SEQ ID NO:4203 is the determined cDNA sequence of clone 63234997

SEQ ID NO:4204 is the determined cDNA sequence of clone 63234998

SEQ ID NO:4205 is the determined cDNA sequence of clone 63234999

SEQ ID NO:4206 is the determined cDNA sequence of clone 63235000

SEQ ID NO:4207 is the determined cDNA sequence of clone 63235001

SEQ ID NO:4208 is the determined cDNA sequence of clone 63235002

SEQ ID NO:4209 is the determined cDNA sequence of clone 63235003

SEQ ID NO:4210 is the determined cDNA sequence of clone 63235004

SEQ ID NO:4211 is the determined cDNA sequence of clone 63235007

SEQ ID NO:4212 is the determined cDNA sequence of clone 63235008

SEQ ID NO:4213 is the determined cDNA sequence of clone 63235009

SEQ ID NO:4214 is the determined cDNA sequence of clone 63235011

SEQ ID NO:4215 is the determined cDNA sequence of clone 63235012

SEQ ID NO:4216 is the determined cDNA sequence of clone 63235014

SEQ ID NO:4217 is the determined cDNA sequence of clone 63235015

SEQ ID NO:4218 is the determined cDNA sequence of clone 63235016

SEQ ID NO:4219 is the determined cDNA sequence of clone 63235019

SEQ ID NO:4220 is the determined cDNA sequence of clone 63235020

SEQ ID NO:4221 is the determined cDNA sequence of clone 63235021

SEQ ID NO:4222 is the determined cDNA sequence of clone 63235022

SEQ ID NO:4223 is the determined cDNA sequence of clone 63235023

SEQ ID NO:4224 is the determined cDNA sequence of clone 63235024

SEQ ID NO:4225 is the determined cDNA sequence of clone 63235025

SEQ ID NO:4226 is the determined cDNA sequence of clone 63235028

SEQ ID NO:4227 is the determined cDNA sequence of clone 63235029

SEQ ID NO:4228 is the determined cDNA sequence of clone 63235031

SEQ ID NO:4229 is the determined cDNA sequence of clone 63235033

SEQ ID NO:4230 is the determined cDNA sequence of clone 63235034

SEQ ID NO:4231 is the determined cDNA sequence of clone 63235037

SEQ ID NO:4232 is the determined cDNA sequence of clone 63235038

SEQ ID NO:4233 is the determined cDNA sequence of clone 63235039

SEQ ID NO:4234 is the determined cDNA sequence of clone 63235043

SEQ ID NO:4235 is the determined cDNA sequence of clone 63235044

SEQ ID NO:4236 is the determined cDNA sequence of clone 63235045

SEQ ID NO:4237 is the determined cDNA sequence of clone 63235046

SEQ ID NO:4238 is the determined cDNA sequence of clone 63235047

SEQ ID NO:4239 is the determined cDNA sequence of clone 63235048

SEQ ID NO:4240 is the determined cDNA sequence of clone 63235049

SEQ ID NO:4241 is the determined cDNA sequence of clone 63235050

SEQ ID NO:4242 is the determined cDNA sequence of clone 63235051

SEQ ID NO:4243 is the determined cDNA sequence of clone 63235052

SEQ ID NO:4244 is the determined cDNA sequence of clone 63235053

SEQ ID NO:4245 is the determined cDNA sequence of clone 63235056

SEQ ID NO:4246 is the determined cDNA sequence of clone 63235057

SEQ ID NO:4247 is the determined cDNA sequence of clone 63235058

SEQ ID NO:4248 is the determined cDNA sequence of clone 63235059

SEQ ID NO:4249 is the determined cDNA sequence of clone 63235060

SEQ ID NO:4250 is the determined cDNA sequence of clone 63235061

SEQ ID NO:4251 is the determined cDNA sequence of clone 63235062

SEQ ID NO:4252 is the determined cDNA sequence of clone 63235063

SEQ ID NO:4253 is the determined cDNA sequence of clone 63235064

SEQ ID NO:4254 is the determined cDNA sequence of clone 63235067

SEQ ID NO:4255 is the determined cDNA sequence of clone 63235068

SEQ ID NO:4256 is the determined cDNA sequence of clone 63235069

SEQ ID NO:4257 is the determined cDNA sequence of clone 63235070

SEQ ID NO:4258 is the determined cDNA sequence of clone 63235071

SEQ ID NO:4259 is the determined cDNA sequence of clone 63235072

SEQ ID NO:4260 is the determined cDNA sequence of clone 63235073

SEQ ID NO:4261 is the determined cDNA sequence of clone 63235074

SEQ ID NO:4262 is the determined cDNA sequence of clone 63235075

SEQ ID NO:4263 is the determined cDNA sequence of clone 63235077

SEQ ID NO:4264 is the determined cDNA sequence of clone 63235078

SEQ ID NO:4265 is the determined cDNA sequence of clone 63235079

SEQ ID NO:4266 is the determined cDNA sequence of clone 63235080

SEQ ID NO:4267 is the determined cDNA sequence of clone 63235081

SEQ ID NO:4268 is the determined cDNA sequence of clone 63235082

SEQ ID NO:4269 is the determined cDNA sequence of clone 63235084

SEQ ID NO:4270 is the determined cDNA sequence of clone 63235085

SEQ ID NO:4271 is the determined cDNA sequence of clone 63235086

SEQ ID NO:4272 is the determined cDNA sequence of clone 63235087

SEQ ID NO:4273 is the determined cDNA sequence of clone 63792101

SEQ ID NO:4274 is the determined cDNA sequence of clone 63792102

SEQ ID NO:4275 is the determined cDNA sequence of clone 63792103

SEQ ID NO:4276 is the determined cDNA sequence of clone 63792104

SEQ ID NO:4277 is the determined cDNA sequence of clone 63792105

SEQ ID NO:4278 is the determined cDNA sequence of clone 63792106

SEQ ID NO:4279 is the determined cDNA sequence of clone 63792107

SEQ ID NO:4280 is the determined cDNA sequence of clone 63792108

SEQ ID NO:4281 is the determined cDNA sequence of clone 63792109

SEQ ID NO:4282 is the determined cDNA sequence of clone 63792110

SEQ ID NO:4283 is the determined cDNA sequence of clone 63792114

SEQ ID NO:4284 is the determined cDNA sequence of clone 63792115

SEQ ID NO:4285 is the determined cDNA sequence of clone 63792116

SEQ ID NO:4286 is the determined cDNA sequence of clone 63792117

SEQ ID NO:4287 is the determined cDNA sequence of clone 63792118

SEQ ID NO:4288 is the determined cDNA sequence of clone 63792119

SEQ ID NO:4289 is the determined cDNA sequence of clone 63792120

SEQ ID NO:4290 is the determined cDNA sequence of clone 63792121

SEQ ID NO:4291 is the determined cDNA sequence of clone 63792124

SEQ ID NO:4292 is the determined cDNA sequence of clone 63792125

SEQ ID NO:4293 is the determined cDNA sequence of clone 63792127

SEQ ID NO:4294 is the determined cDNA sequence of clone 63792128

SEQ ID NO:4295 is the determined cDNA sequence of clone 63792129

SEQ ID NO:4296 is the determined cDNA sequence of clone 63792130

SEQ ID NO:4297 is the determined cDNA sequence of clone 63792131

SEQ ID NO:4298 is the determined cDNA sequence of clone 63792132

SEQ ID NO:4299 is the determined cDNA sequence of clone 63792136

SEQ ID NO:4300 is the determined cDNA sequence of clone 63792137

SEQ ID NO:4301 is the determined cDNA sequence of clone 63792138

SEQ ID NO:4302 is the determined cDNA sequence of clone 63792139

SEQ ID NO:4303 is the determined cDNA sequence of clone 63792140

SEQ ID NO:4304 is the determined cDNA sequence of clone 63792141

SEQ ID NO:4305 is the determined cDNA sequence of clone 63792144

SEQ ID NO:4306 is the determined cDNA sequence of clone 63792145

SEQ ID NO:4307 is the determined cDNA sequence of clone 63792146

SEQ ID NO:4308 Is the determined cDNA sequence of clone 63792149

SEQ ID NO:4309 is the determined cDNA sequence of clone 63792150

SEQ ID NO:4310 is the determined cDNA sequence of clone 63792151

SEQ ID NO:4311 is the determined cDNA sequence of clone 63792152

SEQ ID NO:4312 is the determined cDNA sequence of clone 63792153

SEQ ID NO:4313 is the determined cDNA sequence of clone 63792156

SEQ ID NO:4314 is the determined cDNA sequence of clone 63792157

SEQ ID NO:4315 is the determined cDNA sequence of clone 63792159

SEQ ID NO:4316 is the determined cDNA sequence of clone 63792160

SEQ ID NO:4317 is the determined cDNA sequence of clone 63792161

SEQ ID NO:4318 is the determined cDNA sequence of clone 63792162

SEQ ID NO:4319 is the determined cDNA sequence of clone 63792163

SEQ ID NO:4320 is the determined cDNA sequence of clone 63792165

SEQ ID NO:4321 is the determined cDNA sequence of clone 63792166

SEQ ID NO:4322 is the determined cDNA sequence of clone 63792167

SEQ ID NO:4323 is the determined cDNA sequence of clone 63792168

SEQ ID NO:4324 is the determined cDNA sequence of clone 63792169

SEQ ID NO:4325 is the determined cDNA sequence of clone 63792170

SEQ ID NO:4326 is the determined cDNA sequence of clone 63792171

SEQ ID NO:4327 is the determined cDNA sequence of clone 63792172

SEQ ID NO:4328 is the determined cDNA sequence of clone 63792173

SEQ ID NO:4329 is the determined cDNA sequence of clone 63792174

SEQ ID NO:4330 is the determined cDNA sequence of clone 63792175

SEQ ID NO:4331 is the determined cDNA sequence of clone 63792176

SEQ ID NO:4332 is the determined cDNA sequence of clone 63792177

SEQ ID NO:4333 is the determined cDNA sequence of clone 63792178

SEQ ID NO:4334 is the determined cDNA sequence of clone 63792179

SEQ ID NO:4335 is the determined cDNA sequence of clone 63792180

SEQ ID NO:4336 is the determined cDNA sequence of clone 63792181

SEQ ID NO:4337 is the determined cDNA sequence of clone 63792183

SEQ ID NO:4338 is the determined cDNA sequence of clone 63792184

SEQ ID NO:4339 is the determined cDNA sequence of clone 63792185

SEQ ID NO:4340 is the determined cDNA sequence of clone 63792186

SEQ ID NO:4341 is the determined cDNA sequence of clone 63792187

SEQ ID NO:4342 is the determined cDNA sequence of clone 63792188

SEQ ID NO:4343 is the determined cDNA sequence of clone 63792189

SEQ ID NO:4344 is the determined cDNA sequence of clone 63792190

SEQ ID NO:4345 is the determined cDNA sequence of clone 63792192

SEQ ID NO:4346 is the determined cDNA sequence of clone p0150r10c11

SEQ ID NO:4347 is the determined cDNA sequence of clone p0150r07c17

SEQ ID NO:4348 is the determined cDNA sequence of clone p0150r09c15

SEQ ID NO:4349 is the determined cDNA sequence of clone p0150r07c14

SEQ ID NO:4350 is the determined cDNA sequence of clone p0150r11c05

SEQ ID NO:4351 is the determined cDNA sequence of clone p0160r06c18

SEQ ID NO:4352 is the determined cDNA sequence of clone p0160r02c21

SEQ ID NO:4353 is the determined cDNA sequence of clone p0160r13c02

SEQ ID NO:4354 is the determined cDNA sequence of clone p0150r13c04

SEQ ID NO:4355 is the determined cDNA sequence of clone p0150r02c15

SEQ ID NO:4356 is the determined cDNA sequence of clone p0150r06c12

SEQ ID NO:4357 is the determined cDNA sequence of clone p0151r06c12

SEQ ID NO:4358 is the determined cDNA sequence of clone p0150r14c04

SEQ ID NO:4359 is the determined cDNA sequence of clone p0150r02c06

SEQ ID NO:4360 is the determined cDNA sequence of clone p0150r04c19

SEQ ID NO:4361 is the determined cDNA sequence of clone p0150r03c18

SEQ ID NO:4362 is the determined cDNA sequence of clone p0151r13c03

SEQ ID NO:4363 is the determined cDNA sequence of clone p0150r01c08

SEQ ID NO:4364 is the determined cDNA sequence of clone p0150r11c07

SEQ ID NO:4365 is the determined cDNA sequence of clone p0150r02c11

SEQ ID NO:4366 is the determined cDNA sequence of clone p0150r01c17

SEQ ID NO:4367 is the determined cDNA sequence of clone p0150r05c16

SEQ ID NO:4368 is the determined cDNA sequence of clone p0150r06c04

SEQ ID NO:4369 is the determined cDNA sequence of clone p0150r15c17

SEQ ID NO:4370 is the determined cDNA sequence of clone p0150r15c11

SEQ ID NO:4371 is the determined cDNA sequence of clone p0150r09c13

SEQ ID NO:4372 is the determined cDNA sequence of clone p0150r13c03

SEQ ID NO:4373 is the determined cDNA sequence of clone p0150r09c10

SEQ ID NO:4374 is the determined cDNA sequence of clone p0161r13c17

SEQ ID NO:4375 is the determined cDNA sequence of clone p0159r16c21

SEQ ID NO:4376 is the determined cDNA sequence of clone p0150r02c02

SEQ ID NO:4377 is the determined cDNA sequence of clone p0151r09c19

SEQ ID NO:4378 is the determined cDNA sequence of clone p0151r02c06

SEQ ID NO:4379 is the determined cDNA sequence of clone p0150r16c06

SEQ ID NO:4380 is the determined cDNA sequence of clone p0150r09c12

SEQ ID NO:4381 is the determined cDNA sequence of clone p0150r07c06

SEQ ID NO:4382 is the determined cDNA sequence of clone p0150r06c19

SEQ ID NO:4383 is the determined cDNA sequence of clone p0150r04c05

SEQ ID NO:4384 is the determined cDNA sequence of clone p0150r03c21

SEQ ID NO:4385 is the determined cDNA sequence of clone p0150r02c18

SEQ ID NO:4386 is the determined cDNA sequence of clone p0150r15c07

SEQ ID NO:4387 is the determined cDNA sequence of clone p0150r10c15

SEQ ID NO:4388 is the determined cDNA sequence of clone p0150r07c05

SEQ ID NO:4389 is the determined cDNA sequence of clone p0150r06c08

SEQ ID NO:4390 is the determined cDNA sequence of clone p0150r01c24

SEQ ID NO:4391 is the determined cDNA sequence of clone p0150r07c15

SEQ ID NO:4392 is the determined cDNA sequence of clone p0150r02c23

SEQ ID NO:4393 is the determined cDNA sequence of clone p0151r09c08

SEQ ID NO:4394 is the determined cDNA sequence of clone p0150r16c09

SEQ ID NO:4395 is the determined cDNA sequence of clone p0150r14c11

SEQ ID NO:4396 is the determined cDNA sequence of clone p0151r05c12

SEQ ID NO:4397 is the determined cDNA sequence of clone p0150r12c23

SEQ ID NO:4398 is the determined cDNA sequence of clone p0150r16c02

SEQ ID NO:4399 is the determined cDNA sequence of clone p0155r10c08

SEQ ID NO:4400 is the determined cDNA sequence of clone p0150r02c05

SEQ ID NO:4401 is the determined cDNA sequence of clone p0150r09c04

SEQ ID NO:4402 is the determined cDNA sequence of clone p0150r03c22

SEQ ID NO:4403 is the determined cDNA sequence of clone p0150r15c10

SEQ ID NO:4404 is the determined cDNA sequence of clone p0150r13c20

SEQ ID NO:4405 is the determined cDNA sequence of clone p0150r08c18

SEQ ID NO:4406 is the determined cDNA sequence of clone p0150r09c22

SEQ ID NO:4407 is the determined cDNA sequence of clone p0157r08c09

SEQ ID NO:4408 is the determined cDNA sequence of clone p0163r04c09

SEQ ID NO:4409 is the determined cDNA sequence of clone p0155r01c04

SEQ ID NO:4410 is the determined cDNA sequence of clone p0152r16c22

SEQ ID NO:4411 is the determined cDNA sequence of clone p0152r16c12

SEQ ID NO:4412 is the determined cDNA sequence of clone p0150r01c16

SEQ ID NO:4413 is the determined cDNA sequence of clone p0150r05c23

SEQ ID NO:4414 is the determined cDNA sequence of clone p0150r11c09

SEQ ID NO:4415 is the determined cDNA sequence of clone p0150r13c22

SEQ ID NO:4416 is the determined cDNA sequence of clone p0150r11c17

SEQ ID NO:4417 is the determined cDNA sequence of clone p0159r16c01

SEQ ID NO:4418 is the determined cDNA sequence of clone p0156r16c21

SEQ ID NO:4419 is the determined cDNA sequence of clone p0150r06c15

SEQ ID NO:4420 is the determined cDNA sequence of clone p0151 r05c04

SEQ ID NO:4421 is the determined cDNA sequence of clone p0150r05c21

SEQ ID NO:4422 is the determined cDNA sequence of clone p0150r11c22

SEQ ID NO:4423 is the determined cDNA sequence of clone p0150r01c14

SEQ ID NO:4424 is the determined cDNA sequence of clone p0150r15c08

SEQ ID NO:4425 is the determined cDNA sequence of clone p0150r11c02

SEQ ID NO:4426 is the determined cDNA sequence of clone p0157r07c21

SEQ ID NO:4427 is the determined cDNA sequence of clone p0150r16c17

SEQ ID NO:4428 is the determined cDNA sequence of clone p0155r03c08

SEQ ID NO:4429 is the determined cDNA sequence of clone p0150r07c10

SEQ ID NO:4430 is the determined cDNA sequence of clone p0150r16c13

SEQ ID NO:4431 is the determined cDNA sequence of clone p0150r06c10

SEQ ID NO:4432 is the determined cDNA sequence of clone p0150r02c16

SEQ ID NO:4433 is the determined cDNA sequence of clone p0150r09c06

SEQ ID NO:4434 is the determined cDNA sequence of clone p0150r11c16

SEQ ID NO:4435 is the determined cDNA sequence of clone p0150r01c13

SEQ ID NO:4436 is the determined cDNA sequence of clone p0150r08c10

SEQ ID NO:4437 is the determined cDNA sequence of clone p0150r05c18

SEQ ID NO:4438 is the determined cDNA sequence of clone p0159r14c18

SEQ ID NO:4439 is the determined cDNA sequence of clone p0150r16c22

SEQ ID NO:4440 is the determined cDNA sequence of clone p0152r10c20

SEQ ID NO:4441 is the determined cDNA sequence of clone p0150r13c23

SEQ ID NO:4442 is the determined cDNA sequence of clone p0150r12c10

SEQ ID NO:4443 is the determined cDNA sequence of clone p0150r09c18

SEQ ID NO:4444 is the determined cDNA sequence of clone p0158r09c04

SEQ ID NO:4445 is the determined cDNA sequence of clone p0150r08c09

SEQ ID NO:4446 is the determined cDNA sequence of clone p0150r11c11

SEQ ID NO:4447 is the determined cDNA sequence of clone p0150r15c01

SEQ ID NO:4448 is the determined cDNA sequence of clone p0157r07c01

SEQ ID NO:4449 is the determined cDNA sequence of clone p0150r09c21

SEQ ID NO:4450 is the determined cDNA sequence of clone p0150r12c02

SEQ ID NO:4451 is the determined cDNA sequence of clone p0150r03c17

SEQ ID NO:4452 is the determined cDNA sequence of clone p0157r07c20

SEQ ID NO:4453 is the determined cDNA sequence of clone p0150r05c20

SEQ ID NO:4454 is the determined cDNA sequence of clone p0150r05c04

SEQ ID NO:4455 is the determined cDNA sequence of clone p0150r07c12

SEQ ID NO:4456 is the determined cDNA sequence of clone p0150r05c02

SEQ ID NO:4457 is the determined cDNA sequence of clone p0152r13c24

SEQ ID NO:4458 is the determined cDNA sequence of clone p0150r03c11

SEQ ID NO:4459 is the determined cDNA sequence of clone p0162r13c11

SEQ ID NO:4460 is the determined cDNA sequence of clone p0151r13c15

SEQ ID NO:4461 is the determined cDNA sequence of clone p0150r03c01

SEQ ID NO:4462 is the determined cDNA sequence of clone p0151r08c17

SEQ ID NO:4463 is the determined cDNA sequence of clone p0150r01c02

SEQ ID NO:4464 is the determined cDNA sequence of clone p0150r04c06

SEQ ID NO:4465 is the determined cDNA sequence of clone p0150r09c19

SEQ ID NO:4466 is the determined cDNA sequence of clone p0153r06c10

SEQ ID NO:4467 is the determined cDNA sequence of clone p0150r02c20

SEQ ID NO:4468 is the determined cDNA sequence of clone p0155r09c08

SEQ ID NO:4469 is the determined cDNA sequence of clone p0152r16c01

SEQ ID NO:4470 is the determined cDNA sequence of clone p0150r13c02

SEQ ID NO:4471 is the determined cDNA sequence of clone p0150r12c22

SEQ ID NO:4472 is the determined cDNA sequence of clone p0150r11c23

SEQ ID NO:4473 is the determined cDNA sequence of clone p0155r10c24

SEQ ID NO:4474 is the determined cDNA sequence of clone p0157r06c03

SEQ ID NO:4475 is the determined cDNA sequence of clone p0150r05c24

SEQ ID NO:4476 is the determined cDNA sequence of clone p0150r11c04

SEQ ID NO:4477 is the determined cDNA sequence of clone p0156r16c18

SEQ ID NO:4478 is the determined cDNA sequence of clone p0155r10c19

SEQ ID NO:4479 is the determined cDNA sequence of clone p0150r14c23

SEQ ID NO:4480 is the determined cDNA sequence of clone p0150r10c19

SEQ ID NO:4481 is the determined cDNA sequence of clone p0150r11c19

SEQ ID NO:4482 is the determined cDNA sequence of clone p0150r15c21

SEQ ID NO:4483 is the determined cDNA sequence of clone p0150r11c21

SEQ ID NO:4484 is the determined cDNA sequence of clone p0157r05c22

SEQ ID NO:4485 is the determined cDNA sequence of clone p0157r05c21

SEQ ID NO:4486 is the determined cDNA sequence of clone p0157r06c05

SEQ ID NO:4487 is the determined cDNA sequence of clone p0157r06c05

SEQ ID NO:4488 is the determined cDNA sequence of clone p0150r15c22

SEQ ID NO:4489 is the determined cDNA sequence of clone p0159r03c13

SEQ ID NO:4490 is the determined cDNA sequence of clone p0160r04c18

SEQ ID NO:4491 is the determined cDNA sequence of clone p0150r06c23

SEQ ID NO:4492 is the determined cDNA sequence of clone p0150r02c15

SEQ ID NO:4493 is the determined cDNA sequence of clone p0150r13c24

SEQ ID NO:4494 is the determined cDNA sequence of clone p0150r15c03

SEQ ID NO:4495 is the determined cDNA sequence of clone p0150r05c19

SEQ ID NO:4496 is the determined cDNA sequence of clone p0150r07c01

SEQ ID NO:4497 is the determined cDNA sequence of clone p0150r06c16

SEQ ID NO:4498 is the determined cDNA sequence of clone p0150r02c07

SEQ ID NO:4499 is the determined cDNA sequence of clone p0152r15c23

SEQ ID NO:4500 is the determined cDNA sequence of clone p0150r11c01

SEQ ID NO:4501 is the determined cDNA sequence of clone p0150r03c14

SEQ ID NO:4502 is the determined cDNA sequence of clone p0150r02c12

SEQ ID NO:4503 is the determined cDNA sequence of clone p0150r04c17

SEQ ID NO:4504 is the determined cDNA sequence of clone p0150r01c04

SEQ ID NO:4505 is the determined cDNA sequence of clone p0150r02c22

SEQ ID NO:4506 is the determined cDNA sequence of clone p0150r09c01

SEQ ID NO:4507 is the determined cDNA sequence of clone p0150r08c17

SEQ ID NO:4508 is the determined cDNA sequence of clone p0150r09c17

SEQ ID NO:4509 is the determined cDNA sequence of clone p0150r09c20

SEQ ID NO:4510 is the determined cDNA sequence of clone p0150r08c06

SEQ ID NO:4511 is the determined cDNA sequence of clone p0150r13c19

SEQ ID NO:4512 is the determined cDNA sequence of clone p0150r12c09

SEQ ID NO:4513 is the determined cDNA sequence of clone p0150r11c03

SEQ ID NO:4514 is the determined cDNA sequence of clone p0150r12c08

SEQ ID NO:4515 is the determined cDNA sequence of clone p0150r05c22

SEQ ID NO:4516 is the determined cDNA sequence of clone p0150r09c11

SEQ ID NO:4517 is the determined cDNA sequence of clone p0150r15c23

SEQ ID NO:4518 is the determined cDNA sequence of clone p0157r05c17

SEQ ID NO:4519 is the determined cDNA sequence of clone p0157r07c13

SEQ ID NO:4520 is the determined cDNA sequence of clone p0157r07c14

SEQ ID NO:4521 is the determined cDNA sequence of clone p0157r07c15

SEQ ID NO:4522 is the determined cDNA sequence of clone p0151r01c08

SEQ ID NO:4523 is the determined cDNA sequence of clone p0155r07c16

SEQ ID NO:4524 is the determined cDNA sequence of clone p0152r06c10

SEQ ID NO:4525 is the determined cDNA sequence of clone p0150r04c18

SEQ ID NO:4526 is the determined cDNA sequence of clone p0150r02c03

SEQ ID NO:4527 is the determined cDNA sequence of clone p0150r06c24

SEQ ID NO:4528 is the determined cDNA sequence of clone p0150r11c14

SEQ ID NO:4529 is the determined cDNA sequence of clone p0157r05c18

SEQ ID NO:4530 is the determined cDNA sequence of clone p0157r06c17

SEQ ID NO:4531 is the determined cDNA sequence of clone p0157r07c18

SEQ ID NO:4532 is the determined cDNA sequence of clone p0157r06c08

SEQ ID NO:4533 is the determined cDNA sequence of clone p0157r07c23

SEQ ID NO:4534 is the determined cDNA sequence of clone p0150r01c20

SEQ ID NO:4535 is the determined cDNA sequence of clone p0150r14c15

SEQ ID NO:4536 is the determined cDNA sequence of clone p0150r15c06

SEQ ID NO:4537 is the determined cDNA sequence of clone p0151r12c17

SEQ ID NO:4538 is the determined cDNA sequence of clone p0155r12c15

SEQ ID NO:4539 is the determined cDNA sequence of clone p0157r07c09

SEQ ID NO:4540 is the determined cDNA sequence of clone p0157r08c05

SEQ ID NO:4541 is the determined cDNA sequence of clone p0157r08c17

SEQ ID NO:4542 is the determined cDNA sequence of clone p0160r03c01

SEQ ID NO:4543 is the determined cDNA sequence of clone p0161r16c06

SEQ ID NO:4544 is the determined cDNA sequence of clone p0162r02c05

SEQ ID NO:4545 is the determined cDNA sequence of clone p0157r06c24

SEQ ID NO:4546 is the determined cDNA sequence of clone p0157r07c06

SEQ ID NO:4547 is the determined full length cDNA sequence of Pn1467P

SEQ ID NO:4548 is the determined full length cDNA sequence of Pn1468P

SEQ ID NO:4549 is the determined full length cDNA sequence of Pn1472P

SEQ ID NO:4550 is the determined full length cDNA sequence of Pn1475P

SEQ ID NO:4551 is the full length protein sequence of Pn1467P

SEQ ID NO:4552 is the full length protein sequence of Pn1468P

SEQ ID NO:4553 is the full length protein sequence of Pn1472P

SEQ ID NO:4554 is the full length protein sequence of Pn1475P

SEQ ID NO:4555 is the full length cDNA sequence of Pn1509P.

SEQ ID NO:4556 is the full length cDNA sequence of Pn1510P-short, encoding a 243 amino acid ORF of Pn1510P as set forth in SEQ ID NO:4559.

SEQ ID NO:4557 is the full length cDNA sequence of Pn1510P-long, encoding a 278 amino acid ORF of Pn1510P as set forth in SEQ ID NO:4560.

SEQ ID NO:4558 is the full length protein sequence of Pn1509P, encoded by the cDNA set forth in SEQ ID NO:4555.

SEQ ID NO:4559 is the amino acid sequence of the Pn1510P-243 ORF encoded by the cDNA sequence set forth in SEQ ID NO:4556.

SEQ ID NO:4560 is the amino acid sequence of the Pn1510P-278 ORF encoded by the cDNA sequence set forth in SEQ ID NO:4557.

### DETAILED DESCRIPTION OF THE INVENTION

The present invention is directed generally to compositions and their use in the therapy and diagnosis of cancer, particularly pancreatic cancer. As described further below, illustrative compositions of the present invention include, but are not restricted to, polypeptides, particularly immunogenic polypeptides, polynucleotides encoding such polypeptides, antibodies and other binding agents, antigen presenting cells (APCs) and immune system cells (e.g., T cells).

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook, et al. Molecular Cloning: A Laboratory Manual (2nd Edition, 1989); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984).

All publications, patents and patent applications cited herein, whether supra or infra, are hereby incorporated by reference in their entirety.

As used in this specification and the appended claims, the singular forms “a,” “an” and “the” include plural references unless the content clearly dictates otherwise.

Polypeptide Compositions

As used herein, the term “polypeptide” “is used in its conventional meaning, i.e., as a sequence of amino acids. The polypeptides are not limited to a specific length of the product; thus, peptides, oligopeptides, and proteins are included within the definition of polypeptide, and such terms may be used interchangeably herein unless specifically indicated otherwise. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof. Particular polypeptides of interest in the context of this invention are amino acid subsequences comprising epitopes, i.e., antigenic determinants substantially responsible for the immunogenic properties of a polypeptide and being capable of evoking an immune response.

Particularly illustrative polypeptides of the present invention comprise those encoded by a polynucleotide sequence set forth in any one of SEQ ID NOs:1-66, 75-152, 174-177, 182, 184-452, and 454-4550, or a sequence that hybridizes under moderately stringent conditions, or, alternatively, under highly stringent conditions, to a polynucleotide sequence set forth in any one of SEQ ID NOs:1-66, 75-152, 174-177, 182, 184-452, and 454-4550. Certain other illustrative polypeptides of the invention comprise amino acid sequences as set forth in any one of SEQ ID NOs:67-74, 153-173, 178-181, 183, 453, 4551-4554 and 4558-4560.

The polypeptides of the present invention are sometimes herein referred to as pancreatic tumor proteins or pancreatic tumor polypeptides, as an indication that their identification has been based at least in part upon their increased levels of expression in pancreatic tumor samples. Thus, a “pancreatic tumor polypeptide” or “pancreatic tumor protein,” refers generally to a polypeptide sequence of the present invention, or a polynucleotide sequence encoding such a polypeptide, that is expressed in a substantial proportion of pancreatic tumor samples, for example preferably greater than about 20%, more preferably greater than about 30%, and most preferably greater than about 50% or more of pancreatic tumor samples tested, at a level that is at least two fold, and preferably at least five fold, greater than the level of expression in normal tissues, as determined using a representative assay provided herein. A pancreatic tumor polypeptide sequence of the invention, based upon its increased level of expression in tumor cells, has particular utility both as a diagnostic marker as well as a therapeutic target, as further described below.

In certain preferred embodiments, the polypeptides of the invention are immunogenic, i.e., they react detectably within an immunoassay (such as an ELISA or T-cell stimulation assay) with antisera and/or T-cells from a patient with pancreatic cancer. Screening for immunogenic activity can be performed using techniques well known to the skilled artisan. For example, such screens can be performed using methods such as those described in Harlow and Lane, Antibodies: A Laboratory Manual, Cold Spring Harbor Laboratory, 1988. In one illustrative example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

As would be recognized by the skilled artisan, immunogenic portions of the polypeptides disclosed herein are also encompassed by the present invention. An “immunogenic portion,” as used herein, is a fragment of an immunogenic polypeptide of the invention that itself is immunologically reactive (i.e., specifically binds) with the B-cells and/or T-cell surface antigen receptors that recognize the polypeptide. Immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T-cell lines or clones. As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared as described herein, and using well-known techniques.

In one preferred embodiment, an immunogenic portion of a polypeptide of the present invention is a portion that reacts with antisera and/or T-cells at a level that is not substantially less than the reactivity of the full-length polypeptide (e.g., in an ELISA and/or T-cell reactivity assay). Preferably, the level of immunogenic activity of the immunogenic portion is at least about 50%, preferably at least about 70% and most preferably greater than about 90% of the immunogenicity for the full-length polypeptide. In some instances, preferred immunogenic portions will be identified that have a level of immunogenic activity greater than that of the corresponding full-length polypeptide, e.g., having greater than about 100% or 150% or more immunogenic activity.

In certain other embodiments, illustrative immunogenic portions may include peptides in which an N-terminal leader sequence and/or transmembrane domain have been deleted. Other illustrative immunogenic portions will contain a small N- and/or C-terminal deletion (e.g., 1-30 amino acids, preferably 5-15 amino acids), relative to the mature protein.

In another embodiment, a polypeptide composition of the invention may also comprise one or more polypeptides that are immunologically reactive with T cells and/or antibodies generated against a polypeptide of the invention, particularly a polypeptide having an amino acid sequence disclosed herein, or to an immunogenic fragment or variant thereof.

In another embodiment of the invention, polypeptides are provided that comprise one or more polypeptides that are capable of eliciting T cells and/or antibodies that are immunologically reactive with one or more polypeptides described herein, or one or more polypeptides encoded by contiguous nucleic acid sequences contained in the polynucleotide sequences disclosed herein, or immunogenic fragments or variants thereof, or to one or more nucleic acid sequences which hybridize to one or more of these sequences under conditions of moderate to high stringency.

The present invention, in another aspect, provides polypeptide fragments comprising at least about 5, 10, 15, 20, 25, 50, or 100 contiguous amino acids, or more, including all intermediate lengths, of a polypeptide compositions set forth herein, such as those set forth in SEQ ID NOs:67-74, 153-173, 178-181, 183, 453, 4551-4554 and 4558-4560, or those encoded by a polynucleotide sequence set forth in a sequence of SEQ ID NOs:1-66, 75-152, 174-177, 182, 184-452, and 454-4550.

In another aspect, the present invention provides variants of the polypeptide compositions described herein. Polypeptide variants generally encompassed by the present invention will typically exhibit at least about 70%, 75%, 80%, 85%, 90%, 91%, 92%, 93%, 94%, 95%, 96%, 97%, 98%, or 99% or more identity (determined as described below), along its length, to a polypeptide sequences set forth herein.

In one preferred embodiment, the polypeptide fragments and variants provided by the present invention are immunologically reactive with an antibody and/or T-cell that reacts with a full-length polypeptide specifically set forth herein.

In another preferred embodiment, the polypeptide fragments and variants provided by the present invention exhibit a level of immunogenic activity of at least about 50%, preferably at least about 70%, and most preferably at least about 90% or more of that exhibited by a full-length polypeptide sequence specifically set forth herein.

A polypeptide “variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the invention and evaluating their immunogenic activity as described herein and/or using any of a number of techniques well known in the art.

For example, certain illustrative variants of the polypeptides of the invention include those in which one or more portions, such as an N-terminal leader sequence or transmembrane domain, have been removed. Other illustrative variants include variants in which a small portion (e.g., 1-30 amino acids, preferably 5-15 amino acids) has been removed from the N- and/or C-terminal of the mature protein.

In many instances, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present invention and still obtain a functional molecule that encodes a variant or derivative polypeptide with desirable characteristics, e.g., with immunogenic characteristics. When it is desired to alter the amino acid sequence of a polypeptide to create an equivalent, or even an improved, immunogenic variant or portion of a polypeptide of the invention, one skilled in the art will typically change one or more of the codons of the encoding DNA sequence according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their biological utility or activity.

TABLE 1

| Amino Acids | | | Codons |
|---|---|---|---|
| Alanine | Ala | A | GCA CCC GCG GGU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UGA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

In making such changes, the hydropathic index of amino acids may be considered. The importance of the hydropathic amino acid index in conferring interactive biologic function on a protein is generally understood in the art (Kyte and Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within is preferred, those within ±1 are particularly preferred, and those within ±0.5 are even more particularly preferred. It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

In addition, any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

As noted above, polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

When comparing polypeptide sequences, two sequences are said to be “identical” if the sequence of amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970). Mol. Biol. 48:443, by the search for similarity methods of Pearson and Lipman (1988) Proc. Natl. Acad. Sci. USA 85: 2444, by computerized implementations of these algorithms (GAP,

### BESTFIT, BLAST, FASTA, and TFASTA in the Wisconsin Genetics Software Package Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one preferred approach, the “percentage of sequence identity” is determined by comparing two optimally aligned sequences over a window of comparison of at least 20 positions, wherein the portion of the polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e., the window size) and multiplying the results by 100 to yield the percentage of sequence identity.

Within other illustrative embodiments, a polypeptide may be a xenogeneic polypeptide that comprises an polypeptide having substantial sequence identity, as described above, to the human polypeptide (also termed autologous antigen) which served as a reference polypeptide, but which xenogeneic polypeptide is derived from a different, non-human species. One skilled in the art will recognize that “self” antigens are often poor stimulators of CD8+ and CD4+ T-lymphocyte responses, and therefore efficient immunotherapeutic strategies directed against tumor polypeptides require the development of methods to overcome immune tolerance to particular self tumor polypeptides. For example, humans immunized with prostase protein from a xenogeneic (non human) origin are capable of mounting an immune response against the counterpart human protein, e.g. the human prostase tumor protein present on human tumor cells. Accordingly, the present invention provides methods for purifying the xenogeneic form of the tumor proteins set forth herein, such as the polypeptides set forth in SEQ ID NOs:67-74, 153-173, 178-181, 183, 453, 4551-4554 and 4558-4560, or those encoded by polynucleotide sequences set forth in SEQ ID NOs:1-66, 75-152, 174-177, 182, 184-452 and 454-4550.

Therefore, one aspect of the present invention provides xenogeneic variants of the polypeptide compositions described herein. Such xenogeneic variants generally encompassed by the present invention will typically exhibit at least about 70%, 75%, 80%, 85%, 90%, 91%, 92%, 93%, 94%, 95%, 96%, 97%, 98%, or 99% or more identity along their lengths, to a polypeptide sequences set forth herein.

More particularly, the invention is directed to mouse, rat, monkey, porcine and other non-human polypeptides which can be used as xenogeneic forms of human polypeptides set forth herein, to induce immune responses directed against tumor polypeptides of the invention.

Within other illustrative embodiments, a polypeptide may be a fusion polypeptide that comprises multiple polypeptides as described herein, or that comprises at least one polypeptide as described herein and an unrelated sequence, such as a known tumor protein. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the polypeptide or to enable the polypeptide to be targeted to desired intracellular compartments. Still further fusion partners include affinity tags, which facilitate purification of the polypeptide.

Fusion polypeptides may generally be prepared using standard techniques, including chemical conjugation. Preferably, a fusion polypeptide is expressed as a recombinant polypeptide, allowing the production of increased levels, relative to a non-fused polypeptide, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion polypeptide that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion polypeptide using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

The fusion polypeptide can comprise a polypeptide as described herein together with an unrelated immunogenic protein, such as an immunogenic protein capable of eliciting a recall response. Examples of such proteins include tetanus, tuberculosis and hepatitis proteins (see, for example, Stoute et al. New Engl. J. Med., 336:86-91, 1997).

In one preferred embodiment, the immunological fusion partner is derived from a Mycobacterium sp., such as a Mycobacterium tuberculosis-derived Ra12 fragment. Ra12 compositions and methods for their use in enhancing the expression and/or immunogenicity of heterologous polynucleotide/polypeptide sequences is described in U.S. Patent Application No. 60/158,585, the disclosure of which is incorporated herein by reference in its entirety. Briefly, Ra12 refers to a polynucleotide region that is a subsequence of a Mycobacterium tuberculosis MTB32A nucleic acid. MTB32A is a serine protease of 32 KD molecular weight encoded by a gene in virulent and avirulent strains of M. tuberculosis. The nucleotide sequence and amino acid sequence of MTB32A have been described (for example, U.S. Patent Application No. 60/158,585; see also, Skeiky et al., Infection and Immun. (1999) 67:3998-4007, incorporated herein by reference). C-terminal fragments of the MTB32A coding sequence express at high levels and remain as a soluble polypeptides throughout the purification process. Moreover, Ra12 may enhance the immunogenicity of heterologous immunogenic polypeptides with which it is fused. One preferred Ra12 fusion polypeptide comprises a 14 KD C-terminal fragment corresponding to amino acid residues 192 to 323 of MTB32A. Other preferred Ra12 polynucleotides generally comprise at least about 15 consecutive nucleotides, at least about 30 nucleotides, at least about 60 nucleotides, at least about 100 nucleotides, at least about 200 nucleotides, or at least about 300 nucleotides that encode a portion of a Ra12 polypeptide. Ra12 polynucleotides may comprise a native sequence (i.e., an endogenous sequence that encodes a Ra12 polypeptide or a portion thereof) or may comprise a variant of such a sequence. Ra12 polynucleotide variants may contain one or more substitutions, additions, deletions and/or insertions such that the biological activity of the encoded fusion polypeptide is not substantially diminished, relative to a fusion polypeptide comprising a native Ra12 polypeptide. Variants preferably exhibit at least about 70% identity, more preferably at least about 80% identity and most preferably at least about 90% identity to a polynucleotide sequence that encodes a native Ra12 polypeptide or a portion thereof.

Within other preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenzae virus, NS 1 (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion polypeptide. A repeat portion is found in the C-terminal region starting at residue 178. A particularly preferred repeat portion incorporates residues 188-305.

Yet another illustrative embodiment involves fusion polypeptides, and the polynucleotides encoding them, wherein the fusion partner comprises a targeting signal capable of directing a polypeptide to the endosomal/lysosomal compartment, as described in U.S. Pat. No. 5,633,234. An immunogenic polypeptide of the invention, when fused with this targeting signal, will associate more efficiently with MHC class II molecules and thereby provide enhanced in vivo stimulation of CD4⁺ T-cells specific for the polypeptide.

Polypeptides of the invention are prepared using any of a variety of well known synthetic and/or recombinant techniques, the latter of which are further described below. Polypeptides, portions and other variants generally less than about 150 amino acids can be generated by synthetic means, using techniques well known to those of ordinary skill in the art. In one illustrative example, such polypeptides are synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division (Foster City, Calif.), and may be operated according to the manufacturer's instructions.

In general, polypeptide compositions (including fusion polypeptides) of the invention are isolated. An “isolated” polypeptide is one that is removed from its original environment. For example, a naturally-occurring protein or polypeptide is isolated if it is separated from some or all of the coexisting materials in the natural system. Preferably, such polypeptides are also purified, e.g., are at least about 90% pure, more preferably at least about 95% pure and most preferably at least about 99% pure.

Polynucleotide Compositions

The present invention, in other aspects, provides polynucleotide compositions. The terms “DNA” and “polynucleotide” are used essentially interchangeably herein to refer to a DNA molecule that has been isolated free of total genomic DNA of a particular species. “Isolated,” as used herein, means that a polynucleotide is substantially away from other coding sequences, and that the DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment by the hand of man.

As will be understood by those skilled in the art, the polynucleotide compositions of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

As will be also recognized by the skilled artisan, polynucleotides of the invention may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes a polypeptide/protein of the invention or a portion thereof) or may comprise a sequence that encodes a variant or derivative, preferably and immunogenic variant or derivative, of such a sequence.

Therefore, according to another aspect of the present invention, polynucleotide compositions are provided that comprise some or all of a polynucleotide sequence set forth in any one of SEQ ID NOs:1-66, 75-152, 174-177, 182, 184-452, and 454-4550, complements of a polynucleotide sequence set forth in any one of SEQ ID NOs:1-66, 75-152, 174-177, 182, 184-452, and 454-4550, and degenerate variants of a polynucleotide sequence set forth in any one of SEQ ID NOs:1-66, 75-152, 174-177, 182, 184-452, and 454-4550. In certain preferred embodiments, the polynucleotide sequences set forth herein encode immunogenic polypeptides, as described above.

In other related embodiments, the present invention provides polynucleotide variants having substantial identity to the sequences disclosed herein in SEQ ID NOs:1-66, 75-152, 174-177, 182, 184-452, and 454-4550, for example those comprising at least 70% sequence identity, preferably at least 75%, 80%, 85%, 90%, 95%, 96%, 97%, 98%, or 99% or higher, sequence identity compared to a polynucleotide sequence of this invention using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

Typically, polynucleotide variants will contain one or more substitutions, additions, deletions and/or insertions, preferably such that the immunogenicity of the polypeptide encoded by the variant polynucleotide is not substantially diminished relative to a polypeptide encoded by a polynucleotide sequence specifically set forth herein). The term “variants” should also be understood to encompasses homologous genes of xenogenic origin.

In additional embodiments, the present invention provides polynucleotide fragments comprising various lengths of contiguous stretches of sequence identical to or complementary to one or more of the sequences disclosed herein. For example, polynucleotides are provided by this invention that comprise at least about 10, 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like. A polynucleotide sequence as described here may be extended at one or both ends by additional nucleotides not found in the native sequence. This additional sequence may consist of 1, 2, 3, 4, 5, 6, 7, 8, 9, 10, 11, 12, 13, 14, 15, 16, 17, 18, 19, or 20 nucleotides at either end of the disclosed sequence or at both ends of the disclosed sequence.

In another embodiment of the invention, polynucleotide compositions are provided that are capable of hybridizing under moderate to high stringency conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5× SSC, 0.5% SDS, 1.0 mM EDTA (pH 8.0); hybridizing at 50° C.-60° C., 5× SSC, overnight; followed by washing twice at 65° C. for 20 minutes with each of 2×, 0.5× and 0.2× SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60-65° C. or 65-70° C.

In certain preferred embodiments, the polynucleotides described above, e.g., polynucleotide variants, fragments and hybridizing sequences, encode polypeptides that are immunologically cross-reactive with a polypeptide sequence specifically set forth herein. In other preferred embodiments, such polynucleotides encode polypeptides that have a level of immunogenic activity of at least about 50%, preferably at least about 70%, and more preferably at least about 90% of that for a polypeptide sequence specifically set forth herein.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

When comparing polynucleotide sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides of the invention. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

Preferably, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Therefore, in another embodiment of the invention, a mutagenesis approach, such as site-specific mutagenesis, is employed for the preparation of immunogenic variants and/or derivatives of the polypeptides described herein. By this approach, specific modifications in a polypeptide sequence can be made through mutagenesis of the underlying polynucleotides that encode them. These techniques provides a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments of the present invention, the inventors contemplate the mutagenesis of the disclosed polynucleotide sequences to alter one or more properties of the encoded polypeptide, such as the immunogenicity of a polypeptide vaccine. The techniques of site-specific mutagenesis are well-known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phage are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

In another approach for the production of polypeptide variants of the present invention, recursive sequence recombination, as described in U.S. Pat. No. 5,837,458, may be employed. In this approach, iterative cycles of recombination and screening or selection are performed to “evolve” individual polynucleotide variants of the invention having, for example, enhanced immunogenic activity.

In other embodiments of the present invention, the polynucleotide sequences provided herein can be advantageously used as probes or primers for nucleic acid hybridization. As such, it is contemplated that nucleic acid segments that comprise a sequence region of at least about 15 nucleotide long contiguous sequence that has the same sequence as, or is complementary to, a 15 nucleotide long contiguous sequence disclosed herein will find particular utility. Longer contiguous identical or complementary sequences, e.g., those of about 20, 30, 40, 50, 100, 200, 500, 1000 (including all intermediate lengths) and even up to full length sequences will also be of use in certain embodiments.

The ability of such nucleic acid probes to specifically hybridize to a sequence of interest will enable them to be of use in detecting the presence of complementary sequences in a given sample. However, other uses are also envisioned, such as the use of the sequence information for the preparation of mutant species primers, or primers for use in preparing other genetic constructions.

Polynucleotide molecules having sequence regions consisting of contiguous nucleotide stretches of 10-14, 15-20, 30, 50, or even of 100-200 nucleotides or so (including intermediate lengths as well), identical or complementary to a polynucleotide sequence disclosed herein, are particularly contemplated as hybridization probes for use in, e.g., Southern and Northern blotting. This would allow a gene product, or fragment thereof, to be analyzed, both in diverse cell types and also in various bacterial cells. The total size of fragment, as well as the size of the complementary stretch(es), will ultimately depend on the intended use or application of the particular nucleic acid segment. Smaller fragments will generally find use in hybridization embodiments, wherein the length of the contiguous complementary region may be varied, such as between about 15 and about 100 nucleotides, but larger contiguous complementarity stretches may be used, according to the length complementary sequences one wishes to detect.

The use of a hybridization probe of about 15-25 nucleotides in length allows the formation of a duplex molecule that is both stable and selective. Molecules having contiguous complementary sequences over stretches greater than 15 bases in length are generally preferred, though, in order to increase stability and selectivity of the hybrid, and thereby improve the quality and degree of specific hybrid molecules obtained. One will generally prefer to design nucleic acid molecules having gene-complementary stretches of 15 to 25 contiguous nucleotides, or even longer where desired.

Hybridization probes may be selected from any portion of any of the sequences disclosed herein. All that is required is to review the sequences set forth herein, or to any continuous portion of the sequences, from about 15-25 nucleotides in length up to and including the full length sequence, that one wishes to utilize as a probe or primer. The choice of probe and primer sequences may be governed by various factors. For example, one may wish to employ primers from towards the termini of the total sequence.

Small polynucleotide segments or fragments may be readily prepared by, for example, directly synthesizing the fragment by chemical means, as is commonly practiced using an automated oligonucleotide synthesizer. Also, fragments may be obtained by application of nucleic acid reproduction technology, such as the PCR™ technology of U.S. Pat. No. 4,683,202 (incorporated herein by reference), by introducing selected sequences into recombinant vectors for recombinant production, and by other recombinant DNA techniques generally known to those of skill in the art of molecular biology.

The nucleotide sequences of the invention may be used for their ability to selectively form duplex molecules with complementary stretches of the entire gene or gene fragments of interest. Depending on the application envisioned, one will typically desire to employ varying conditions of hybridization to achieve varying degrees of selectivity of probe towards target sequence. For applications requiring high selectivity, one will typically desire to employ relatively stringent conditions to form the hybrids, e.g., one will select relatively low salt and/or high temperature conditions, such as provided by a salt concentration of from about 0.02 M to about 0.15 M salt at temperatures of from about 50° C. to about 70° C. Such selective conditions tolerate little, if any, mismatch between the probe and the template or target strand, and would be particularly suitable for isolating related sequences.

Of course, for some applications, for example, where one desires to prepare mutants employing a mutant primer strand hybridized to an underlying template, less stringent (reduced stringency) hybridization conditions will typically be needed in order to allow formation of the heteroduplex. In these circumstances, one may desire to employ salt conditions such as those of from about 0.15 M to about 0.9 M salt, at temperatures ranging from about 20° C. to about 55° C. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions can be readily manipulated, and thus will generally be a method of choice depending on the desired results.

According to another embodiment of the present invention, polynucleotide compositions comprising antisense oligonucleotides are provided. Antisense oligonucleotides have been demonstrated to be effective and targeted inhibitors of protein synthesis, and, consequently, provide a therapeutic approach by which a disease can be treated by inhibiting the synthesis of proteins that contribute to the disease. The efficacy of antisense oligonucleotides for inhibiting protein synthesis is well established. For example, the synthesis of polygalactauronase and the muscarine type 2 acetylcholine receptor are inhibited by antisense oligonucleotides directed to their respective mRNA sequences (U.S. Pat. No. 5,739,119 and U.S. Pat. No. 5,759,829). Further, examples of antisense inhibition have been demonstrated with the nuclear protein cyclin, the multiple drug resistance gene (MDG1), ICAM-1, E-selectin, STK-1, striatal GABAA receptor and human EGF (Jaskulski et al., Science. 1988 Jun 10;240(4858):1544-6; Vasanthakumar and Ahmed, Cancer Commun. 1989;1(4):225-32; Peris et al., Brain Res Mol Brain Res. 1998 Jun 15;57(2):310-20; U.S. Pat. No. 5,801,154; U.S. Pat. No. 5,789,573; U.S. Pat. No. 5,718,709 and U.S. Pat. No. 5,610,288). Antisense constructs have also been described that inhibit and can be used to treat a variety of abnormal cellular proliferations, e.g. cancer (U.S. Pat. No. 5,747,470; U.S. Pat. No. 5,591,317 and U.S. Pat. No. 5,783,683).

Therefore, in certain embodiments, the present invention provides oligonucleotide sequences that comprise all, or a portion of, any sequence that is capable of specifically binding to polynucleotide sequence described herein, or a complement thereof. In one embodiment, the antisense oligonucleotides comprise DNA or derivatives thereof. In another embodiment, the oligonucleotides comprise RNA or derivatives thereof. In a third embodiment, the oligonucleotides are modified DNAs comprising a phosphorothioated modified backbone. In a fourth embodiment, the oligonucleotide sequences comprise peptide nucleic acids or derivatives thereof. In each case, preferred compositions comprise a sequence region that is complementary, and more preferably substantially-complementary, and even more preferably, completely complementary to one or more portions of polynucleotides disclosed herein. Selection of antisense compositions specific for a given gene sequence is based upon analysis of the chosen target sequence and determination of secondary structure, Tm, binding energy, and relative stability. Antisense compositions may be selected based upon their relative inability to form dimers, hairpins, or other secondary structures that would reduce or prohibit specific binding to the target mRNA in a host cell. Highly preferred target regions of the mRNA, are those which are at or near the AUG translation initiation codon, and those sequences which are substantially complementary to 5′ regions of the mRNA. These secondary structure analyses and target site selection considerations can be performed, for example, using v.4 of the OLIGO primer analysis software and/or the BLASTN 2.0.5 algorithm software (Altschul et al., Nucleic Acids Res. 1997, 25(17):3389-402).

The use of an antisense delivery method employing a short peptide vector, termed MPG (27 residues), is also contemplated. The MPG peptide contains a hydrophobic domain derived from the fusion sequence of HIV gp41 and a hydrophilic domain from the nuclear localization sequence of SV40 T-antigen (Morris et al., Nucleic Acids Res. 1997 Jul 15;25(14):2730-6). It has been demonstrated that several molecules of the MPG peptide coat the antisense oligonucleotides and can be delivered into cultured mammalian cells in less than 1 hour with relatively high efficiency (90%). Further, the interaction with MPG strongly increases both the stability of the oligonucleotide to nuclease and the ability to cross the plasma membrane.

According to another embodiment of the invention, the polynucleotide compositions described herein are used in the design and preparation of ribozyme molecules for inhibiting expression of the tumor polypeptides and proteins of the present invention in tumor cells. Ribozymes are RNA-protein complexes that cleave nucleic acids in a site-specific fashion. Ribozymes have specific catalytic domains that possess endonuclease activity (Kim and Cech, Proc Natl Acad Sci U S A. 1987 Dec;84(24):8788-92; Forster and Symons, Cell. 1987 Apr 24;49(2):211-20). For example, a large number of ribozymes accelerate phosphoester transfer reactions with a high degree of specificity, often cleaving only one of several phosphoesters in an oligonucleotide substrate (Cech et al., Cell. 1981 Dec;27(3 Pt 2):487-96; Michel and Westhof, J Mol Biol. 1990 Dec 5;216(3):585-610; Reinhold-Hurek and Shub, Nature. 1992 May 14;357(6374):173-6). This specificity has been attributed to the requirement that the substrate bind via specific base-pairing interactions to the internal guide sequence (“IGS”) of the ribozyme prior to chemical reaction.

Six basic varieties of naturally-occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nature of a ribozyme is advantageous over many technologies, such as antisense technology (where a nucleic acid molecule simply binds to a nucleic acid target to block its translation) since the concentration of ribozyme necessary to affect a therapeutic treatment is lower than that of an antisense oligonucleotide. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base pairing mechanism of binding to the target RNA, but also on the mechanism of target RNA cleavage. Single mismatches, or base-substitutions, near the site of cleavage can completely eliminate catalytic activity of a ribozyme. Similar mismatches in antisense molecules do not prevent their action (Woolf et al., Proc Natl Acad Sci U S A. 1992 Aug 15;89(16):7305-9). Thus, the specificity of action of a ribozyme is greater than that of an antisense oligonucleotide binding the same RNA site.

The enzymatic nucleic acid molecule may be formed in a hammerhead, hairpin, a hepatitis δ virus, group I intron or RNaseP RNA (in association with an RNA guide sequence) or Neurospora VS RNA motif. Examples of hammerhead motifs are described by Rossi et al Nucleic Acids Res. 1992 Sep 11;20(17):4559-65. Examples of hairpin motifs are described by Hampel et al. (Eur. Pat. Appl. Publ. No. EP 0360257), Hampel and Tritz, Biochemistry 1989 Jun 13;28(12):4929-33; Hampel et al., Nucleic Acids Res. 1990 Jan 25;18(2):299-304 and U.S. Pat. No. 5,631,359. An example of the hepatitis δ virus motif is described by Perrotta and Been, Biochemistry. 1992 Dec 1;31(47):11843-52; an example of the RNaseP motif is described by Guerrier-Takada et al., Cell. 1983 Dec;35(3 Pt 2):849-57; Neurospora VS RNA ribozyme motif is described by Collins (Saville and Collins, Cell. 1990 May 18;61(4):685-96; Saville and Collins, Proc Natl Acad Sci U S A. 1991 Oct 1;88(19):8826-30; Collins and Olive, Biochemistry. 1993 Mar 23;32(11):2795-9); and an example of the Group I intron is described in (U.S. Pat. No. 4,987,071). All that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule. Thus the ribozyme constructs need not be limited to specific motifs mentioned herein.

Ribozymes may be designed as described in Int. Pat. Appl. Publ. No. WO 93/23569 and Int. Pat. Appl. Publ. No. WO 94/02595, each specifically incorporated herein by reference) and synthesized to be tested in vitro and in vivo, as described. Such ribozymes can also be optimized for delivery. While specific examples are provided, those in the art will recognize that equivalent RNA targets in other species can be utilized when necessary.

Ribozyme activity can be optimized by altering the length of the ribozyme binding arms, or chemically synthesizing ribozymes with modifications that prevent their degradation by serum ribonucleases (see e.g, Int. Pat. Appl. Publ. No. WO 92/07065; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

Sullivan et al. (Int. Pat. Appl. Publ. No. WO 94/02595) describes the general methods for delivery of enzymatic RNA molecules. Ribozymes may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, ribozymes may be directly delivered ex vivo to cells or tissues with or without the aforementioned vehicles. Alternatively, the RNA/vehicle combination may be locally delivered by direct inhalation, by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of ribozyme delivery and administration are provided in Int. Pat. Appl. Publ. No. WO 94/02595 and Int. Pat. Appl. Publ. No. WO 93/23569, each specifically incorporated herein by reference.

Another means of accumulating high concentrations of a ribozyme(s) within cells is to incorporate the ribozyme-encoding sequences into a DNA expression vector. Transcription of the ribozyme sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters may also be used, providing that the prokaryotic RNA polymerase enzyme is expressed in the appropriate cells. Ribozymes expressed from such promoters have been shown to function in mammalian cells. Such transcription units can be incorporated into a variety of vectors for introduction into mammalian cells, including but not restricted to, plasmid DNA vectors, viral DNA vectors (such as adenovirus or adeno-associated vectors), or viral RNA vectors (such as retroviral, semliki forest virus, sindbis virus vectors).

In another embodiment of the invention, peptide nucleic acids (PNAs) compositions are provided. PNA is a DNA mimic in which the nucleobases are attached to a pseudopeptide backbone (Good and Nielsen, Antisense Nucleic Acid Drug Dev. 1997 7(4) 431-37). PNA is able to be utilized in a number methods that traditionally have used RNA or DNA. Often PNA sequences perform better in techniques than the corresponding RNA or DNA sequences and have utilities that are not inherent to RNA or DNA. A review of PNA including methods of making, characteristics of, and methods of using, is provided by Corey (Trends Biotechnol 1997 Jun;15(6):224-9). As such, in certain embodiments, one may prepare PNA sequences that are complementary to one or more portions of the ACE mRNA sequence, and such PNA compositions may be used to regulate, alter, decrease, or reduce the translation of ACE-specific mRNA, and thereby alter the level of ACE activity in a host cell to which such PNA compositions have been administered.

PNAs have 2-aminoethyl-glycine linkages replacing the normal phosphodiester backbone of DNA (Nielsen et al., Science 1991 Dec 6;254(5037):1497-500; Hanvey et al., Science. 1992 Nov 27;258(5087):1481-5; Hyrup and Nielsen, Bioorg Med Chem. 1996 Jan;4(1):5-23). This chemistry has three important consequences: firstly, in contrast to DNA or phosphorothioate oligonucleotides, PNAs are neutral molecules; secondly, PNAs are achiral, which avoids the need to develop a stereoselective synthesis; and thirdly, PNA synthesis uses standard Boc or Fmoc protocols for solid-phase peptide synthesis, although other methods, including a modified Merrifield method, have been used.

PNA monomers or ready-made oligomers are commercially available from PerSeptive Biosystems (Framingham, Mass.). PNA syntheses by either Boc or Fmoc protocols are straightforward using manual or automated protocols (Norton et al., Bioorg Med Chem. 1995 Apr;3(4):437-45). The manual protocol lends itself to the production of chemically modified PNAs or the simultaneous synthesis of families of closely related PNAs.

As with peptide synthesis, the success of a particular PNA synthesis will depend on the properties of the chosen sequence. For example, while in theory PNAs can incorporate any combination of nucleotide bases, the presence of adjacent purines can lead to deletions of one or more residues in the product. In expectation of this difficulty, it is suggested that, in producing PNAs with adjacent purines, one should repeat the coupling of residues likely to be added inefficiently. This should be followed by the purification of PNAs by reverse-phase high-pressure liquid chromatography, providing yields and purity of product similar to those observed during the synthesis of peptides.

Modifications of PNAs for a given application may be accomplished by coupling amino acids during solid-phase synthesis or by attaching compounds that contain a carboxylic acid group to the exposed N-terminal amine. Alternatively, PNAs can be modified after synthesis by coupling to an introduced lysine or cysteine. The ease with which PNAs can be modified facilitates optimization for better solubility or for specific functional requirements. Once synthesized, the identity of PNAs and their derivatives can be confirmed by mass spectrometry. Several studies have made and utilized modifications of PNAs (for example, Norton et al., Bioorg Med Chem. 1995 Apr;3(4):437-45; Petersen et al., J Pept Sci. 1995 May-Jun;1(3):175-83; Orum et al., Biotechniques. 1995 Sep;19(3):472-80; Footer et al., Biochemistry. 1996 Aug 20;35(33):10673-9; Griffith et al., Nucleic Acids Res. 1995 Aug 11;23(15):3003-8; Pardridge et al., Proc Natl Acad Sci U S A. 1995 Jun 6;92(12):5592-6; Boffa et al., Proc Natl Acad Sci U S A. 1995 Mar 14;92(6):1901-5; Gambacorti-Passerini et al., Blood. 1996 Aug 15;88(4):1411-7; Armitage et al., Proc Natl Acad Sci U S A. 1997 Nov 11;94(23):12320-5; Seeger et al., Biotechniques. 1997 Sep;23(3):512-7). U.S. Pat. No. 5,700,922 discusses PNA-DNA-PNA chimeric molecules and their uses in diagnostics, modulating protein in organisms, and treatment of conditions susceptible to therapeutics.

Methods of characterizing the antisense binding properties of PNAs are discussed in Rose (Anal Chem. 1993 Dec 15;65(24):3545-9) and Jensen et al. (Biochemistry. 1997 Apr 22;36(16):5072-7). Rose uses capillary gel electrophoresis to determine binding of PNAs to their complementary oligonucleotide, measuring the relative binding kinetics and stoichiometry. Similar types of measurements were made by Jensen et al. using BIAcore™ technology.

Other applications of PNAs that have been described and will be apparent to the skilled artisan include use in DNA strand invasion, antisense inhibition, mutational analysis, enhancers of transcription, nucleic acid purification, isolation of transcriptionally active genes, blocking of transcription factor binding, genome cleavage, biosensors, in situ hybridization, and the like.

Polynucleotide Identification, Characterization and Expression

Polynucleotides compositions of the present invention may be identified, prepared and/or manipulated using any of a variety of well established techniques (see generally, Sambrook et al., Molecular Cloning: A Laboratory Manual, Cold Spring Harbor Laboratories, Cold Spring Harbor, N.Y., 1989, and other like references). For example, a polynucleotide may be identified, as described in more detail below, by screening a microarray of cDNAs for tumor-associated expression (i.e., expression that is at least two fold greater in a tumor than in normal tissue, as determined using a representative assay provided herein). Such screens may be performed, for example, using the microarray technology of Affymetrix, Inc. (Santa Clara, Calif.) according to the manufacturer's instructions (and essentially as described by Schena et al., Proc. Natl. Acad. Sci. USA 93:10614-10619, 1996 and Heller et al., Proc. Natl. Acad. Sci. USA 94:2150-2155, 1997). Alternatively, polynucleotides may be amplified from cDNA prepared from cells expressing the proteins described herein, such as tumor cells.

Many template dependent processes are available to amplify a target sequences of interest present in a sample. One of the best known amplification methods is the polymerase chain reaction (PCR™) which is described in detail in U.S. Pat. Nos. 4,683,195, 4,683,202 and 4,800,159, each of which is incorporated herein by reference in its entirety. Briefly, in PCR™, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR™ amplification procedure may be performed in order to quantify the amount of mRNA amplified. Polymerase chain reaction methodologies are well known in the art.

Any of a number of other template dependent processes, many of which are variations of the PCR™ amplification technique, are readily known and available in the art. Illustratively, some such methods include the ligase chain reaction (referred to as LCR), described, for example, in Eur. Pat. Appl. Publ. No. 320,308 and U.S. Pat. No. 4,883,750; Qbeta Replicase, described in PCT Intl. Pat. Appl. Publ. No. PCT/US87/00880; Strand Displacement Amplification (SDA) and Repair Chain Reaction (RCR). Still other amplification methods are described in Great Britain Pat. Appl. No. 2202328, and in PCT Intl. Pat. Appl. Pubi. No. PCT/US89/01025. Other nucleic acid amplification procedures include transcription-based amplification systems (TAS) (PCT Intl. Pat. Appl. Publ. No. WO 88/10315), including nucleic acid sequence based amplification (NASBA) and 3SR. Eur. Pat. Appl. Publ. No. 329,822 describes a nucleic acid amplification process involving cyclically synthesizing single-stranded RNA (“ssRNA”), ssDNA, and double-stranded DNA (dsDNA). PCT Intl. Pat. Appl. Publ. No. WO 89/06700 describes a nucleic acid sequence amplification scheme based on the hybridization of a promoter/primer sequence to a target single-stranded DNA (“ssDNA”) followed by transcription of many RNA copies of the sequence. Other amplification methods such as “RACE” (Frohman, 1990), and “one-sided PCR” (Ohara, 1989) are also well-known to those of skill in the art.

An amplified portion of a polynucleotide of the present invention may be used to isolate a full length gene from a suitable library (e.g., a tumor cDNA library) using well known techniques. Within such techniques, a library (cDNA or genomic) is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences.

For hybridization techniques, a partial sequence may be labeled (e.g., by nick-translation or end-labeling with ³²P) using well known techniques. A bacterial or bacteriophage library is then generally screened by hybridizing filters containing denatured bacterial colonies (or lawns containing phage plaques) with the labeled probe (see Sambrook et al., Molecular Cloning: A Laboratory Manual, Cold Spring Harbor Laboratories, Cold Spring Harbor, N.Y., 1989). Hybridizing colonies or plaques are selected and expanded, and the DNA is isolated for further analysis. cDNA clones may be analyzed to determine the amount of additional sequence by, for example, PCR using a primer from the partial sequence and a primer from the vector. Restriction maps and partial sequences may be generated to identify one or more overlapping clones. The complete sequence may then be determined using standard techniques, which may involve generating a series of deletion clones. The resulting overlapping sequences can then assembled into a single contiguous sequence. A full length cDNA molecule can be generated by ligating suitable fragments, using well known techniques.

Alternatively, amplification techniques, such as those described above, can be useful for obtaining a full length coding sequence from a partial cDNA sequence. One such amplification technique is inverse PCR (see Triglia et al., Nucl. Acids Res. 16:8186, 1988), which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1:111-19, 1991) and walking PCR (Parker et al., Nucl. Acids. Res. 19:3055-60, 1991). Other methods employing amplification may also be employed to obtain a full length cDNA sequence.

In certain instances, it is possible to obtain a full length cDNA sequence by analysis of sequences provided in an expressed sequence tag (EST) database, such as that available from GenBank. Searches for overlapping ESTs may generally be performed using well known programs (e.g., NCBI BLAST searches), and such ESTs may be used to generate a contiguous full length sequence. Full length DNA sequences may also be obtained by analysis of genomic fragments.

In other embodiments of the invention, polynucleotide sequences or fragments thereof which encode polypeptides of the invention, or fusion proteins or functional equivalents thereof, may be used in recombinant DNA molecules to direct expression of a polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

Moreover, the polynucleotide sequences of the present invention can be engineered using methods generally known in the art in order to alter polypeptide encoding sequences for a variety of reasons, including but not limited to, alterations which modify the cloning, processing, and/or expression of the gene product. For example, DNA shuffling by random fragmentation and PCR reassembly of gene fragments and synthetic oligonucleotides may be used to engineer the nucleotide sequences. In addition, site-directed mutagenesis may be used to insert new restriction sites, alter glycosylation patterns, change codon preference, produce splice variants, or introduce mutations, and so forth.

In another embodiment of the invention, natural, modified, or recombinant nucleic acid sequences may be ligated to a heterologous sequence to encode a fusion protein. For example, to screen peptide libraries for inhibitors of polypeptide activity, it may be useful to encode a chimeric protein that can be recognized by a commercially available antibody. A fusion protein may also be engineered to contain a cleavage site located between the polypeptide-encoding sequence and the heterologous protein sequence, so that the polypeptide may be cleaved and purified away from the heterologous moiety.

Sequences encoding a desired polypeptide may be synthesized, in whole or in part, using chemical methods well known in the art (see Caruthers, M. H. et al. (1980) Nucl. Acids Res. Symp. Ser. 215-223, Horn, T. et al. (1980) Nucl. Acids Res. Symp. Ser. 225-232). Alternatively, the protein itself may be produced using chemical methods to synthesize the amino acid sequence of a polypeptide, or a portion thereof. For example, peptide synthesis can be performed using various solid-phase techniques (Roberge, J. Y. et al. (1995) Science 269:202-204) and automated synthesis may be achieved, for example, using the ABI 431A Peptide Synthesizer (Perkin Elmer, Palo Alto, Calif.).

A newly synthesized peptide may be substantially purified by preparative high performance liquid chromatography (e.g., Creighton, T. (1983) Proteins, Structures and Molecular Principles, WH Freeman and Co., New York, N.Y.) or other comparable techniques available in the art. The composition of the synthetic peptides may be confirmed by amino acid analysis or sequencing (e.g., the Edman degradation procedure). Additionally, the amino acid sequence of a polypeptide, or any part thereof, may be altered during direct synthesis and/or combined using chemical methods with sequences from other proteins, or any part thereof, to produce a variant polypeptide.

In order to express a desired polypeptide, the nucleotide sequences encoding the polypeptide, or functional equivalents, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described, for example, in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y.

A variety of expression vector/host systems may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, any of a number of expression vectors may be selected depending upon the use intended for the expressed polypeptide. For example, when large quantities are needed, for example for the induction of antibodies, vectors which direct high level expression of fusion proteins that are readily purified may be used. Such vectors include, but are not limited to, the multifunctional E. coli cloning and expression vectors such as pBLUESCRIPT (Stratagene), in which the sequence encoding the polypeptide of interest may be ligated into the vector in frame with sequences for the amino-terminal Met and the subsequent 7 residues of .beta.-galactosidase so that a hybrid protein is produced; pIN vectors (Van Heeke, G. and S. M. Schuster (1989) J. Biol. Chem. 264:5503-5509); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al. (1987) Methods Enzymol. 153:516-544.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, N. (1987) EMBO J. 6:307-311. Alternatively, plant promoters such as the small subunit of RUBISCO or heat shock promoters may be used (Coruzzi, G et al. (1984) EMBO J 3:1671-1680; Broglie, R. et al. (1984) Science 224:838-843; and Winter, J. et al. (1991) Results Probl. Cell Differ. 17:85-105). These constructs can be introduced into plant cells by direct DNA transformation or pathogen-mediated transfection. Such techniques are described in a number of generally available reviews (see, for example, Hobbs, S. or Murry, L. E. in McGraw Hill Yearbook of Science and Technology (1992) McGraw Hill, New York, N.Y.; pp. 191-196).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard, E. K. et al. (1994) Proc. Natl. Acad Sci. 91:3224-3227).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, COS, HeLa, MDCK, HEK293, and WI38, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler, M. et al. (1977) Cell 11:223-32) and adenine phosphoribosyltransferase (Lowy, I. et al. (1990) Cell 22:817-23) genes which can be employed in tk.sup.- or aprt.sup.-cells, respectively. Also, antimetabolite, antibiotic or herbicide resistance can be used as the basis for selection; for example, dhfr which confers resistance to methotrexate (Wigler, M. et al. (1980) Proc. Natl. Acad. Sci. 77:3567-70); npt, which confers resistance to the aminoglycosides, neomycin and G-418 (Colbere-Garapin, F. et al (1981) J. Mol. Biol. 150:1-14); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman, S. C. and R. C. Mulligan (1988) Proc. Natl. Acad. Sci. 85:8047-51). The use of visible markers has gained popularity with such markers as anthocyanins, beta-glucuronidase and its substrate GUS, and luciferase and its substrate luciferin, being widely used not only to identify transformants, but also to quantify the amount of transient or stable protein expression attributable to a specific vector system (Rhodes, C. A. et al. (1995) Methods Mol. Biol. 55:121-131).

Although the presence/absence of marker gene expression suggests that the gene of interest is also present, its presence and expression may need to be confirmed. For example, if the sequence encoding a polypeptide is inserted within a marker gene sequence, recombinant cells containing sequences can be identified by the absence of marker gene function. Alternatively, a marker gene can be placed in tandem with a polypeptide-encoding sequence under the control of a single promoter. Expression of the marker gene in response to induction or selection usually indicates expression of the tandem gene as well.

Alternatively, host cells that contain and express a desired polynucleotide sequence may be identified by a variety of procedures known to those of skill in the art. These procedures include, but are not limited to, DNA-DNA or DNA-RNA hybridizations and protein bioassay or immunoassay techniques which include, for example, membrane, solution, or chip based technologies for the detection and/or quantification of nucleic acid or protein.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton, R. et al. (1990; Serological Methods, a Laboratory Manual, APS Press, St Paul. Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor XA or enterokinase (Invitrogen. San Diego, Calif.) between the purification domain and the encoded polypeptide may be used to facilitate purification. One such expression vector provides for expression of a fusion protein containing a polypeptide of interest and a nucleic acid encoding 6 histidine residues preceding a thioredoxin or an enterokinase cleavage site. The histidine residues facilitate purification on IMIAC (immobilized metal ion affinity chromatography) as described in Porath, J. et al. (1992, Prot. Exp. Purif 3:263-281) while the enterokinase cleavage site provides a means for purifying the desired polypeptide from the fusion protein. A discussion of vectors which contain fusion proteins is provided in Kroll, D. J. et al. (1993; DNA Cell Biol. 12:441-453).

In addition to recombinant production methods, polypeptides of the invention, and fragments thereof, may be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full length molecule.

Antibody Compositions, Fragments Thereof and Other Binding Agents

According to another aspect, the present invention further provides binding agents, such as antibodies and antigen-binding fragments thereof, that exhibit immunological binding to a tumor polypeptide disclosed herein, or to a portion, variant or derivative thereof. An antibody, or antigen-binding fragment thereof, is said to “specifically bind,” “immunogically bind,” and/or is “immunologically reactive” to a polypeptide of the invention if it reacts at a detectable level (within, for example, an ELISA assay) with the polypeptide, and does not react detectably with unrelated polypeptides under similar conditions.

Immunological binding, as used in this context, generally refers to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity, and is thus equal to the dissociation constant K_{d}. See, generally, Davies et al. (1990) Annual Rev. Biochem. 59:439-473.

An “antigen-binding site,” or “binding portion” of an antibody refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “FR” refers to amino acid sequences which are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Binding agents may be further capable of differentiating between patients with and without a cancer, such as pancreatic cancer, using the representative assays provided herein. For example, antibodies or other binding agents that bind to a tumor protein will preferably generate a signal indicating the presence of a cancer in at least about 20% of patients with the disease, more preferably at least about 30% of patients. Alternatively, or in addition, the antibody will generate a negative signal indicating the absence of the disease in at least about 90% of individuals without the cancer. To determine whether a binding agent satisfies this requirement, biological samples (e.g., blood, sera, sputum, urine and/or tumor biopsies) from patients with and without a cancer (as determined using standard clinical tests) may be assayed as described herein for the presence of polypeptides that bind to the binding agent. Preferably, a statistically significant number of samples with and without the disease will be assayed. Each binding agent should satisfy the above criteria; however, those of ordinary skill in the art will recognize that binding agents may be used in combination to improve sensitivity.

Any agent that satisfies the above requirements may be a binding agent. For example, a binding agent may be a ribosome, with or without a peptide component, an RNA molecule or a polypeptide. In a preferred embodiment, a binding agent is an antibody or an antigen-binding fragment thereof. Antibodies may be prepared by any of a variety of techniques known to those of ordinary skill in the art. See, e.g., Harlow and Lane, Antibodies. A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

A number of therapeutically useful molecules are known in the art which comprise antigen-binding sites that are capable of exhibiting immunological binding properties of an antibody molecule. The proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′)₂” fragment which comprises both antigen-binding sites. An “Fv” fragment can be produced by preferential proteolytic cleavage of an IgM, and on rare occasions IgG or IgA immunoglobulin molecule. Fv fragments are, however, more commonly derived using recombinant techniques known in the art. The Fv fragment includes a non-covalent V_{H}::V_{L} heterodimer including an antigen-binding site which retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

A single chain Fv (“sFv”) polypeptide is a covalently linked V_{H}::V_{L} heterodimer which is expressed from a gene fusion including V_{H}- and V_{L}-encoding genes linked by a peptide-encoding linker. Huston et al. (1988) Proc. Nat. Acad. Sci. USA 85(16):5879-5883. A number of methods have been described to discern chemical structures for converting the naturally aggregated—but chemically separated—light and heavy polypeptide chains from an antibody V region into an sFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

Each of the above-described molecules includes a heavy chain and a light chain CDR set, respectively interposed between a heavy chain and a light chain FR set which provide support to the CDRS and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRS. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

A number of “humanized” antibody molecules comprising an antigen-binding site derived from a non-human immunoglobulin have been described, including chimeric antibodies having rodent V regions and their associated CDRs fused to human constant domains (Winter et al. (1991) Nature 349:293-299; Lobuglio et al. (1989) Proc. Nat. Acad. Sci. USA 86:4220-4224; Shaw et al. (1987) J Immunol. 138:4534-4538; and Brown et al. (1987) Cancer Res. 47:3577-3583), rodent CDRs grafted into a human supporting FR prior to fusion with an appropriate human antibody constant domain (Riechmann et al. (1988) Nature 332:323-327; Verhoeyen et al. (1988) Science 239:1534-1536; and Jones et al. (1986) Nature 321:522-525), and rodent CDRs supported by recombinantly veneered rodent FRs (European Patent Publication No. 519,596, published Dec. 23, 1992). These “humanized” molecules are designed to minimize unwanted immunological response toward rodent antihuman antibody molecules which limits the duration and effectiveness of therapeutic applications of those moieties in human recipients.

As used herein, the terms “veneered FRs” and “recombinantly veneered FRs” refer to the selective replacement of FR residues from, e.g., a rodent heavy or light chain V region, with human FR residues in order to provide a xenogeneic molecule comprising an antigen-binding site which retains substantially all of the native FR polypeptide folding structure. Veneering techniques are based on the understanding that the ligand binding characteristics of an antigen-binding site are determined primarily by the structure and relative disposition of the heavy and light chain CDR sets within the antigen-binding surface. Davies et al. (1990) Ann. Rev. Biochem. 59:439-473. Thus, antigen binding specificity can be preserved in a humanized antibody only wherein the CDR structures, their interaction with each other, and their interaction with the rest of the V region domains are carefully maintained. By using veneering techniques, exterior (e.g., solvent-accessible) FR residues which are readily encountered by the immune system are selectively replaced with human residues to provide a hybrid molecule that comprises either a weakly immunogenic, or substantially non-immunogenic veneered surface.

The process of veneering makes use of the available sequence data for human antibody variable domains compiled by Kabat et al., in Sequences of Proteins of Immunological Interest, 4th ed., (U.S. Dept. of Health and Human Services, U.S. Government Printing Office, 1987), updates to the Kabat database, and other accessible U.S. and foreign databases (both nucleic acid and protein). Solvent accessibilities of V region amino acids can be deduced from the known three-dimensional structure for human and murine antibody fragments. There are two general steps in veneering a murine antigen-binding site. Initially, the FRs of the variable domains of an antibody molecule of interest are compared with corresponding FR sequences of human variable domains obtained from the above-identified sources. The most homologous human V regions are then compared residue by residue to corresponding murine amino acids. The residues in the murine FR which differ from the human counterpart are replaced by the residues present in the human moiety using recombinant techniques well known in the art. Residue switching is only carried out with moieties which are at least partially exposed (solvent accessible), and care is exercised in the replacement of amino acid residues which may have a significant effect on the tertiary structure of V region domains, such as proline, glycine and charged amino acids.

In this manner, the resultant “veneered” murine antigen-binding sites are thus designed to retain the murine CDR residues, the residues substantially adjacent to the CDRs, the residues identified as buried or mostly buried (solvent inaccessible), the residues believed to participate in non-covalent (e.g., electrostatic and hydrophobic) contacts between heavy and light chain domains, and the residues from conserved structural regions of the FRs which are believed to influence the “canonical” tertiary structures of the CDR loops. These design criteria are then used to prepare recombinant nucleotide sequences which combine the CDRs of both the heavy and light chain of a murine antigen-binding site into human-appearing FRs that can be used to transfect mammalian cells for the expression of recombinant human antibodies which exhibit the antigen specificity of the murine antibody molecule.

In another embodiment of the invention, monoclonal antibodies of the present invention may be coupled to one or more therapeutic agents. Suitable agents in this regard include radionuclides, differentiation inducers, drugs, toxins, and derivatives thereof. Preferred radionuclides include ⁹⁰Y, ¹²³I, ¹²⁵I, ¹³¹I, ¹⁸⁶Re, ¹⁸⁸Re, ²¹¹At, and ²¹²Bi. Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Where a therapeutic agent is more potent when free from the antibody portion of the immunoconjugates of the present invention, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers that provide multiple sites for attachment can be used. Alternatively, a carrier can be used.

A carrier may bear the agents in a variety of ways, including covalent bonding either directly or via a linker group. Suitable carriers include proteins such as albumins (e.g., U.S. Pat. No. 4,507,234, to Kato et al.), peptides and polysaccharides such as aminodextran (e.g., U.S. Pat. No. 4,699,784, to Shih et al.). A carrier may also bear an agent by noncovalent bonding or by encapsulation, such as within a liposome vesicle (e.g., U.S. Pat. Nos. 4,429,008 and 4,873,088). Carriers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis.

T Cell Compositions

The present invention, in another aspect, provides T cells specific for a tumor polypeptide disclosed herein, or for a variant or derivative thereof. Such cells may generally be prepared in vitro or ex vivo, using standard procedures. For example, T cells may be isolated from bone marrow, peripheral blood, or a fraction of bone marrow or peripheral blood of a patient, using a commercially available cell separation system, such as the Isolex™ System, available from Nexell Therapeutics, Inc. (Irvine, Calif.; see also U.S. Pat. No. 5,240,856; U.S. Pat. No. 5,215,926; WO 89/06280; WO 91/16116 and WO 92/07243). Alternatively, T cells may be derived from related or unrelated humans, non-human mammals, cell lines or cultures.

T cells may be stimulated with a polypeptide, polynucleotide encoding a polypeptide and/or an antigen presenting cell (APC) that expresses such a polypeptide. Such stimulation is performed under conditions and for a time sufficient to permit the generation of T cells that are specific for the polypeptide of interest. Preferably, a tumor polypeptide or polynucleotide of the invention is present within a delivery vehicle, such as a microsphere, to facilitate the generation of specific T cells.

T cells are considered to be specific for a polypeptide of the present invention if the T cells specifically proliferate, secrete cytokines or kill target cells coated with the polypeptide or expressing a gene encoding the polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070, 1994. Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a tumor polypeptide (100 ng/ml-100 μg/ml, preferably 200 ng/ml-25 μg/ml) for 3-7 days will typically result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1, Wiley Interscience (Greene 1998)). T cells that have been activated in response to a tumor polypeptide, polynucleotide or polypeptide-expressing APC may be CD4⁺ and/or CD8⁺. Tumor polypeptide-specific T cells may be expanded using standard techniques. Within preferred embodiments, the T cells are derived from a patient, a related donor or an unrelated donor, and are administered to the patient following stimulation and expansion.

For therapeutic purposes, CD4⁺ or CD8⁺ T cells that proliferate in response to a tumor polypeptide, polynucleotide or APC can be expanded in number either in vitro or in vivo. Proliferation of such T cells in vitro may be accomplished in a variety of ways. For example, the T cells can be re-exposed to a tumor polypeptide, or a short peptide corresponding to an immunogenic portion of such a polypeptide, with or without the addition of T cell growth factors, such as interleukin-2, and/or stimulator cells that synthesize a tumor polypeptide. Alternatively, one or more T cells that proliferate in the presence of the tumor polypeptide can be expanded in number by cloning. Methods for cloning cells are well known in the art, and include limiting dilution.

T Cell Receptor Compositions

The T cell receptor (TCR) consists of 2 different, highly variable polypeptide chains, termed the T-cell receptor α and β chains, that are linked by a disulfide bond (Janeway, Travers, Walport. Immunobiology. Fourth Ed., 148-159. Elsevier Science Ltd/Garland Publishing. 1999). The α/β heterodimer complexes with the invariant CD3 chains at the cell membrane. This complex recognizes specific antigenic peptides bound to MHC molecules. The enormous diversity of TCR specificities is generated much like immunoglobulin diversity, through somatic gene rearrangement. The β chain genes contain over 50 variable (V), 2 diversity (D), over 10 joining (J) segments, and 2 constant region segments (C). The α chain genes contain over 70 V segments, and over 60 J segments but no D segments, as well as one C segment. During T cell development in the thymus, the D to J gene rearrangement of the β chain occurs, followed by the V gene segment rearrangement to the DJ. This functional VDJβ exon is transcribed and spliced to join to a Cβ. For the α chain, a Vα gene segment rearranges to a Jα gene segment to create the functional exon that is then transcribed and spliced to the Cα. Diversity is further increased during the recombination process by the random addition of P and N-nucleotides between the V, D, and J segments of the β chain and between the V and J segments in the □ chain (Janeway, Travers, Walport. Immunobiology. Fourth Ed., 98 and 150. Elsevier Science Ltd/Garland Publishing. 1999).

The present invention, in another aspect, provides TCRs specific for a polypeptide disclosed herein, or for a variant or derivative thereof. In accordance with the present invention, polynucleotide and amino acid sequences are provided for the V-J or V-D-J junctional regions or parts thereof for the alpha and beta chains of the T-cell receptor which recognize tumor polypeptides described herein. In general, this aspect of the invention relates to T-cell receptors which recognize or bind tumor polypeptides presented in the context of MHC. In a preferred embodiment the tumor antigens recognized by the T-cell receptors comprise a polypeptide of the present invention. For example, cDNA encoding a TCR specific for a pancreatic tumor peptide can be isolated from T cells specific for a tumor polypeptide using standard molecular biological and recombinant DNA techniques.

This invention further includes the T-cell receptors or analogs thereof having substantially the same function or activity as the T-cell receptors of this invention which recognize or bind tumor polypeptides. Such receptors include, but are not limited to, a fragment of the receptor, or a substitution, addition or deletion mutant of a T-cell receptor provided herein. This invention also encompasses polypeptides or peptides that are substantially homologous to the T-cell receptors provided herein or that retain substantially the same activity. The term “analog” includes any protein or polypeptide having an amino acid residue sequence substantially identical to the T-cell receptors provided herein in which one or more residues, preferably no more than 5 residues, more preferably no more than 25 residues have been conservatively substituted with a functionally similar residue and which displays the functional aspects of the T-cell receptor as described herein.

The present invention further provides for suitable mammalian host cells, for example, non-specific T cells, that are transfected with a polynucleotide encoding TCRs specific for a polypeptide described herein, thereby rendering the host cell specific for the polypeptide. The α and β chains of the TCR may be contained on separate expression vectors or alternatively, on a single expression vector that also contains an internal ribosome entry site (IRES) for cap-independent translation of the gene downstream of the IRES. Said host cells expressing TCRs specific for the polypeptide may be used, for example, for adoptive immunotherapy of pancreatic cancer as discussed further below.

In further aspects of the present invention, cloned TCRs specific for a polypeptide recited herein may be used in a kit for the diagnosis of pancreatic cancer. For example, the nucleic acid sequence or portions thereof, of tumor-specific TCRs can be used as probes or primers for the detection of expression of the rearranged genes encoding the specific TCR in a biological sample. Therefore, the present invention further provides for an assay for detecting messenger RNA or DNA encoding the TCR specific for a polypeptide.

Pharmaceutical Compositions

In additional embodiments, the present invention concerns formulation of one or more of the polynucleotide, polypeptide, T-cell, TCR, and/or antibody compositions disclosed herein in pharmaceutically-acceptable carriers for administration to a cell or an animal, either alone, or in combination with one or more other modalities of therapy.

It will be understood that, if desired, a composition as disclosed herein may be administered in combination with other agents as well, such as, e.g., other proteins or polypeptides or various pharmaceutically-active agents. In fact, there is virtually no limit to other components that may also be included, given that the additional agents do not cause a significant adverse effect upon contact with the target cells or host tissues. The compositions may thus be delivered along with various other agents as required in the particular instance. Such compositions may be purified from host cells or other biological sources, or alternatively may be chemically synthesized as described herein. Likewise, such compositions may further comprise substituted or derivatized RNA or DNA compositions.

Therefore, in another aspect of the present invention, pharmaceutical compositions are provided comprising one or more of the polynucleotide, polypeptide, antibody,TCR, and/or T-cell compositions described herein in combination with a physiologically acceptable carrier. In certain preferred embodiments, the pharmaceutical compositions of the invention comprise immunogenic polynucleotide and/or polypeptide compositions of the invention for use in prophylactic and theraputic vaccine applications. Vaccine preparation is generally described in, for example, M. F. Powell and M. J. Newman, eds., “Vaccine Design (the subunit and adjuvant approach),” Plenum Press (NY, 1995). Generally, such compositions will comprise one or more polynucleotide and/or polypeptide compositions of the present invention in combination with one or more immunostimulants.

It will be apparent that any of the pharmaceutical compositions described herein can contain pharmaceutically acceptable salts of the polynucleotides and polypeptides of the invention. Such salts can be prepared, for example, from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

In another embodiment, illustrative immunogenic compositions, e.g., vaccine compositions, of the present invention comprise DNA encoding one or more of the polypeptides as described above, such that the polypeptide is generated in situ. As noted above, the polynucleotide may be administered within any of a variety of delivery systems known to those of ordinary skill in the art. Indeed, numerous gene delivery techniques are well known in the art, such as those described by Rolland, Crit. Rev. Therap. Drug Carrier Systems 15:143-198, 1998, and references cited therein. Appropriate polynucleotide expression systems will, of course, contain the necessary regulatory DNA regulatory sequences for expression in a patient (such as a suitable promoter and terminating signal). Alternatively, bacterial delivery systems may involve the administration of a bacterium (such as Bacillus-Calmette-Guerrin) that expresses an immunogenic portion of the polypeptide on its cell surface or secretes such an epitope.

Therefore, in certain embodiments, polynucleotides encoding immunogenic polypeptides described herein are introduced into suitable mammalian host cells for expression using any of a number of known viral-based systems. In one illustrative embodiment, retroviruses provide a convenient and effective platform for gene delivery systems. A selected nucleotide sequence encoding a polypeptide of the present invention can be inserted into a vector and packaged in retroviral particles using techniques known in the art. The recombinant virus can then be isolated and delivered to a subject. A number of illustrative retroviral systems have been described (e.g., U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

In addition, a number of illustrative adenovirus-based systems have also been described. Unlike retroviruses which integrate into the host genome, adenoviruses persist extrachromosomally thus minimizing the risks associated with insertional mutagenesis (Haj-Ahmad and Graham (1986) J. Virol. 57:267-274; Bett et al. (1993) J. Virol. 67:5911-5921; Mittereder et al. (1994) Human Gene Therapy 5:717-729; Seth et al. (1994) J. Virol. 68:933-940; Barr et al. (1994) Gene Therapy 1:51-58; Berkner, K. L. (1988) BioTechniques 6:616-629; and Rich et al. (1993) Human Gene Therapy 4:461-476).

Various adeno-associated virus (AAV) vector systems have also been developed for polynucleotide delivery. AAV vectors can be readily constructed using techniques well known in the art. See, e.g., U.S. Pat. Nos. 5,173,414 and 5,139,941; International Publication Nos. WO 92/01070 and WO 93/03769; Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

Additional viral vectors useful for delivering the polynucleotides encoding polypeptides of the present invention by gene transfer include those derived from the pox family of viruses, such as vaccinia virus and avian poxvirus. By way of example, vaccinia virus recombinants expressing the novel molecules can be constructed as follows. The DNA encoding a polypeptide is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the polypeptide of interest into the viral genome. The resulting TK.sup.(-) recombinant can be selected by culturing the cells in the presence of 5-bromodeoxyuridine and picking viral plaques resistant thereto.

A vaccinia-based infection/transfection system can be conveniently used to provide for inducible, transient expression or coexpression of one or more polypeptides described herein in host cells of an organism. In this particular system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide or polynucleotides of interest, driven by a T7 promoter. The polymerase expressed in the cytoplasm from the vaccinia virus recombinant transcribes the transfected DNA into RNA which is then translated into polypeptide by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al. Proc. Natl. Acad. Sci. USA (1986) 83:8122-8126.

Alternatively, avipoxviruses, such as the fowlpox and canarypox viruses, can also be used to deliver the coding sequences of interest. Recombinant avipox viruses, expressing immunogens from mammalian pathogens, are known to confer protective immunity when administered to non-avian species. The use of an Avipox vector is particularly desirable in human and other mammalian species since members of the Avipox genus can only productively replicate in susceptible avian species and therefore are not infective in mammalian cells. Methods for producing recombinant Avipoxviruses are known in the art and employ genetic recombination, as described above with respect to the production of vaccinia viruses. See, e.g., WO 91/12882; WO 89/03429; and WO 92/03545.

Any of a number of alphavirus vectors can also be used for delivery of polynucleotide compositions of the present invention, such as those vectors described in U.S. Pat. Nos. 5,843,723; 6,015,686; 6,008,035 and 6,015,694. Certain vectors based on Venezuelan Equine Encephalitis (VEE) can also be used, illustrative examples of which can be found in U.S. Pat. Nos. 5,505,947 and 5,643,576.

Moreover, molecular conjugate vectors, such as the adenovirus chimeric vectors described in Michael et al. J. Biol. Chem. (1993) 268:6866-6869 and Wagner et al. Proc. Natl. Acad. Sci. USA (1992) 89:6099-6103, can also be used for gene delivery under the invention.

Additional illustrative information on these and other known viral-based delivery systems can be found, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad Sci. USA 90:11498-11502, 1993; Guzman et al., Circulation 88:2838-2848, 1993; and Guzman et al., Cir. Res. 73:1202-1207, 1993.

In certain embodiments, a polynucleotide may be integrated into the genome of a target cell. This integration may be in the specific location and orientation via homologous recombination (gene replacement) or it may be integrated in a random, non-specific location (gene augmentation). In yet further embodiments, the polynucleotide may be stably maintained in the cell as a separate, episomal segment of DNA. Such polynucleotide segments or “episomes” encode sequences sufficient to permit maintenance and replication independent of or in synchronization with the host cell cycle. The manner in which the expression construct is delivered to a cell and where in the cell the polynucleotide remains is dependent on the type of expression construct employed.

In another embodiment of the invention, a polynucleotide is administered/delivered as “naked” DNA, for example as described in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

In still another embodiment, a composition of the present invention can be delivered via a particle bombardment approach, many of which have been described. In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500799. This approach offers a needle-free delivery approach wherein a dry powder formulation of microscopic particles, such as polynucleotide or polypeptide particles, are accelerated to high speed within a helium gas jet generated by a hand held device, propelling the particles into a target tissue of interest.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

According to another embodiment, the pharmaceutical compositions described herein will comprise one or more immunostimulants in addition to the immunogenic polynucleotide, polypeptide, antibody, T-cell, TCR, and/or APC compositions of this invention. An immunostimulant refers to essentially any substance that enhances or potentiates an immune response (antibody and/or cell-mediated) to an exogenous antigen. One preferred type of immunostimulant comprises an adjuvant. Many adjuvants contain a substance designed to protect the antigen from rapid catabolism, such as aluminum hydroxide or mineral oil, and a stimulator of immune responses, such as lipidA, Bortadella pertussis or Mycobacterium tuberculosis derived proteins. Certain adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

Within certain embodiments of the invention, the adjuvant composition is preferably one that induces an immune response predominantly of the Th1 type. High levels of Th1-type cytokines (e.g, IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989.

Certain preferred adjuvants for eliciting a predominantly Th1-type response include, for example, a combination of monophosphoryl lipid A, preferably 3-de-O-acylated monophosphoryl lipid A, together with an aluminum salt. MPL® adjuvants are available from Corixa Corporation (Seattle, Wash.; see, for example, U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins. Other preferred formulations include more than one saponin in the adjuvant combinations of the present invention, for example combinations of at least two of the following group comprising QS21, QS7, Quil A, β-escin, or digitonin.

Alternatively the saponin formulations may be combined with vaccine vehicles composed of chitosan or other polycationic polymers, polylactide and polylactide-co-glycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs. Furthermore, the saponins may be formulated together with a polyoxyethylene ether or ester, in either a non-particulate solution or suspension, or in a particulate structure such as a paucilamelar liposome or ISCOM. The saponins may also be formulated with excipients such as Carbopol^{R} to increase viscosity, or may be formulated in a dry powder form with a powder excipient such as lactose.

In one preferred embodiment, the adjuvant system includes the combination of a monophosphoryl lipid A and a saponin derivative, such as the combination of QS21 and 3D-MPL® adjuvant, as described in WO 94/00153, or a less reactogenic composition where the QS21 is quenched with cholesterol, as described in WO 96/33739. Other preferred formulations comprise an oil-in-water emulsion and tocopherol. Another particularly preferred adjuvant formulation employing QS21, 3D-MPL® adjuvant and tocopherol in an oil-in-water emulsion is described in WO 95/17210.

Another enhanced adjuvant system involves the combination of a CpG-containing oligonucleotide and a saponin derivative particularly the combination of CpG and QS21 is disclosed in WO 00/09159. Preferably the formulation additionally comprises an oil in water emulsion and tocopherol.

Additional illustrative adjuvants for use in the pharmaceutical compositions of the invention include Montanide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Enhanzyn®) (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1.

Other preferred adjuvants include adjuvant molecules of the general formula

HO(CH₂CH₂O)ₙ—A—R, (1)

wherein, n is 1-50, A is a bond or —C(O)—, R is C₁₅₀ alkyl or Phenyl C₁₋₅₀ alkyl.

One embodiment of the present invention consists of a vaccine formulation comprising a polyoxyethylene ether of general formula (I), wherein n is between 1 and 50, preferably 4-24, most preferably 9; the R component is C₁₋₅₀, preferably C₄-C₂₀ alkyl and most preferably C₁₂ alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (₁₂th edition: entry 7717). These adjuvant molecules are described in WO 99/52549.

The polyoxyethylene ether according to the general formula (I) above may, if desired, be combined with another adjuvant. For example, a preferred adjuvant combination is preferably with CpG as described in the pending UK patent application GB 9820956.2.

According to another embodiment of this invention, an immunogenic composition described herein is delivered to a host via antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic antitumor immunity (see Timmerman and Levy, Ann. Rev. Med. 50:507-529, 1999). In general, dendritic cells may be identified based on their typical shape (stellate in situ, with marked cytoplasmic processes (dendrites) visible in vitro), their ability to take up, process and present antigens with high efficiency and their ability to activate naive T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with a polynucleotide of the invention (or portion or other variant thereof) such that the encoded polypeptide, or an immunogenic portion thereof, is expressed on the cell surface. Such transfection may take place ex vivo, and a pharmaceutical composition comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with the tumor polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

While any suitable carrier known to those of ordinary skill in the art may be employed in the pharmaceutical compositions of this invention, the type of carrier will typically vary depending on the mode of administration. Compositions of the present invention may be formulated for any appropriate manner of administration, including for example, topical, oral, nasal, mucosal, intravenous, intracranial, intraperitoneal, subcutaneous and intramuscular administration.

Carriers for use within such pharmaceutical compositions are biocompatible, and may also be biodegradable. In certain embodiments, the formulation preferably provides a relatively constant level of active component release. In other embodiments, however, a more rapid rate of release immediately upon administration may be desired. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

In another illustrative embodiment, biodegradable microspheres (e.g., polylactate polyglycolate) are employed as carriers for the compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344, 5,407,609 and 5,942,252. Modified hepatitis B core protein carrier systems. such as described in WO/99 40934, and references cited therein, will also be useful for many applications. Another illustrative carrier/delivery system employs a carrier comprising particulate-protein complexes, such as those described in U.S. Pat. No. 5,928,647, which are capable of inducing a class I-restricted cytotoxic T lymphocyte responses in a host.

In another illustrative embodiment, calcium phosphate core particles are employed as carriers, vaccine adjuvants, or as controlled release matrices for the compositions of this invention. Exemplary calcium phosphate particles are disclosed, for example, in published patent application No. WO/0046147.

The pharmaceutical compositions of the invention will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate.

The pharmaceutical compositions described herein may be presented in unit-dose or multi-dose containers, such as sealed ampoules or vials. Such containers are typically sealed in such a way to preserve the sterility and stability of the formulation until use. In general, formulations may be stored as suspensions, solutions or emulsions in oily or aqueous vehicles. Alternatively, a pharmaceutical composition may be stored in a freeze-dried condition requiring only the addition of a sterile liquid carrier immediately prior to use.

The development of suitable dosing and treatment regimens for using the particular compositions described herein in a variety of treatment regimens, including e.g., oral, parenteral, intravenous, intranasal, and intramuscular administration and formulation, is well known in the art, some of which are briefly discussed below for general purposes of illustration.

In certain applications, the pharmaceutical compositions disclosed herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

The active compounds may even be incorporated with excipients and used in the form of ingestible tablets, buccal tables, troches, capsules, elixirs, suspensions, syrups, wafers, and the like (see, for example, Mathiowitz et al., Nature 1997 Mar 27;386(6623):410-4; Hwang et al., Crit Rev Ther Drug Carrier Syst 1998;15(3):243-84; U.S. Pat. No. 5,641,515; U.S. Pat. No. 5,580,579 and U.S. Pat. No. 5,792,451). Tablets, troches, pills, capsules and the like may also contain any of a variety of additional components, for example, a binder, such as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Of course, any material used in preparing any dosage unit form should be pharmaceutically pure and substantially non-toxic in the amounts employed. In addition, the active compounds may be incorporated into sustained-release preparation and formulations.

Typically, these formulations will contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

For oral administration the compositions of the present invention may alternatively be incorporated with one or more excipients in the form of a mouthwash, dentifrice, buccal tablet, oral spray, or sublingual orally-administered formulation. Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

In certain circumstances it will be desirable to deliver the pharmaceutical compositions disclosed herein parenterally, intravenously, intramuscularly, or even intraperitoneally. Such approaches are well known to the skilled artisan, some of which are further described, for example, in U.S. Pat. No. 5,543,158; U.S. Pat. No. 5,641,515 and U.S. Pat. No. 5,399,363. In certain embodiments, solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations generally will contain a preservative to prevent the growth of microorganisms.

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and/or by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In one embodiment, for parenteral administration in an aqueous solution, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. Moreover, for human administration, preparations will of course preferably meet sterility, pyrogenicity, and the general safety and purity standards as required by FDA Office of Biologics standards.

In another embodiment of the invention, the compositions disclosed herein may be formulated in a neutral or salt form. Illustrative pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

In certain embodiments, the pharmaceutical compositions may be delivered by intranasal sprays, inhalation, and/or other aerosol delivery vehicles. Methods for delivering genes, nucleic acids, and peptide compositions directly to the lungs via nasal aerosol sprays has been described, e.g., in U.S. Pat. No. 5,756,353 and U.S. Pat. No. 5,804,212. Likewise, the delivery of drugs using intranasal microparticle resins (Takenaga et al., J Controlled Release 1998 Mar 2;52(1-2):81-7) and lysophosphatidyl-glycerol compounds (U.S. Pat. No. 5,725,871) are also well-known in the pharmaceutical arts. Likewise, illustrative transmucosal drug delivery in the form of a polytetrafluoroetheylene support matrix is described in U.S. Pat. No. 5,780,045.

In certain embodiments, liposomes, nanocapsules, microparticles, lipid particles, vesicles, and the like, are used for the introduction of the compositions of the present invention into suitable host cells/organisms. In particular, the compositions of the present invention may be formulated for delivery either encapsulated in a lipid particle, a liposome, a vesicle, a nanosphere, or a nanoparticle or the like. Alternatively, compositions of the present invention can be bound, either covalently or non-covalently, to the surface of such carrier vehicles.

The formation and use of liposome and liposome-like preparations as potential drug carriers is generally known to those of skill in the art (see for example, Lasic, Trends Biotechnol 1998 Jul;16(7):307-21; Takakura, Nippon Rinsho 1998 Mar;56(3):691-5; Chandran et al., Indian J Exp Biol. 1997 Aug;35(8):801-9; Margalit, Crit Rev Ther Drug Carrier Syst. 1995;12(2-3):233-61; U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

Liposomes have been used successfully with a number of cell types that are normally difficult to transfect by other procedures, including T cell suspensions, primary hepatocyte cultures and PC 12 cells (Renneisen et al., J Biol Chem. 1990 Sep 25;265(27):16337-42; Muller et al., DNA Cell Biol. 1990 Apr;9(3):221-9). In addition, liposomes are free of the DNA length constraints that are typical of viral-based delivery systems. Liposomes have been used effectively to introduce genes, various drugs, radiotherapeutic agents, enzymes, viruses, transcription factors, allosteric effectors and the like, into a variety of cultured cell lines and animals. Furthermore, he use of liposomes does not appear to be associated with autoimmune responses or unacceptable toxicity after systemic delivery.

In certain embodiments, liposomes are formed from phospholipids that are dispersed in an aqueous medium and spontaneously form multilamellar concentric bilayer vesicles (also termed multilamellar vesicles (MLVs).

Alternatively, in other embodiments, the invention provides for pharmaceutically-acceptable nanocapsule formulations of the compositions of the present invention. Nanocapsules can generally entrap compounds in a stable and reproducible way (see, for example, Quintanar-Guerrero et al., Drug Dev Ind Pharm. 1998 Dec;24(12):1113-28). To avoid side effects due to intracellular polymeric overloading, such ultrafine particles (sized around 0.1 μm) may be designed using polymers able to be degraded in vivo. Such particles can be made as described, for example, by Couvreur et al., Crit Rev Ther Drug Carrier Syst. 1988;5(l):1-20; zur Muhlen et al., Eur J Pharm Biopharm. 1998 Mar;45(2):149-55; Zambaux et al. J Controlled Release. 1998 Jan 2;50(1-3):31-40; and U.S. Pat. No. 5,145,684.

Cancer Therapeutic Methods

Immunologic approaches to cancer therapy are based on the recognition that cancer cells can often evade the body's defenses against aberrant or foreign cells and molecules, and that these defenses might be therapeutically stimulated to regain the lost ground, e.g. pgs. 623-648 in Klein, Immunology (Wiley-Interscience, New York, 1982). Numerous recent observations that various immune effectors can directly or indirectly inhibit growth of tumors has led to renewed interest in this approach to cancer therapy, e.g. Jager, et al., Oncology 2001;60(1):1-7; Renner, et al., Ann Hematol 2000 Dec;79(12):651-9.

Four-basic cell types whose function has been associated with antitumor cell immunity and the elimination of tumor cells from the body are: i) B-lymphocytes which secrete immunoglobulins into the blood plasma for identifying and labeling the nonself invader cells; ii) monocytes which secrete the complement proteins that are responsible for lysing and processing the immunoglobulin-coated target invader cells; iii) natural killer lymphocytes having two mechanisms for the destruction of tumor cells, antibody-dependent cellular cytotoxicity and natural killing; and iv) T-lymphocytes possessing antigen-specific receptors and having the capacity to recognize a tumor cell carrying complementary marker molecules (Schreiber, H., 1989, in Fundamental Immunology (ed). W. E. Paul, pp. 923-955).

Cancer immunotherapy generally focuses on inducing humoral immune responses, cellular immune responses, or both. Moreover, it is well established that induction of CD4⁺ T helper cells is necessary in order to secondarily induce either antibodies or cytotoxic CD8+T cells. Polypeptide antigens that are selective or ideally specific for cancer cells, particularly pancreatic cancer cells, offer a powerful approach for inducing immune responses against pancreatic cancer, and are an important aspect of the present invention.

In further aspects of the present invention, the pharmaceutical compositions described herein may be used for the treatment of cancer, particularly for the immunotherapy of pancreatic cancer. Within such methods, the pharmaceutical compositions described herein are administered to a patient, typically a warm-blooded animal, preferably a human. A patient may or may not be afflicted with cancer. Accordingly, the above pharmaceutical compositions may be used to prevent the development of a cancer or to treat a patient afflicted with a cancer. Pharmaceutical compositions and vaccines may be administered either prior to or following surgical removal of primary tumors and/or treatment such as administration of radiotherapy or conventional chemotherapeutic drugs. As discussed above, administration of the pharmaceutical compositions may be by any suitable method, including administration by intravenous, intraperitoneal, intramuscular, subcutaneous, intranasal, intradermal, anal, vaginal, topical and oral routes.

Within certain embodiments, immunotherapy may be active immunotherapy, in which treatment relies on the in vivo stimulation of the endogenous host immune system to react against tumors with the administration of immune response-modifying agents (such as polypeptides and polynucleotides as provided herein).

Within other embodiments, immunotherapy may be passive immunotherapy, in which treatment involves the delivery of agents with established tumor-immune reactivity (such as effector cells or antibodies) that can directly or indirectly mediate antitumor effects and does not necessarily depend on an intact host immune system. Examples of effector cells include T cells as discussed above, T lymphocytes (such as CD8⁺ cytotoxic T lymphocytes and CD4⁺ T-helper tumor-infiltrating lymphocytes), killer cells (such as Natural Killer cells and lymphokine-activated killer cells), B cells and antigen-presenting cells (such as dendritic cells and macrophages) expressing a polypeptide provided herein. T cell receptors and antibody receptors specific for the polypeptides recited herein may be cloned, expressed and transferred into other vectors or effector cells for adoptive immunotherapy. The polypeptides provided herein may also be used to generate antibodies or anti-idiotypic antibodies (as described above and in U.S. Pat. No. 4,918,164) for passive immunotherapy.

Monoclonal antibodies may be labeled with any of a variety of labels for desired selective usages in detection, diagnostic assays or therapeutic applications (as described in U.S. Pat. Nos. 6,090,365; 6,015,542; 5,843,398; 5,595,721; and 4,708,930, hereby incorporated by reference in their entirety as if each was incorporated individually). In each case, the binding of the labelled monoclonal antibody to the determinant site of the antigen will signal detection or delivery of a particular therapeutic agent to the antigenic determinant on the non-normal cell. A further object of this invention is to provide the specific monoclonal antibody suitably labelled for achieving such desired selective usages thereof.

Effector cells may generally be obtained in sufficient quantities for adoptive immunotherapy by growth in vitro, as described herein. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. As noted above, immunoreactive polypeptides as provided herein may be used to rapidly expand antigen-specific T cell cultures in order to generate a sufficient number of cells for immunotherapy. In particular, antigen-presenting cells, such as dendritic, macrophage, monocyte, fibroblast and/or B cells, may be pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., Immunological Reviews 157:177, 1997).

Alternatively, a vector expressing a polypeptide recited herein may be introduced into antigen presenting cells taken from a patient and clonally propagated ex vivo for transplant back into the same patient. Transfected cells may be reintroduced into the patient using any means known in the art, preferably in sterile form by intravenous, intracavitary, intraperitoneal or intratumor administration.

Routes and frequency of administration of the therapeutic compositions described herein, as well as dosage, will vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration) or orally. Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an anti-tumor immune response, and is at least 10-50% above the basal (i.e., untreated) level. Such response can be monitored by measuring the anti-tumor antibodies in a patient or by vaccine-dependent generation of cytolytic effector cells capable of killing the patient's tumor cells in vitro. Such vaccines should also be capable of causing an immune response that leads to an improved clinical outcome (e.g., more frequent remissions, complete or partial or longer disease-free survival) in vaccinated patients as compared to non-vaccinated patients. In general, for pharmaceutical compositions and vaccines comprising one or more polypeptides, the amount of each polypeptide present in a dose ranges from about 25 μg to 5 mg per kg of host. Suitable dose sizes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Increases in preexisting immune responses to a tumor protein generally correlate with an improved clinical outcome. Such immune responses may generally be evaluated using standard proliferation, cytotoxicity or cytokine assays, which may be performed using samples obtained from a patient before and after treatment.

Cancer Detection and Diagnostic Compositions, Methods and Kits

In general, a cancer may be detected in a patient based on the presence of one or more pancreatic tumor proteins and/or polynucleotides encoding such proteins in a biological sample (for example, blood, sera, sputum urine and/or tumor biopsies) obtained from the patient. In other words, such proteins may be used as markers to indicate the presence or absence of a cancer such as pancreatic cancer. In addition, such proteins may be useful for the detection of other cancers. The binding agents provided herein generally permit detection of the level of antigen that binds to the agent in the biological sample. Polynucleotide primers and probes may be used to detect the level of mRNA encoding a tumor protein, which is also indicative of the presence or absence of a cancer. In general, a pancreatic tumor sequence should be present at a level that is at least two-fold, preferably three-fold, and more preferably five-fold or higher in tumor tissue than in normal tissue of the same type from which the tumor arose. Expression levels of a particular tumor sequence in tissue types different from that in which the tumor arose are irrelevant in certain diagnostic embodiments since the presence of tumor cells can be confirmed by observation of predetermined tissue differential expression levels, e.g., 2-fold, 5-fold, etc, in tumor tissue to expression levels in normal tissue of the same type.

Other differential expression patterns can be utilized advantageously for diagnostic purposes. For example, in one aspect of the invention, overexpression of a tumor sequence in tumor tissue and normal tissue of the same type, but not in other normal tissue types, e.g. PBMCs, can be exploited diagnostically. In this case, the presence of metastatic tumor cells, for example in a sample taken from the circulation or some other tissue site different from that in which the tumor arose, can be identified and/or confirmed by detecting expression of the tumor sequence in the sample, for example using RT-PCR analysis. In many instances, it will be desired to enrich for tumor cells in the sample of interest, e.g., PBMCs, using cell capture or other like techniques.

There are a variety of assay formats known to those of ordinary skill in the art for using a binding agent to detect polypeptide markers in a sample. See, e.g., Harlow and Lane, Antibodies: A Laboratory Manual, Cold Spring Harbor Laboratory, 1988. In general, the presence or absence of a cancer in a patient may be determined by (a) contacting a biological sample obtained from a patient with a binding agent; (b) detecting in the sample a level of polypeptide that binds to the binding agent; and (c) comparing the level of polypeptide with a predetermined cut-off value.

In a preferred embodiment, the assay involves the use of binding agent immobilized on a solid support to bind to and remove the polypeptide from the remainder of the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent, such as an anti-immunoglobulin, protein G, protein A or a lectin. Alternatively, a competitive assay may be utilized, in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length pancreatic tumor proteins and polypeptide portions thereof to which the binding agent binds, as described above.

The solid support may be any material known to those of ordinary skill in the art to which the tumor protein may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 μg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In certain embodiments, the assay is a two-antibody sandwich assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art, such as bovine serum albumin or Tween 20™ (Sigma Chemical Co., St. Louis, Mo.). The immobilized antibody is then incubated with the sample, and polypeptide is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of polypeptide within a sample obtained from an individual with pancreatic cancer. Preferably, the contact time is sufficient to achieve a level of binding that is at least about 95% of that achieved at equilibrium between bound and unbound polypeptide. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. The second antibody, which contains a reporter group, may then be added to the solid support. Preferred reporter groups include those groups recited above.

The detection reagent is then incubated with the immobilized antibody-polypeptide complex for an amount of time sufficient to detect the bound polypeptide. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of a cancer, such as pancreatic cancer, the signal detected from the reporter group that remains bound to the solid support is generally compared to a signal that corresponds to a predetermined cut-off value. In one preferred embodiment, the cut-off value for the detection of a cancer is the average mean signal obtained when the immobilized antibody is incubated with samples from patients without the cancer. In general, a sample generating a signal that is three standard deviations above the predetermined cut-off value is considered positive for the cancer. In an alternate preferred embodiment, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer.

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound is immersed in a solution containing the sample. The sample migrates along the membrane through a region containing second binding agent and to the area of immobilized binding agent. Concentration of second binding agent at the area of immobilized antibody indicates the presence of a cancer. Typically, the concentration of second binding agent at that site generates a pattern, such as a line, that can be read visually. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 μg, and more preferably from about 50 ng to about 500 ng. Such tests can typically be performed with a very small amount of biological sample.

Of course, numerous other assay protocols exist that are suitable for use with the tumor proteins or binding agents of the present invention. The above descriptions are intended to be exemplary only. For example, it will be apparent to those of ordinary skill in the art that the above protocols may be readily modified to use tumor polypeptides to detect antibodies that bind to such polypeptides in a biological sample. The detection of such tumor protein specific antibodies may correlate with the presence of a cancer.

A cancer may also, or alternatively, be detected based on the presence of T cells that specifically react with a tumor protein in a biological sample. Within certain methods, a biological sample comprising CD4⁺ and/or CD8⁺ T cells isolated from a patient is incubated with a tumor polypeptide, a polynucleotide encoding such a polypeptide and/or an APC that expresses at least an immunogenic portion of such a polypeptide, and the presence or absence of specific activation of the T cells is detected. Suitable biological samples include, but are not limited to, isolated T cells. For example, T cells may be isolated from a patient by routine techniques (such as by Ficoll/Hypaque density gradient centrifugation of peripheral blood lymphocytes). T cells may be incubated in vitro for 2-9 days (typically 4 days) at 37° C. with polypeptide (e.g., 5-25 μg/ml). It may be desirable to incubate another aliquot of a T cell sample in the absence of tumor polypeptide to serve as a control. For CD4⁺ T cells, activation is preferably detected by evaluating proliferation of the T cells. For CD8⁺ T cells, activation is preferably detected by evaluating cytolytic activity. A level of proliferation that is at least two fold greater and/or a level of cytolytic activity that is at least 20% greater than in disease-free patients indicates the presence of a cancer in the patient.

As noted above, a cancer may also, or alternatively, be detected based on the level of mRNA encoding a tumor protein in a biological sample. For example, at least two oligonucleotide primers may be employed in a polymerase chain reaction (PCR) based assay to amplify a portion of a tumor cDNA derived from a biological sample, wherein at least one of the oligonucleotide primers is specific for (i.e., hybridizes to) a polynucleotide encoding the tumor protein. The amplified cDNA is then separated and detected using techniques well known in the art, such as gel electrophoresis. Similarly, oligonucleotide probes that specifically hybridize to a polynucleotide encoding a tumor protein may be used in a hybridization assay to detect the presence of polynucleotide encoding the tumor protein in a biological sample.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding a tumor protein of the invention that is at least 10 nucleotides, and preferably at least 20 nucleotides, in length. Preferably, oligonucleotide primers and/or probes hybridize to a polynucleotide encoding a polypeptide described herein under moderately stringent conditions, as defined above. Oligonucleotide primers and/or probes which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule having a sequence as disclosed herein. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, NY, 1989).

One preferred assay employs RT-PCR, in which PCR is applied in conjunction with reverse transcription. Typically, RNA is extracted from a biological sample, such as biopsy tissue, and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a cancer. The amplification reaction may be performed on several dilutions of cDNA spanning two orders of magnitude. A two-fold or greater increase in expression in several dilutions of the test patient sample as compared to the same dilutions of the non-cancerous sample is typically considered positive.

In another aspect of the present invention, cell capture technologies may be used in conjunction, with, for example, real-time PCR to provide a more sensitive tool for detection of metastatic cells expressing pancreatic tumor antigens. Detection of pancreatic cancer cells in biological samples, e.g., bone marrow samples, peripheral blood, and small needle aspiration samples is desirable for diagnosis and prognosis in pancreatic cancer patients.

Immunomagnetic beads coated with specific monoclonal antibodies to surface cell markers, or tetrameric antibody complexes, may be used to first enrich or positively select cancer cells in a sample. Various commercially available kits may be used, including Dynabeads® Epithelial Enrich (Dynal Biotech, Oslo, Norway), StemSep™ (StemCell Technologies, Inc., Vancouver, BC), and RosetteSep (StemCell Technologies). A skilled artisan will recognize that other methodologies and kits may also be used to enrich or positively select desired cell populations. Dynabeads® Epithelial Enrich contains magnetic beads coated with mAbs specific for two glycoprotein membrane antigens expressed on normal and neoplastic epithelial tissues. The coated beads may be added to a sample and the sample then applied to a magnet, thereby capturing the cells bound to the beads. The unwanted cells are washed away and the magnetically isolated cells eluted from the beads and used in further analyses.

RosetteSep can be used to enrich cells directly from a blood sample and consists of a cocktail of tetrameric antibodies that targets a variety of unwanted cells and crosslinks them to glycophorin A on red blood cells (RBC) present in the sample, forming rosettes. When centrifuged over Ficoll, targeted cells pellet along with the free RBC. The combination of antibodies in the depletion cocktail determines which cells will be removed and consequently which cells will be recovered. Antibodies that are available include, but are not limited to: CD2, CD3, CD4, CD5, CD8, CD10, CD11b, CD14, CD15, CD16, CD19, CD20, CD24, CD25, CD29, CD33, CD34, CD36, CD38, CD41, CD45, CD45RA, CD45RO, CD56, CD66B, CD66e, HLA-DR, IgE, and TCRαβ.

Additionally, it is contemplated in the present invention that mAbs specific for pancreatic tumor antigens can be generated and used in a similar manner. For example, mAbs that bind to tumor-specific cell surface antigens may be conjugated to magnetic beads, or formulated in a tetrameric antibody complex, and used to enrich or positively select metastatic %%% tumor cells from a sample. Once a sample is enriched or positively selected, cells may be lysed and RNA isolated. RNA may then be subjected to RT-PCR analysis using %%% tumor-specific primers in a real-time PCR assay as described herein. One skilled in the art will recognize that enriched or selected populations of cells may be analyzed by other methods (e.g in situ hybridization or flow cytometry).

In another embodiment, the compositions described herein may be used as markers for the progression of cancer. In this embodiment, assays as described above for the diagnosis of a cancer may be performed over time, and the change in the level of reactive polypeptide(s) or polynucleotide(s) evaluated. For example, the assays may be performed every 24-72 hours for a period of 6 months to 1 year, and thereafter performed as needed. In general, a cancer is progressing in those patients in whom the level of polypeptide or polynucleotide detected increases over time. In contrast, the cancer is not progressing when the level of reactive polypeptide or polynucleotide either remains constant or decreases with time.

Certain in vivo diagnostic assays may be performed directly on a tumor. One such assay involves contacting tumor cells with a binding agent. The bound binding agent may then be detected directly or indirectly via a reporter group. Such binding agents may also be used in histological applications. Alternatively, polynucleotide probes may be used within such applications.

As noted above, to improve sensitivity, multiple tumor protein markers may be assayed within a given sample. It will be apparent that binding agents specific for different proteins provided herein may be combined within a single assay. Further, multiple primers or probes may be used concurrently. The selection of tumor protein markers may be based on routine experiments to determine combinations that results in optimal sensitivity. In addition, or alternatively, assays for tumor proteins provided herein may be combined with assays for other known tumor antigens.

The present invention further provides kits for use within any of the above diagnostic methods. Such kits typically comprise two or more components necessary for performing a diagnostic assay. Components may be compounds, reagents, containers and/or equipment. For example, one container within a kit may contain a monoclonal antibody or fragment thereof that specifically binds to a tumor protein. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

Alternatively, a kit may be designed to detect the level of mRNA encoding a tumor protein in a biological sample. Such kits generally comprise at least one oligonucleotide probe or primer, as described above, that hybridizes to a polynucleotide encoding a tumor protein. Such an oligonucleotide may be used, for example, within a PCR or hybridization assay. Additional components that may be present within such kits include a second oligonucleotide and/or a diagnostic reagent or container to facilitate the detection of a polynucleotide encoding a tumor protein.

SEQ ID NOs of the instant application claim priority according to the following:

| | | | |
|---|---|---|---|
| application Ser. No. 60/265,305 | filed Jan. 30, 2001 | SEQ ID NOs: 1-74 | 566P1 |
| application Ser. No. 60/305,484 | filed Jul. 12, 2001 | SEQ ID NOs: 75-453 | 566P2 |
| application Ser. No. 60/333,626 | filed Nov. 27, 2001 | SEQ ID NOs: 454-455 | 566P3 |
| application Ser. No. 60/267,568 | filed Feb. 9, 2001 | SEQ ID NOs: 456-528 | 570P1 |
| application Ser. No. 60/265,682 | filed Jan. 31, 2001 | SEQ ID NOs: 529-4272 | 567P1 |
| application Ser. No. 60/278,651 | filed Mar. 21, 2001 | SEQ ID NOs: 4273-4345 | 567P2 |
| application Ser. No. 60/287,121 | filed Apr. 27, 2001 | SEQ ID NOs: 4346-4546 | 567P3 |
| application Ser. No. 60/313,999 | filed Aug. 20, 2001 | SEQ ID NOs: 4547-4554 | 567P5 |

The following Examples are offered by way of illustration and not by way of limitation.

### EXAMPLES

### Example 1

### Identification of Pancreatic Tumor Protein cDNAs from a PCR-Based Subtraction Library

This Example discloses the isolation and identification of cDNA molecules from a cDNA library enriched in polynucleotides encoding secreted and transmembrane proteins.

A cDNA library was constructed by the method of Kopcznski et al. from mRNA purified from rough endoplasmic reticulum (RER) isolated from primary pancreatic tumor cells (PANC 391-34). Proc. Natl. Acad. Sci. 95:9973-9978 (1998) (incorporated herein by reference in its entirety). Briefly, cDNA was prepared from isolated mRNA by employing standard methodology. See, e.g., Ausubel et al., “Short Protocols in Molecular Biology” (4^{th} ed., 1999). The resulting cDNAs were ligated into a LAMBDA ZAP EXPRESS™ vector (Stratagene; La Jolla, Calif.) and mass excision was then employed to generate a plasmid library in E. coli. Individual E. coli colonies were isolated and the cDNA clones subjected to nucleic acid sequencing. The nucleotide sequences of exemplary clones are disclosed herein as SEQ ID NOs:1-66, 75-152, 174-177, 182, 184-452, 454-4550. The predicted amino acid sequences of these clones are presented as SEQ ID NOs:67-74, 153-173, 178-181, 183, 453, 4551-4554 and 4558-4560.

A cDNA library was also constructed and cloned into the PCR2.1 vector (Invitrogen, Carlsbad, Calif.) by subtracting a pool of one or more tumor cDNAs with a pool of cDNA from normal tissues, for example, colon, spleen, brain, liver, kidney, lung, stomach and small intestine, using PCR subtraction methodologies (Clontech, Palo Alto, Calif.). The subtraction is performed using a PCR-based protocol, which is modified to generate larger fragments. Within this protocol, tester and driver double stranded cDNA are separately digested with five restriction enzymes that recognize six-nucleotide restriction sites (MluI, MscI, PvuII, SalI and StuI). This digestion results in an average cDNA size of 600 bp, rather than the average size of 300 bp that results from digestion with RsaI according to the Clontech protocol. This modification does not affect the subtraction efficiency. Two tester populations are then created with different adapters, and the driver library remains without adapters.

The tester and driver libraries are then hybridized using excess driver cDNA. In the first hybridization step, driver is separately hybridized with each of the tester cDNA populations. This results in populations of (a) unhybridized tester cDNAs, (b) tester cDNAs hybridized to other tester cDNAs, (c) tester cDNAs hybridized to driver cDNAs, and (d) unhybridized driver cDNAs. The two separate hybridization reactions are then combined, and rehybridized in the presence of additional denatured driver cDNA. Following this second hybridization, in addition to populations (a) through (d), a fifth population (e) is generated in which tester cDNA with one adapter is hybridized to tester cDNA with the second adapter. Accordingly, the second hybridization step results in enrichment of differentially expressed sequences which can be used as templates for PCR amplification with adapter-specific primers. The ends are then filled in, and PCR amplification is performed using adapter-specific primers. Only population (e), which contained tester cDNA that do not hybridize to driver cDNA, are amplified exponentially. A second PCR amplification step is then performed, to reduce background and further enrich differentially expressed sequences. This PCR-based subtraction technique normalizes differentially expressed cDNAs so that rare transcripts that are over-expressed in pancreatic tumor tissue may be recoverable. Such transcripts would be difficult to recover by traditional subtraction methods.

### Example 2

### Analysis of cDNA Expression of Pancreatic Tumor cDNAs Using Microarray Technology

To determine mRNA expression levels of the isolated cDNA clones, cDNA clones from the pancreatic tumor subtraction library were randomly picked and colony PCR amplified. Their mRNA expression levels in pancreatic tumor, normal pancreas and various other normal tissues were determined using microarray technology (Rosetta Inpharmatics, Inc., Kirkland, Wash.). Briefly, the PCR amplification products were arrayed onto slides into an array format, with each product occupying a unique location in the array. To do this, mRNA was extracted from the tissue sample to be tested, reverse transcribed, and fluorescent-labeled cDNA probes were generated. The microarrays were probed with the labeled cDNA probes, the slides scanned and fluorescence intensity was measured. Data was analyzed using software provided by the manufacturer.

In additional studies, sequences disclosed herein were evaluated for overexpression in specific tumor tissues by microarray analysis. Using this approach, clones from the cDNA library described in Example 1 were randomly picked, PCR amplified, and their mRNA expression profiles in tumor and normal tissues were examined using cDNA microarray technology essentially as described (Shena, M. et al., 1995 Science 270:467-70). In brief, the clones were arrayed onto glass slides as multiple replicas, with each location corresponding to a unique cDNA clone (as many as 5500 clones can be arrayed on a single slide, or chip). Each chip was hybridized with a pair of cDNA probes that were fluorescence-labeled with Cy3 and Cy5, respectively. Typically, 1 μg of polyA⁺ RNA was used to generate each cDNA probe. After hybridization, the chips were scanned and the fluorescence intensity recorded for both Cy3 and Cy5 channels. There were multiple built-in quality control steps. First, the probe quality was monitored using a panel of ubiquitously expressed genes. Secondly, the control plate also included yeast DNA fragments of which complementary RNA may be spiked into the probe synthesis for measuring the quality of the probe and the sensitivity of the analysis. Currently, the technology offers a sensitivity of 1 in 100,000 copies of mRNA. Finally, the reproducibility of this technology was ensured by including duplicated control cDNA elements at different locations.

Those cDNAs showing at least two-fold overexpression in tumor samples as compared to normal samples, and/or demonstrating overexpression based on visual analysis of the microarray data, were searched against Genbank and the results are shown in Table 2. These sequences are set forth in SEQ ID NOs:75-129. Full-length cDNA and protein sequences for 28 of these clones are dislosed in SEQ ID NOs:130-183 and are shown in Table 3.

TABLE 2
Microarray and Genbank Search Results for Pancreas cDNAs

| | | | | Pancrease Chip 2 Microarray Data | | | | |
|---|---|---|---|---|---|---|---|---|
| SEQ ID | | Well | Plate:well | | | Median | Median | |
| NO: | Element (384 | 384 | 96 | Clone ID | Ratio | Signal 1 | Signal 2 | Blastn |
| 75 | PCX352_r01c15 | a 15 | 838:A8 | 80150 | 2.37 | 0.648 | 0.273 | Hu. p53-induced protein PIGPC1, |
| | | | | | | | | transmembrane protein (THW gene) |
| 76 | PCX352_r01c16 | a 16 | 838:B8 | 80151 | 1.88 | 0.292 | 0.156 | Hu. serine (or cysteine) proteinase |
| | | | | | | | | inhibitor, clade E |
| 77 | PCX352_r01c23 | a 23 | 838:A12 | 80152 | 1.83 | 0.906 | 0.496 | Hu. keratin 18 (KRT18) |
| 78 | PCX352_r03c11 | c 11 | 838:E6 | 80153 | 1.06 | 0.481 | 0.456 | Hu. Rab geranylgeranyltransferase, beta |
| | | | | | | | | subunit (RABGGTB) |
| 79 | PCX352_r04c03 | d 3 | 838:G2 | 80154 | 1.06 | 0.193 | 0.183 | Hu. FAT tumor suppressor (Drosophila) |
| | | | | | | | | homolog (FAT) |
| 80 | PCX352_r04c11 | d 11 | 838:G6 | 80155 | 1.55 | 0.999 | 0.645 | Hu. mRNA for transmembrane protein |
| | | | | | | | | (THW gene), p53-induced protein |
| | | | | | | | | PIGPC1 (PIGPC1) |
| 81 | PCX352_r07c03 | g 3 | 839:E2 | 80156 | 0.95 | 0.253 | 0.266 | Hu. fibrillarin (FBL) |
| 82 | PCX352_r08c06 | h 6 | 839:H3 | 80157 | 0.76 | 0.273 | 0.359 | Hu. fibrillarin (FBL) |
| 83 | PCX352_r10c16 | j 16 | 840:D8 | 80158 | 2.75 | 0.937 | 0.34 | Hu. p53-induced protein PIGPC1, |
| | | | | | | | | transmembrane protein (THW gene) |
| 84 | PCX352_r10c24 | j 24 | 840:D12 | 80159 | 5.14 | 3.94 | 0.767 | Hu. similar to collagen, type I, alpha 1; |
| | | | | | | | | Collagen I, alpha-1 polypeptide |
| 85 | PCX352_r12c04 | l 4 | 840:H2 | 80161 | 1.89 | 0.51 | 0.27 | Hu. serine (or cysteine) proteinase |
| | | | | | | | | inhibitor, clade E |
| 86 | PCX352_r12c07 | l 7 | 840:G4 | 80162 | 0.77 | 0.314 | 0.407 | Hu. pM5 protein (PM5) |
| 87 | PCX352_r12c20 | l 20 | 840:H10 | 80163 | 1.55 | 0.72 | 0.466 | Hu. fibrillarin (FBL) |
| 88 | PCX352_r15c03 | o 3 | 841:E2 | 80164 | 1.5 | 0.178 | 0.119 | Hu. type I transmembrane receptor |
| | | | | | | | | (seizure-related protein)(PSK-1) |
| 89 | PCX352_r15c17 | o 17 | 841:E9 | 80165 | 2.14 | 1.219 | 0.568 | Hu. CD24 antigen (small cell lung |
| | | | | | | | | carcinoma cluster 4antigen) (CD24) |
| 90 | PCX352_r16c08 | p 8 | 841:H4 | 80166 | 0.59 | 0.192 | 0.329 | Hu. fibrillarin (FBL) |
| 91 | PCX353_r01c03 | a 3 | 842:A2 | 80167 | 2.68 | 0.071 | 0.027 | Hu. highly similar to glucose-6-phosphate |
| | | | | | | | | dehydrogenase; ubiquitin-like protein |
| | | | | | | | | (GdX) |
| 92 | PCX353_r02c11 | b 11 | 842:C6 | 80168 | 2.87 | 1.156 | 0.403 | Hu. p53-induced protein PIGPC1, |
| | | | | | | | | transmembrane protein (THW gene) |
| 93 | PCX353_r02c23 | b 23 | 842:C12 | 80169 | 2.72 | 1.001 | 0.368 | Hu. p53-induced protein PIGPC1, |
| | | | | | | | | transmembrane protein (THW gene) |
| 94 | PCX353_r06c08 | f 8 | 843:D4 | 80170 | 1.84 | 1.602 | 0.87 | Hu. keratin 18 (KRT18) |
| 95 | PCX353_r07c22 | g 22 | 843:F11 | 80171 | 1.6 | 0.588 | 0.368 | Hu. plastin 3 (T isoform) (PLS3) |
| 96 | PCX353_r08c06 | h 6 | 843:H3 | 80173 | 1.55 | 0.378 | 0.244 | Hu. liver-specific bHLH-Zip transcription |
| | | | | | | | | factor (LISCH7) |
| 97 | PCX353_r13c20 | m 20 | 845:B10 | 80175 | 1.22 | 0.26 | 0.214 | Hu. collagen, type XVIII, alpha 1 |
| | | | | | | | | (COL18A1) |
| 98 | PCX353_r13c23 | m 23 | 845:A12 | 80176 | 1.59 | 0.432 | 0.272 | Hu. tissue factor pathway inhibitor 2 |
| | | | | | | | | (TFPI2), placental protein 5 (PP5) |
| 99 | PCX353_r16c10 | p 10 | 845:H5 | 80178 | 1.72 | 1.116 | 0.649 | Hu. tumor antigen (L6) |
| 100 | PCX354_r01c03 | a 3 | 846:A2 | 80180 | 1.57 | 0.095 | 0.06 | Hu. small EDRK-rich factor 1B |
| | | | | | | | | (centromeric) (SERF1B) |
| 101 | PCX354_r03c02 | c 2 | 846:F1 | 80181 | 1.8 | 0.951 | 0.53 | Hu. p53-induced protein PIGPC1, |
| | | | | | | | | transmembrane protein (THW gene) |
| 102 | PCX354_r04c04 | d 4 | 846:H2 | 80183 | 1.17 | 0.301 | 0.258 | Hu. fibrillarin (FBL) |
| 103 | PCX354_r04c10 | d 10 | 846:H5 | 80184 | 1.76 | 0.545 | 0.31 | Hu. BAC clone RP11-549B18 from 18 |
| 104 | PCX354_r05c14 | e 14 | 847:B7 | 80185 | 2 | 1.475 | 0.739 | Hu. tumor antigen (L6) |
| 105 | PCX354_r05c21 | e 21 | 847:A11 | 80186 | 3.6 | 1.049 | 0.291 | Novel (short/poor sequence) |
| 106 | PCX354_r08c03 | h 3 | 847:G2 | 80187 | 3.03 | 0.335 | 0.11 | Hu. thrombospondin 2 (THBS2) |
| 107 | PCX354_r08c14 | h 14 | 847:H7 | 80188 | 1.58 | 1.032 | 0.654 | Hu. clone RP11-527G2, |
| | | | | | | | | DKFZP564A2416 protein |
| 108 | PCX354_r08c23 | h 23 | 847:G12 | 80189 | 2.38 | 1.421 | 0.596 | Hu. tumor antigen (L6) |
| 109 | PCX354_r09c15 | i 15 | 848:A8 | 80191 | 2.44 | 0.602 | 0.247 | Hu. p53-induced protein PIGPC1, |
| | | | | | | | | transmembrane protein (THW gene) |
| 110 | PCX354_r09c19 | i 19 | 848:A10 | 80192 | 2.22 | 0.753 | 0.339 | Hu. p53-induced protein PIGPC1, |
| | | | | | | | | transmembrane protein (THW gene) |
| 111 | PCX354_r11c13 | k 13 | 848:E7 | 80193 | 2 | 0.391 | 0.196 | Hu. sema domain, immunoglobulin |
| | | | | | | | | domain (Ig), short basicdomain, secreted, |
| | | | | | | | | (semaphorin) 3C (SEMA3C) |
| 112 | PCX354_r11c19 | k 19 | 848:E10 | 80194 | 2.2 | 1.718 | 0.783 | Hu. connective tissue growth factor |
| | | | | | | | | (CTGF) |
| 113 | PCX354_r12c15 | l 15 | 848:G8 | 80195 | 2.15 | 1.144 | 0.533 | Hu. p53-induced protein PIGPC1, |
| | | | | | | | | transmembrane protein (THW gene) |
| 114 | PCX355_r01c03 | a 3 | 850:A2 | 80196 | 1.79 | 0.092 | 0.051 | Human mitochondrion |
| 115 | PCX355_r02c11 | b 11 | 850:C6 | 80197 | 1.96 | 1.442 | 0.735 | Hu. connective tissue growth factor |
| | | | | | | | | (CTGF) |
| 116 | PCX355_r07c23 | g 23 | 851:E12 | 80198 | 1.91 | 1.295 | 0.679 | Hu. keratin 18 (KRT18) |
| 117 | PCX355_r10c23 | j 23 | 852:C12 | 80199 | 1.68 | 0.823 | 0.489 | Hu. keratin 18 (KRT18) |
| 118 | PCX353_r08c01 | h 1 | 843:G1 | 80172 | 1.65 | 0.482 | 0.291 | Hu. serine (or cysteine) proteinase |
| | | | | | | | | inhibitor, clade E |
| 119 | PCX353_r10c11 | j 11 | 844:C6 | 80174 | 1.45 | 0.412 | 0.285 | Hu. FAT tumor suppressor (Drosophila) |
| | | | | | | | | homolog (FAT) |
| 120 | PCX353_r15c02 | o 2 | 845:F1 | 80177 | 1.05 | 0.47 | 0.446 | Hu. similar to heterochromatin-like |
| | | | | | | | | protein 1 |
| 121 | PCX353_r16c16 | p 16 | 845:H8 | 80179 | 1.76 | 1.259 | 0.715 | Hu. transmembrane protein (THW gene), |
| | | | | | | | | PIGPC1 |
| 122 | PCX354_r09c03 | i 3 | 848:A2 | 80190 | 2.35 | 0.789 | 0.336 | Hu. p53-induced protein PIGPC1, |
| | | | | | | | | transmembrane protein (THW g |
| 123 | PCX355_r11c11 | k 11 | 852:E6 | 80200 | 1.9 | 0.881 | 0.463 | Hu. tumor antigen (L6) |
| 124 | PCX355_r15c13 | o 13 | 853:E7 | 80201 | 1.67 | 0.414 | 0.248 | Hu. stearoyl-CoA desaturase (SCD) |
| 125 | PCX356_r03c06 | c 6 | 854:F3 | 80203 | 1.58 | 0.59 | 0.373 | Hu. chromogranin B (secretogranin 1) |
| 126 | PCX356_r04c20 | d 20 | 854:H10 | 80204 | 3.1 | 0.062 | 0.02 | Hu. serine (or cysteine) proteinase |
| | | | | | | | | inhibitor, clade E |
| 127 | PCX356_r06c11 | f 11 | 855:C6 | 80205 | 2.01 | 1.117 | 0.556 | Hu. tumor antigen (L6), transmembrane 4 |
| | | | | | | | | superfamily member 1 |
| 128 | PCX356_r07c24 | g 24 | 855:F12 | 80207 | 1.69 | 0.301 | 0.178 | Novel - short sequence |
| 129 | PCX356_r12c21 | l 21 | 856:G11 | 80208 | 1.5 | 0.254 | 0.169 | Hu. Fer-1 (C. elegans)-like 3 (myoferlin) |

TABLE 3
Full-length cDNA and Protein sequences for Pancreas cDNAs

| SEQ ID NO: | | | |
|---|---|---|---|
| (Full-Length | | CLONE | |
| cDNA/Pro) | CLONE NAME | ID | GENBANK IDENTITY/SEQUENCE FROM BLASTN OF SEQID |
| 130/153 | IodesPancChip2-1 | 80150 | Hu. p53-induced protein PICPC1, transmembrane protein (THW gene) |
| 131/154 | IodesPancChip2-2 | 80151 | Hu. serine (or cysteine) proteinase inhibitor, clade E |
| 132/155 | IodesPancChip2-3 | 81052 | Hu. keratin 18 (KRT18) |
| 133/156 | IodesPancChip2-4 | 81053 | Hu. Rab geranylgeranyltransferase, beta subunit (RABGGTB) |
| 134/157 | IodesPancChip2-5 | 81054 | Hu. FAT tumor suppressor (Drosophila) homolog (FAT) |
| 135/158 | IodesPancChip2-7 | 80156 | Hu. fibrallarin (FBL) |
| 136/159 | IodesPancChip2-10 | 80159 | Hu. similar to collagen, type I, alpha 1; Collagen I, alpha-1 |
| | | | polypeptide |
| 137/160 | IodesPancChip2-13 | 80162 | Hu. pM5 protein (PM5) |
| 138/161 | IodesPancChip2-15 | 80164 | Hu. type I transmembrane receptor (seizure-related protein) (PSK-1) |
| 139/162 | IodesPancChip2-16 | 80165 | Hu. CD24 antigen (small cell lung carcinoma cluster 4antigen) (CD24) |
| 140 | IodesPancChip2-18 | 80167 | Hu. highly similar to glucose-6-phosphate dehydrogenase |
| 141/163 | | 80167 | Hu. ubiquitin-like protein (GdX) |
| 142/164 | IodesPancChip2-22 | 80171 | Hu. plastin 3 (T isoform) (PLS3) |
| 143/165 | IodesPancChip2-24 | 80173 | Hu. liver-specific bHLH-Zip transcription factor (LISCH7) |
| 144/166 | IodesPancChip2-26 | 80175 | Hu. collagen, type XVIII, alpha 1 (COL18A1) |
| 145/167 | IodesPancChip2-27 | 80176 | Hu. tissue factor pathway inhibitor 2 (TFPI2), placental protein 5 |
| | | | (PP5) |
| 146/168 | IodesPancChip2-29 | 80178 | Hu. tumor antigen (L6) |
| 147/169 | IodesPancChip2-31 | 80180 | (SERF1A) |
| 148/170 | LodesPancChip2-31 | 80180 | Hu. small EDRK-rich factor 1B (centromeric) (SERF1B) |
| 149/171 | IodesPancChip2-38 | 80187 | Hu. thrombospondin 2 (THBS2) |
| 150/172 | IodesPancChip2-44 | 80193 | Hu. sema domain, immunoglobulin domain (lg), short basicdomain, |
| | | | secreted, (semaphorin) 3C (SEMA3C) |
| 151/173 | IodesPancChip2-45 | 80194 | Hu. connective tissue growth factor (CTGF) |
| 152 | IodesPancChip2-47 | 80196 | Human pHL-1 gene, c-myc oncogene containing coxIII sequence |
| 174/178 | IodesPancChip2-28 | 80177 | Heterochromatin-like protein 1 |
| 175/179 | IodesPancChip2-52 | 80201 | Hu. stearoyl-CoA desaturase (SCD) |
| 176/180 | IodesPancChip2-54 | 80203 | Hu. chromogranin B (secretogranin 1) |
| 177/181 | IodesPancChip2-59 | 80208 | Hu. Fer-1 (C. elegans)-like 3 (myoferlin) |
| 182/183 | IodesPancChip2-11 | 80160 | Hu. clone MGC:15409; 93% homolgy to NF-IL6 |

### Example 3

### Identification of Additional Pancreatic Tumor Protein cDNAs from an Expression Library

The PCR-based subtraction library described in Example I was further screened to isolate additional cDNAs expressed in pancreatic tumor cells. An additional 268 clones were identified and are disclosed in SEQ ID NOs:184-452. The clones were sequenced and the sequences used in a BLAST search against Genbank. Those sequences showing some degree of similarity to sequences in Genbank are described in Table 4. Those sequences showing no significant similarity to sequences in Genbank are listed in Table 5.

TABLE 4
Pancreas Tumor Sequences Showing Some Degree of Similarity to Sequences in Genbank

| SEQ ID NO: | | | |
|---|---|---|---|
| (Full-Length | | CLONE | GENBANK IDENTITY/SEQUENCE FROM BLASTN OF |
| cDNA) | CLONE NAME | ID | SEQID |
| 185 | PNCM-2 | 71232 | Hu. putative ionotropic glutamate receptor |
| | | | GLURR2 |
| 186 | PNCM-3 | 71233 | Hu. accessory proteins BAP31/BAP29, 6C6-Ag, |
| | | | CDM |
| 188 | PNCM-5 | 71235 | Hu. prosaposin (PSAP), sphingolipid activator |
| | | | protein 1 |
| 189 | PNCM-6 | 71236 | Hu. similar to adaptor-related protein complex 3, |
| | | | sigma 2subunit |
| | | | Hu. alanyl (membrane) aminopeptidase |
| | | | (aminopeptidase N) |
| 190 | PNCM-7 | 71237 | Hu. prosaposin |
| 191 | PNCM-8 | 73408 | Hu. ribosome binding protein 1, ES/130 |
| 192 | PNCM-9 | 73409 | Hu. kinectin 1 (kinesin receptor) (KTN1) |
| 193 | PNCM-11 | 73410 | Hu. Protein A kinase (PPKA) anchor protein |
| | | | (gravin) 12 |
| 194 | PNCM-12 | 71238 | Hu. golgi autoantigen, golgin subfamily a, 4 |
| | | | (GOLGA4) |
| 195 | PNCM-13 | 73411 | Hu. kinectin 1 (kinesin receptor) (KTN1) |
| 196 | PNCM-14 | 71239 | Hu. prosaposin |
| 197, 198 | PNCM-15 | 73412 | Hu. prosaposin |
| 199 | PNCM-16 | 71240 | Hu. prosaposin |
| 200 | PNCM-18 | 71241 | Hu. prosaposin |
| 201 | PNCM-19 | 71242 | Hu. centromere protein F (350/400kD, mitosin) |
| | | | (CENPF) |
| 202 | PNCM-20 | 73413 | Hu. lamin A/C (nuclear env. protein) |
| 203 | PNCM-21 | 71243 | Hu. prosaposin |
| 204 | PNCM-22 | 71244 | Hu. prosaposin |
| 205 | PNCM-23 | 71245 | Hu. methyl-CpG binding domain protein 2 (MBD2) |
| 206 | PNCM-24 | 71246 | Hu. prosaposin |
| 207 | PNCM-25 | 71247 | Hu. prosaposin |
| 208 | PNCM-26 | 71248 | Hu. prosaposin |
| 209 | PNCM-27 | 71249 | Hu. E3 ubiquitin ligase SMURF2 |
| 210 | PNCM-28 | 73414 | Hu. Kinectin 1 |
| 211 | PNCM-29 | 71250 | Hu. fer-1 (C. elegans)-like 3 (myoferlin) (FER1L3) |
| 214 | PNCM-32 | 71253 | Hu. ECSIT (Toll/IL-1 signal transduction) |
| 215 | PNCM-33 | 71254 | Hu. villin 2, cytovillin 2, (ezrin) (VIL2) |
| 216, 217 | PNCM-34 | 71255 | Hu. mitotic checkpoint protein isoform MAD1a |
| | | | (MAD1) |
| 218 | PNCM-35 | 71256 | Hu. Homer-2A |
| 219 | PNCM-36 | 73415 | Macaca fascicularis brain cDNA, clone:QflA-11332 |
| 220 | PNCM-37 | 71257 | Hu. prosaposin |
| 221 | PNCM-38 | 71258 | Hu. methyl-CpG binding domain protein 2 (MBD2) |
| 222 | PNCM-39a | 71259 | Hu. lamin A/C (nuclear env. protein) |
| 223 | PNCM-39b | 73416 | Hu. sphingolipid activator proteins 1 and 2 |
| | | | processedmutant |
| 224 | PNCM-40 | 71260 | Hu. rabaptin-5 (RAB5EP) (endocytic transport) |
| 225 | PNCM-41 | 73376 | Hu. fer-1 |
| 226 | PNCM-42 | 73377 | Hu. CD36 antigen-like 2, lysosomal |
| | | | sialoglycoprotein |
| 227 | PNCM-43 | 73378 | Hu. Protein A kinase (PPKA) anchor protein |
| | | | (gravin) 12 |
| 228 | PNCM-44 | 73379 | Hu. Prosaposin |
| 229, 230 | PNCM-47 | 73381 | Hu. mitochondrion |
| 231 | PNCM-48 | 73382 | Hu. vimentin (VIM) |
| 232, 233 | PNCM-49 | 73383 | Hu. hydroxysteroid (17-beta) dehydrogenase 4 |
| 234, 235 | PNCM-50 | 73384 | Hu. amyloid beta (A4) precursor-like protein 2 |
| | | | (APLP2) |
| 236 | PNCM-51 | 73385 | Hu. Ran binding protein 2 |
| 237 | PNCM-53 | 73386 | Hu. alanyl (membrane) aminopeptidase |
| | | | (aminopeptidase N) |
| 238 | PNCM-56 | 73387 | Hu. kinectin 1 (kinesin receptor) (KTN1) |
| 239, 240 | PNCM-57 | 73388 | Hu. kinectin 1 (Reverse orientation) |
| 241, 242 | PNCM-58 | 73389 | Hu. vimentin (VIM) |
| 243 | PNCM-59 | 73390 | Hu. glutahione-S-transferase like; |
| | | | glutahionetransferase omega |
| 244 | PNCM-60 | 73391 | Hu. ribosome binding protein 1, ES/130, KIAA |
| | | | 1398 |
| 245 | PNCM-61 | 73417 | Hu. Ran binding protein 2 |
| 246 | PNCM-62 | 73392 | Hu. putative ionotropic glutamate receptor |
| | | | GLURR2 |
| 247 | PNCM-63 | 73393 | Hu. fer-1 (C. elegans)-like 3 (myoferlin) (FER1L3) |
| 248 | PNCM-65 | 73418 | Hu. transferrin receptor (p90, CD71) (TFRC) |
| 249 | PNCM-66 | 73395 | Hu. ribosome binding protein 1, ES/130, KIAA |
| | | | 1398 |
| 250 | PNCM-67 | 73396 | Hu. Similar to glucose regulated protein, 58kDa, |
| | | | cloneMGC:3178 |
| 251 | PNCM-68 | 73397 | Hu. kinectin 1 (kinesin receptor) (KTN1) |
| 252, 253 | PNCM-69 | 73419 | Hu. cDNA FLJ10480 fis, clone NT2RP2000126 |
| 254 | PNCM-70 | 73398 | Hu. kinectin 1 (kinesin receptor) (KTN1) |
| 255, 256 | PNCM-71 | 73399 | Hu. TATA element modulatory factor 1 (TMF1) |
| 257 | PNCM-72 | 73400 | Hu. enoyl Coenzyme A hydratase 1, peroxisomal |
| | | | (ECH1) |
| 258, 259 | PNCM-73 | 73420 | Hu. prosaposin |
| 260 | PNCM-74 | 73401 | Hu. vimentin (VIM) [bp] |
| 261 | PNCM-77 | 73404 | Hu. prosaposin |
| 262 | PNCM-78 | 73405 | Hu. golgi autoantigen, golgin subfamily a, 4 |
| | | | (GOLGA4) |
| 263, 264 | PNCM-80 | 73407 | Hu. kinectin 1 (kinesin receptor) (KTN1) |
| 265 | PNCM-81 | 72174 | Hu. ribosomal protein L9 |
| 266 | PNCM-82 | 72175 | Hu. putative transmembrane protein |
| 267 | PNCM-83 | 72176 | Hu. kinectin 1 (kinesin receptor) (KTN1) |
| 268 | PNCM-84 | 72177 | Hu. prosaposin |
| 269 | PNCM-85 | 72178 | Hu. ORF (LOC51035) w/CpGIsland and Ubiq.- |
| | | | binding domains |
| 270 | PNCM-86 | 72179 | Hu. Protein A kinase (PRKA) anchor protein |
| | | | (gravin) 12 (AKAP12) |
| 271, 272 | PNCM-87 | 73421 | Hu. heat shock 105kD, antigen NY-CO-25 |
| 273 | PNCM-88 | 72180 | Hu. heat shock 105kD, antigen NY-CO-25 (Colon |
| | | | cancer Ag.) |
| 274 | PNCM-89 | 72181 | Hu. ferritin, heavy polypeptide 1 (FTH1) |
| 275 | PNCM-90 | 72182 | Hu. frizzled (Drosophila) homolog 6 (FZD6) |
| 276 | PNCM-91 | 72183 | Hu. vimentin (VIM) |
| 277 | PNCM-92 | 72184 | Hu. Ran binding protein 2 [bp 198-610], sperm |
| | | | membrane protein BS-63, nucleoporin (NUP358) |
| 278 | PNCM-93 | 72185 | Hu. kinectin 1 (kinesin receptor) (KTN1) |
| 279 | PNCM-94 | 72186 | Hu. Tax-1 (T-cell leukemia virus type I) |
| | | | bindingprotein 1 (TAX1BP1) [bp 923-1335]; |
| | | | TRAF6-binding protein T6BP (IL-1 signaling) |
| 280 | PNCM-95 | 72187 | Hu. kinectin 1 (kinesin receptor) (KTN1) [bp 813- |
| | | | 1223] |
| 281 | PNCM-96 | 72188 | Hu. prosaposin [bp 608-1018] |
| 282 | PNCM-97 | 72189 | Hu. heat shock 105kD . . . [bp 1-412] |
| 283 | PNCM-98 | 72190 | Hu. clone IMAGE:3449323 |
| 284 | PNCM-99 | 72191 | Hu. rabaptin-5 [bp 1578-1990] |
| 285 | PNCM-100 | 72192 | Hu. TNF receptor-1 associated protein (TRADD) |
| 286 | PNCM-101 | 72193 | Hu. trans-Golgi network protein |
| 287 | PNCM-102 | 72194 | Hu. IMAGE:3355762, chromodomain helicase |
| | | | DNA binding protein 1-like |
| 288 | PNCM-103 | 72195 | Hu. ribosome binding protein 1, ES/130 [bp 1563- |
| | | | 1975], KIAA1398 |
| 289 | PNCM-106 | 72196 | Hu. ubiquinol-cytochrome c reductase core protein |
| | | | II (UQCRC2) |
| 290 | PNCM-110 | 72197 | Hu. ribosome binding protein 1, ES/130 [bp 717- |
| | | | 1129], KIAA1398 |
| 291 | PNCM-112 | 72198 | Hu. accessory proteins BAP31/BAP29 [bp 237-648], |
| | | | 6C6-Ag, CDM |
| 292 | PNCM-113 | 72199 | Hu. M-phase phosphoprotein 1 (MPHOSPH1) |
| 293 | PNCM-114 | 72200 | Hu. serine palmitoyltransferase, subunit I (enzyme |
| | | | in sphingolipid biosynth.) |
| 294 | PNCM-115 | 72201 | Hu. kinectin 1 (kinesin receptor) (KTN1) [bp 1896- |
| | | | 2306] |
| 295 | PNCM-116 | 72202 | Hu. Tax-1 [bp 1-380] |
| 296 | PNCM-117 | 72203 | Hu. methyl-CpG binding domain protein 2 (MDB2) |
| 297 | PNCM-118 | 72204 | Hu. cDNA: FLJ23027 fis, clone LNG01826 |
| 298 | PNCM-119 | 72205 | Hu. cDNA DKFZp586F1918 |
| 299, 300 | PNCM-120 | 72206 | Macaca fascicularis brain cDNA, clone QflA-11332 |
| 301 | PNCM-122 | 73422 | Hu. heat shock 105kD, antigen NY-CO-25 |
| 302 | PNCM-123 | 73423 | Hu. IMAGE:3355762, chromodomain helicase |
| | | | DNA binding protein 1-like |
| 303 | PNCM-124 | 73424 | Hu. kinectin 1 (kinesin receptor) KTN1) [bp] |
| 304, 305 | PNCM-125 | 73425 | Hu. vimentin (VIM) [bp] |
| 306 | PNCM-126 | 74597 | Hu. prosaposin |
| 307, 308 | PNCM-128 | 73426 | Hu. cleavage stimulation factor, subunit 3, |
| | | | 77kD (CSTF3) |
| 309, 310 | PNCM-129 | 73427 | Hu. diazepam binding inhibitor (GABA receptor |
| | | | modulator, acyl-Coenzyme A binding protein) |
| 311 | PNCM-131 | 73428 | Hu. prosaposin |
| 312 | PNCM-132 | 73429 | Hu., Similar to glucose regulated protein, 58kDa, |
| | | | cloneMGC:3178 |
| 313, 314 | PNCM-133 | 74598 | Hu. kinectin 1 (kinesin receptor) (KTN1) |
| 315 | PNCM-134 | 74599 | Hu. prosaposin |
| 316 | PNCM-135 | 73430 | Hu., Similar to glucose regulated protein, 58 kDa, |
| | | | cloneMGC:3178 |
| 317, 318 | PNCM-136 | 74600 | Hu. ferritin, heavy polypeptide 1 (FTH1) |
| 319, 320 | PNCM-137 | 74601 | Hu. accessory proteins BAP31/BAP29, 6C6- |
| | | | Ag, CDM |
| 321, 322 | PNCM-138 | 73437 | Hu. RER1 protein (RER1) |
| 323, 324 | PNCM-139 | 73438 | Hu. prosaposin |
| 325, 326 | PNCM-141 | 73439 | Hu. Tax-1 (T-cell leukemia virus type I) |
| | | | bindingprotein (TAX1BP1) |
| 327, 328 | PNCM-142 | 73440 | Hu. Protein A kinase (PRKA) anchor protein |
| | | | (gravin) 12 |
| 329 | PNCM-143 | 73441 | Hu. prosaposin |
| 330, 331 | PNCM-144 | 73442 | Hu. prosaposin |
| 332, 333 | PNCM-145 | 73443 | Hu., Similar to glucose regulated protein, 58 kDa, |
| | | | cloneMGC:3178 |
| 336 | PNCM-147 | 74602 | Hu. fer-1 (C. elegans)-like 3 (myoferlin) (FER1L3) |
| 337 | PNCM-148 | 73445 | Hu. prosaposin (PSAP), sphingolipid activator |
| | | | protein 1 |
| 338 | PNCM-150 | 73456 | Hu. heat shock 105kD, antigen NY-CO-25 |
| 339 | PNCM-151 | 73585 | Hu. heat shock 105kD (HSP-105B) |
| 340, 341 | PNCM-152 | 73586 | Hu. Protein A kinase (PRKA) anchor protein |
| | | | (gravin) 12 |
| 342, 343 | PNCM-153 | 73587 | Hu. cleavage stimulation factor, subunit 3, |
| | | | 77kD (CSTF3) |
| 344, 345 | PNCM-154 | 73457 | Hu. methyl-CpG binding domain protein 2 (MBD2) |
| 346, 347 | PNCM-155 | 74603 | Hu. Protein A kinase (PRKA) anchor protein |
| | | | (gravin) 12 |
| 348 | PNCM-157 | 73458 | Hu. Protein A kinase (PRKA) anchor protein |
| | | | (gravin) 12 |
| 349, 350 | PNCM-158 | 73459 | Hu. low density lipoprotein-related protein- |
| | | | associated protein 1 |
| 351, 352 | PNCM-159 | 73460 | Hu. prosaposin |
| 353 | PNCM-160 | 73461 | Hu. accessory proteins BAP31/BAP29 [bp 155- |
| | | | 662], 6C6-Ag, CDM |
| 354, 355 | PNCM-161 | 74604 | Hu. cleavage stimulation factor, subunit 3, |
| | | | 77kD (CSTF3) |
| 356, 357 | PNCM-162 | 74605 | Hu. prosaposin, Hu. Tax-1 |
| 358, 359 | PNCM-163 | 74606 | Hu. leucine zipper-EF-hand containing |
| | | | transmembrane protein 1 (LETM1) |
| 360, 361 | PNCM-164 | 74607 | Hu. CD36 antigen-like 2, lysosomal |
| | | | sialoglycoprotein |
| 362, 363 | PNCM-165 | 74608 | Hu. prosaposin |
| 364 | PNCM-167 | 74610 | Hu. transmembrane protein (63kD) |
| 365 | PNCM-169 | 74611 | Hu. IMAGE:3355762, FLJ22530 fis, clone |
| | | | HRC12866, Hu. prosaposin, Hu. Tax-1 |
| 366, 367 | PNCM-171 | 74613 | Hu. leucine rich repeat (in FLII) interacting protein |
| | | | 1 (LRRFIP1) |
| 368, 369 | PNCM-172 | 74614 | Hu. prosaposin |
| 370, 371 | PNCM-173 | 74640 | Hu. prosaposin |
| 372, 373 | PNCM-174 | 74615 | Hu., Similar to glucose regulated protein, 58 kDa, |
| | | | cloneMGC:3178 |
| 374, 375 | PNCM-175 | 74616 | Hu., Similar to glucose regulated protein, 58 kDa, |
| | | | cloneMGC:3178 |
| 376 | PNCM-176 | 74617 | Hu. prosaposin |
| 377 | PNCM-177 | 77101 | Hu. cleavage stimulation factor subunit 3 |
| | | | 77kD (CSTF3) |
| 378 | PNCM-178 | 77102 | Hu. Protein Protein A kinase (PRKA) anchor |
| | | | protein (gravin) 12 |
| 379 | PNCM-180 | 77104 | Hu. prosaposin |
| 380, 381 | PNCM-182 | 74618 | Hu. prosaposin |
| 382 | PNCM-183 | 74619 | Hu. Ran binding protein 2, sperm membrane protein |
| | | | BS-63, nucleoporin |
| 383, 384 | PNCM-185 | 74620 | Hu. prosaposin |
| 385, 386 | PNCM-186 | 74621 | Hu. ribosome binding protein 1, KIAA1398, clone |
| | | | RP11-462D18 (5-prime) |
| 387, 388 | PNCM-188 | 74623 | Hu. IMAGE:3355762, FLJ22530 fis, clone |
| | | | HRC12866 |
| 389, 390 | PNCM-189 | 74624 | Hu., Similar to glucose regulated protein, 58 kDa, |
| | | | cloneMGC:3178 |
| 391, 392 | PNCM-190 | 74625 | Hu. Ran binding protein 2, sperm membrane protein |
| | | | BS-63, nucleoporin |
| 393 | PNCM-191 | 74631 | Hu. diazepam binding inhibitor (GABA receptor |
| | | | modulator, acyl-Coenzyme A binding protein) |
| 394, 395 | PNCM-193 | 74632 | Hu. endozepine, vimentin |
| 396 | PNCM-202 | 77105 | Hu. glucose-regulated protein, 58kD (GRP58) |
| 397 | PNCM-208 | 77108 | Hu. rabaptin-5 (RAB5EP) |
| 398 | PNCM-210 | 77109 | Hu. vimentin (VIM) |
| 399 | PNCM-215 | 77114 | Hu. hypothetical protein FLJ10634, clone |
| | | | MGC:944 |
| 400 | PNCM-219 | 77118 | Hu. alanyl (membrane) amineopeptidase |
| | | | (aminopeptidase N) |
| 401 | PNCM-221 | 77120 | Hu. similar to RAN binding protein 2 [bp |
| | | | 576 . . . ], nucleoporin (NUP358), sperm-binding |
| | | | protein |
| 402 | PNCM-223 | 77122 | Hu. cell surface glycoprotein/cell adhesion |
| | | | molecule CD44 |
| 403 | PNCM-224 | 77123 | Hu. sperm membrane protein BS-63, RANBP2 |
| 404 | PNCM-226 | 77125 | Hu. Protein Protein A kinase (PRKA) anchor |
| | | | protein (gravin) 12 |
| 405 | PNCM-229 | 77127 | Prosaposin |
| 406 | PNCM-231 | 77129 | Hu. Vimentin |
| 407 | PNCM-232 | 77130 | Prosaposin |
| 408 | PNCM-234 | 77132 | Prosaposin |
| 409 | PNCM-237 | 77134 | Hu. Vimentin |
| 410 | PNCM-238 | 77135 | Hu. accessory proteins BAP31/BAP29, 6C6- |
| | | | Ag, CDM |
| 411 | PNCM-239 | 77136 | Hu. ribosomal protein P0 |
| 412 | PNCM-243 | 77139 | Hu. rabaptin-5 (RAB5EP) |
| 413 | PNCM-244 | 77140 | Prosaposin |
| 414 | PNCM-245 | 77141 | Hu., Similar to glucose regulated protein, 58 kDa, |
| | | | cloneMGC:3178 |
| 415 | PNCM-248 | 77144 | Hu. ORF (LOC51035); Similar to ORF, clone |
| | | | MGC:2274; clone MGC:5321 |
| 416 | PNCM-250 | 77146 | Hu. accessory proteins BAP31/BAP29 [bp 404], 6C6- |
| | | | Ag, CDM |
| 417 | PNCM-253 | 77149 | Hu. methyl-CpG binding domain protein 2 (MBD2) |
| 418 | PNCM-258 | 77474 | Hu. Sim. to glucose-reg. protein [bp 1-99]; 60bp: |
| | | | cGMP-specific phosphodiesterase |
| 419 | PNCM-266 | 77153 | Hu. fer-1 (C. elegans)-like 3 (myoferlin) (FER1L3) |
| 420 | PNCM-267 | 77479 | Hu. Kinectin 1 |
| 421 | PNCM-268 | 77154 | Hu. accessory proteins BAP31/BAP29, 6C6- |
| | | | Ag, CDM |
| 422 | PNCM-269 | 77155 | Hu. Kinectin 1 |
| 423 | PNCM-271 | 77157 | Hu. Kinectin 1 |
| 424 | PNCM-272 | 77480 | Hu. accessory proteins BAP31/BAP29, 6C6- |
| | | | Ag, CDM |
| 425 | PNCM-278 | 77485 | Prosaposin |
| 426 | PNCM-282 | 77487 | Hu. methyl-CpG binding domain protein 2 |
| | | | (MBD2), antigen NY-CO-41 |
| 427 | PNCM-283 | 77488 | Hu. Kinectin 1 |
| 428 | PNCM-287 | 77490 | Hu. Kinectin 1 |
| 429 | PNCM-293 | 77494 | Hu. ribosome binding protein 1, ES/130 |
| 430 | PNCM-294 | 77495 | Hu. leucine zipper-EF-hand containing |
| | | | transmembraneprotein 1 (LETM1) |
| 431 | PNCM-298 | 77499 | Hu. ribosome binding protein 1, ES/130 |
| 432 | PNCM-300 | 77500 | Prosaposin |
| 433 | PNCM-310 | 77160 | Hu. uveal autoantigen |
| 434 | PNCM-311 | 77504 | Hu. accessory proteins BAP31/BAP29, 6C6- |
| | | | Ag, CDM |
| 435 | PNCM-314 | 77506 | Prosaposin |
| 436 | PNCM-316 | 77507 | Hu. Protein Protein A kinase (PRKA) anchor |
| | | | protein (gravin) 12 |
| 437 | PNCM-318 | 77508 | Prosaposin |
| 438 | PNCM-320 | 77509 | Hu. ribosome binding protein 1, ES/130 |
| 439 | PNCM-321 | 77162 | Hu. uveal autoantigen |
| 440 | PNCM-322 | 77163 | Hu. chromodomain helicase DNA binding protein 1- |
| | | | like (CHD1L) |
| 441 | PNCM-324 | 77165 | Hu. accessory proteins BAP31/BAP29, 6C6- |
| | | | Ag, CDM |
| 442 | PNCM-326 | 77167 | Hu. eukaryotic translation initiation factor 2, subunit |
| | | | 2 (beta, 38kD) (EIF2S2) |
| 443 | PNCM-329 | 77169 | Hu. uveal autoantigen |
| 444 | PNCM-331 | 77171 | Hu. Prosaposin |
| 445 | PNCM-332 | 77172 | Hu. alanyl (membrane) aminopeptidase |
| | | | (aminopeptidase N) |
| 446 | PNCM-333 | 77173 | Hu. Prosaposin |
| 447 | PNCM-337 | 77175 | Hu., Similar to glucose regulated protein, 58kDa, |
| | | | cloneMGC:3178 |
| 448 | PNCM-338 | 77176 | Hu. M-phase phosphoprotein 1 |
| 449 | PNCM-341 | 77178 | Hu. cDNA DKFZp586F1918 |
| 450 | PNCM-345 | 77180 | Hu. accessory proteins BAP31/BAP29 6C6- |
| | | | Ag, CDM |
| 451 | PNCM-348 | 77510 | Prosaposin |

TABLE 5
Pancreas Tumor Sequences Showing no Significant Similarity to Sequences in Genbank

| SEQ ID NO: | | | |
|---|---|---|---|
| (Full-Length | | CLONE | GENBANK IDENTITY/SEQUENCE FROM BLASTN OF |
| cDNA/Pro) | CLONE NAME | ID | SEQID |
| 184 | PNCM-1 | 71231 | |
| 187 | PNCM-4 | 71234 | No match to bp376 − 50bp @ 92% w/prosaposin − |
| | | | 100bp no matchl |
| 212 | PNCM-30 | 71251 | |
| 213 | PNCM-31 | 71252 | |
| 334, 335 | PNCM-146 | 73444 | |

### Example 4

### Full- Length Sequence and Expression Analysis of the PN80E Pancreatic Tumor Protein cDNA

The full-length sequence of the cDNA clone 80186, (partial sequence disclosed in SEQ ID NO:105), was determined and is disclosed in SEQ ID NO:454. This sequence as used to search against public databases and the results are described in Table 6.

TABLE 6
Database Search Results for clone 80186, pancreatic tumor candidate Pn80E

| SEQ ID NO | | | |
|---|---|---|---|
| (Partial/Full- | | Candidate | |
| length): | Clone ID | Name | GenBank Blastn Results |
| 105/454 | 80186 | Pn80E | 89% Mus musculus 18 days embryo cDNA, RIKEN full- |
| | | | length enriched library, clone:1110014B07-a secreted |
| | | | protein, with a C1q domain |

The mRNA expression profile was then further analyzed by real-time PCR. The first-strand cDNA used in the quantitative real-time PCR was synthesized from 20 μg of total RNA that was treated with DNase I (Amplification Grade, Gibco BRL Life Technology, Gaithersburg, Md.), using Superscript Reverse Transcriptase (RT) (Gibco BRL Life Technology, Gaithersburg, Md.). Real-time PCR was performed with a GeneAmpTM 5700 sequence detection system (PE Biosystems, Foster City, Calif.). The 5700 system uses SYBR™ green, a fluorescent dye that only intercalates into double stranded DNA, and a set of gene-specific forward and reverse primers. The increase in fluorescence was monitored during the whole amplification process. The optimal concentration of primers was determined using a checkerboard approach and a pool of cDNAs from breast tumors was used in this process. The PCR reaction was performed in 25 μl volumes that included 2.5 μl of SYBR™ green buffer, 2 μl of cDNA template and 2.5 μl each of the forward and reverse primers for the gene of interest. The cDNAs used for RT reactions were diluted 1:10 for each gene of interest and 1:100 for the β-actin control. In order to quantitate the amount of specific cDNA (and hence initial mRNA) in the sample, a standard curve was generated for each run using the plasmid DNA containing the gene of interest. Standard curves were generated using the Ct values determined in the real-time PCR which were related to the initial cDNA concentration used in the assay. Standard dilution ranging from 20-2×10⁶ copies of the gene of interest was used for this purpose. In addition, a standard curve was generated for β-actin ranging from 200 fg-2000 fg. This enabled standardization of the initial RNA content of a tissue sample to the amount of β-actin for comparison purposes. The mean copy number for each group of tissues tested was normalized to a constant amount of β-actin, allowing the evaluation of the over-expression levels seen with each of the genes.

Real-time PCR analysis as described above showed that Pn80E is over-expressed in a panel of pancreatic tumors, including metastatic tumors. Pn80E is also expressed in normal pancreas tissue, adrenal gland, aorta, skin and trachea. Low levels of expression were observed in bone, brain, bronchus, colon, esophagus, heart, kidney, liver, lung, pituitary, skeletal muscle, spinal cord, and spleen. These differential expression patterns indicate that this antigen may be used for immunotherapeutic purposes and/or as a diagnostic marker in individuals with pancreatic cancer.

### Example 5

### Full-Length Sequence and Expression Analysis of the PN81E Pancreatic Tumor Protein cDNA

The full-length sequence of the cDNA clone 80207, (partial sequence disclosed in SEQ ID NO:128), was determined and is disclosed in SEQ ID NO:455. No matches were identified when this sequence was used to search agains public databases.

The mRNA expression profile of Pn81E was then analyzed by real-time PCR as described in Example 4. This analysis showed that Pn81E is over-expressed in a panel of pancreatic tumors and normal pancreas samples as compared to a panel of normal tissues including bone marrow, esophagus, gall bladder, heart, kidney, lung, skeletal muscle, small intestine, and stomach. Expression was observed in bone, PBMC, and spleen. Lower levels of expression were observed in brain, bronchus, colon, liver, pituitary gland, skin, spinal chord, and trachea. These results indicate that the Pn81E antigen may be used in diagnostic and immunotherapy applications.

### Example 6

### Preparation of a PCR-Based cDNA Subtraction Library from Pancreatic Tumors

A cDNA subtraction library containing cDNA from primary pancreatic tumors subtracted with cDNA from normal tissues (liver, salivary gland, small intestine, stomach, heart, brain, bone marrow and normal lung) was constructed as follows. Total RNA was extracted from primary tissues using Trizol reagent (Gibco BRL Life Technologies, Gaithersburg, Md.) as described by the manufacturer. The polyA+ RNA was purified using an oligo(dT) cellulose column according to standard protocols. First strand cDNA was synthesized using the primer supplied in a Clontech PCR-Select cDNA Subtraction Kit (Clontech, Palo Alto, Calif.). The driver DNA consisted of cDNAs from normal tissues with the tester cDNA being from two primary pancreatic tumors. Double-stranded cDNA was synthesized for both tester and driver, and digested with a combination of endonucleases (MluI, MscI, PvuII, SalI and StuI) which recognize six-nucleotide restriction sites. This modification of the digestion procedure resulted in an average cDNA size of 600 bp, rather than the average size of 300 bp that results from digestion with RsaI according to the Clontech protocol. This modification did not affect the subtraction efficiency. The digested tester cDNAs were ligated to two different adaptors and the subtraction was performed according to Clontech's protocol.

The tester and driver libraries were then hybridized using excess driver cDNA. In the first hybridization step, driver was separately hybridized with each of the two tester cDNA populations. This resulted in populations of (a) unhybridized tester cDNAs, (b) tester cDNAs hybridized to other tester cDNAs, (c) tester cDNAs hybridized to driver cDNAs and (d) unhybridized driver cDNAs. The two separate hybridization reactions were then combined, and rehybridized in the presence of additional denatured driver cDNA. Following this second hybridization, in addition to populations (a) through (d), a fifth population (e) was generated in which tester cDNA with one adapter hybridized to tester cDNA with the second adapter. Accordingly, the second hybridization step resulted in enrichment of differentially expressed sequences which could be used as templates for PCR amplification with adaptor-specific primers.

The ends were then filled in, and PCR amplification was performed using adaptor-specific primers. Only population (e), which contained tester cDNA that did not hybridize to driver cDNA, was amplified exponentially. A second PCR amplification step was then performed, to reduce background and further enrich differentially expressed sequences.

This PCR-based subtraction technique normalizes differentially expressed cDNAs so that r are transcripts that are overexpressed in pancreatic tumor tissue may be recoverable. Such transcripts would be difficult to recover by traditional subtraction methods.

The resulting PCR products were subcloned into the TA cloning vector, pCRII (Invitrogen, San Diego, Calif.) and transformed into ElectroMax E. coli DH10B cells (Gibco BRL Life, Technologies) by electroporation. DNA was isolated from independent clones and sequenced using a Perkin Elmer/Applied Biosystems Division (Foster City, Calif.) Automated Sequencer Model 373A.

Seventy three randomly selected cDNA clones in the subtracted pancreatic tumor-specific cDNA library were characterized by DNA sequencing and by subsequent Genbank and EST Blast database searches. Sequences of these partial cDNAs are provided in SEQ ID NO:456-528.

Three thousand seven hundred forty four randomly selected cDNA clones in the subtracted pancreatic tumor-specific cDNA library were characterized by DNA sequencing and by subsequent Genbank and EST Blast database searches. Sequences of these partial cDNAs are provided in SEQ ID NO:529-4346.

### Example 7

### Microarray and Sequence Analysis of cDNA Clones Overexpressed in Pancreas Tumors

One hundred thirty-eight individual clones analyzed using microarray technology as described in Example 2 showed between 3 to 4.5-fold overexpression in pancreas tumors as compared to normal tissues. These cDNA clones were sequenced using standard protocols and compared to public databases (SEQ ID NO:4347-4484). Those cDNAs that showed some degree of similarity to sequences in the databases are described in Table 7. Those cDNAs showing no significant similarity to known sequences in the database are described in Table 8.

TABLE 7
Pancreas Tumor cDNA Clones Showing Some Degree of Similarity to Sequences in Genbank

| SEQ ID | | | | Median | Median | | | |
|---|---|---|---|---|---|---|---|---|
| NO: | Element (384) | Element (96) | Ratio | Signal 1 | Signal 2 | Genset | Contig | Genbank |
| 4346 | p0150r10c11 | R0580 C6 | 4.18 | 0.341 | 0.082 | 61 | 1 | Secretogranin II (Chromogranin C) |
| 4347 | p0150r07c17 | R0579 E9 | 3.68 | 0.163 | 0.044 | 42 | 1 | Secretogranin II (Chromogranin C) |
| 4348 | p0150r09c15 | R0580 A8 | 3.63 | 0.236 | 0.065 | 53 | 1 | Secretogranin II (Chromogranin C) |
| 4349 | p0150r07c14 | R0579 F7 | 3.01 | 0.214 | 0.071 | 46 | 1 | Secretogranin II (Chromogranin C) |
| 4350 | p0150r11c05 | R0580 E3 | 4.38 | 0.096 | 0.022 | 66 | 1 | Secretogranin II (Chromogranin C) |
| 4351 | p0160r06c18 | R0619 D9 | 3.43 | 0.266 | 0.077 | 134 | 2 | Pancreatic Lipase |
| 4352 | p0160r02c21 | R0618 C11 | 3.6 | 0.202 | 0.056 | 133 | 2 | Pancreatic Lipase |
| 4353 | p0160r13c02 | R0621 B1 | 3.24 | 0.202 | 0.062 | 135 | 2 | Pancreatic Lipase |
| 4354 | p0150r13c04 | R0581 B2 | 3.66 | 0.188 | 0.051 | 84 | 3 | Secretogranin II (Chromogranin C) |
| 4355 | p0150r02c15 | R0578 C8 | 4.45 | 0.131 | 0.029 | 11 | 3 | Secretogranin II (Chromogranin C) |
| 4356 | P0150r06c12 | R0579 D6 | 3.02 | 0.274 | 0.091 | 39 | 4 | ATP binding cassette, subfamily B (MDR 1) |
| 4357 | p0151r06c12 | R0583 D6 | 3.4 | 0.112 | 0.033 | 104 | 5 | Human gene for JKTBP2, JKTBP1 |
| 4358 | P0150r14c04 | R0581 D2 | 3.17 | 0.161 | 0.051 | 87 | 6 | 26S proteasome regulatory subunit |
| 4359 | p0150r02c06 | R0578 D3 | 3.28 | 0.193 | 0.059 | 14 | 7 | Chromosome 5 CTC-487M23 |
| 4360 | p0150r04c19 | R0578 G10 | 3.91 | 0.192 | 0.049 | 23 | 8 | cDNA clone FLJ21238 fis, clone COL01115 |
| 4361 | p0150r03c18 | R0578 F9 | 3.94 | 0.163 | 0.041 | 22 | 9 | GTP cyclohydrolase 1 |
| 4362 | p0151r13c03 | R0585 A2 | 3.51 | 0.249 | 0.071 | 108 | 10 | Clone RP11-353C18 on chromosome 20 |
| 4363 | p0150r01c08 | R0578 B4 | 3.86 | 0.171 | 0.044 | 5 | 11 | Spectrin SH3 domain binding protein 1 |
| 4364 | p0150r11c07 | R0580 E4 | 3.02 | 0.143 | 0.048 | 67 | 12 | Human decay accelerating factor (CD55) gene |
| 4365 | P0150r02c11 | R0578 C6 | 3.33 | 0.148 | 0.045 | 10 | 13 | BAC clone GS1-66B6 7p15.2-p13 |
| 4366 | p0150r01c17 | R0578 A9 | 4.14 | 0.202 | 0.049 | 2 | 14 | RNA (guanine-7-) methyltransferase |
| 4367 | p0150r05c16 | R0579 B8 | 3.63 | 0.131 | 0.036 | 32 | 15 | Clone RP1-154G14 on chromosome 6q15-16.3 |
| 4368 | p0150r06c04 | R0579 D2 | 3.82 | 0.315 | 0.083 | 36 | 16 | Human sarcomeric muscle protein |
| 4369 | p0150r15c17 | R0581 E9 | 3.18 | 0.104 | 0.033 | 92 | 17 | Clone 20I3 on chromosome Xq25-26 |
| 4370 | p0150r15c11 | R0581 E6 | 3.47 | 0.147 | 0.043 | 91 | 18 | Human cytoplasmic dynein intermediate chain |
| | | | | | | | | isoform IC-2 |
| 4371 | p0150r09c13 | R0580 A7 | 3.9 | 0.143 | 0.037 | 52 | 19 | Human aldehyde dehydrogenase 1 |
| 4372 | p0150r13c03 | R0581 A2 | 3.14 | 0.222 | 0.071 | 80 | 20 | cDNA DKFZp434C2112 |
| 4373 | p0150r09c10 | R0580 B5 | 3.03 | 0.226 | 0.075 | 57 | 21 | Human RAD21 homolog |
| 4374 | p0161r13c17 | R0625 A9 | 3.39 | 0.217 | 0.064 | 136 | 22 | Pancreatic carboxypeptidase B1 |
| 4375 | p0159r16c21 | R0617 G11 | 4.1 | 0.2 | 0.049 | 132 | 23 | Human matix metalloproteinase 2 |
| 4377 | p0151r09c19 | R0584 A10 | 3.31 | 0.139 | 0.042 | 106 | 25 | Clone RP1-315G1 on chromosome Xq24-25 |
| 4378 | p0151r02c06 | R0582 D3 | 3.46 | 0.326 | 0.094 | 101 | 26 | cDNA FLJ20882 fis, clone ADKA03206 |
| 4379 | p0150r16c06 | R0581 H3 | 3.37 | 0.155 | 0.046 | 100 | 27 | Human potassium channel modulatory factor |
| 4380 | p0150r09c12 | R0580 B6 | 4 | 0.111 | 0.028 | 58 | 28 | Human proprotein convertase subtilisin/kexin |
| | | | | | | | | type 2 |
| 4381 | p0150r07c06 | R0579 F3 | 3.37 | 0.195 | 0.058 | 43 | 29 | KIAA0205 gene |
| 4382 | p0150r06c19 | R0579 C10 | 3.34 | 0.178 | 0.053 | 34 | 30 | Human thymidylate synthase |
| 4383 | p0150r04c05 | R0578 G3 | 3.72 | 0.218 | 0.059 | 24 | 31 | Clone RP11-179B15 |
| 4384 | p0150r03c21 | R0578 E11 | 4.17 | 0.2 | 0.048 | 18 | 32 | KIAA0970 protein |
| 4385 | p0150r15c07 | R0581 E4 | 3.83 | 0.102 | 0.027 | 90 | 33 | Human myosin X |
| 4386 | p0150r02c18 | R0578 D9 | 4.48 | 0.143 | 0.032 | 16 | 33 | Human myosin X |
| 4387 | p0150r10c15 | R0580 C8 | 4.46 | 0.247 | 0.056 | 62 | 34 | KIAA1360 protein |
| 4388 | p0150r07c05 | R0579 E3 | 3.12 | 0.135 | 0.043 | 40 | 35 | cDNA DKFZp586E2023 |
| 4389 | p0150r06c08 | R0579 D4 | 3.85 | 0.177 | 0.046 | 37 | 36 | Human hypothetical protein FLJ10134 |
| 4390 | p0150r01c24 | R0578 B12 | 3.74 | 0.18 | 0.048 | 4 | 37 | Human ALEX1 protein |
| 4391 | p0150r07c15 | R0579 E8 | 3.13 | 0.272 | 0.087 | 41 | 38 | Human tropomodulin |
| 4392 | p0150r02c23 | R0578 C12 | 4.34 | 0.163 | 0.037 | 8 | 39 | Human Kreisler maf-related leucine zipper |
| | | | | | | | | homolog |
| 4393 | p0151r09c08 | R0584 B4 | 4.44 | 0.393 | 0.089 | 107 | 40 | Human histone acetyltransferase |
| 4394 | p0150r16c09 | R0581 G5 | 3.55 | 0.238 | 0.067 | 95 | 41 | Human coronin, actin-binding protein 1C |
| 4395 | p0150r14c11 | R0581 C6 | 3.99 | 0.096 | 0.024 | 86 | 42 | cDNA DKFZp761G2423 |
| 4396 | p0151r05c12 | R0583 B6 | 3.49 | 0.221 | 0.063 | 103 | 43 | Human X-prolyl aminopeptidase-like |
| 4397 | p0150r12c23 | R0580 G12 | 4.06 | 0.14 | 0.034 | 75 | 44 | Clone RP4-758N20 on chromosome 1p31.3-32 |
| 4398 | p0150r16c02 | R0581 H1 | 3.09 | 0.093 | 0.03 | 98 | 45 | KIAA1228 protein |
| 4399 | p0155r10c08 | R0600 D4 | 3.07 | 0.245 | 0.08 | 121 | 46 | cDNA DKFZp586I1419 |
| 4400 | p0150r02c05 | R0578 C3 | 3.82 | 0.174 | 0.046 | 9 | 47 | Human uncharacterized hypothalamus protein |
| | | | | | | | | HARP11 |
| 4401 | p0150r09c04 | R0580 B2 | 3.09 | 0.197 | 0.064 | 55 | 48 | Human aldehyde dehydrogenase 1 |
| 4402 | p0150r03c22 | R0578 F11 | 3.45 | 0.133 | 0.039 | 21 | 49 | Human secretory granule, neuroendocrine |
| | | | | | | | | protein 1 |
| 4403 | p0150r15c10 | R0581 F5 | 3.1 | 0.111 | 0.036 | 94 | 50 | Human I-beta-1,3-N-acetylglucosaminyltransferase |
| 4404 | p0150r13c20 | R0581 B10 | 3.94 | 0.111 | 0.028 | 82 | 51 | Human 12p13.3 BAC RPCI11-350L7 |
| 4405 | p0150r08c18 | R0579 H9 | 3.35 | 0.173 | 0.052 | 49 | 52 | Human tumor differentially expressed 1 |
| 4406 | p0150r09c22 | R0580 B11 | 3.39 | 0.195 | 0.057 | 54 | 53 | Human deoxycytidine kinase |
| 4408 | p0163r04c09 | R0630 G5 | 3 | 0.283 | 0.094 | 138 | 55 | Human hypothetical protein FLJ10540 |
| 4409 | p0155r01c04 | R0598 B2 | 4.2 | 0.377 | 0.09 | 116 | 56 | Human collagen, type III, alpha 1 |
| 4410 | p0152r16c22 | R0589 H11 | 3.74 | 0.363 | 0.097 | 113 | 57 | Human transmembrane 4 superfamily member 4 |
| | | | | | | | | (TM4SF4) |
| 4411 | p0152r16c12 | R0589 H6 | 3.09 | 0.292 | 0.095 | 114 | 58 | Human chloride intracellular channel 1 |
| 4413 | p0150r05c23 | R0579 A12 | 4.1 | 0.189 | 0.046 | 27 | 60 | Human N33 protein form 2 |
| 4414 | p0150r11c09 | R0580 E5 | 3.16 | 0.191 | 0.06 | 68 | 61 | Human hypothetical protein (FLJ11127) |
| 4415 | p0150r13c22 | R0581 B11 | 3.1 | 0.12 | 0.039 | 83 | 62 | DNA sequence from clone 422G23 on |
| | | | | | | | | chromosome 6q24 |
| 4416 | p0150r11c17 | R0580 E9 | 3.66 | 0.244 | 0.067 | 70 | 63 | cDNA FLJ20935 fis, clone ADSE01534 |
| 4417 | p0159r16c01 | R0617 G1 | 4.4 | 0.183 | 0.042 | 131 | 64 | Human carboxypeptidase A2 |
| 4418 | p0156r16c21 | R0605 G11 | 3.37 | 0.206 | 0.061 | 122 | 65 | Human guanine nucleotide exchange factor |
| 4419 | p0150r06c15 | R0579 C8 | 3.21 | 0.168 | 0.052 | 35 | 66 | Human chromosome 5 clone CTC-315O24 |
| 4422 | p0150r11c22 | R0580 F11 | 4.29 | 0.17 | 0.04 | 72 | 69 | Human RNA helicase II/Gu protein gene |
| 4425 | p0150r11c02 | R0580 F1 | 3.65 | 0.207 | 0.057 | 71 | 72 | Human carboxypeptidase E |
| 4426 | p0157r07c21 | R0607 E11 | 3.88 | 0.192 | 0.049 | 126 | 73 | Human cutaneous T-cell lymphoma tumor |
| | | | | | | | | antigen se70-2 |
| 4427 | p0150r16c17 | R0581 G9 | 3.28 | 0.101 | 0.031 | 97 | 74 | KIAA0393 protein |
| 4428 | p0155r03c08 | R0598 F4 | 3.25 | 0.305 | 0.094 | 117 | 75 | Human laminin, gamma 2 |
| 4429 | p0150r07c10 | R0579 F5 | 3.27 | 0.223 | 0.068 | 44 | 76 | Human chromosome 5 clone CTC-576H9 |
| 4430 | p0150r16c13 | R0581 G7 | 3.58 | 0.166 | 0.046 | 96 | 77 | Clone RP11-71J12 on chromosome 13 |
| 4431 | p0150r06c10 | R0579 D5 | 3.05 | 0.243 | 0.08 | 38 | 78 | Human 3q26.2-27 BAC RPCI11-469J4 |
| 4432 | p0150r02c16 | R0578 D8 | 4.47 | 0.11 | 0.025 | 15 | 79 | Human potassium inwardly-rectifying channel, |
| | | | | | | | | subfamily J |
| 4433 | p0150r09c06 | R0580 B3 | 4.25 | 0.176 | 0.042 | 56 | 80 | KIAA1699 protein |
| 4434 | p0150r11c16 | R0580 F8 | 3.49 | 0.178 | 0.051 | 74 | 81 | Human somatostatin receptor 2 |
| 4435 | p0150r01c13 | R0578 A7 | 4.49 | 0.411 | 0.092 | 1 | 82 | Humna claudin-12 |
| 4436 | p0150r08c10 | R0579 H5 | 3.29 | 0.238 | 0.073 | 48 | 83 | Human tumor rejection antigen (gp96) 1 |
| 4438 | p0159r14c18 | R0617 D9 | 3.07 | 0.101 | 0.033 | 130 | 85 | Human carboxypeptidase A2 |
| 4439 | p0150r16c22 | R0581 H11 | 3.09 | 0.304 | 0.098 | 99 | 86 | Human cDNA FLJ12280 fis clone |
| | | | | | | | | MAMMA1001744 |
| 4440 | p0152r10c20 | R0588 D10 | 4.4 | 0.157 | 0.036 | 110 | 87 | Human guanine nucleotide-binding protein alpha- |
| | | | | | | | | subunit |
| 4441 | p0150r13c23 | R0581 A12 | 3.48 | 0.248 | 0.071 | 79 | 88 | Human nuclear factor kappa-B DNA binding |
| | | | | | | | | subunit |
| 4442 | p0150r12c10 | R0580 H5 | 3.7 | 0.158 | 0.043 | 78 | 89 | Human CD164 isoform delta 4 |
| 4443 | p0150r09c18 | R0580 B9 | 3.67 | 0.214 | 0.058 | 59 | 90 | Human galectin-8 gene |
| 4444 | p0158r09c04 | R0612 B2 | 4.14 | 0.14 | 0.034 | 129 | 91 | Human cathepsin C |
| 4445 | p0150r08c09 | R0579 G5 | 3.69 | 0.226 | 0.061 | 47 | 92 | Human imidazoline receptor candidate |
| 4446 | p0150r11c11 | R0580 E6 | 4.15 | 0.237 | 0.057 | 69 | 93 | Human glutaminase isoform C |
| 4447 | p0150r15c01 | R0581 E1 | 4.12 | 0.099 | 0.024 | 88 | 94 | Human chromosome 5 clone CTD-2314G24 |
| 4448 | p0157r07c01 | R0607 E1 | 3.67 | 0.254 | 0.069 | 125 | 95 | DNA from chromosome 19, cosmid F21856 |
| 4449 | p0150r09c21 | R0580 A11 | 3.49 | 0.169 | 0.048 | 51 | 96 | Human false p73 target protein gene |
| 4450 | p0150r12c02 | R0580 H1 | 3.31 | 0.173 | 0.052 | 76 | 97 | cDNA FLJ12946 fis, clone NT2RP2005254 |
| 4451 | p0150r03c17 | R0578 E9 | 4.16 | 0.212 | 0.051 | 20 | 98 | cDNA DKFZp434L1715 |
| 4453 | p0150r05c20 | R0579 B10 | 3.43 | 0.147 | 0.043 | 29 | 100 | Human large conductance calcium-activated |
| | | | | | | | | potassium channel beta |
| 4454 | p0150r05c04 | R0579 B2 | 3.01 | 0.1 | 0.033 | 31 | 101 | Human prostate tumor over expressed gene 1 |
| 4455 | p0150r07c12 | R0579 F6 | 3.59 | 0.191 | 0.053 | 45 | 102 | Human alpha-L-fucosidase gene |
| 4456 | p0150r05c02 | R0579 B1 | 3.15 | 0.205 | 0.065 | 28 | 103 | Human N-terminal acetyltransferase complex |
| | | | | | | | | ard1 subunit |
| 4457 | p0152r13c24 | R0589 B12 | 3.44 | 0.207 | 0.06 | 111 | 104 | Chromosome 19, cosmid F23669 |
| 4458 | p0150r03c11 | R0578 E6 | 3.05 | 0.159 | 0.052 | 19 | 105 | Human IK cytokine, down-regulator of HLA II |
| 4459 | p0162r13c11 | R0629 A6 | 3.07 | 0.133 | 0.043 | 137 | 106 | Human thrombospondin 2 |
| 4460 | p0151r13c15 | R0585 A8 | 3.71 | 0.171 | 0.046 | 109 | 107 | cDNA FLJ23160 fis, clone LNG09682 |
| 4461 | p0150r03c01 | R0578 E1 | 4.03 | 0.145 | 0.036 | 17 | 108 | Human Na+/glucose cotransporter gene |
| 4462 | p0151r08c17 | R0583 G9 | 3.57 | 0.131 | 0.037 | 105 | 109 | Human death-associated protein |
| 4463 | p0150r01c02 | R0578 B1 | 3.1 | 0.287 | 0.093 | 3 | 110 | Human guanine nucleotide binding protein |
| 4464 | p0150r04c06 | R0578 H3 | 3.93 | 0.158 | 0.04 | 25 | 111 | Clone RP4-813B7 on chromosome 1 |
| 4465 | p0150r09c19 | R0580 A10 | 3.86 | 0.197 | 0.051 | 50 | 112 | Human mitochondrion |
| 4466 | p0153r06c10 | R0591 D5 | 3.78 | 0.294 | 0.078 | 115 | 113 | Human DEAD/H box polypeptide 5 |
| 4467 | p0150r02c20 | R0578 D10 | 3.82 | 0.198 | 0.052 | 13 | 114 | KIAA1317 protein |
| 4468 | p0155r09c08 | R0600 B4 | 4.13 | 0.393 | 0.095 | 118 | 115 | KIAA0883 protein |
| 4471 | p0150r13c02 | R0581 B1 | 3.13 | 0.163 | 0.052 | 81 | 117 | RNA binding motif protein 5 |
| 4473 | p0150r11c23 | R0580 E12 | 3.57 | 0.237 | 0.067 | 65 | 119 | Human GTP cyclohydrolase 1 |
| 4474 | p0155r10c24 | R0600 D12 | 3.55 | 0.205 | 0.058 | 120 | 120 | Human mitochondrial DNA control region |
| 4475 | p0157r06c03 | R0607 C2 | 4.14 | 0.172 | 0.042 | 124 | 121 | Ras-related C3 botulinum toxin substrate 1 |
| 4476 | p0150r05c24 | R0579 B12 | 3.83 | 0.157 | 0.041 | 30 | 122 | Human hypothetical protein FLJ20391 |
| 4479 | p0155r10c19 | R0600 C10 | 3.73 | 0.274 | 0.073 | 119 | 125 | KIAA0292 protein |
| 4480 | p0150r14c23 | R0581 C12 | 3.51 | 0.14 | 0.04 | 85 | 126 | ISL1 transcription factor, LIM/homeodomain |
| 4481 | p0150r10c19 | R0580 C10 | 3.56 | 0.188 | 0.053 | 60 | 127 | KIAA0766 gene |
| 4482 | p0150r11c19 | R0580 E10 | 3.28 | 0.152 | 0.046 | 63 | 128 | KIAA1554 protein |
| 4483 | p0150r15c21 | R0581 E11 | 3.25 | 0.114 | 0.035 | 89 | 129 | Chromosome 11p14.3 PAC clone pDJ239b22 |
| 4484 | p0150r11c21 | R0580 E11 | 3.07 | 0.165 | 0.054 | 64 | 130 | Clone RP1-309F20 on chromosome 20 |
| 4412 | p0150r01c16 | R0578 B8 | 3.06 | 0.242 | 0.079 | 7 | 59 | Human thyroid hormone receptor coactivating |
| | | | | | | | | protein |

TABLE 8
Pancreas Tumor cDNAs Showing no Significant
Similarity to Known Sequences in Genbank

| SEQ ID | | | | Median | Median | | |
|---|---|---|---|---|---|---|---|
| NO: | Element (384) | Element (96) | Ratio | Signal 1 | Signal 2 | Genset | Contig |
| 4376 | p0150r02c02 | R0578 D1 | 3.62 | 0.193 | 0.053 | 12 | 24 |
| 4407 | p0157r08c09 | R0607 G5 | 3.18 | 0.155 | 0.049 | 128 | 54 |
| 4420 | p0151r05c04 | R0583 B2 | 3.01 | 0.145 | 0.048 | 102 | 67 |
| 4421 | p0150r05c21 | R0579 A11 | 3.4 | 0.217 | 0.064 | 26 | 68 |
| 4423 | p0150r01c14 | R0578 B7 | 3.67 | 0.19 | 0.052 | 6 | 70 |
| 4424 | p0150r15c08 | R0581 F4 | 3.06 | 0.111 | 0.036 | 93 | 71 |
| 4437 | p0150r05c18 | R0579 B9 | 3.98 | 0.164 | 0.041 | 33 | 84 |
| 4452 | p0157r07c20 | R0607 F10 | 3.09 | 0.158 | 0.051 | 127 | 99 |
| 4469 | p0152r16c01 | R0589 G1 | 3.13 | 0.242 | 0.077 | 112 | 116 |
| 4471 | p0150r12c22 | R0580 H11 | 4 | 0.15 | 0.037 | 77 | 118 |
| 4476 | p0150r11c04 | R0580 F2 | 4.03 | 0.175 | 0.043 | 73 | 123 |
| 4477 | p0156r16c18 | R0605 H9 | 3.03 | 0.201 | 0.066 | 123 | 124 |

### Example 8

### Microarray and Sequence Analysis of Additional cDNA Clones Overexpressed in Pancreas Tumors

Forty eight hundred clones from three PCR-based subtracted cDNA libraries were PCR amplified and arrayed on DNA chips. They were hybridized with fluorescently labeled probes which were generated from pancreatic tumors and a varity of normal tissues including normal pancreas. The array data were analyzed by computer and by visual analysis. Sixty-three clones with 4.5-fold overexpression in pancreatic tumors were selected their sequences were determined by DNA sequencing (SEQ ID NO:44854547). The sequences were then searched against public databases including Genbank and EST. Those cDNAs that showed some degree of similarity to sequences in the databases are described in Table 9. Those cDNAs showing no significant similarity to known sequences in the database are described in Table 10. Several of these cDNAs have been selected as promising candidates for therapeutic and diagnostic purposes. The candidate names are also shown in Tables 9 and 10.

TABLE 9
Additional Pancreas Tumor cDNA Clones Showing Some Degree of Similarity to Sequences in the Genbank

| SEQ ID | | | | Median | Median | | | |
|---|---|---|---|---|---|---|---|---|
| NO: | Element (384) | Element (96) | Ratio | Signal 1 | Signal 2 | Contig | Identity | Candidate |
| 4533 | p0157r07c23 | R0607 E12 | 15.8 | 0.097 | 0.006 | 46 | BAC clone CTA-271G13 from 7 | |
| 4518 | p0157r05c17 | R0607 A9 | 16.36 | 0.169 | 0.01 | 31 | cDNA FLJ12849 fis, clone NT2RP2003393 | |
| 4506 | p0150r09c01 | R0580 A1 | 9.73 | 0.106 | 0.011 | 19 | cDNA FLJ21368 fis, clone COL03056 | |
| | | | | | | | Glutaminase isoform C | |
| 4519 | p0157r07c13 | R0607 E7 | 46.35 | 0.12 | 0.003 | 32 | cDNA FLJ21410 fis, clone COL03938 | |
| 4521 | p0157r07c15 | R0607 E8 | 12.34 | 0.137 | 0.011 | 34 | cDNA FLJ23607 fis, clone LNG16050 | Pn1474P |
| 4507 | p0150r08c17 | R0579 G9 | 5.2 | 0.137 | 0.026 | 20 | Chromogranin B | Pn1468P |
| 4514 | p0150r12c08 | R0580 H4 | 7.88 | 0.046 | 0.006 | 27 | Chromosome 12 clone 917O5 | |
| 4524 | p0152r06c10 | R0587 D5 | 11.83 | 0.038 | 0.003 | 37 | Chromosome 5 clone CTC-534A2 | |
| 4509 | p0150r09c20 | R0580 B10 | 6.1 | 0.131 | 0.021 | 22 | Chromosome 5 clone CTD-2031P19 | |
| 4500 | p0150r11c01 | R0580 E1 | 6.34 | 0.14 | 0.022 | 13 | Chromosome 5 clone CTD-2122K7 | Pn1469P |
| 4523 | p0155r07c16 | R0599 F8 | 4.7 | 0.107 | 0.023 | 36 | Chromosome 7 clone RP11-248K17 | |
| | | | | | | | Caldesmon, 3′ UTR | |
| 4487 | p0157r07c17 | R0607 E9 | 8.97 | 0.148 | 0.017 | 4 | Clone 27K12 on chromosome 6p11.2-12.3 | |
| 4539 | p0157r07c09 | R0607 E5 | 5.64 | 0.262 | 0.046 | | Clone RP1-122P22 on chromosome 20 | Pn1473P |
| 4501 | p0150r03c14 | R0578 F7 | 9.86 | 0.138 | 0.014 | 14 | Clone RP11-239L20 on chromosome 6 | |
| 4529 | p0157r05c18 | R0607 B9 | 24.3 | 0.174 | 0.007 | 42 | Discoidin domain receptor family. Human | Pn1472P |
| | | | | | | | mammary carcinoma kinase | |
| 4498 | p0150r02c07 | R0578 C4 | 4.99 | 0.264 | 0.053 | 11 | DKFZp434M1616 | |
| 4508 | p0150r09c17 | R0580 A9 | 16.97 | 0.121 | 0.007 | 21 | Guanine nucleotide binding protein | |
| | | | | | | | (neuroendocrine secretory protein 55) | |
| 4510 | p0150r08c06 | R0579 H3 | 5.32 | 0.113 | 0.021 | 23 | Hormone-regulated Repro-PC 1.0 gene | |
| | | | | | | | Reprogen Inc. KIAA1342 | |
| 4537 | p0151r12c17 | R0584 G9 | 5.37 | 0.937 | 0.174 | | Human breast tumor protein immunogenic | |
| | | | | | | | fragment | |
| 4513 | p0150r11c03 | R0580 E2 | 19.29 | 0.096 | 0.005 | 26 | Human CGI-86 protein | |
| 4546 | p0157r07c06 | R0607 F3 | 16.6 | 0.18 | 0.011 | | Human clone PP722 unknown mRNA | |
| 4540 | p0157r08c05 | R0607 G3 | 20.8 | 0.353 | 0.017 | | Human colon cancer nucleotide sequence, NCA | Pn1475P |
| 4526 | p0150r02c03 | R0578 C2 | 5.65 | 0.166 | 0.029 | 39 | Human decidual protein induced by | |
| | | | | | | | progesterone | |
| 4517 | p0150r15c23 | R0581 E12 | 7.72 | 0.143 | 0.019 | 30 | Human desmoplakin | |
| 4530 | p0157r06c17 | R0607 C9 | 8.13 | 0.222 | 0.027 | 43 | Human filamin gene (FLNB) | |
| 4504 | p0150r01c04 | R0578 B2 | 10.45 | 0.095 | 0.009 | 17 | Human foetal brain secreted protein | |
| 4534 | p0150r01c20 | R0578 B10 | 4.82 | 0.295 | 0.061 | | Human Hexosaminidase B beta-subunit | |
| 4496 | p0150r07c01 | R0579 E1 | 8.7 | 0.107 | 0.012 | 9 | Human HLA gene for MHC class I antigen, | |
| | | | | | | | B4701 allele MDM2 gene | |
| 4484 | p0157r05c22 | R0607 B11 | 8.32 | 0.211 | 0.025 | 1 | Human long-chain fatty acid coenzyme A ligase | |
| | | | | | | | 5 | |
| 4488 | p0150r15c22 | R0581 F11 | 9.35 | 0.115 | 0.012 | 5 | Human putative helicase RUVBL (LOC56897) | |
| 4511 | p0150r13c19 | R0581 A10 | 5.09 | 0.217 | 0.043 | 24 | Human PVR gene | |
| 4503 | p0150r04c17 | R0578 G9 | 6.29 | 0.404 | 0.064 | 16 | Human secreted protein gene 79 | Pn1467P |
| 4582 | p0150r11c14 | R0580 F7 | 31.67 | 0.1 | 0.003 | 41 | Human secretory granule, neuroendocrine | |
| | | | | | | | protein 1 | |
| 4485 | p0157r05c21 | R0607 A11 | 5.18 | 0.149 | 0.029 | 2 | Human sucrase-isomaltase | |
| 4531 | p0157r07c18 | R0607 F9 | 38.09 | 0.183 | 0.005 | 44 | Human transgelin | |
| 4544 | p0162r02c05 | R0626 C3 | 5.16 | 0.434 | 0.084 | | hXAG | |
| 4486 | p0157r06c05 | R0607 C3 | 9.62 | 0.128 | 0.013 | 3 | KIAA0071 | |
| 4527 | p0150r06c24 | R0579 D12 | 4.75 | 0.112 | 0.024 | 40 | KIAA0804 | |
| 4516 | p0150r09c11 | R0580 A6 | 5.32 | 0.227 | 0.043 | 29 | KIAA1289 | |
| 4541 | p0157r08c17 | R0607 G9 | 5.59 | 0.213 | 0.038 | | Malate dehydrogenase 2, NAD | |
| 4495 | p0150r05c19 | R0579 A10 | 11.78 | 0.112 | 0.01 | 9 | MDM2 gene | |
| 4536 | p0150r15c06 | R0581 F3 | 6.86 | 0.227 | 0.033 | | Neuroendocrine secretory protein 55 | |
| 4489 | p0159r03c13 | R0614 E7 | 5.97 | 0.549 | 0.092 | 6 | Pancreatic lipase | |
| 4490 | p0160r04c18 | R0618 H9 | 4.68 | 0.374 | 0.08 | 6 | Pancreatic lipase | |
| 4545 | p0157r06c24 | R0607 D12 | 18.84 | 0.157 | 0.008 | | Pro alpha 1 (I) collagen | |
| 4515 | p0150r05c22 | R0579 B11 | 4.63 | 0.187 | 0.04 | 28 | PRO2000 protein | |
| 4520 | p0157r07c14 | R0607 F7 | 26.74 | 0.149 | 0.006 | 33 | Protein identified by the signal sequence trap | |
| | | | | | | | polyA site DNA | |
| 4543 | p0161r16c06 | R0625 H3 | 5.96 | 1.13 | 0.19 | | Regenerating islet-derived 1 beta | |
| 4491 | p0150r06c23 | R0579 C12 | 4.79 | 0.133 | 0.028 | 7 | RP11-287F15 chromosome 9 | |
| 4492 | p0150r02c15 | R0578 C8 | 4.45 | 0.131 | 0.029 | 7 | RP11-287F15 chromosome 9 | |
| 4493 | p0150r13c24 | R0581 B12 | 6.41 | 0.138 | 0.021 | 8 | Secretogranin II | |
| 4494 | p0150r15c03 | R0581 E2 | 6.13 | 0.129 | 0.021 | 8 | Secretogranin II | |
| 4502 | p0150r02c12 | R0578 D6 | 6.07 | 0.149 | 0.025 | 15 | Secretogranin II | |
| 4505 | p0150r02c22 | R0578 D11 | 5.65 | 0.147 | 0.026 | 18 | Secretogranin II | |

TABLE 10
Additional Pancreas Tumor cDNAs Showing no Significant
Similarity to Known Sequences in Genbank

| | | | | Median | Median | | |
|---|---|---|---|---|---|---|---|
| SEQ ID NO: | Element (384) | Element (96) | Ratio | Signal 1 | Signal 2 | Contig | Candidate |
| 4497 | p0150r06c16 | R0579 D8 | 4.93 | 0.105 | 0.021 | 10 | |
| 4499 | p0152r15c23 | R0589 E12 | 4.91 | 0.186 | 0.038 | 12 | Pn1471P |
| 4512 | p0150r12c09 | R0580 G5 | 4.6 | 0.083 | 0.018 | 25 | |
| 4522 | p0151r01c08 | R0582 B4 | 5.1 | 0.158 | 0.031 | 35 | |
| 4525 | p0150r04c18 | R0578 H9 | 5.3 | 0.145 | 0.027 | 38 | |
| 4532 | p0157r06c08 | R0607 D4 | 11.23 | 0.093 | 0.008 | 45 | |
| 4535 | p0150r14c15 | R0581 C8 | 5.19 | 0.102 | 0.02 | | Pn1470P |
| 4538 | p0155r12c15 | R0600 G8 | 5.35 | 0.772 | 0.145 | | |
| 4542 | p0160r03c01 | R0618 E1 | 4.73 | 0.489 | 0.104 | | Pn1476P |

### Example 9

### Quantitative Real-Time PCR Analysis of cDNA Clones Overexpressed in Pancreatic Tumors

Four pancreas clones selected by cDNA microarray and subtracted cDNA library as described in Examples 2 and 6-8 were analyzed by real-time PCR to confirm their expression level in a varity of tissues. Pancreatic tumors and normal pancreas tissues along with other normal tissues were tested in quantitative real-time PCR. Briefly, the first-strand cDNA was synthesized from 20 μg of total RNA that had been treated with DNase I (Amplification Grade, Gibco BRL Life Technology, Gaitherburg, Md.), using Superscript Reverse Transcriptase (RT) (Gibco BRL Life Technology, Gaitherburg, Md.). Real-time PCR was performed with a GeneAmp™ 5700 sequence detection system (PE Biosystems, Foster City, Calif.). The 5700 system uses SYBR™ green, a fluorescent dye that only intercalates into double stranded DNA, and a set of gene-specific forward and reverse primers. The increase in fluorescence is monitored during the whole amplification process. The optimal concentration of primers was determined using a checkerboard approach and a pool of cDNAs from pancreas tumors was used in this process. The PCR reaction was performed in 25 μl volumes that include 2.5μl of SYBR green buffer, 2 μl of cDNA template and 2.5μl each of the forward and reverse primers for the gene of interest. The cDNAs used for RT reactions were diluted 1:10 for each gene of interest and 1:100 for the β-actin control. In order to quantitate the amount of specific cDNA (and hence initial mRNA) in the sample, a standard curve is generated for each run using the plasmid DNA containing the gene of interest. Standard curves were generated using the Ct values determined in the real-time PCR which were related to the initial cDNA concentration used in the assay. Standard dilution ranging from 20-2×10⁶ copies of the gene of interest was used for this purpose. In addition, a standard curve was generated for β-actin ranging from 200fg-2000fg. This enabled standardization of the initial RNA content of a tissue sample to the amount of β-actin for comparison purposes. The mean copy number for each group of tissues tested was normalized to a constant amount of β-actin, allowing the evaluation of the over-expression levels seen with each of the genes.

Pn1467P (SEQ ID NO:4503) was found to be over-expressed in grade IV pancreas tumors and the majority of grade III tumors. Moderate expression was observed in grade II tumors, normal pancreas, and in most normal tissues. Over-expression of Pn1470P (SEQ ID NO:4535) was seen in grade III tumors and normal pancreas. Moderate expression of this gene was observed in grade IV tumors. Over-expression was also seen in bronchus. Low to moderate expression was observed in gall bladder, PBMC, stomach testis, and thymus. Pn1475P (SEQ ID NO:4540) was over-expressed in grades II-IV pancreas tumors and expressed at a low level in normal pancreas. Expression was not seen in metastatic tumors or in pancreatitis. Some Pn1475P expression was observed in bronchus, colon, esophagus, gall bladder, lung, salivary gland, small intestine, stomach, and trachea. Pn1476 (SEQ ID NO:4542) was overexpressed in 4 of 11 grade III tumors, 1 grade II, and I grade IV tumor. It was also overexpressed in 3 of 4 normal pancreas samples. No expression of this gene was observed in any normal tissues. Thus, this candidate will be valuable for both vaccine and diagnostic purposes. Pn1468P (SEQ ID NO:4507) was over-expressed in pancreas tumor metastases but not in other pancreas tumors. Over-expression was also observed in adrenal gland and pituitary, but was absent from all other normal tissues. Pn1473P (SEQ ID NO:4539) was highly over-expressed in pancreas tumor sample T795A. It was also over-expressed in 1 of 3 grade II pancreas tumor samples, 2 of 6 grade 4 tumor samples, and 7 of 11 grade III tumor samples. It was also over-expressed in tumor metastases but was not expressed in normal pancreas tissue. Pn1473P expression was also observed in skeletal muscle, stomach, testis, and trachea. The expression profiles of all of these candidate genes suggest that they will be valuable for therapeutic vaccine and/or diagnostic purposes.

### Example 10

### Full Length cDNA and Protein Sequence for 4 Antigens Overexpressed in Pacreatic Tumors

The sequences for Pn1468P, Pn1472P, and Pn1475P (SEQ ID NOs:4507, 4529, 4540, respectively), shown to be overexpressed in pancreas tumor samples, were searched against Genbank and the full length sequences identified. The full length sequences from Genbank are set forth in SEQ ID NOs:4548, 4549, and 4550, respectively. The corresponding protein sequences are set forth in SEQ ID NOs:4552-4554. The sequence for Pn1467, also overexpressed in pancreas tumor samples, was searched against the Genseq database and the full length sequence identified. The full length cDNA sequence from Genseq is set forth in SEQ ID NO:4547 and the corresponding protein sequence is set forth in SEQ ID NO:4551.

### Example 11

### Full Length cDNA and Protein Sequence for 2 Antigens Overexpressed in Pacreatic Tumors

Disclosed herein are the full-length DNA and protein sequences for the Pn1509P and Pn1510P antigens, both overexpressed in pancreatic tumors as compared to normal tissues.

A partial sequence for Pn1509P was shown to be overexpressed by 3.74 fold in pancreatic tumors as compared to normal tissues (see SEQ ID NO:4410, Table 2). The full-length DNA sequence for Pn1509P was identified by searching the 493 base pair fragment of Pn1509P set forth in SEQ ID NO:4410 against the Genbank database. The full-length extended DNA sequence for Pn1509P (identified in GenBank accession number NM 004617) is set forth in SEQ ID NO:4555 and the corresponding protein is set forth in SEQ ID NO:4558 . Based on sequence analysis, Pn1509 is predicted to be a tetraspan protein (it has four predicted membrane spanning domains) and has two potential sites for N-linked glycosylation.

A partial sequence for Pn1510P was shown to be overexpressed by 3.03 fold in pancreatic tumors as compared to normal tissues (see SEQ ID NO:4477, Table 2). The partial Pn1510P sequence set forth in SEQ ID NO:4477 was used in a search of the GeneSeq DNA database and matched 5 GeneSeq DNA records: A26456, A37144, A26424, V84525, and T22133. When the 5 protein coding regions of these DNA sequences were aligned using the DNAStar Seqman program, it was found that record A37144 had an additional C at position 35. This resulted in a 243 amino acid ORF. In the absence of this C at position 35, the DNA sequence encodes a 278 amino acid ORF. Disclosed herein are the DNA sequences that encode for both the 243 amino acid ORF and the 278 amino acid ORF (SEQ ID NOs:4556 and 4557, respectively). Also disclosed herein are the protein sequences for the 243 (Pn1510P-243) and 278 (Pn1510P-278) amino acid ORFs (SEQ ID NOs:4559 and 4560, respectively). In addition, transmembrane prediction programs were run to determine whether or not the Pn1510 protein may contain a transmembrane domain. Analysis of the Pn1510P-278 and Pn1510P-243 amino acid sequences using the PSORT and PSORTII programs revealed no potential transmembrane domains. However, analysis of these 2 protein sequences using a transmembrane prediction program identified a stretch of 20 hydrophobic amino acids, suggesting a possible transmembrane domain. This transmembrane domain occurs at amino acids 233-252 of the Pn150P-278 ORF and at amino acids 198-217 of the Pn1510P-243 ORF.

### Example 12

### Synthesis of Peptides

Polypeptides are synthesized on a Perkin Elmer/Applied Biosystems Division 430A peptide synthesizer using FMOC chemistry with HPTU (O-Benzotriazole-N,N,N′,N′-tetramethyluronium hexafluorophosphate) activation. A Gly-Cys-Gly sequence is attached to the amino terminus of the peptide to provide a method of conjugation, binding to an immobilized surface, or labeling of the peptide. Cleavage of the peptides from the solid support is carried out using the following cleavage mixture: trifluoroacetic acid:ethanedithiol:thioanisole:water:phenol (40:1:2:2:3). After cleaving for 2 hours, the peptides are precipitated in cold methyl-t-butyl-ether. The peptide pellets are then dissolved in water containing 0.1% trifluoroacetic acid (TFA) and lyophilized prior to purification by C18 reverse phase HPLC. A gradient of 0%-60% acetonitrile (containing 0.1% TFA) in water (containing 0.1% TFA) is used to elute the peptides. Following lyophilization of the pure fractions, the peptides are characterized using electrospray or other types of mass spectrometry and by amino acid analysis.

### Example 13

### Peptide Priming of T-Helper Lines

Generation of CD4⁺ T helper lines and identification of peptide epitopes derived from tumor-specific antigens that are capable of being recognized by CD4⁺ T cells in the context of HLA class II molecules, is carried out as follows:

Fifteen-mer peptides overlapping by 10 amino acids, derived from a tumor-specific antigen, are generated using standard procedures. Dendritic cells (DC) are derived from PBMC of a normal donor using GM-CSF and IL-4 by standard protocols. CD4⁺ T cells are generated from the same donor as the DC using MACS beads (Miltenyi Biotec, Auburn, Calif.) and negative selection. DC are pulsed overnight with pools of the 15-mer peptides, with each peptide at a final concentration of 0.25 μg/ml. Pulsed DC are washed and plated at 1×10⁴ cells/well of 96-well V-bottom plates and purified CD4⁺ T cells are added at 1×10⁵/well. Cultures are supplemented with 60 ng/ml IL-6 and 10 ng/ml IL-12 and incubated at 37° C. Cultures are restimulated as above on a weekly basis using DC generated and pulsed as above as antigen presenting cells, supplemented with 5 ng/ml IL-7 and 10 U/ml IL-2. Following 4 in vitro stimulation cycles, resulting CD4⁺ T cell lines (each line corresponding to one well) are tested for specific proliferation and cytokine production in response to the stimulating pools of peptide with an irrelevant pool of peptides used as a control.

### Example 14

### Generation of Tumor-Specific CTL Lines Using in vitro Whole-Gene Priming

Using in vitro whole-gene priming with tumor antigen-vaccinia infected DC (see, for example, Yee et al, The Journal of Immunology, 157(9):4079-86, 1996), human CTL lines are derived that specifically recognize autologous fibroblasts transduced with a specific tumor antigen, as determined by interferon-γ ELISPOT analysis. Specifically, dendritic cells (DC) are differentiated from monocyte cultures derived from PBMC of normal human donors by growing for five days in RPMI medium containing 10% human serum, 50 ng/ml human GM-CSF and 30 ng/ml human IL-4. Following culture, DC are infected overnight with tumor antigen-recombinant vaccinia virus at a multiplicity of infection (M.O.I) of five, and matured overnight by the addition of 3 μg/ml CD40 ligand. Virus is then inactivated by UV irradiation. CD8+ T cells are isolated using a magnetic bead system, and priming cultures are initiated using standard culture techniques. Cultures are restimulated every 7-10 days using autologous primary fibroblasts retrovirally transduced with previously identified tumor antigens. Following four stimulation cycles, CD8+ T cell lines are identified that specifically produce interferon-γ when stimulated with tumor antigen-transduced autologous fibroblasts. Using a panel of HLA-mismatched B-LCL lines transduced with a vector expressing a tumor antigen, and measuring interferon-γ production by the CTL lines in an ELISPOT assay, the HLA restriction of the CTL lines is determined.

### Example 15

### Generation and Characterization of Anti-Tumor Antigen Monoclonal Antibodies

Mouse monoclonal antibodies are raised against E. coli derived tumor antigen proteins as follows: Mice are immunized with Complete Freund's Adjuvant (CFA) containing 50 μg recombinant tumor protein, followed by a subsequent intraperitoneal boost with Incomplete Freund's Adjuvant (IFA) containing 10 μg recombinant protein. Three days prior to removal of the spleens, the mice are immunized intravenously with approximately 50 μg of soluble recombinant protein. The spleen of a mouse with a positive titer to the tumor antigen is removed, and a single-cell suspension made and used for fusion to SP2/O myeloma cells to generate B cell hybridomas. The supernatants from the hybrid clones are tested by ELISA for specificity to recombinant tumor protein, and epitope mapped using peptides that spanned the entire tumor protein sequence. The mAbs are also tested by flow cytometry for their ability to detect tumor protein on the the surface of cells stably transfected with the cDNA encoding the tumor protein.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed:

1. An isolated polynucleotide comprising a sequence selected from the group consisting of:
(a) sequences provided in SEQ ID NOs:1-66, 75-152, 174-177, 182, 184-452, and 454-4550;
(b) complements of the sequences provided in SEQ ID NOs:1-66, 75-152, 174-177, 182, 184-452, and 454-4550;
(c) sequences consisting of at least 20 contiguous residues of a sequence provided in SEQ ID NOs:1-66, 75-152, 174-177, 182, 184-452, and 454-4550;
(d) sequences that hybridize to a sequence provided in SEQ ID NOs:1-66, 75-152, 174-177, 182, 184-452, and 454-4550, under moderately stringent conditions;
(e) sequences having at least 75% identity to a sequence of SEQ ID NOs:1-66, 75-152, 174-177, 182, 184-452, and 454-4550;
(f) sequences having at least 90% identity to a sequence of SEQ ID NOs:1-66, 75-152, 174-177, 182, 184-452, and 454-4550; and
(g) degenerate variants of a sequence provided in SEQ ID NOs:1-66, 75-152, 174-177, 182, 184-452, and 454-4550.

2. An isolated polypeptide comprising an amino acid sequence selected from the group consisting of:
(a) sequences encoded by a polynucleotide of claim 1;
(b) sequences having at least 70% identity to a sequence encoded by a polynucleotide of claim 1;
(c) sequences having at least 90% identity to a sequence encoded by a polynucleotide of claim 1; and
(d) a polypeptide sequence set forth in SEQ ID NOs: SEQ ID NOs:67-74, 153-173, 178-181, 183, 453, 4551-4554, 4558-4560;
(e) sequences having at least 70% identity to a polypeptide sequence set forth in SEQ ID NOs:67-74, 153-173, 178-181, 183, 453, 4551-4554, 4558-4560; and
(f) sequences having at least 90% identity to a polypeptide sequence set forth in SEQ ID NOs:67-74, 153-173, 178-181, 183, 453, 4551-4554, 4558-4560.

3. An expression vector comprising a polynucleotide of claim 1 operably linked to an expression control sequence.

4. A host cell transformed or transfected with an expression vector according to claim 3.

5. An isolated antibody, or antigen-binding fragment thereof, that specifically binds to a polypeptide of claim 2.

6. A method for detecting the presence of a cancer in a patient, comprising the steps of:
(a) obtaining a biological sample from the patient;
(b) contacting the biological sample with a binding agent that binds to a polypeptide of claim 2;
(c) detecting in the sample an amount of polypeptide that binds to the binding agent; and
(d) comparing the amount of polypeptide to a predetermined cut-off value and therefrom determining the presence of a cancer in the patient.

7. A fusion protein comprising at least one polypeptide according to claim 2.

8. An oligonucleotide that hybridizes to a sequence recited in SEQ ID NOs:1-66, 75-152, 174-177, 182, 184-452, and 454-4550 under moderately stringent conditions.

9. A method for stimulating and/or expanding T cells specific for a tumor protein, comprising contacting T cells with at least one component selected from the group consisting of:
(a) polypeptides according to claim 2;
(b) polynucleotides according to claim 1; and
(c) antigen-presenting cells that express a polypeptide according to claim 2,
under conditions and for a time sufficient to permit the stimulation and/or expansion of T cells.

10. An isolated T cell population, comprising T cells prepared according to the method of claim 9.

11. A composition comprising a first component selected from the group consisting of physiologically acceptable carriers and immunostimulants, and a second component selected from the group consisting of:
(a) polypeptides according to claim 2;
(b) polynucleotides according to claim 1;
(c) antibodies according to claim 5;
(d) fusion proteins according to claim 7;
(e) T cell populations according to claim 10; and
(f) antigen presenting cells that express a polypeptide according to claim 2.

12. A method for stimulating an immune response in a patient, comprising administering to the patient a composition of claim 11.

13. A method for the treatment of a pancreatic cancer in a patient, comprising administering to the patient a composition of claim 11.

14. A method for determining the presence of a cancer in a patient, comprising the steps of:
(a) obtaining a biological sample from the patient;
(b) contacting the biological sample with an oligonucleotide according to claim 8;
(c) detecting in the sample an amount of a polynucleotide that hybridizes to the oligonucleotide; and
(d) comparing the amount of polynucleotide that hybridizes to the oligonucleotide to a predetermined cut-off value, and therefrom determining the presence of the cancer in the patient.

15. A diagnostic kit comprising at least one oligonucleotide according to claim 8.

16. A diagnostic kit comprising at least one antibody according to claim 5 and a detection reagent, wherein the detection reagent comprises a reporter group.

17. A method for inhibiting the development of a cancer in a patient, comprising the steps of:
(a) incubating CD4+ and/or CD8+ T cells isolated from a patient with at least one component selected from the group consisting of: (i) polypeptides according to claim 2; (ii) polynucleotides according to claim 1; and (iii) antigen presenting cells that express a polypeptide of claim 2, such that T cell proliferate;
(b) administering to the patient an effective amount of the proliferated T cells,
and thereby inhibiting the development of a cancer in the patient.
